(19) United States
(12) Patent Application Publication
(10) Pub. No.: US 2022/0118416 A1
(43) Pub. Date: Apr. 21, 2022

(54) CROSSLINKED MULTILAYERED MATERIAL COMPOSITIONS, METHODS FOR THEIR PREPARATION AND APPLICATIONS THEREOF

(71) Applicant: Pathak Holdings LLC, Phoenix, AZ (US)

(72) Inventors: Chandrashekhar P. Pathak, Phoenix, AZ (US); Milind M. Thigle, Kalyan (IN)

(21) Appl. No.: 17/503,063

(22) Filed: Oct. 15, 2021

Related U.S. Application Data
(60) Provisional application No. 63/093,271, filed on Oct. 18, 2020.
(60) Provisional application No. 63/143,884, filed on Jan. 31, 2021.

(51) Int. Cl. B01J 13/00; C08F 2/50; A61K 47/69

(52) U.S. Cl. CPC B01J 13/00; A61K 47/6903; C08F 2/50

(57) ABSTRACT

Novel composite materials and their methods of preparation comprising a crosslinked biodegradable polymer and non-crosslinked biodegradable polymers are disclosed. The composite materials can be formed into injectable compositions comprising composite microparticles or microspheres. The non-crosslinked biodegradable polymer in the composite material is synthesized or precipitated in situ inside the particle. The non-crosslinked biodegradable polymer in the composite may be liquid, low melting solid or polymer with thermosensitive or pH sensitive gelation properties. Also disclosed are injectable compositions comprising microparticles or microspheres for controlled drug delivery wherein such particles are delivered using organic solvents. Also disclosed are methods and compositions for making two or more layered particles for medical and industrial use.

### CROSS-REFERENCE TO RELATED APPLICATIONS

This application claims priority to U.S. Patent Application 63/093,271 filed on Oct. 18, 2020 and U.S. Patent Application 63/143,884 filed on Jan. 31, 2021 .

### FIELD OF THE INVENTION

The present invention generally relates to compositions and methods for biomedical devices and controlled drug delivery systems.

### BACKGROUND OF THE INVENTION

Conventional biodegradable synthetic polymer drug delivery microsphere preparation methods (C. Wischke et. al. and references therein) involve many critical steps. Many common steps involve dissolving the biodegradable polymers in the organic solvent, mixing the solution to obtain a droplet form either in emulsion or in a droplet forming apparatus; removing the solvent in a controlled manner to form precipitated polymer in a microsphere form. The polymer solution droplets tend to agglomerate or split during the solvent removal step and this can potentially lead to particle size distribution in the resultant formed microspheres. The microspheres thus formed are generally suspended in a biocompatible aqueous buffered solution and injected in the body as a suspension/emulsion. Biocompatible organic solvents such as DMSO cannot be used to disperse the microspheres formed using conventional methods because the microspheres tend to dissolve and lose their shape and cause the release of the encapsulated drug. New compositions and their methods of preparation of microspheres/microparticles are needed where the ability to agglomerate during the solvent removal stage is minimized or eliminated. It will be a valuable contribution to the art if drug encapsulated microspheres can be injected in the body using biocompatible organic solvents without dissolution in the organic solvent.

Organic solvent gels (OSG) are known in the art and have various applications in medicine, chemical reactions and other industrial applications. OSG have been explored for drug delivery and other applications (A. Vintiloiu et. al., cited herein for reference only). Prior art on organic solvent gels or organogels generally use temperature as a stimulus to form organogels from liquid solutions. However prior art generally lacks compositions and methods where organic solvent gels are made by the stimulus of light, preferably gamma ray, electron beam, UV and visible light. Prior art also lacks OSG comprising biodegradable polymers for drug delivery and other applications. In this invention, new photopolymerizable organic solvent gels compositions, preferably biodegradable compositions comprising PEG and methods of their preparation are disclosed.

Layered microparticles for various medical and non-medical applications have been widely researched. Please refer to a recent review on multilayered particles by S. Saha et. al. and references therein cited herein for reference only. Newer compositions and methods are needed to make multilayered microparticles especially using organic solvent gels or hydrogels or a combination thereof. This invention provides several methods and compositions for preparing multilayered particles. In modern minimally invasive surgical methods, local drug delivery is achieved using modern tissue imaging techniques such as x-ray imaging, MRI imaging and the like. Injectable microparticles with the ability to deliver the drug/s as well as having visualization agent/s will be highly useful in local drug delivery applications. This invention provides new compositions and methods wherein drug/s and/or visualization agent/s are stored in a single particle with separate zones or layers or compartments. The separation of visualization agent and drug layers in the same particles reduces and/or eliminates the interference of visualization agent in the controlled release profile of a drug but at the same time visualization agent can be useful in the detection and monitoring of devices in clinical practice.

Thermoreversible gels have been used for sustained drug delivery (Review by L. Klouda et. al.). Thermoreversible gels such as those made using Pluronic polymers like Pluronic F127 have generally poor mechanical properties. Gel microspheres made using Pluronic F127 gels (30 percent solution in PBS) cannot maintain the spherical shape when subjected to shear stress or subjected to routine handling during the implantation or injection procedure. In this invention, a composite of crosslinked biodegradable hydrogel microspheres and thermoreversible gels is disclosed as an injectable drug delivery system. The encapsulation of thermoreversible gels in the biodegradable hydrogel microspheres improves mechanical and other handling properties of thermoreversible gels.

Neat liquid carriers such as Vitamin E acetate, oleic acid, liquid polycaprolactone, sucrose acetate isobutyrate have been reported as carriers for controlled drug delivery. When liquid compositions are injected into the tissue for systemic or local drug delivery, there is little control over the area of the tissue contact. The variation in tissue contact area has the potential to affect the drug release rate. Liquids also do not have the ability to hold shapes such as solid microspheres. In this invention, liquid carriers are first encapsulated in the biodegradable hydrogel microparticle/microsphere wherein biodegradable hydrogels microspheres provide a scaffold/matrix which improves their handling ability and provides mechanical structural integrity which helps to maintain its shape during the injection and after the injection. Since microparticles have well defined areas, liquids carrier infused in the microparticles have a defined area for tissue contact.

Microneedle arrays for controlled drug delivery have been known in the art for many years. Newer methods and compositions are needed to make them more useful in controlled drug delivery applications. In this invention, microneedle arrays with drugs/visualization agents made using organic solvent gels have been disclosed. Also disclosed are the compositions and methods that use solid state polymerization techniques to make inventive microneedle arrays.

DEXTENZA® is an ophthalmic implant (0.4 mg dexamethasone ophthalmic insert) that is recently approved for the treatment of ocular inflammation and pain. DEXTENZA is inserted in the lower lacrimal punctum and into the canaliculus for effective management of ocular inflammation and pain. Improvements in the ophthalmic implants such as DEXTENZA wherein the drug can is released at two different rates, improved implant shapes that help to reduce implant movement, use multiple drugs in the same implant, newer methods for their manufacturing and the like are needed. This invention discloses new ophthalmic implants for various ophthalmic indications.

Gelatin and other materials capsules are currently used for oral drug delivery applications. In this invention, the gelatin and other empty capsules are used as a scaffold to cast single layer or multilayer organogel or hydrogel materials for oral drug delivery application.

The following patents are hereby incorporated herein by reference for all purposes. In case of conflict, the current specification is controlling. U.S. Pat. Nos. 5,410,016, 5,529,914, 5,874,500, 6,004,573, 6,534,591, 6,566,406, 6,887,974, 7,009,034, 7,740,877, 7,790,141, 8,067,031, 6,201,065, 9,023,379, 6,387,977, 9,789,073, 9,498,557, 6,201,065 and US Patent Applications 20140256617, 20190046479, 20050069572, 20160166504.

The present invention addresses the foregoing need for better compositions and methods for sustained delivery of drugs and other applications. Accordingly, there is a need for such compositions, methods and devices as summarized herein in some detail.

An embodiment of the invention is to provide a composition comprising a biodegradable crosslinked polymer and non-crosslinked biodegradable polymer (NBP) wherein the non-crosslinked biodegradable polymer is generated and/or precipitated in situ inside the biodegradable crosslinked polymer. The non-crosslinked biodegradable polymer may be present as a neat liquid or has thermosensitive or pH sensitive gel forming properties at body temperature (around 37° C.) or around physiological pH (around pH 7.4) respectively.

Another embodiment of the invention is to provide a composition comprising a biodegradable crosslinked polymer and non-crosslinked biodegradable polymer wherein the non-crosslinked biodegradable polymer is reinforced by the biodegradable crosslinked polymer and is not covalently linked to the crosslinked polymer.

Another embodiment of the invention is to provide an elastomeric biodegradable organic solvent gel composition comprising a biodegradable crosslinked polymer, and biocompatible water miscible organic solvent wherein the gel can stretch from 10 percent to 400 or longer percent when stretched along its length.

Another embodiment of the invention is to provide a microneedle array composition comprising a composite of the biodegradable crosslinked polymer, and non-crosslinked biodegradable polymer wherein the biodegradable polymer is a neat liquid or has thermosensitive/pH sensitive gel forming properties at body temperature (around 37° C.) or around physiological pH (around pH 7.4).

Another embodiment of the invention is to provide a composition comprising a biodegradable crosslinked polymer unibody implant or microparticle comprising two or more layers wherein one layer comprises a drug and the other layer comprises a visualization agent. The preferred crosslinked material is organic solvent gel or hydrogel and is made using either condensation polymerization or free radical polymerization.

Another embodiment of the present invention is to provide a composition comprising a biodegradable crosslinked hydrogel unibody implant or microparticle comprising two or more layers wherein one layer has a drug and the other layer has a visualization agent wherein the drug and/or visualization are encapsulated in a biodegradable polymer.

Another embodiment of the present invention is to provide a composition comprising a biodegradable crosslinked hydrogel unibody implant or microparticle comprising two or more layers wherein one layer has a drug and the other layer has a tissue targeting chemical entity that can locate and attach to the diseased tissue site.

Another embodiment of the present invention is to provide a composition comprising a biodegradable crosslinked polymer unibody implant or microparticle comprising two or more layers wherein one layer has a drug and the other layer has a sensor. The preferred sensor is chemical or biochemical.

Another embodiment of the present invention is to provide a composition comprising a biodegradable or biostable crosslinked hydrogel unibody implant or microparticle comprising two or more layers wherein one layer has a drug and the other layer has magnetic properties.

Another embodiment of the present invention is to provide a composition comprising a biodegradable crosslinked hydrogel unibody implant or microparticle comprising two or more layers wherein one layer has a fast releasing drug and the other layer has a slow releasing drug.

Another embodiment of the present invention is to provide a composition comprising a biodegradable crosslinked hydrogel unibody implant or microparticle comprising two or more layers wherein one is a hydrogel and the other layer is an organic solvent gel.

Another embodiment of the present invention is to provide a composition comprising a biodegradable crosslinked hydrogel unibody implant or microparticle comprising two or more layers wherein at least one layer has a different crosslinking density than the other layer/s.

Another embodiment of the present invention is to provide a composition comprising a biodegradable crosslinked hydrogel unibody implant or microparticle comprising two or more layers wherein at least one layer is a hydrogel that degrades by hydrolysis mechanism or by enzymatic degradation mechanism.

Another embodiment of the present invention is to provide a composition comprising a biodegradable crosslinked hydrogel unibody implant or microparticle comprising two or more layers wherein at least one layer comprises synthetic hydrogel and the preferred synthetic hydrogel comprises PEG.

Another embodiment of the present invention is to provide a composition comprising a biodegradable crosslinked hydrogel unibody implant or microparticle comprising two or more layers wherein at least one layer comprises crosslinked polymer made from natural macromolecule and preferred natural macromolecule comprises glycosaminoglycan, polysaccharide, cellulose or cellulose derivative, or protein.

Another embodiment of the present invention is to provide a composition comprising a biodegradable crosslinked based hydrogel unibody implant or microparticle comprising two or more layers wherein at least one layer comprises a synthetic hydrogel that degrades within 2 months to 2 years upon implantation in the human or animal body.

Another embodiment of the present invention is to provide a composition comprising a biodegradable crosslinked based hydrogel unibody implant or microparticle comprising two or more layers wherein at least one layer comprises a synthetic hydrogel that degrades within 1 to 60 days.

Another embodiment of the present invention is to provide a composition comprising a biodegradable crosslinked hydrogel unibody implant or microparticle comprising two or more layers wherein at least one layer comprises macromolecule/polymer with functional reactive groups. The preferred functional reactive groups are hydroxy, carboxylic acid, epoxy, isocyanate, amine, thiol or activated carboxylic acid.

Another embodiment of the present invention is to provide a composition comprising a biodegradable crosslinked hydrogel unibody implant or microparticle comprising two or more layers wherein at least one layer comprises macromolecule/polymer that is grafted with biodegradable polymer.

Another embodiment of the present invention is to provide a composition comprising a biodegradable crosslinked hydrogel unibody implant or microparticle comprising two or more layers wherein at least one of the layers have alkyl side chains with 4 to 22 carbon atoms.

Another embodiment of the present invention is to provide a composition comprising a crosslinked unibody polymer particle comprising two or more layers wherein at least one layer is magnetic and the other layer comprises an enzyme or catalyst. The enzyme or catalyst may be physically entrapped or encapsulated or covalently linked.

Another embodiment of the invention is to provide a crosslinked biodegradable composition, wherein the macromonomer has at least two free radical polymerizable groups capable of polymerization in an organic solvent and the polymerizable groups are present between the biodegradable groups; and the macromonomer is effectively polymerized and crosslinked in an organic solvent at a concentration of 2 g/100 g to 50 g/100 g.

Another embodiment of the invention is to provide a crosslinked biodegradable composition, wherein the macromonomer comprises (a) has solubility of at least 1 g/100 ml in an organic solvent, (b) has at least one biodegradable region which is hydrolyzable under in vivo conditions, and (c) has at least two free radical polymerizable groups capable of polymerization in an organic solvent and the polymerizable groups are present between the biodegradable groups; and the macromonomer is effectively polymerized and crosslinked in an organic solvent at a concentration of 2 g/100 g to 50 g/100.

Another embodiment of the invention is to provide a crosslinked biodegradable composition, wherein the macromonomer used has a solubility of at least 1 g/100 ml in an organic solvent and has at least one biodegradable region which is hydrolyzable under in vivo conditions, and has at least two free radical polymerizable groups capable of polymerization in an organic solvent and the polymerizable groups are present between the biodegradable groups; and the macromonomer is effectively polymerized and crosslinked in an organic solvent at a concentration of 2 g/100 g to 50 g/100 g in presence of biodegradable polymer that is present at 0.1 g/100 g to 60 g/100 g concentration.

Another embodiment of the invention is to provide a crosslinked biodegradable macromonomer composition with a volume of 0.05 ml to one picoliter, wherein the macromonomer used has a solubility of at least 1 g/100 ml in the organic solvent and has at least one biodegradable region which is hydrolyzable under in vivo conditions, and has at least two free radical polymerizable groups capable of effective polymerization in an organic solvent and the polymerizable groups are present between the biodegradable groups; and the macromonomer is effectively polymerized and crosslinked in an organic solvent at a concentration of 3 g/100 g to 50 g/100 g in presence of biodegradable polymer with a molecular weight between 2000 to 2 million Daltons.

Another embodiment of the invention is to provide a biodegradable crosslinked composition with a volume of 0.05 ml to one picoliter wherein said composition is formed by effective condensation polymerization of two precursors in an organic solvent in presence of non-crosslinked biodegradable polymer with a molecular weight range between 2000 to 2 million Daltons wherein at least one precursor has two or more nucleophilic groups; the second precursor has two or more electrophilic groups; a total of the nucleophilic and electrophilic group is at least five; molar concentration of electrophilic and nucleophilic groups is substantially same; at least one biodegradable bond between nucleophilic and/or electrophilic groups.

Another embodiment of the invention is to provide a biodegradable crosslinked composition with a volume of 0.05 ml to one picoliter and the biodegradable crosslinked composition is grafted with a biodegradable polymer. The preferred crosslinked composition is made by synthetic precursors or macromonomers.

Another embodiment of the invention is to provide a microparticle based injectable pharmaceutical composition comprising a biodegradable crosslinked polymer and non-crosslinked biodegradable polymer that is physically entrapped in the crosslinked polymer. The non-crosslinked biodegradable polymer is thermosensitive, pH sensitive or neat liquid.

Another embodiment of the invention is to provide an injectable composition comprising: a fluid carrier; a plurality of crosslinked polymeric microparticles comprising non-crosslinked biodegradable polymer wherein the non-crosslinked biodegradable polymer is a neat liquid or thermosensitive/pH sensitive gel.

Another embodiment of the invention is to provide a composition comprising a biodegradable crosslinked polymer and non-crosslinked biodegradable polymer wherein the non-crosslinked biodegradable polymer is not covalently bonded to the crosslinked polymer and the said non-crosslinked polymer is generated/precipitated in situ inside the biodegradable crosslinked polymer. The composition further comprises a drug or visualization agent.

Another embodiment of the invention is to provide a composition comprising a biodegradable crosslinked polymer and non-crosslinked biodegradable polymer wherein the non-crosslinked biodegradable polymer is reinforced by biodegradable crosslinked polymer and the said non-crosslinked biodegradable polymer may be a neat liquid or has thermosensitive/pH sensitive gel forming properties at 37° C. temperature or around physiological pH (around pH 7.4) respectively.

Another embodiment of the invention is to provide a unibody wound covering composition comprising two or more layers wherein one of the layers comprises polymers/macromolecules or its derivatives selected from the group consisting of: polyethylene oxide, cellulose, hydroxymethyl cellulose, hydroxyethylpropyl cellulose, hyaluronic acid, aloe vera, polydimethylsiloxane, gelatin, collagen, fibrinogen, decellularized tissue.

Another object of the invention is to provide a unibody implant composition comprising two or more layers wherein one of the layers has microencapsulated drug/s. Preferred implant is an ophthalmic implant. Even more preferred implant is a punctal plug.

Another embodiment of the invention is to provide a unibody implant composition comprising two or more layers wherein one of the layers has only drug and another layer has only visualization agent.

Another embodiment of the invention is to provide a unibody oral tablet for drug delivery comprising crosslinked polymer. Preferably the composition comprises PEG. Even more preferably, the composition comprises two or more layers.

Another embodiment of the invention is to provide a sealed oral gelatin or HPMA capsule for oral drug delivery comprising a drug, a liquid or solid carrier and a crosslinked polymer that is enclosed in the capsule. Preferably the crosslinked polymer comprises PEG and liquid carrier is editable oil, canola oil, glycerol, PEG with molecular weight 400 to 1000. The solid carrier is sugar or inorganic salt such as sodium chloride.

In yet another embodiment of the present invention, a method for making sustained drug delivery composition is provided wherein the method comprises the steps: (a) providing the precursors of the biodegradable crosslinked polymer, organic solvent soluble biodegradable polymer, and biocompatible organic solvent; (b)dissolving the provided compositions in an organic solvent at a concentration of 2 to 60 percent to obtain a homogeneous solution;(c) initiating effective polymerization and crosslinking of precursors and forming an organic solvent gel;(d) removing the organic solvent and precipitating the biodegradable polymer in the crosslinked network. The composition further comprises a drug or visualization agent.

In yet another embodiment of the present invention, a method for making microparticles for sustained drug delivery is provided wherein the method comprises: mixing precursor/s of the biodegradable crosslinked polymer, a non-crosslinked synthetic biodegradable polymer in the organic solvent or water or aqueous solution at a concentration of 5 to 60 percent to obtain a homogenous solution; providing a desired shape to the solution; initiating effective polymerization and crosslinking of precursor/s and forming crosslinked composite particle of the desired shape; washing the crosslinked microparticle with an organic solvent or aqueous solvent to remove impurities; removing the solvent and precipitating the non-crosslinked polymer in the microparticle. The composition further comprises a drug or visualization agent.

In yet another embodiment of the present invention, a method for making microparticles for sustained drug delivery is provided wherein the method comprises: dissolving precursor/s of the biodegradable crosslinked polymer, a non-crosslinked biodegradable polymer in the organic solvent or water or aqueous solution to form a homogenous 5 to 60 percent solution; forming 0.2 microns to 2000 microns diameter spherical droplets of the solution; initiating effective polymerization and crosslinking of precursor/s in the droplets and forming crosslinked composite microspheres; washing the crosslinked microspheres with organic solvent or water to remove impurities; removing the solvent to precipitate the non-crosslinked polymer in microspheres.

In yet another embodiment of the present invention, a method for making sustained drug delivery biodegradable crosslinked microparticle composition is provided wherein the method comprises: providing crosslinked biodegradable particles; incubating the particles in an organic solvent or aqueous solutions comprising thermosensitive polymer, pH sensitive polymer or neat liquid carrier for sufficient time to infuse about 1 to 60 percent (relative to total weight of the microparticle) thermosensitive polymer, pH sensitive polymer or neat liquid carrier in the microparticle. Removing the solvent and leaving behind the thermosensitive polymer, pH sensitive polymer or neat liquid carrier in the microparticles.

In yet another embodiment of the present invention, a method for making sustained drug delivery biodegradable crosslinked composition is provided wherein the method comprises: dissolving free radically polymerizable macromonomer and photoinitiator in water and organic solvent respectively to form a 2 to 60 g/100 g macromonomer solution and 0.05 to 5 g/100 g photoinitiator solution respectively; freezing the solution at 15 to -269° C. to form a frozen solid; exposing the frozen solid to long UV or visible light to initiate effective polymerization and crosslinking in the frozen state.

In yet another embodiment of the present invention, a method for making sustained drug delivery biodegradable crosslinked composition is provided wherein the method comprises: dissolving a macromonomer and photoinitiator in water and/or in the organic solvent to form a homogeneous solution wherein the final macromonomer concentration is 2 to 60 g/100 ml and final photoinitiator concentration is 0.05 to 3 g/100 ml; removing 5 to 99.999 percent of the solvent and exposing the solution to long UV or visible light to initiate effective polymerization and crosslinking of the solution.

In another embodiment of the present invention, a method for making crosslinked biodegradable polymer comprising the steps of a) preparing an aqueous or organic solvent solution comprising biodegradable macromonomer and a photoinitiator, said biodegradable macromonomer comprising a water soluble polymer having at least 2 polymerizable groups separated by at least one biodegradable group; b) forming small geometric shapes of the mix in (a); c) freezing the geometric shapes to form frozen solid shapes and d) effectively polymerizing the macromonomer in the frozen state by exposing the geometric shapes to light or electromagnetic radiation.

In another embodiment of the present invention, a method for encapsulation of biological material comprising the steps of a) mixing the biological material in an aqueous solution comprising macromonomer and a photoinitiator, said macromonomer comprising at least two polymerizable groups; b) forming small geometric shapes of the mix obtained in step (a); c) freezing the geometric shapes to form frozen solid shapes and d) effectively polymerizing the macromonomer in the frozen state by exposing the geometric shapes to light or electromagnetic radiation. The preferred aqueous solution is buffered at a pH range of 6 to 9, preferably around pH 7.4.

In another embodiment of the present invention, a method for preparation of controlled drug delivery composition comprising the steps of a) mixing the drug in an aqueous or organic solvent macromonomer solution comprising macromonomer and a photoinitiator, the said macromonomer water soluble polymer having at least two polymerizable groups; b) forming small geometric shapes of the mix in (a); c) freezing the geometric shapes at 15° C. or lower temperature to form frozen solid shapes and d) effectively polymerizing the macromonomer in the frozen state by exposing the geometric shapes to light radiation.

In another embodiment of the present invention, a method for preparation of two or more layered unibody particle comprising the steps of a) providing a first aqueous macromonomer solution suspended with drug or visualization agent encapsulated particles, the said macromonomer comprising a water soluble polymer having at least two polymerizable groups; b) providing a second aqueous macromonomer solution suspended with drug or visualization agent encapsulated particles, the said macromonomer comprising a water soluble polymer having at least two polymerizable groups; c) dispensing the solutions in a and b sequentially in a mold cavity without substantially mixing; d) effectively polymerizing the macromonomer solutions by exposing the solution to light radiation; e) removing the crosslinked polymer from the mold or capillary.

In another embodiment of the present invention, a method for preparation of multilayered unibody particle comprising the steps of a) preparing a first aqueous or organic solvent solution comprising macromonomer and a photoinitiator, said macromonomer comprising a water soluble polymer having at least two polymerizable groups; b) preparing a second aqueous or organic solvent solution comprising macromonomer and photoinitiator, said macromonomer comprising a water soluble polymer having at least two polymerizable groups; c) dispensing first and the second solution sequentially in a mold cavity without substantially mixing wherein the total volume of the dispensed solution is 0.05 ml to one picoliter; d) effectively polymerizing both the solutions exposing them to light; e) removing the crosslinked polymer from the mold or capillary as a unibody crosslinked material.

In another embodiment of the present invention, a method for drug encapsulation in a biodegradable crosslinked polymer comprising the steps of a) mixing the drug in an aqueous or organic solvent macromonomer solution comprising macromonomer and a photoinitiator, the said biodegradable macromonomer having at least two polymerizable groups; b) filling the solution in a mold cavity of desired shape and size; c) freezing the solution at 15 to −269° C. to form frozen solid shapes and d) effectively polymerizing the macromonomer in the frozen state by exposing the geometric shapes to light radiation.

In another embodiment of the present invention, a method for drug encapsulation in a biodegradable crosslinked polymer comprising the steps of a) providing aqueous or organic solvent macromonomer solution comprising drug or visualization agent encapsulated particles, the said macromonomer comprising a water soluble polymer having at least two polymerizable groups; b) coating the macromonomer solution on a flat inert surface forming a uniform coating and removing the solvent (a); c) exposing the coating through a photomask to light to selectively and effectively polymerize and crosslinking the coating; d) removing the unpolymerized coating from the substrate using aqueous or organic solution; e) removing the crosslinked polymer from the inert surface.

The foregoing discussion summarizes some of the more pertinent embodiments of the present invention. These embodiments should be construed to be merely illustrative of some of the more prominent features and applications of the invention. Applying or modifying the disclosed invention in a different manner can attain many other beneficial results and are considered as part of this invention or modifying the invention,

as will be described. Accordingly, referring to the following drawings, in conjunction with the ensuing description, may provide a complete understanding of the invention.

### BRIEF DESCRIPTION OF THE SEVERAL VIEWS OF THE DRAWINGS

The above-mentioned and other features and advantages of this present disclosure, and the manner of attaining them, will become more apparent and the present disclosure will be better understood by reference to the following description of embodiments of the present disclosure taken in conjunction with the accompanying drawings, wherein:

FIG. 1 shows a partial schematic representation of a method for making drug and/or visualization agent loaded composite biodegradable materials or microparticles.

FIG. 2 shows schematic structures of preferred organic solvent gel/hydrogel precursor compositions described in this invention.

FIG. 3 shows a partial schematic representation of illustrative chemical reactions involved in making composite biodegradable materials/microparticles using free radical polymerizable precursors.

FIG. 4 shows a partial schematic representation of illustrative chemical reactions involved in making composite biodegradable polymers using condensation polymerization reaction.

FIG. 5 shows a partial schematic representation of a method for creating composite microparticles of various shapes.

FIG. 6 shows photographs of the composite microspheres, elastomeric organogels and microneedle array prepared using methods described in this invention.

FIG. 7 shows a partial schematic representation of composite and/or multilayered materials described in this invention.

FIG. 8 shows a partial schematic representation of methods to make drug loaded microparticles or microspheres.

FIG. 9 shows a controlled release profile of rifampin from composite microparticles comprising PEG-based crosslinked polymer with in situ precipitated PLGA polymer.

FIG. 10 shows the controlled release profile of rifampin from composite microparticles with a liquid carrier or thermosensitive polymer as a carrier.

FIG. 11 shows a partial schematic representation of methods for making multilayered microparticles/implants using two or more molds.

FIG. 12 shows a partial schematic representation of a method for making drug or live cell encapsulated multilayered microparticles using glass capillaries and liquid precursor compositions. It also shows a method for making multilayered compositions prepared using frozen or solid state precursor compositions.

FIG. 13 shows a partial schematic representation of a method to make unibody materials with diverse compositions in the desired 2 or 3-dimensional patterns.

FIG. 14 shows illustrative photographs of the multilayered unibody materials prepared using methods and compositions described in this invention.

FIG. 15 shows a partial schematic representation of a method for making drug or live cell encapsulated microparticles using frozen precursor compositions.

FIG. 16 shows a partial schematic representation of composite materials made using mechanically and covalently bonded composite materials. It also shows preferred ophthalmic or punctal implant configurations. It also shows the use of inventive multilayered materials in clinical diagnostics.

FIG. 17 shows a schematic representation of a method for making biostable or biodegradable multilayered microparticles.

FIG. 18 shows a schematic representation of methods for making multilayered oral drug delivery compositions/capsules/tablets.

### DETAILED DESCRIPTION OF THE INVENTION

It is to be understood that the present disclosure is not limited in its application to the details of construction and the arrangement of components set forth in the following description or illustrated in the drawings. The present disclosure is capable of other embodiments and of being practiced or of being carried out in various ways. Also, it is to be understood that the phraseology and terminology used herein is for the purpose of description and should not be regarded as limiting. Exemplary embodiments of the present invention are directed towards compositions, methods and devices for facilitating local and sustained drug delivery, other medical and non-medical applications.

It is advantageous to define several terms, phrases and acronyms before describing the invention in detail. It should be appreciated that the following terms are used throughout this application. Where the definition of terms departs from the commonly used meaning of the term, the applicant intends to utilize the definitions provided below, unless specifically indicated. The following definitions are provided to illustrate the terminology used in the present invention. Unless defined otherwise, all technical and scientific terms used herein have the same meaning as is commonly understood by one who is skilled in the art. All scientific literature and patent citations in this invention are incorporated herein for reference use only.

“Crosslinked material” is meant to denote the formation of intermolecular or intramolecular covalent bonds in the precursor or macromolecule or polymer. The crosslinked material may be lightly or highly swollen in organic solvents or aqueous solutions without dissolution.

A “crosslinking agent” is defined as a compound capable of forming crosslinkages. For example, glutaraldehyde is generally known in the art as a crosslinking agent for albumin or collagen.

“In situ” is meant to denote a local site, especially within or in contact with living organisms, tissue, skin, organs, or the body. In some instances, the term in situ is also used to mark a local site within the microparticle or microimplant material body.

“Bioactive” refers to one or all of the activities of a compound that show pharmacological or biological activity in living systems such as the human or animal body. Such biological activity is preferred to have a therapeutic effect. Substances or compounds that are bioactive are referred to as “drugs” or “bioactive compounds.” The bioactive compounds that can be used include, but are not limited to; antiinfectives such as, by way of example, antibiotics; antifungal agents, antiviral agents, antibacterial agents, antipruritics; anticancer agents, antipsychotics; cholesterol- or lipid-reducing agents; cell cycle inhibitors; antiparkinsonism drugs; HMG-CoA inhibitors; antirestenosis agents; antiinflammatory agents; antiasthmatic agents; anthelmintic; immunosuppressives; muscle relaxants; antidiuretic agents; vasodilators; nitric oxide; nitric oxide-releasing compounds; beta-blockers; hormones; antidepressants; decongestants; calcium channel blockers; growth factors such as, by way of example, and not limitation, bone growth factors or bone morphogenic proteins; wound healing agents; analgesics and analgesic combinations; local anesthetic agents; antihistamines; sedatives; angiogenesis-promoting agents; angiogenesis-inhibiting agents; tranquilizers and the like; cellular elements, which can be used for therapeutic use, include, but are not limited to mammalian cells including stem cells; cellular components or fragments, enzymes, DNA, RNA, and genes may also be included as bioactive components or drugs. An extensive list of bioactive compounds or drugs that may be used can be found in U.S. Pat. No. 8,067,031 cited herein for reference only.

The terms “Biodegradable”, “Bioerodible” and “Bioabsorbable” have the same meaning unless specified. The terms are meant to denote a material or substance, that will degrade in a biological environment such as the human body by either a biologically assisted mechanism, such as an enzyme catalyzed reaction or by a chemical mechanism which can occur in a biological medium, such as hydrolysis or by a dissolution mechanism in which the substance dissolves and is removed safely without any degradation.

“Biostable” is meant to denote the high chemical stability of a compound in an aqueous environment, which is similar to the environment found in the human body such as phosphate buffered saline (pH 7.4).

The term “biodegradable polymers” may include polymers or macromolecules which degrade/dissolve safely in the biological environment such as the human body. The term applies to polymers that are hydrophobic or hydrophilic. The term is applicable to polymers that are crosslinked or not-crosslinked. The crosslinking may be done via condensation polymerization or via free radical polymerization or via ionic bonding. The biodegradable polymers may be random or block or graft copolymers. The biodegradable polymers may be linear, graft, dendrimer or branched. The hydrophobic biodegradable polymers include, but are not limited to, polymers, dendrimers, copolymers or oligomers of glycolide, dI-lactide, d-lactide, I-lactide, caprolactone, dioxanone and trimethylene carbonate; degradable polyurethanes; degradable polyurethanes made by block copolymers of degradable polylactone such as polycaprolactone and polycarbonate such as poly(hexamethylene carbonate); tyrosine-derived polycarbonates, tyrosine-derived polyacrylates, polyamides; polyesters; polypeptides; polyhydroxyacids; polylactic acid; polyglycolic acid; polyanhydrides; and polylactones. Biodegradable polymers also include polyhydroxyalkanoates which are polyesters produced by microorganisms including and not limited to poly(3-hydroxybutyrate), 3-hydroxyvalerate, 4-hydroxybutyrate, 3-hydroxyhexanoate, 3-hydroxyoctanoate. The term applies to hydrophilic polymers, which include, but are not limited to, polyethylene glycol-polyhydroxy acid or polyethylene glycol-polylactide copolymers (PEG-PL copolymers); polyvinyl alcohol-co-polylactone copolymers; and derivatives of cellulose; collagen or modified collagen derivatives; gelatin; albumin or crosslinked albumin; fibrinogen; keratin; starch; hyaluronic acid and dextran.

The term “biostable polymers” include but are not limited to aliphatic and aromatic polyurethanes; polycarbonate polyurethane; polyether polyurethane; silicone polyurethane block copolymers; silicone rubbers; polydimethylsiloxane copolymers; polytetrafluoroethylene and other fluorinated polymers; expanded polytetrafluoroethylene; polyethylene; polyesters, polyethylene terephthalate, polyimides, polypropylene; polyamide; polyamide block copolymers and the like.

The term “non-crosslinked polymer/s” refers to any polymers or macromolecules that are not crosslinked and do not have the capability to form a covalent bond with precursor components under effective polymerization and crosslinking conditions. Generally, non-crosslinked polymers are linear, branched or dendrimer like organic solvent or water soluble polymers. PLGA is a typical example of a “non-crosslinked polymer” linear polymer that is soluble in many organic solvents and is biodegradable. Preferred non-crosslinked polymers are biodegradable and end capped.

“Sustained release” or “controlled drug delivery” or “long term release” are phrases used interchangeably herein, to mean longer than the expected delivery of a bioactive compound from the inventive composition.

Typically, delivery will be at least for one hour or more, two to six hours or more, and may extend to one day, a few days, weeks, months to a few years.

A “hydrogel” as used herein, refers to a semisolid composition constituting a substantial amount of water, and in which polymers, macromolecules or non-polymeric materials or mixtures thereof are dissolved or dispersed. The polymers may be physically or chemically crosslinked or not crosslinked.

An “organic solvent gel” or “Organic Solvent Gel (OSG)” refers to a semisolid composition constituting a substantial amount of organic solvent, and in which polymers, macromolecules or non-polymeric materials or mixtures thereof are dissolved or dispersed. The gel compositions may be physically or chemically crosslinked or not crosslinked.

Polyethylene glycol (PEG) or polyethylene oxide (PEO) refers to the polymer made by polymerization of ethylene oxide.

Polypropylene glycol (PPG) or polypropylene oxide (PPO) refers to the polymer made by the polymerization of propylene oxide.

Polymeric nomenclature used in this patent application such as poly (ethylene glycol) or polyethylene glycol or polyethyleneglycol refers to the same polymer unless otherwise stated clearly. This is also true for all other polymers referred in this patent application.

The term “micron” means a length of 1/1000000 of a meter; one μl means 1/1000000 of a liter, 1 nanoliter means 1/1000000000 of a liter, and one picoliter means 1/1000000000000 of a liter.

The term “macromonomer” or “macromer” refers to oligomeric or polymeric materials capable of undergoing free radical polymerization or addition polymerization.

The term “hydrophobic” is defined as a property of materials or polymers or macromolecules having a low degree of water absorption or attraction.

The terms “coloring compositions” include any coloring composition or chemical that is suitable for human or animal implantation and are preferably approved by FDA for use in implantable medical devices or in pharmaceutical preparations, especially injectable pharmaceutical preparations. The compounds include but are not limited to: Methylene blue; Indocyanine green, Eosin Y; Ethyl Eosin, Fluorescein sodium; Chromium-cobalt-aluminum oxide; Ferric ammonium citrate; Pyrogallol; Logwood extract; 1,4-Bis[(2-hydroxy-ethyl)amino]-9,10-anthracenedione bis(2-propenoic)ester copolymers(3; 1,4-Bis [(2-methylphenyl)amino] -9,10-anthracenedione; 1,4-Bis[4-(2-methacryloxyethyl) phenylamino] anthraquinone copolymers; Carbazole violet; Chlorophyllin-copper complex, oil soluble; Chromium-cobalt-aluminum oxide; Chromium oxide greens; C.I. Vat Orange 1; 2-[[2,5-Diethoxy-4-[(4-methylphenyl)thiol] phenyl]azo] -1,3,5-benzenetriol; 16,23-Dihydrodinaphtho [2,3-a:2′,3′-i] naphth [2′,3′:6,7] indolo [2,3-c] carbazole-5,10,15,17,22,24-hexone; N,N′-(9,10-Dihydro-9,10-dioxo-1,5-anthracenediyl) bis benzamide; 7,16-Dichloro-6,15-dihydro-5,9,14,18-anthrazinetetrone; 16,17-Dimethoxydinaphtho (1,2,3-cd:3′,2′,1′-lm) perylene-5,10-dione; Poly(hydroxyethyl methacrylate) -dye copolymers: one or more of Reactive Black 5; Reactive Blue 21; Reactive Orange 78; Reactive Yellow 15; Reactive Blue No. 19; Reactive Blue No. 4; C.I. Reactive Red 11; C.I. Reactive Yellow 86; C.I. Reactive Blue 163; C.I. Reactive Red 180; 4-[(2,4-dimethylphenyl)azo]-2,4-dihydro-5-methyl-2-phenyl-3H-pyrazol-3-one; 6-Ethoxy-2-(6-ethoxy-3-oxobenzo[b] thien-2(3H)-ylidene) benzo[b]thiophen-3(2H)-one; Phthalocyanine green; Iron oxides; Titanium dioxide; Vinyl alcohol/methyl methacrylate-dye reaction products; one or more of: (1) C.I. Reactive Red 180; C.I. Reactive Black 5; C.I. Reactive Orange 78; C.I. Reactive Yellow 15; C.I. Reactive Blue No. 19; C.I. Reactive Blue 21; Mica-based pearlescent pigments; Disodium 1-amino-4-[[4-[(2-bromo-1-oxoallyl)amino]-2-sulphonatophenyl]amino]-9,10-dihydro-9,10-dioxoanthracene-2-sulphonate (Reactive Blue 69); D&C Blue No. 9; D&C Green No. 5; [Phthalocyaninato(2-)] copper; FD&C Blue No. 2; D&C Blue No. 6; D&C Green No. 6; D&C Red No. 17; D&C Violet No. 2; D&C Yellow No. 10; and the like. Preferred colored compositions are biodegradable.

The term “minimally invasive surgery” or (MIS) used herein includes, but is not limited to, surgical techniques such as, by way of example, and not limitation, laparoscopy, thoracoscopy, arthroscopy, intraluminal endoscopy, endovascular techniques, catheter-based cardiac techniques (such as, by way of example, and not limitation, balloon angioplasty), and interventional radiology.

The terms “molecular mass” “molecular weight” and “molar mass” has often been used interchangeably and is generally referred to as mass of a given molecule measured in Daltons (Da) or atomic mass units. The molecular weight is also expressed as g/mole in some instances and is same as Daltons.

“Polylactic acid” or “poly(lactic acid)” or “poly(lactide)” or PLA is a term used for a polymer which is made from lactide or lactic acid. Similarly, PGA is a term used for polyglycolic acid or polyglycolate. Some synthetic biodegradable polyester polymers are generally referred to as polylactones or polyhydroxyacids. The terms “PLGA” and “PDLG” refer to the same polymer and is a copolymer of PLA and PGA.

The term “exposing” refers to soaking the desired material in a fluid comprising the treatment agent for a period sufficient to treat the desired material. The soaking may be performed by, but is not limited to, incubation, swirling, immersion, mixing, or vortexing.

The term “Precursor/s” refers to chemical/s or materials that are transformed into another compound via a chemical reaction. It is generally referred to a compound that is converted into a physically or chemically crosslinked hydrogel or organic solvent gel via physical interactions or chemical reactions. More specifically, the term generally refers to monomer/s or macromolecules that are transferred into a molecular crosslinked network via free radical/addition polymerization reaction or condensation polymerization reaction. The term also applies to macromolecules such as gelatin or albumin that are crosslinked using a crosslinker such as glutaraldehyde to form a crosslinked polymer or hydrogel.

The term “polymerizable” denotes the characteristic of molecules that have the capacity to form additional covalent bonds resulting in interlinking or covalent bonding of monomers and/or polymers to form oligomer or polymer. For example, molecules that contain acrylate type bonds form polymer using a free radical polymerization mechanism under effective reaction conditions. Molecules that have functional groups capable of reacting with each other leading to oligomer or polymer formation using condensation polymerization under effective reaction conditions.

“Effective Polymerization” refers to the chemical reaction of polymerization that leads to successful polymerization of polymerizable monomers to form polymer, oligomer or crosslinked networks. Certain physical and chemical conditions must be provided for effective polymerization of monomers.

The term “water soluble” generally refers to the solubility of a compound in water wherein the compound has a solubility of greater than 1 g/100 g, preferably greater than 5 g/100 g in water or buffered water solutions.

The term “water insoluble” generally refers to the solubility of a compound in water wherein the compound has a solubility of less than 5 g/100 g, preferably less than 1 g/100 g in water or buffered water solutions.

The term “organic soluble” or “organic solvent soluble” generally refers to the solubility of a compound in an organic solvent wherein the compound has a solubility of greater than 1 g/100 g, preferably greater than 5 g/100 g in an organic solvent.

The terms “imaging agent(s)” or “visualization agent(s)” include any medical imaging agent that helps to visualize the human body/tissue using the naked human eye or using machine assisted viewing. The term generally applies to but not limited to: coloring compositions that introduce color to medical devices and drug delivery compositions, radio-opaque contrast agents that help to visualize organs/tissues using x-ray imaging techniques, NMR contrast agents that assist in MRI imaging techniques, an ultrasonic contrast agent that improve imaging using ultrasound techniques and the like.

The term “Porosity” is defined as the presence of pores, voids, cavities, grooves, pockets and indentations within a material. The phrases “creation of artificial cavities” and “creation of artificial porosities” have been used synonymously in this application and mean the same.

A micromanipulator is a device which is used to physically manipulate a sample under a microscope. For additional information please refer to: https://en.wikipedia.org/wiki/Micromanipulator

The present invention is now described with reference to the drawings.

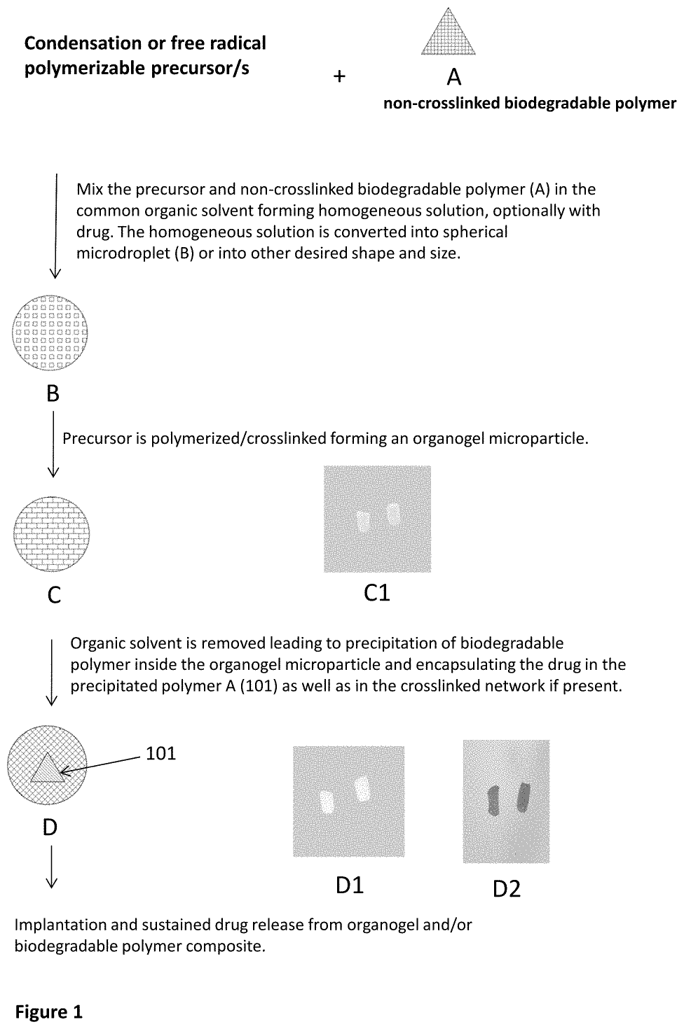

FIG. 1 shows a partial schematic representation of a method for making drug and/or visualization agent loaded composite biodegradable materials or microparticles. A biodegradable photocrosslinkable precursor/s or condensation polymerization precursors, non-crosslinked biodegradable polymer such as PLA or PLGA (A) are dissolved in a common organic solvent to make a homogeneous solution. The solution may also contain a drug/visualization agent at the desired concentration provided it can tolerate the precursor gelling/crosslinking conditions. The solution is converted into microdroplets or filled into a mold with microcavities of the desired shape and size (B). Effective crosslinking of precursor solution in the shape such as microdroplets (B) is then initiated using UV or visible light or other precursor crosslinking reactions; converting the solution droplets into organic solvent gel particles or crosslinked material swelled in an organic solvent wherein biodegradable polymer (A) and/or drug remains entrapped inside the crosslinked gel microparticle (C). C1 shows a picture of cylindrical organogel particles comprising crosslinked PEG based macromer, PLGA as non-crosslinked polymer that is swollen in dimethyl sulfoxide (DMSO) as a solvent. C1 is made according to the methods described in this invention. The composite gel microparticles may be optionally exposed to drug solution under the effective diffusion conditions wherein the drug can diffuse inside the crosslinked microparticles and biodegradable polymer. This step may be useful if the drug cannot tolerate precursor crosslinking conditions such as exposure to high intensity light. Diffusion conditions are chosen such that a substantial amount of the biodegradable polymer does not diffuse out of the crosslinked network during the drug loading process. The organic solvent is removed and non-crosslinked biodegradable polymer is solidified/precipitated in situ inside the crosslinked polymer structure entrapping the drug in the precipitated polymer and crosslinked microparticle. The microparticle with a precipitated biodegradable polymer (101) inside the crosslinked gel is schematically shown in D. D1 is similar to C1 wherein the solvent is partially removed which precipitates the PLGA inside the C1 (101). The precipitation leads to increased opacity of the particle D1 as compared to C1. D2 is the same as D1 wherein drug rifampin is infused into particles (in a crosslinked network as well as biodegradable polymer PLGA) via the solvent diffusion process described in this invention. The red color indicates the presence of rifampin in the particle. The entrapped drug is released in a sustained manner from the solidified PLGA polymer and crosslinked polymer upon implantation in the body.

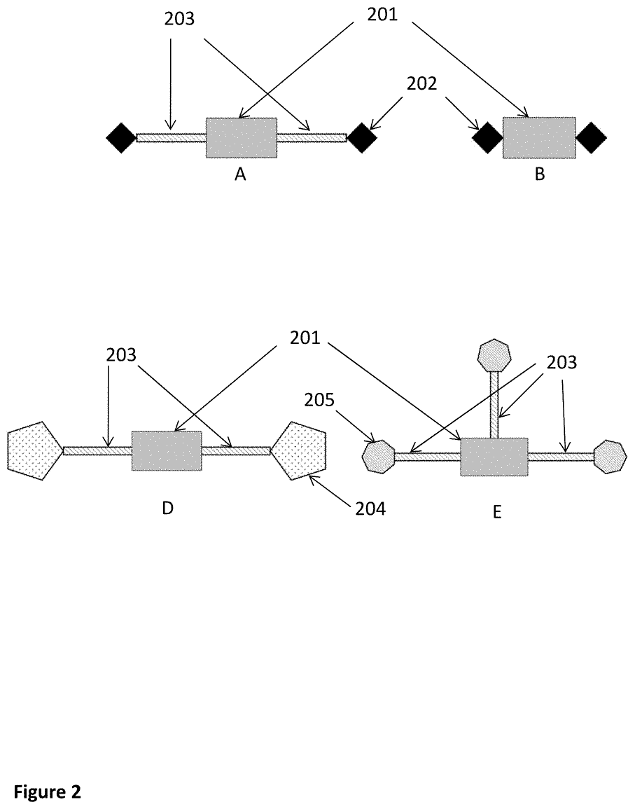

FIG. 2 shows schematic structures of preferred organic solvent gel/hydrogel precursor compositions described in this invention. FIG. A shows a free radically crosslinkable precursor or macromonomer wherein the precursor has a central core (201) and unsaturated polymerizable groups at the end or at the side chain (202) of the central core. The polymerizable groups (202) are separated from the central core (201) via a biodegradable polymer block (203). FIG. B shows a macromonomer with 201 and 202 and no biodegradable blocks. This macromonomer (B) produces biostable crosslinked networks upon effective polymerization and crosslinking. Precursors shown in D and E form crosslinked networks when reacted and polymerized via condensation or step growth polymerization under effective polymerization conditions. D has a central core (201) and biodegradable blocks (203) but has 2 or more end or side chain electrophilic groups (204) between central core (201) and biodegradable blocks (204). Precursor in FIG. E also has a central core (201), biodegradable blocks (203) and nucleophilic end groups (205) or side chain groups. The number of electrophilic groups in D and nucleophilic groups in E must be greater than or equal to two. The total number of nucleophilic and electrophilic groups (204 plus 205) must be greater than or equal to five to form an effective crosslinked network when reacted under effective crosslinking conditions. Either D or E must have biodegradable blocks (203) to form a biodegradable crosslinked polymer network.

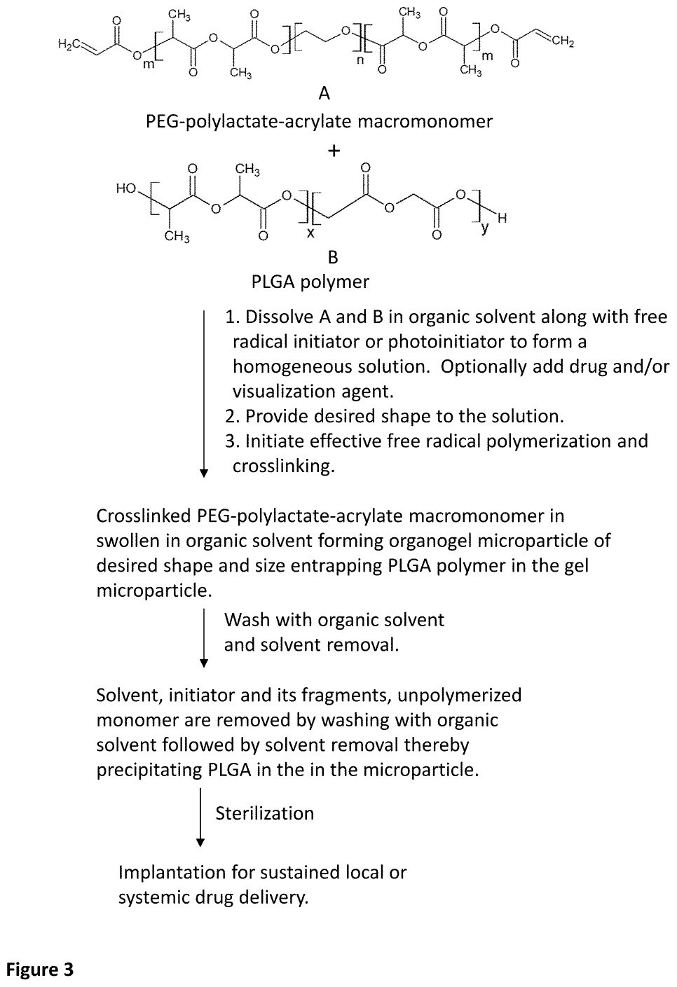

FIG. 3 shows a partial schematic representation of illustrative chemical reactions involved in making composite biodegradable materials/microparticles using free radical polymerizable precursors. PEG based biodegradable macromonomers such as PEG-polylactate-acrylate macromonomer (A) and PLGA copolymer (B, illustrative NBP), thermal or photoinitiator, and optionally drugs are mixed in an organic solvent such as THF or DMSO to form a homogeneous solution. The mixture is poured into mold cavities to provide a desired shape or sprayed to form microdroplets and then irradiated with light or heated to induce effective free radical polymerization and crosslinking of the macromonomer. The effective polymerization and crosslinking of the macromonomer converts the solution into organic solvent gel particles wherein biodegradable polymer PLGA remains entrapped in the organic solvent gel comprising crosslinked PEG-polylactate acrylate macromonomer and solvent. The gel particles are removed from the mold. The particles may be washed with an organic solvent to remove initiator fragments, unreacted macromonomer and other undesirable reaction products and impurities. The solvent is removed via solvent exchange or evaporation and the PLGA in the particle is precipitated “in situ” inside the microparticle. The precipitated particle encapsulates the drug which is then released via diffusion and/or erosion of biodegradable polymer and crosslinked gel. In some embodiments, if a biocompatible water miscible organic solvent and initiator is used, then the solvent swollen organic solvent gel microparticles can be directly injected into the tissue. The solvent is dispersed in the tissue, precipitating the PLGA in the composite microparticles. The precipitated PLGA encapsulates the drug and provides sustained drug delivery in the tissue.

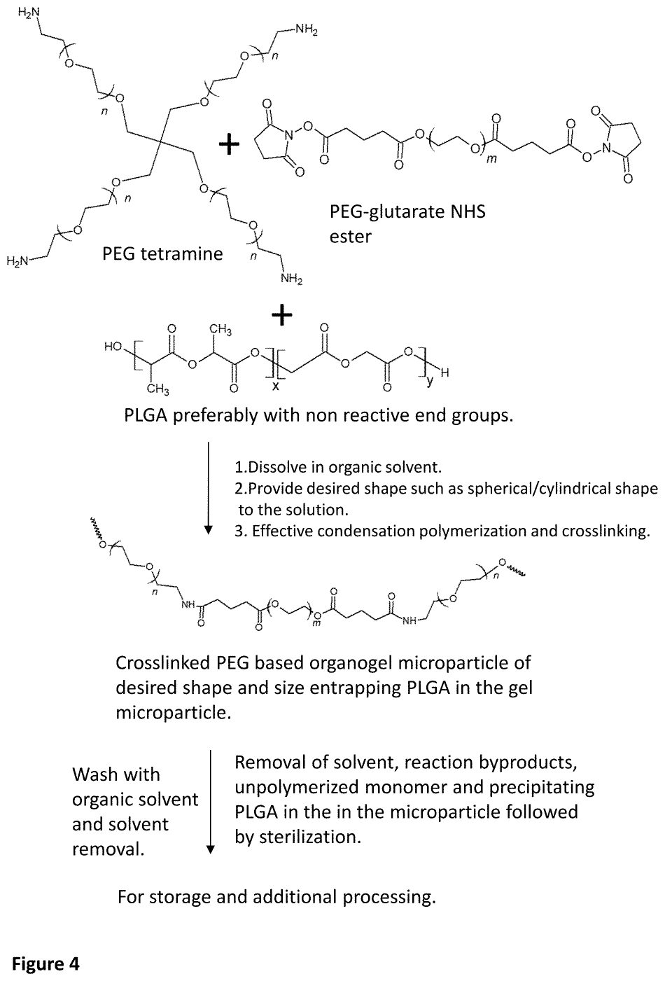

FIG. 4 shows a partial schematic representation of illustrative chemical reactions involved in making composite biodegradable polymers using condensation polymerization reaction. PEG based precursors such as PEG-glutarate-NHS ester, PEG tetramine and a non-crosslinked hydrophobic polymer such as PLGA with non-reactive end groups, organic solvent and optionally a drug/visualization agent with non-reactive chemical functional groups (cannot participate in precursor crosslinking reaction) are mixed in an organic solvent like THF to form a homogeneous solution. The mixture is then poured into mold cavities before effective crosslinking and gelation formation to provide a desired shape to the precursor solution. Under effective crosslinking conditions, the precursors in the mold cavity undergo effective condensation polymerization and crosslinking to form organic solvent gel microparticles in which hydrophobic polymer PLGA remains entrapped in the crosslinked polymer. The gel particles are removed from the mold. The gel particles may be optionally washed with organic solvents to remove unwanted crosslinking reaction products such as n-hydroxysuccinimide and low molecular weight unreacted precursors and the like. The solvent is removed and the PLGA in the particle is precipitated “in situ” inside the crosslinked microparticle. The precipitated particle encapsulates the drug which is then released via diffusion and/or erosion from a biodegradable polymer and crosslinked gel. In some cases, if a biocompatible water miscible solvent such as NMP or DMSO is used for drug infusion, the solvent swollen microparticles can be directly injected into the tissue. The solvent is dispersed in the tissue, precipitating the PLGA in the composite microparticles. The precipitated PLGA encapsulates the drug and provides sustained drug delivery in the tissue.

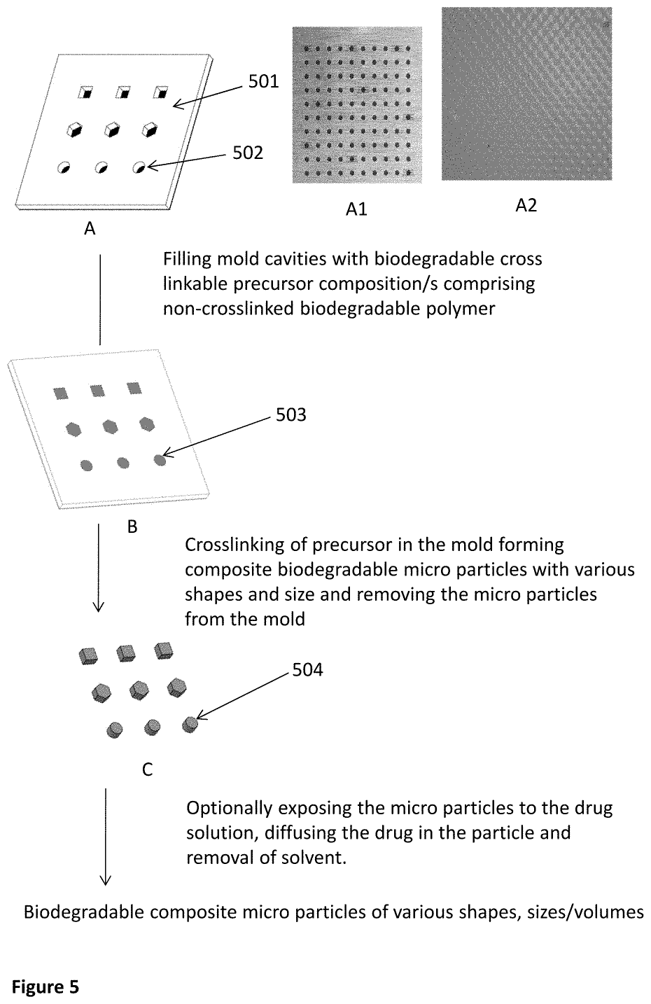

FIG. 5 shows a partial schematic representation of a method for creating composite microparticles of various shapes comprising a biodegradable crosslinked polymer and biodegradable polymer made using organic gelling compositions described in this invention. FIG. A shows a mold made out of silicone rubber or gelatin hydrogel or polyvinyl alcohol hydrogel and the like with microcavities of the desired volume and shape. 501 shows mold material body with top and bottom surfaces and 502 shows cubical, cylindrical and hexagonal prism shaped cavities in the 501 body. A1 shows an illustrative aluminum based mold with 300 microns diameter cylindrical holes and A2 shows gelatin based dissolvable hydrogel mold with 50 microns size cylindrical cavities. FIG. B shows 502 mold cavities filled with precursors that produce organic solvent gel or hydrogel materials (503) described in this invention which may optionally comprise non-crosslinked biodegradable polymer/carrier such as PLGA. The precursor optionally may also contain drug, cells, visualization agent or porogen to create porosity. The precursor composition is effectively crosslinked under effective crosslinking conditions and the crosslinked composition (504) takes the shape of the mold

cavity. The crosslinked microparticles (504) of various shapes are removed from the mold, isolated, optionally washed to remove crosslinking reaction byproducts and other undesirable components (C). The crosslinked hydrogel/ organic solvent gel microparticles are stored for future processing and use. In some embodiments, the precursor solution with drug/s or cells is frozen or dried in the mold. The frozen/dry shaped particles are taken out of the mold cavity and then crosslinked in a frozen/dry state to form a crosslinked product.

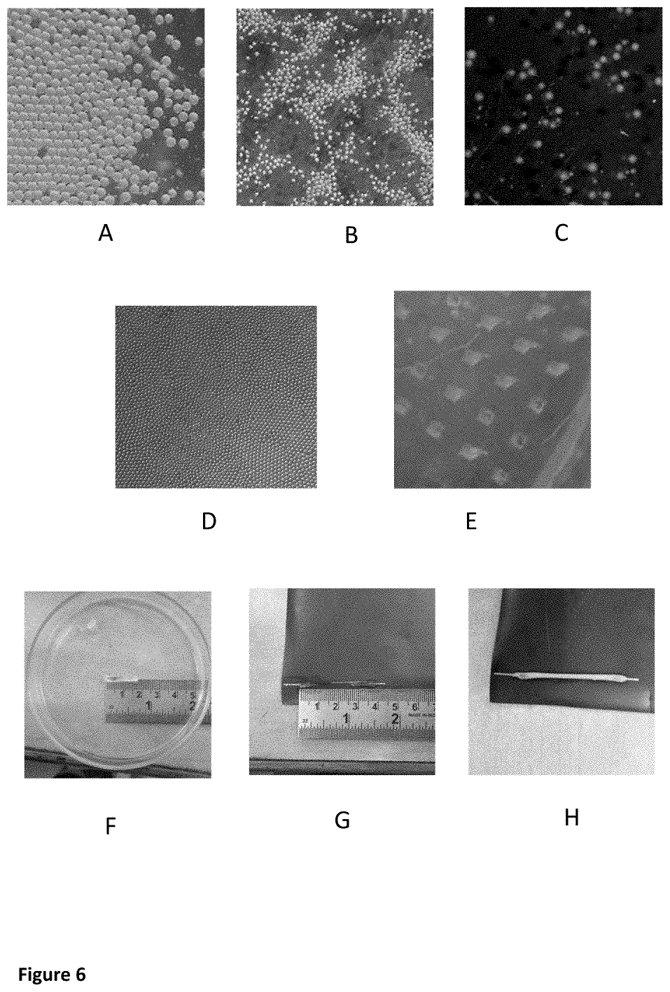

FIG. 6 shows photographs of the composite microspheres prepared using methods described in this invention. FIG. A shows composite organic solvent gel microspheres prepared from photocrosslinked PEG macromonomer and PLGA (molecular weight 10000-15000 Daltons, endcapped) that are swollen in solvent dichloromethane (size around 150 microns). The microspheres were prepared using the coacervation/microfluidic chip method. The crosslinked polymer nature does not permit the droplets to merge or coalesce or to form bigger droplets/particles or split to form smaller particles even though the particle comprises solvent dichloromethane and PLGA polymer. FIG. B shows a photograph of the same particles as in A wherein solvent dichloromethane is substantially removed by evaporation and PLGA in the gel particles is almost completely precipitated “in situ” inside the microspheres making them substantially opaque in nature. FIG. C shows photograph composite microspheres wherein solvent dichloromethane was completely removed by evaporation and then incubated in water. Crosslinked PEG Hydrogel swells in water (substantially transparent) and PLGA in the microspheres is seen as opaque particles within the swollen PEG hydrogel microspheres. FIG. D shows a photograph of blank PLGA microspheres prepared using microfluidic devices showing uniform particle size around 110 microns. These and other drug/visualization agent loaded microspheres were used to make multilayered drug delivery compositions as described in this invention. FIG. E shows a section of a photograph of a microneedle array prepared according to one of the illustrative embodiments. The array needles are made using composite materials comprising crosslinked PEG based biodegradable polymer and PLGA as a non-crosslinked biodegradable polymer entrapped in the needle material and is precipitated in situ inside the needle. FIG. 6F shows an exemplary elastic composite organogel comprising crosslinked PEG based polymer with PLGA as non-crosslinked biodegradable polymer swollen in organic solvent (DMSO). The organogel is substantially transparent. FIG. 6G shows implant 6F in stretched condition without breakage showing its elastic nature of the composite organogel. FIG. 6H shows implant 6F in stretched condition with solvent partially removed and the PLGA in the implant is precipitated in the stretched implant locking its stretched shape. The precipitated polymer makes the implant 6H opaque.

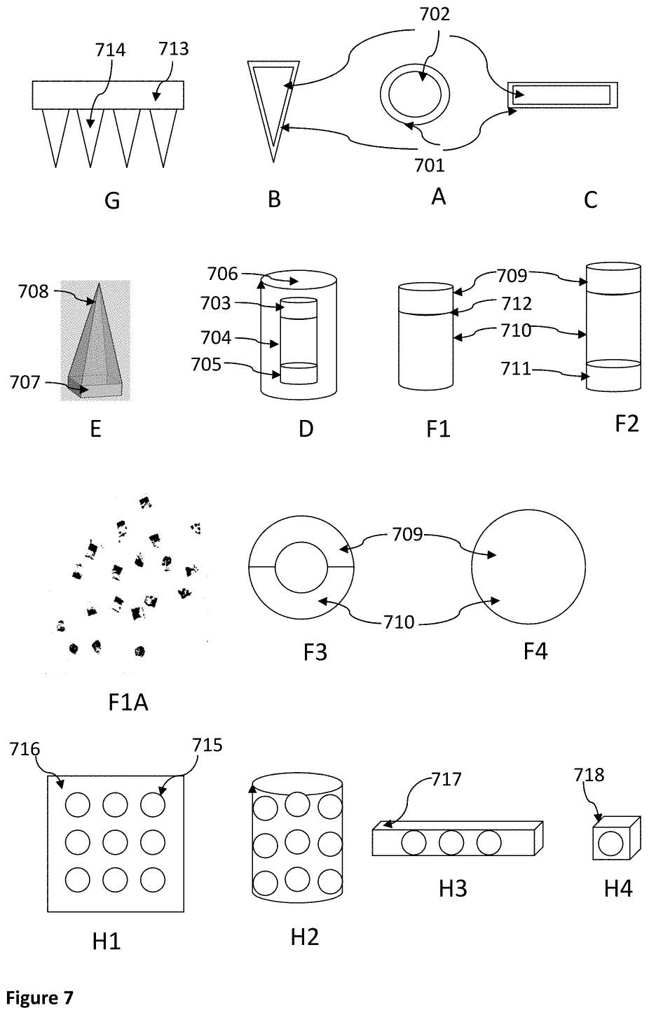

FIG. 7 shows a partial schematic representation of composite and/or multilayered materials described in this invention. FIG. 7A, B, C show composite particles of various types. The spherical shaped composite material A (microsphere) comprises crosslinked material (701), preferably biodegradable crosslinked material with a non-crosslinked biodegradable polymer (702) wherein the biodegradable polymer 702 is precipitated in situ inside the crosslinked material 701. B is the same as A except in a microneedle shape. C is similar to A in the form of a coating on a medical device. FIG. D shows multilayered particles wherein layers 703, 704 and 705 are completely encapsulated in a separate encapsulating material layer (706). The layers 703, 704 and 705 present as separate layers in the 706 encapsulating layer and are not covalently bonded. FIG. E shows a microneedle of a microneedle array device with two layers, a base layer (707) and microneedle layer 708 and each layer may be loaded with a drug and/or visualization agent. FIG. F1 shows a single cylindrical unibody particle with two layers (709, 710) wherein layers 709 and 710 are covalently linked. FIG. F1A shows a microscopic image of two layered unibody particles (diameter around 300 microns, length around 500 microns) similar to F1 wherein the blue layer is a crosslinked PEG macromonomer hydrogel layer with blue colorant additive and the transparent layer is a crosslinked gelatin methacrylate hydrogel layer and both layers are covalently linked. F1A also shows two vastly different chemical compositions in a single unibody particle. FIG. F2 shows a unibody three layered particle with 709, 710 and 711 as unique layers that are covalently linked wherein 712 shows the interface between two layers. F3 is similar to F1 except in the form of torus or ring shape with two layers. F4 shows spherical particles with two layers comprising crosslinked materials. H1 shows a top view of sphere/disk/cylinder shaped (715) particles that are covalently attached to the film or sheet surface (716) to form an array unibody material. 715 is projected upwards perpendicular to the 716 surface and is arranged as a 3 by 3 array. 715 and 716 may have different crosslinked compositions. H2 is similar to H1 wherein 715 is attached to the luminal surface of a tubular body and 176 is projecting towards the center of the lumen. H3 is similar to H1 wherein 715 is embedded/encapsulated in a hydrogel or polymer film (717). H4 is a cubical particle (718) with an embedded spherical particle (715). 715 may be physically entrapped or covalently linked to 717 or 718. Each layer in all particles described above may have a different composition or encapsulant or physical/chemical property.

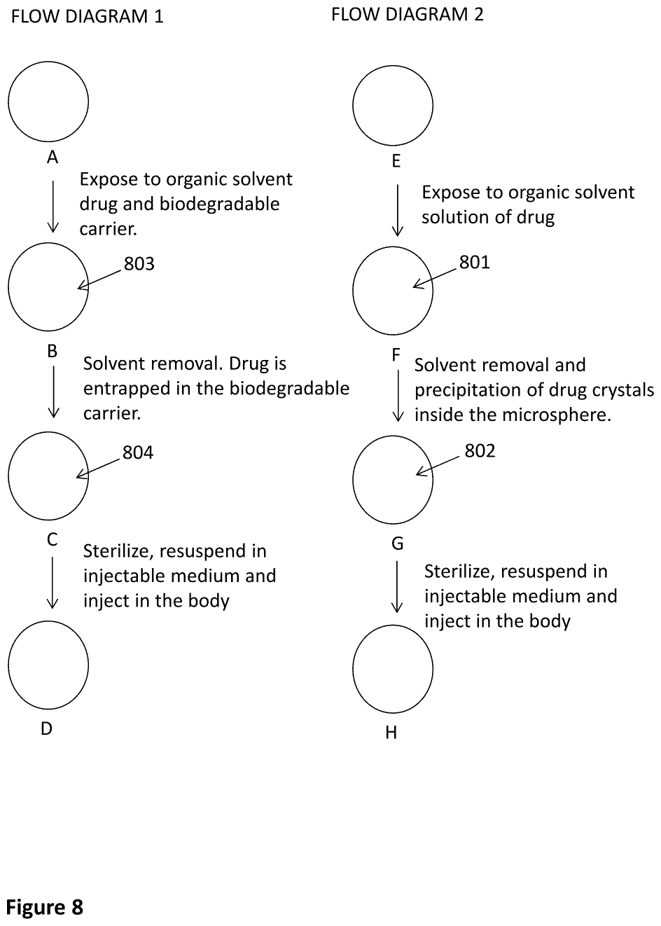

FIG. 8 shows a partial schematic representation of methods to make drug loaded microparticles or microspheres. FLOW DIAGRAM 1 shows a method to infuse drug and biodegradable carriers in crosslinked microspheres/microparticles. A schematically shows a crosslinked microsphere preferably crosslinked biodegradable microsphere. Particle A is then exposed to the homogeneous solution of drug and biodegradable carrier in an organic solvent. The organic solvent is capable of swelling the crosslinked microsphere A but incapable of dissolving A. The particle is incubated in the drug solution for sufficient time until the desired amount of drug and the biodegradable carrier is infused inside the particle (B). Particle B is optionally washed with solvent to remove surface bound drug and carrier without substantial loss of drug and/or carrier. The biodegradable carrier used can be solid, semisolid, gel or liquid at room temperature or body temperature (37 degree C.). The solvent is removed from the swollen particle by evaporation or lyophilization of B and drug and biodegradable carrier remain entrapped in the microsphere (C). The drug loaded particles are sterilized (D), suspended in an injectable medium like PBS solution (pH 7.4) and then injected into the body for local or systemic drug delivery. FLOW DIAGRAM 2 shows a method to prepare drug loaded biodegradable hydrogel microspheres wherein the drug is substantially insoluble in water. E schematically shows a crosslinked microsphere preferably crosslinked biodegradable hydrogel microsphere. The particle E is exposed to a homogeneous solution of the drug in an organic solvent. The organic solvent is capable of swelling the crosslinked microsphere E but incapable of dissolving E and the organic solvent has higher or substantially higher solubility of the drug as compared to water. The particle E is incubated in the drug solution for sufficient time until the desired amount of drug is infused inside the particle E (F). Particle F is optionally washed with solvent to remove surface bound drug without substantial loss of drug from F. The solvent is removed from the swollen particle by evaporation or lyophilization which precipitates/crystallizes drug particles inside E (G). The drug loaded particles are sterilized (H), suspended in an injectable medium like PBS solution (pH 7.4) and then injected into the body for local or systemic drug delivery. The crystallized drug within particles slowly dissolves under physiological conditions and provides a systemic or local therapeutic effect. The biocompatible crosslinked polymer also protects the drug particle surface from foreign body reaction.

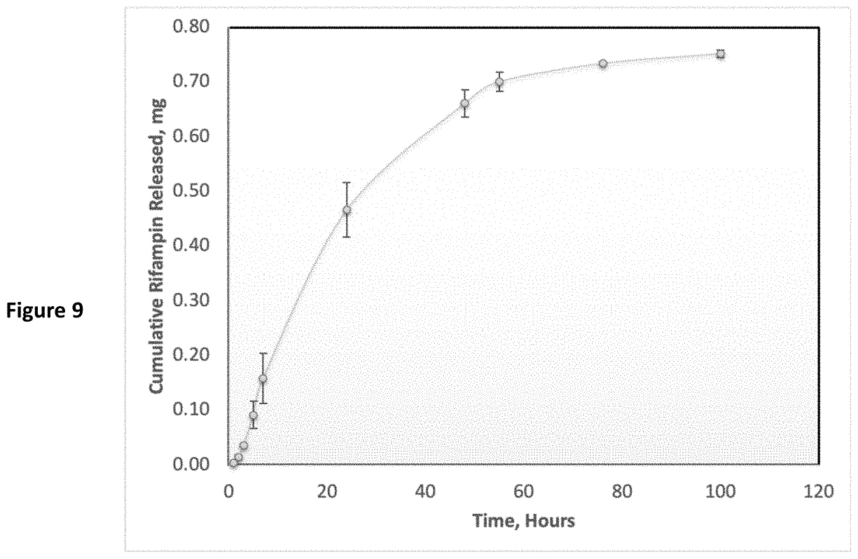

FIG. 9 shows a controlled release profile of rifampin from composite microparticles comprising PEG based crosslinked polymer with in situ precipitated PLGA polymer. The drug rifampin was infused in the crosslinked microparticles by solvent diffusion technique. The rifampin encapsulated in PLGA is released in a controlled manner for over 4 days.

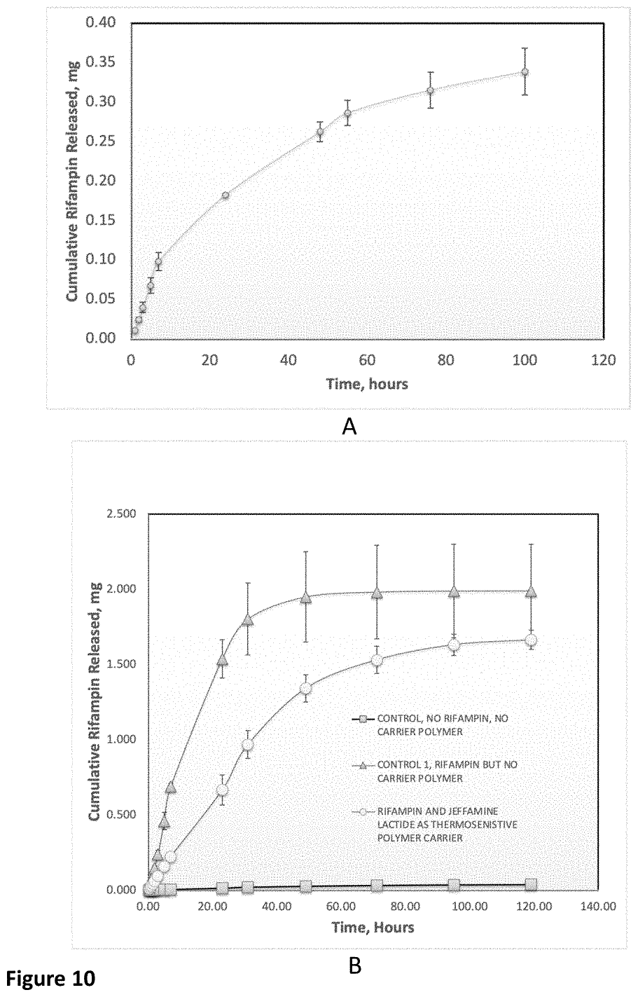

FIG. 10A shows the controlled release profile of rifampin from composite microparticles comprising PEG based crosslinked polymer with vitamin E as an illustrative liquid drug carrier. The drug rifampin and Vitamin E were infused in the crosslinked microspheres via solvent diffusion technique. Vitamin E helps to release the drug in a controlled manner for 5 days. FIG. 10B shows a controlled release profile of rifampin from composite microcylinders comprising PEG based crosslinked polymer and Jeffamine lactide copolymer as an exemplary thermosensitive polymer carrier. The drug rifampin and Jeffamine lactide were infused in the PEG crosslinked microcylinders via solvent diffusion technique. Crosslinked microcylinders with no rifampin and no Jeffamine lactide polymers were used as control and crosslinked microcylinders with rifampin but no Jeffamine lactide polymer was used as control 1. As expected, control microcylinders do not show the release of rifampin. Control 1 microcylinders show a fast release in 50 hours and samples with Jeffamine lactide show a controlled release up to 120 hours.

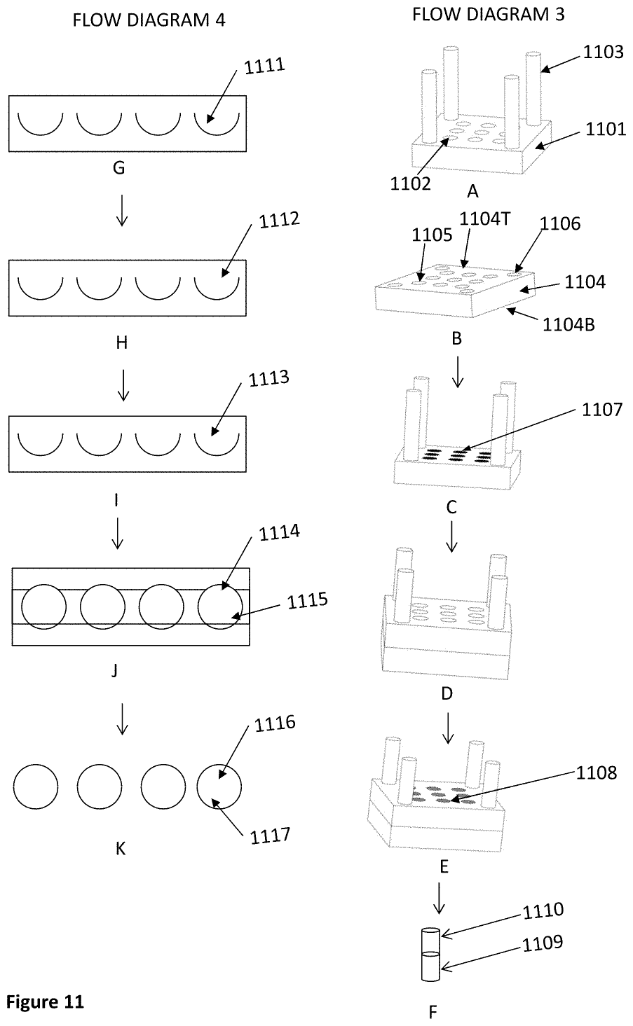

FIG. 11 shows a partial schematic representation of methods for making multilayered microparticles/implants using two or more molds. FLOW DIAGRAM 3 describes a method of using two or more molds to make cylindrical two layered particles and FLOW DIAGRAM 4 describes a modified version of FLOW DIAGRAM 3 to make spherical two layered particles. FIG. A and B schematically show molds used for casting of multilayered particles using methods and compositions described in this invention. A shows a rectangular body (1101) made out of silicone rubber or gelatin hydrogel or other desirable mold material and the mold body has one or more microcavities (1102) created for the casting of crosslinkable precursor microparticles. Mold A also has 4 cylindrical columns or guideposts (1103) used for alignment and holding the second mold B. Mold B is similar to A and has a base body (1104) with top surface (1104T) and bottom surface (1104B) and mold cavities (1105) that are open on both surfaces (1104T and 1104B). B also has additional 4 holes (1106, open on 1104T and 1104B surfaces) on each corner whose diameter is slightly bigger than the diameter of guideposts on A but the center of guideposts on A (1103) and center of corner holes on B (1106) are aligned when placed on top of each other. The size, shape and arrangement of cavities on molds A and B are preferred to be identical. FIG. C shows mold cavities on mold A filled with precursor solution such as macromonomer solution with photoinitiator (1107) and excess solution is wiped off. Optionally, the precursor solution may be frozen or thermoreversibly gelled to reduce diffusion of components within each layer. The mold B is then inserted in mold A via guideposts (1103) through the corner holes (1106) aligning the mold cavities of A and B (FIG. D). The mold cavities of B are then filled with a second photocrosslinkable precursor solution (1108) which may be different from the first one (1107). The excess solution is wiped off. First or both solutions may be partially or completely liquified and the mold cavity is exposed to UV/visible light to induce effective photopolymerization and crosslinking of precursor 1107 and 1108 compositions. The polymerization and crosslinking occur in both molds forming a singular crosslinked unibody implant (F). The crosslinked gels are removed from the mold. The particle thus produced has a single body or unibody (F) with two layers of (1109 and 1110) crosslinked compositions. Another variation of this process is used to make spherical shaped bilayered particles (FLOW DIAGRAM 4). G shows a semispherical (half-spherical) shaped transparent or semi-transparent mold cavity (1111). The cavity 1111 is filled with precursor solution 1112 (H). I show a second precursor solution (1113) filled in a second mold. Solutions 1112 and 1113 are reversibly gelled and/or frozen to form gels/solids (1115 and 1114). The mold H and I are aligned and kept on top of each other such that open surfaces of 1112 and 1113 touch each other and the resultant shape is spherical in nature (J). Precursor solutions are exposed to light to effectively polymerize and crosslink both solutions without substantial mixing. The precursor may be in a frozen state, liquid state or physically crosslinked gel state or combination thereof. H shows polymerized unibody spherical particles with two layers 1116 and 1117 wherein 1116 is crosslinked 1112 and 1117 is crosslinked 1113.

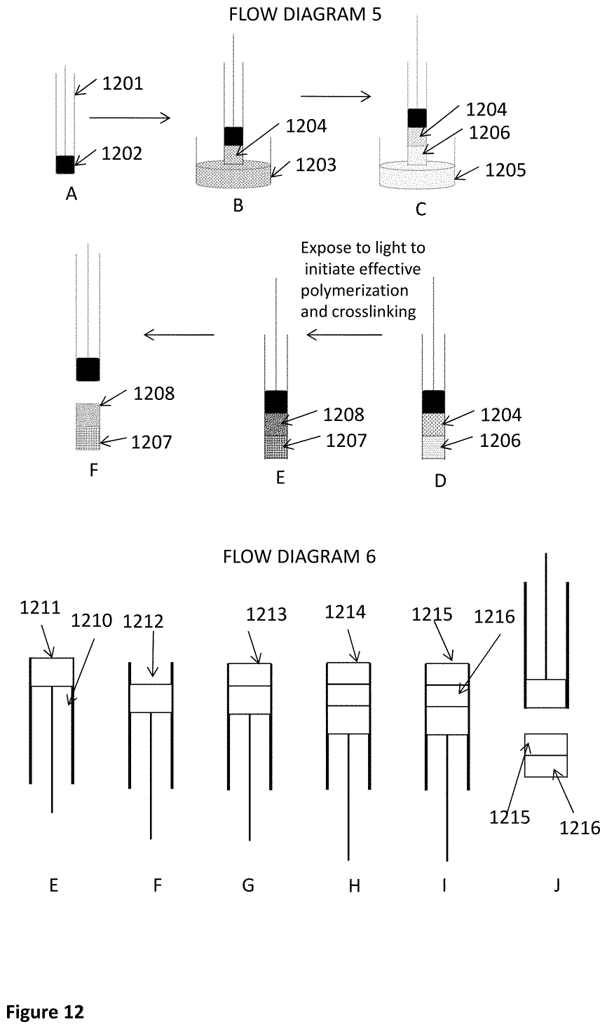

FIG. 12 shows a partial schematic representation of a method for making a drug or live cell encapsulated multilayered microparticles. In this method (FLOW DIAGRAM 5), a transparent glass capillary tube or mold cavity (1201) is inserted with a piston (1202) which can be moved along the length of the capillary tube to suck/pull a controlled amount of the desired precursor solution (FIG. A). FIG. B shows the glass capillary described in FIG. A inserted in a photopolymerizable precursor solution A reservoir (1203). The piston is moved upward to create a controlled amount of space in 1201 by sucking out a measured amount of precursor solution A in the capillary (1204). Depending on the composition used, capillary action (surface tension) may also be used to drive a measured amount of solution in the capillary without the use of a piston. The solution on the external walls of capillary 1201 is wiped out to prevent contamination. The assembly is then transferred to photopolymerizable precursor solution B (reservoir (C, 1205). The piston is moved upwards and a measured/controlled amount of precursor solution B is then pulled/sucked in the glass capillary, schematically shown as 1206. The assembly is then taken out, excess solution B is wiped out from external walls of the capillary (D) and then exposed to UV or visible light to induce effective photopolymerization and crosslinking of the precursor solutions A and B. The effectively polymerized precursor solutions A and B are schematically shown as 1208 and 1207 respectively (E). The piston is then pushed downwards to inject a polymerized microcylinder particle (F) from the capillary which exists as a single unibody particle but having two distinct layers 1208 and 1207 within a single unibody structure. FLOW DIAGRAM 6 shows a modification of the FLOW DIAGRAM 5 method wherein cold precursor microparticles/microspheres are used to form layered composite unibody material. A needle connector portion of a syringe is cut off leaving behind a syringe barrel and plunger. E shows a glass syringe barrel (1210) with a glass syringe plunger (1211). The syringe is cooled in the freezer at −20 degree C. The syringe plunger is moved down to create a space in the barrel (1212). This space is then filled with cold frozen microspheres of precursor solution (1213) with a photoinitiator with an ability to effectively polymerize in the frozen state such as gelatin methacrylate solution in PBS (GM). After filling space 1212 completely, the plunger is pulled down again to create additional space in the barrel. The additional space is then filled with a second layer of cold frozen microspheres/microparticles (1214) of different or same precursor with photoinitiator with an ability to effectively polymerize in the frozen state such as PEG1OKUM in PBS. The frozen microspheres in both the layers are exposed to light until effective polymerization of precursor microspheres in the syringe barrel. The polymerization takes place within the microspheres and between the microspheres forming a unibody material. The effective polymerization produces two layered unibody objects comprising polymerized crosslinked precursors (I, 1215 and 1216). The polymerized bilayer unibody object comprising layers of 1215 and 1216 is pushed out of the syringe (J) and stored until use. In another variation of this method, two frozen microspheres precursors (1213 and 1214) are mixed using equal volume and then filled into the empty space of a barrel (1212) and exposed to light to effectively polymerize both the precursors. This yields a composite of crosslinked unibody material wherein precursors 1213 and 1214 are covalently linked with each other within the crosslinked unibody structure. The crosslinking takes place within and between microspheres forming a composite material and crosslinked 1213 and 1214 are randomly distributed in the unibody structure.

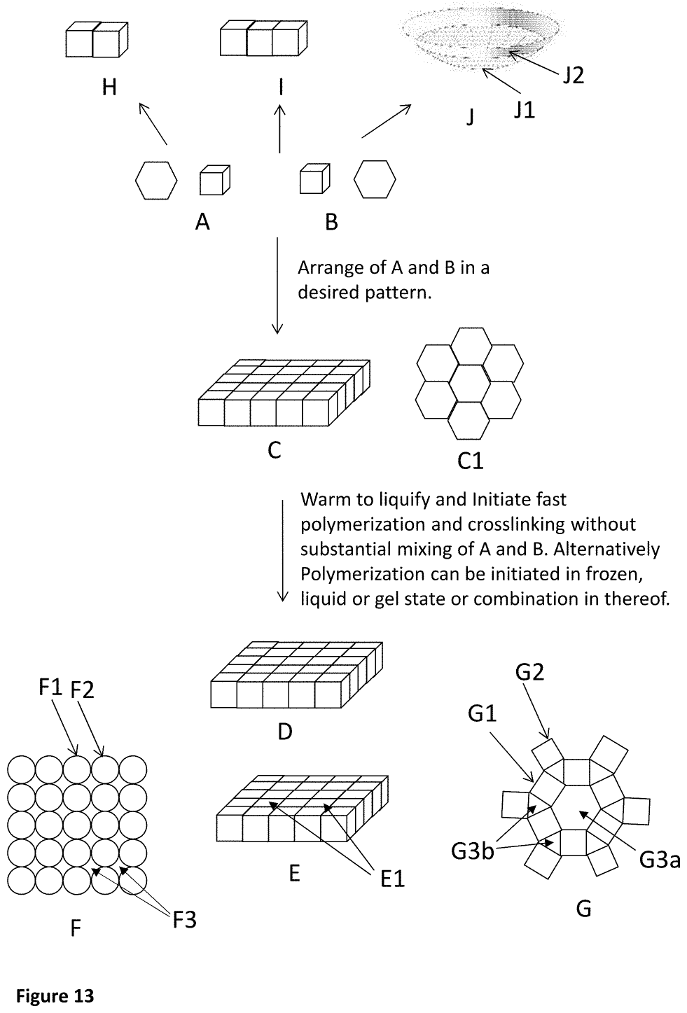

FIG. 13 shows a partial schematic representation of a method to make unibody materials with diverse compositions in the desired 2 or 3-dimensional patterns. A photopolymerizable precursor (P1) solution with a photoinitiator is first filled into a mold and then frozen/dried to form a cubical or hexagonal prism shaped solid (A). B shows another precursor composition (P2) with a photoinitiator in a frozen cubical shape or hexagonal prism shape. A and B generally have the same shape and dimensions. A and B are arranged in a checked pattern in a frozen state (C) preferably in another mold so that at least one of the surfaces of A and B touch each other. C1 shows hexagonal prism shaped arrangement of precursor P1 and P2 wherein one side of the prism touches with another side. A micromanipulator tool may be used for smaller sized frozen particles to achieve a desired pattern or arrangement in the frozen state. Compositions P1 and P2 may be warmed to liquefy (partially or completely) without substantial mixing. The compositions P1 and P2 in C are then exposed to light to effectively crosslink P1 and P2. The polymerization takes place within and between P1 and P2. P1 and P2 may be completely melted or partially melted or maybe in a frozen solid-state or combination thereof before crosslinking. The effective polymerization leads to the formation of a unibody structure where all cubes are covalently linked to each other to form a single unibody structure (D). The physical and chemical properties associated with crosslinked P1 and P2 are dispersed in the body of D in the desired pattern. E is similar to D wherein some cubes are intentionally removed from the frozen arrangement or not placed during the arrangement prior to crosslinking leaving behind the void or porous space (E1) in the crosslinked structure. FIG. H shows a bilayered unibody particle made by the fusion of A and B via photopolymerization and crosslinking as described above. FIG. I shows a unibody three layered particle (ABA) made similar to H. FIG. J shows a unibody concentric disk like design in which inner circle/disk (J1) and outer circle/disk (J2) are covalently linked to form a two layered unibody composite material. FIG. J can be considered as a contact lens wherein J1 could be a contact lens part that provides refractive properties and an outer layer (I2) provides drug delivery or other useful function/property. FIG. F shows cylindrical or spherical shaped frozen/dried precursor solids/solutions with two different frozen compositions (F1 and F2) arranged in the desired pattern and then crosslinked to preserve the pattern arrangement and produce unibody porous structure. The empty space between the spheres (F3) may be used to create the desired amount of porous space in the crosslinked unibody structure by controlling frozen particle size, shape and pattern. FIG. G shows diamond shaped frozen particles with compositions G1 and G2 arranged to create a hexagonal shaped cavity (G3a) and triangular cavity (G3b). The arrangement is locked in place by photocrosslinking of the G1 and G2 to create a crosslinked unibody structure with hexagonal and triangular shaped cavities/porous space in the unibody material.

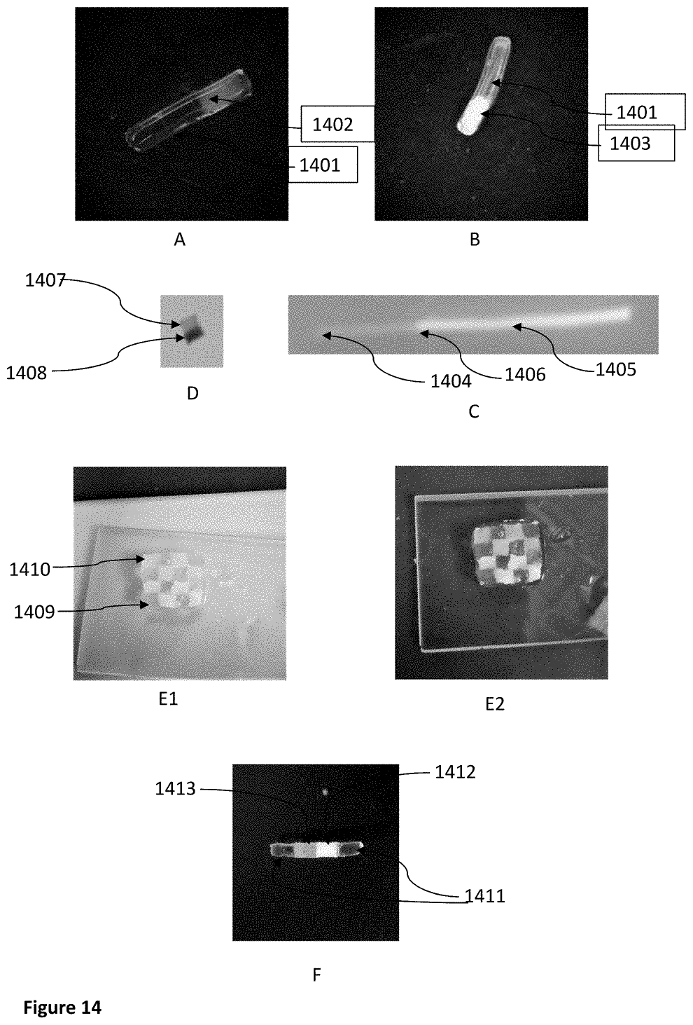

FIG. 14 shows microscopic photographs of the multilayered unibody materials prepared using methods and compositions described in this invention. FIG. A shows cylindrical shaped two layered unibody particles wherein one layer is an organogel layer that has crosslinked polymer swollen in organic solvent and the other layer has the same crosslinked polymer with a non-crosslinked biodegradable polymer (NBP) dissolved in the organic solvent. About the bottom two-thirds part of the particle is a semitransparent organic solvent gel (1401) made from crosslinked PEG20KUA with no NBP. The opaque portion (top one third, 1402) is a composite material comprising crosslinked PEG20KUA with in situ precipitated NBP (PLGA polymer). FIG. B is similar to A except the PLGA portion of the layer is loaded with coumarin as a model drug with fluorescent properties (1403). The green fluorescence of coumarin encapsulated in NBP under long UV light (1403) shows a bilayered structure with sharp boundaries between two layers. FIG. C shows a unibody two-layered crosslinked cylindrical implant made using PEG20KUA macromonomer (1404) and gelatin methacrylate (1405). This particle is then treated with fluorescein and lodixanol using EDC and n-hydroxysuccinimide which covalently bonds gelatin to fluorescein and lodixanol but not to crosslinked PEG2OUA. The crosslinked gelatin portion (1405) shows green fluorescence due to covalently bonded fluorescein and the crosslinked PEG20KUA macromonomer shows no fluorescence due to lack of covalent sites (functional groups) to bind fluorescein and lodixanol. 1406 shows a sharp boundary between 1404 and 1405 showing a bilayered structure in a unibody mass wherein 1404 is a synthetic hydrogel and 1405 is natural polymer hydrogel. FIG. C is also an example of a bilayer unibody particle wherein one layer (1404) is a biostable hydrogel and the other layer (1405) is a biodegradable hydrogel that degrades by an enzymatic pathway. FIG. D shows a microscopic image of two layer unibody magnetic microparticles wherein 1407 shows non-magnetic crosslinked PEG20KUA layer with no magnetic particles and 1408 shows magnetic layer with crosslinked PEG20KUA loaded with iron oxide. FIG. E1 shows the unibody gelatin hydrogel sheet with alternate bands of clear and opaque hydrogel in 4 by 4 format. 1409 shows bands of crosslinked gelatin methacrylate with no magnesium carbonate (transparent gel) and 1410 shows bands of crosslinked gelatin methacrylate with magnesium carbonate (opaque gel). E2 is the same as E1 wherein E1 was incubated in the warm water for 20 minutes. The non-dissolution of crosslinked gelatin hydrogel and slight swelling of E1 shows an effective crosslinking process. The transparent and opaque gel parts also remain attached in the desired pattern and do not separate from each other indicating covalent bonding of cubes and its unibody structure. F shows two microcylinders (1412 and 1413) encapsulated in a hydrogel matrix (1411). The 1412 microcylindrical part comprises fluorescent particles encapsulated in a hydrogel matrix. The 1413 microcylindrical part comprises a hydrogel matrix wherein PLGA is precipitated in situ inside the hydrogel. 1412 and 1413 are made separately first and then encapsulated in the 1411 matrix.

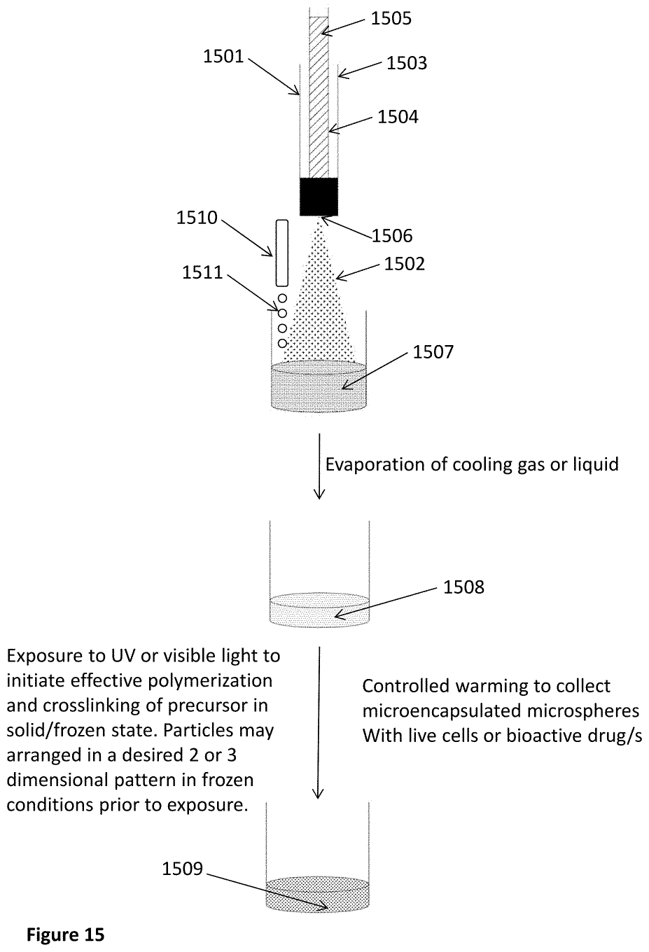

FIG. 15 shows a partial schematic representation of a method to make drug/live cell encapsulated microparticles/microspheres using frozen or dry precursor microparticles. A photopolymerizable precursor solution is injected via sprayer (1501) to form microdroplets (1502) in the air. The sprayer 1501 has a “tube in tube” like arrangement wherein an outer tube (1503) is used to carry pressurized air or an inert gas like nitrogen or argon. The inner tube (1504) of the sprayer is used for transportation of precursor solution (1505) such as photopolymerizable macromonomer solution in water or organic solvents with a photoinitiator. The precursor solution may comprise a drug and/or live cells depending on the solvent used. Both the solution and gas exit from a small sprayer nozzle (1506) forming the solution droplets (1502) in the air. Optionally, the droplet emitting nozzle tip may be vibrated ultrasonically or other mechanisms to control droplet size. In some cases, the sprayer may be replaced with a droplet generating apparatus (1510) which generates picoliter to microliter size droplets (1511) of the desired volume. The droplets are collected in freezing liquid (1507) preferably in liquid gases like liquid nitrogen or liquid helium which converts the liquid droplets into frozen microparticles/microspheres. Electric potential difference between droplet emitting tip and droplet collection beaker may be used as an additional tool to control droplet size. Upon evaporation of liquid gas, the frozen droplets (1508) are exposed to UV/visible light to initiate effective polymerization and crosslinking of precursor solution in the frozen state. The crosslinked microspheres (1509) are stored in a frozen state or at ambient temperature until further use. For living cells, microfluid apparatus may be used in place of sprayer and cooling rates may be controlled to maintain substantial cell viability. In some embodiments, the frozen microspheres of desirable compositions may be arranged in a desirable 2D or 3D pattern and then exposed to light to lock the frozen state arrangement to make a unibody 2D or 3D materials. Alternatively, a volatile organic solvent such as dichloromethane is used to form precursor solution with drug and then sprayed and collected with or without freezing liquids to form dry or substantially dry (without solvent dichloromethane) precursor particles. The collected substantially dry particles are then exposed to light for effective crosslinking in dry state.

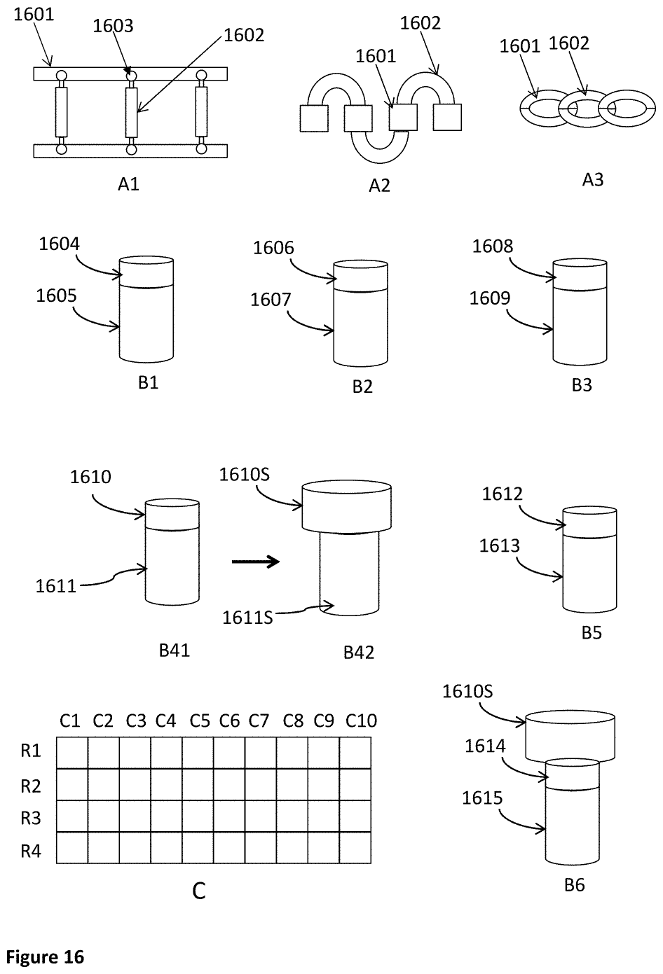

FIG. 16 shows a partial schematic representation of composite materials made using mechanically and covalently bonded composite materials. It also shows preferred ophthalmic or punctal bilayered or multilayered implants. A1, A2 and A3 show unibody composite materials made using methods and compositions described in this invention. 1601 is a hard crosslinked material and 1602 is a soft elastomeric crosslinked material that serves as a bridge between 1601 material. 1603 is a portion of 1602 that is mechanically embedded as well as covalently locked into 1601 material. A2 is similar to A1 wherein the 1602 bridge has a curvature connecting 1601 hard material. When A2 is stretched, the curvature of 1602 provides space to expand the material. A3 shows a chain-like arrangement between 1601 and 1602 materials. Implant B1 is a two layered single body cylindrical punctal/medical implant wherein the top portion of the implant has microspheres with visualization agent (1604) and the bottom portion of the implant has drug encapsulated microspheres (1605). FIG. B2 shows bilayered (1606 and 1607) punctal implant comprising different drugs present in two different layers (1606 and 1607). Preferably drugs in 1606 and 1607 layers are microencapsulated in a biodegradable polymer. FIG. B3 shows a bilayered (1608 and 1609) punctal implant comprising the same drug in each layer but has a different controlled release rate/profile. A drug in the 1608 layer releases all the drug quickly in 0.1 to 7 days (fast release) and a drug in the 1609 layer releases the drug from 15 days to one year (slow release). B4 shows a bilayered (1610 and 1611) unibody implant wherein one of the layers (1610) has substantially more ability to absorb water and swell under physiological conditions (pH 7.4, 37 degree C.) than the other layer (1611). B41 shows the size and shape of the implant immediately after inserting into the punctal cavity. B42 shows B41 implant after absorption of water within 24-72 hours after implantation. The layer 1610S swells significantly more than the layer 1611S which helps to immobilize the implant in the punctal cavity and prevents its migration in the punctal cavity. FIG. B5 shows bilayered (1606 and 1607) punctal implant comprising a visualization agent such as fluorescent agent (1612) and ophthalmic drugs such as dexamethasone (1613). Preferably, drugs and visualization agents in the 1612 and 1613 layers are microencapsulated in a biodegradable polymer like PLGA. FIG. B6 shows a trilayered implant wherein the implant has a swellable layer 1610S and two layers for controlled drug delivery. Preferred drugs are antiinflammatory drugs and antibiotics. Layer 1614 may comprise dexamethasone and 1615 may comprise moxifloxacin. FIG. C shows a unibody hydrogel grid-like sheet similar to shown in FIG. 14E. The sheet can be used for clinical diagnostics of disease like virus detection, cancer biomarkers and other clinical diagnostic applications. The illustrative sheet has 10 columns and 4 rows. At position R1C1, an antigen-specific to the disease to be detected is infused in the hydrogel matrix. This location serves as a positive control. R1C2 and R1C8 intentionally left blank. R1C3, R1C4, R1C5, R1C6 and R1C7 have known concentrations of antigen and it can be used for internal calibration purposes. R1C9 and R1C10 contain antibodies specific to the antigen being analyzed in duplicate. Row two is a duplicate of R1. When the grid is exposed to blood or serum to be analyzed, the antigen in the sample interact with the antibodies in the R1C9 and R2C10 and upon subsequent processing with enzyme and other specific test-related reagents produce response such as color, fluorescence or luminescence which can be used for qualitative and/or quantitative diagnostics use.

Using a calibration curve created by the C3-C7 blocks, quantitative analysis can be made. The location of the sample and standard inside the matrix can also be used as a tool for clinical diagnosis.

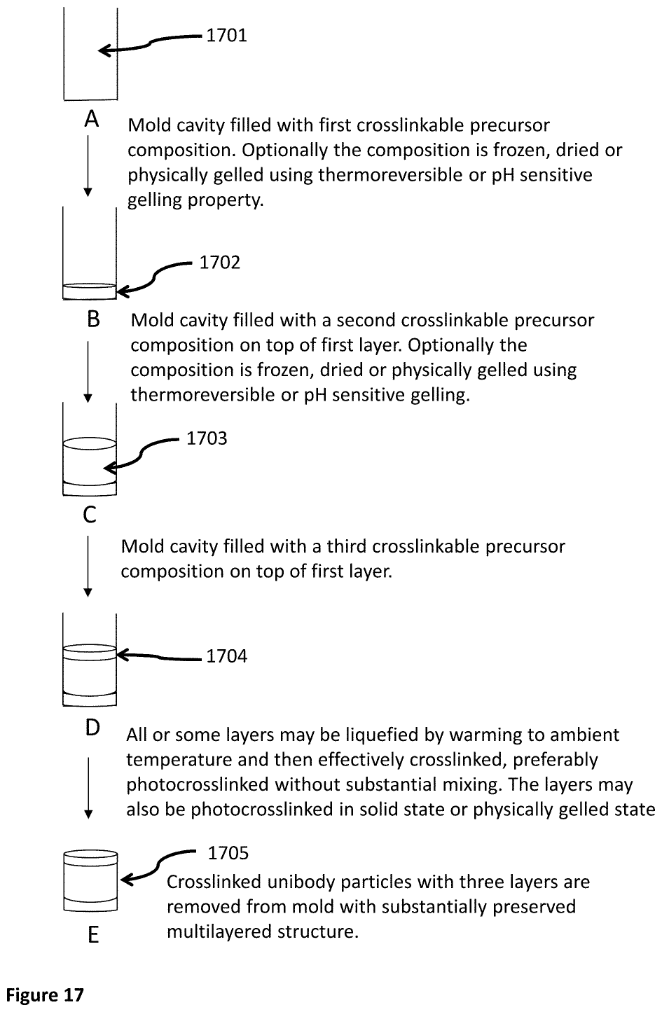

FIG. 17 shows a schematic representation of a method for making biostable or biodegradable multilayered microparticles. FIG. 17A schematically shows a cylindrical mold cavity (1701). FIG. 17B shows the schematic mold cavity 1701 that is partially filled with a precursor composition (1702). The illustrative 1702 precursor composition may comprise a biodegradable polymer and visualization agent such as colored or fluorescent compound. The composition in 1702 is optionally frozen or reversibly gelled or dried to prevent mixing with an additional layer of crosslinkable precursor composition to be added as a second layer. In FIG. C, a second layer precursor composition (1703) is added. The illustrative 1703 precursor composition may comprise a biodegradable polymer and drug. Both layers (1702 and 1703) are optionally frozen or reversibly gelled to add a third layer of crosslinkable precursor. In FIG. D, a third layer precursor composition (1704) is added. The illustrative 904 precursor composition may comprise a biodegradable polymer and radio-opaque compound. All layers may be partially or completely liquefied by warming to room temperature if frozen or converted into liquid/solution state if reversibly gelled and then effective crosslinking is initiated before components in each layer substantially diffuse into other layers. The crosslinked three layered unibody particles are removed from the mold with preserved multilayered structure (E). In some embodiments, the crosslinking may be initiated when some or all the layers are partially or completely dried, frozen to solid state, in physical gel form or a liquid state. The crosslinking of precursors occurs in all layers producing layered microparticles with distant layers wherein each layer may comprise different composition or has different chemical/physical properties. All layers in the particle E are not separable and are fused into each other and combinations of all layers exist as one single unibody particle.

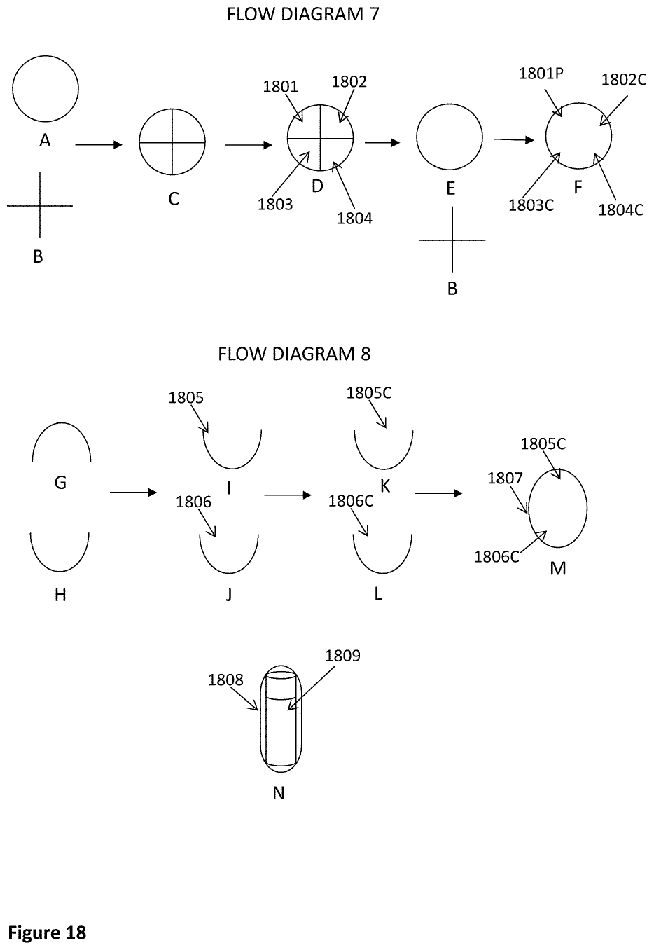

FIG. 18 shows a schematic representation of methods for making oral drug delivery systems. FLOW DIAGRAM 7 shows a method to make multilayered (4) layered oral drug delivery pharmaceutical tablets. FIG. A represents a circular mold cavity and B represents a removable spacer or divider which can be placed inside cavity A to make two or more cavities/compartments without leakage. C shows a cavity A with spacer B with 4 equally divided compartments/cavities. C is then filled with a precursor solution comprising a drug or visualization agent or sensors such as RFID tag or other biochemical sensors. Compartments in C are then filled with 4 different types of precursor compositions (1801, 1802, 1803 and 1804) that have the capacity to form unibody material upon effective crosslinking. E is the same as D except spacer B is removed and the precursor may be in liquid, solid or frozen state or combination thereof. E is subjected to effective crosslinking conditions such as exposure to UV light to crosslink and fuse all 4 layers to form a unibody circular disk shaped tablet that can be swallowed (F) for oral drug therapy. F shows 1801P, 1802P, 1803P and 1804P as crosslinked separate layers of a unibody material made from precursors 1801 to 1804 respectively. F may be further processed such as lyophilized or dehydrated and then coated with taste masking agents and the like to make an oral pharmaceutical tablet composition. FLOW DIAGRAM 8 shows a method to make oral drug delivery pharmaceutical capsules using hard or soft shell empty capsule shells as molds. G and H schematically show two halves/parts of standard gelatin or HPMC based hard/soft capsule shells (standard commercial sizes range from 000 to 4) that are commonly used in the pharmaceutical industry. The cavities in the shell are filled with the same or different types of precursor compositions (I, 1805; J, 1806). Preferably the precursor compositions can form unibody material upon effective crosslinking. 1805 and 1806 comprises drug or visualization agent or sensors such as RFID tag or other biochemical sensors. The components of precursors are inert towards capsule materials Precursors in 1 and J are subjected to effective precursor crosslinking conditions to form crosslinked compositions (1805C and 1806C). The shells are then fused to form the oral capsule. Alternatively, the precursor can be frozen/dehydrated first and shells are fused to form a capsule and crosslinking is initiated to form a unibody two layered body inside the capsule. N shows standard gelatin or HPMC oral capsule (1808) that is enclosed with two or multilayered drug delivery particles (1809) made according to methods described in this invention.

Description of Preferred Embodiments

Implantable Biodegradable Organic Solvent Gels.

We have discovered new organic solvent gelling compositions that can form biodegradable or biostable gels in many organic solvents, also referred to as “organogel” or “organic solvent gels” (OSG). The compositions are preferably made via chain growth polymerization reaction even more preferably free radical polymerization reaction. We used a combination of organogels and non-crosslinked biodegradable/biostable polymers to form composite materials that have many pharmaceutical and medical device applications. In this invention, precursors of free radical polymerization or condensation polymerization that preferably produce biodegradable crosslinked polymer networks are polymerized in an organic solvent in presence of non-crosslinked biodegradable polymer (NBP) such as PLGA to produce composite crosslinked materials that are swollen in organic solvent or organogel. The precursor polymerization may be conducted in presence of the drug and/or visualization agent to produce an organogel comprising drug or visualization agent. The entrapped drug is then released from the crosslinked polymer and/or NBP of the organogel.

Organic solvent means commonly used organic solvents in pharmaceutical or medical device preparation. A preferred class of organic solvents include but not limited to: hydrocarbons, ketones, alcohols, carbonates, esters, amides, halogenated hydrocarbons, ethers, organic acids, organic bases, aromatic solvents and the like or their mixtures in any proportions. Commonly used organic solvents and their mixtures in any proportions include but not limited to: dimethyl carbonate, methyl ethyl ketone (MEK), tert-butyl acetate, acetone, acetonitrile, cyclohexanone, dimethyl sulfoxide (DMSO), n-methyl pyrrolidinone (NMP), dimethylacetamide (DMAC), dimethyl formamide (DMF), methanol, ethanol, isopropanol, PEG molecular weight 400-1000 g/mol, PEG endcapped with methyl ether (molecular weight 200 to 1000 Daltons), PEG based solvents may be linear or 3-25 branches, dichloromethane, trichloromethane, chloroform, dioxane, ethyl acetate, dimethyl ether (DME), tripropionin (triprop), tetraglycol, pyrrolidone-2, ethyl lactate, triacetin, triethylene glycol dimethyl ether (triglyme), glycerol formal, ethylene glycol monoethyl ether acetate, benzyl alcohol, tributyrin, benzyl benzoate, acetic acid, diethylene glycol dimethyl ether (diglyme), ethyl benzoate, dimethyl isosorbide (DMI), polyethylene glycol dimethyl ether, glycofurol, glycerol, ethyl acetate,), 1,3 propanediol, 1,4 butanediol, 1-6-hexanediol, tetrahydrofuran (THF) and the like. US Patent 8506856 and other patents cited therein provide an additional list of monomers, macromonomers, organic solvents, reactive solvents, photoinitiators, light sources which may also be used to make composite materials described in this application. The solubility in organic solvent means forming a homogeneous solution with a solubility greater than 1 g per 100 g of the solvent, preferably greater than 5 g per 100 g of the solvent. For implantable organic solvent gel applications, organic solvents that are biocompatible and biodegradable are preferred, these include but not limited to dimethyl sulfoxide, n-methyl pyrrolidinone, ethanol, glycerol, PEG molecular weight 400-1000 g/mol, PEG end-capped with methyl ether (200 to 1000 g/mol), liquid PEG-polylactone polymers, and the like. Organic solvents with a melting point between 5 to 40 degree C. and boiling point between 40 to 180 degree C. are preferred. In some embodiments, especially in applications involving non-medical applications like optical arrays, monomers like methyl methacrylate, other acrylic and methacrylic esters, acrylic acid and its derivatives, styrene and its derivatives, vinyl acetate, n-vinylpyrrolidone and the like have been used as solvents as well as reactive monomers or comonomers.

The OSG are classified under two main groups according to the type of crosslinking mechanism; chemically and physically cross-linked gels. Chemical crosslinking involves covalent linking for the formation of macromolecules which include but not limited to: chain growth polymerization including anionic, cationic and free radical polymerization also known as addition polymerization; condensation polymerization; reactions involving Diels-Alder reaction like cycloaddition of dienes and dienophiles; hydrogen abstraction reaction catalyzed by benzophenone derivatives and light, Michael-type addition reaction (reactions involving vinyl-thiol crosslinking), reactions involved in click-chemistry like 1,3-dipolar cycloaddition of organic azides and alkynes, protein or natural biomolecules crosslinking using bi-or polyfunctional compounds like glutaraldehyde, 1,6-hexamethylene diisocyanate, catalysts like 1-Ethyl-3-(3-dimethylaminopropyl)carbodiimide, enzymes and the like. Physical crosslinking of gel generally involves non-covalent crosslinking of macromolecules which include but not limited to: ionic crosslinking of alginate gel with barium or calcium ion, ionic crosslinking of polyvinyl alcohol; Pluronic or Gelatin gels formed by hydrophobic interactions; gels formed from molecular self-assembly in response to external stimuli like light, pH, metabolite, pressure, temperature, electric current or charge, hydrophobic interactions and the like. The preferred OSG are physically and/or chemically crosslinked or their combination in any proportion are preferred. Most preferably, OSG gels are covalently crosslinked. The crosslinked network or organic gels created by new gelling systems reported in this invention are dimensionally stable and do not fuse with each other when exposed to organic solvent enabling the formed materials to be handled in processes such as filtration, drug infusion. FIG. 1 shows a partial schematic representation of a method for making drug and/or visualization agent loaded composite biodegradable materials or microparticles. A biodegradable photocrosslinkable precursor/s or condensation polymerization precursor/s, a non-crosslinked biodegradable polymer such as PLA or PLGA (A) are dissolved in the common organic solvent. The solution may also contain a drug/visualization agent at the desired concentration provided it can tolerate the precursor gelling/crosslinking conditions. The solution is converted into microdroplets or filled into a mold with microcavities of a desired shape and size (B). Effective crosslinking of precursor solution in the shape such as microdroplets (B) is then initiated using UV or visible light or other precursor crosslinking reactions; converting the solution droplets into organic solvent gel particles or crosslinked material swollen in the organic solvent wherein biodegradable polymer (A) and/or drug remains entrapped inside the crosslinked gel microparticle (C). C1 shows a picture of cylindrical organogel particles comprising crosslinked PEG-based macromonomer, PLGA as a non-crosslinked polymer. The gel is swollen in dimethyl sulfoxide (DMSO) as an exemplary organic solvent. C1 is made according to the methods described in this invention. The composite gel microparticles may be optionally exposed to drug solution under the effective diffusion conditions wherein the drug can diffuse inside the crosslinked microparticles and biodegradable polymer. This step may be useful if the drug cannot tolerate precursor crosslinking conditions such as exposure to high-intensity light. Diffusion conditions are chosen such that a substantial amount of the biodegradable polymer cannot diffuse out of the crosslinked network during the drug infusion/loading process. The organic solvent is removed and the non-crosslinked biodegradable polymer is solidified/precipitated in situ inside the crosslinked polymer structure entrapping the drug in the precipitated polymer and crosslinked microparticle. The microparticle with a precipitated biodegradable polymer (101) inside the crosslinked gel is schematically shown in D. D1 is similar to C1 wherein the solvent is partially removed which precipitates the PLGA inside the C1 (101). The precipitation leads to increased opacity of the particle D1 as compared to C1. D2 is the same as D1 wherein drug rifampin is infused into particles (in a crosslinked network as well as biodegradable polymer PLGA) via the solvent diffusion process described in this invention. The red color indicates the presence of infused rifampin in the particle. The entrapped drug is released in a sustained manner from the solidified PLGA polymer and crosslinked polymer upon implantation in the body.

FIG. 2 shows schematic structures of preferred organic solvent gel/hydrogel precursor compositions described in this invention. FIG. A shows a free radically crosslinkable precursor or macromonomer wherein the precursor has a central core (201) and unsaturated polymerizable groups at the end or at the side chain (202) of the central core. The polymerizable groups (202) are separated from the central core (201) via a biodegradable polymer block (203). FIG. B shows a macromonomer with 201 and 202 and no biodegradable blocks. This macromonomer (B) produces biostable crosslinked networks upon effective polymerization and crosslinking. Precursors shown in D and E form crosslinked networks when reacted and polymerized via condensation or step growth polymerization under effective polymerization conditions. D has a central core (201) and biodegradable blocks (203) but has 2 or more end or side chain electrophilic groups (204) between central core (201) and biodegradable blocks (204). Precursors in FIG. E also have central core (201), biodegradable blocks (203) and nucleophilic end groups (205) or side chain groups. The number of electrophilic groups in D and nucleophilic groups in E must be greater than two. The total number of nucleophilic and electrophilic groups (204 plus 205) must be equal to or greater than five to form an effective crosslinked network when reacted under effective crosslinking conditions. Either D or E must have biodegradable blocks (203) to form a biodegradable crosslinked polymer network.

FIG. 3 shows a partial schematic representation of illustrative chemical reactions involved in making composite biodegradable materials/microparticles using free radical polymerizable precursors. PEG based illustrative biodegradable macromonomers such as PEG-polylactate-acrylate macromonomer (A) and PLGA copolymer (B, illustrative NBP), thermal or photoinitiator, and optionally drugs are mixed in an organic solvent such as THF or DMSO to form a homogeneous solution. The degree of polymerization of PEG in A is shown as n and the degree of polymerization of lactide in A is shown as m. The degree of polymerization of lactide in B is shown as x and the degree of polymerization of glycolide in B is shown as y. The mixture is poured into mold cavities to provide a desired shape or sprayed to form microdroplets and then irradiated with light or heated to induce effective free radical polymerization and crosslinking of the macromonomer. The effective polymerization and crosslinking of the macromonomer converts the solution into organic solvent gel particle wherein biodegradable polymer PLGA remains entrapped in the organic solvent gel comprising crosslinked PEG-polylactate acrylate macromonomer and solvent. The gel particles are removed from the mold. Optionally, the particles may be washed with the organic solvent to remove initiator fragments, unreacted macromonomer and other undesirable reaction products and impurities. The solvent is removed via solvent exchange or evaporation and the PLGA in the particle is precipitated “in situ” inside the microparticle. The precipitated particle encapsulates the drug which is then released via diffusion and/or erosion of biodegradable polymer and crosslinked macromonomer. In some embodiments, if a biocompatible water miscible organic solvent and initiator is used, then the solvent swollen organic solvent gel microparticles can be directly injected in the human/animal body or tissue. The solvent is dispersed in the tissue, precipitating the PLGA in the composite microparticles. The precipitated PLGA encapsulates the drug and provides sustained drug delivery in the tissue.

The molecular weight of a precursor or macromonomer generally ranges from 400 Daltons to 2 million Daltons, preferably 1000 to 200000 Daltons. The organic solubility of preferred precursor compositions is generally greater than one percent, preferably greater than 5 percent with respect to weight of organic solvents. Preferred macromonomer comprises 3 main parts/sections linked covalently with each other, wherein the composition has a central core (201) that provides organic solvent solubility to the overall composition. The 203 block undergoes biodegradation when implanted in the human or animal body. Generally, each composition in FIG. 2A has at least one or more biodegradable blocks. The free radical polymerizable groups are generally at the end of the molecular side branch or at the terminal end of the molecule. Macromonomers with a central core and polymerizable groups (FIG. 2B) only result in biostable networks upon crosslinking. The central core 201 can be any polymer that assists in organic solvent solubility and preferably is biocompatible and biodegradable in nature. Examples of 201 include polyethylene glycol, polyethylene oxide, and polyethylene oxide-polypropylene block copolymers, PLGA, PLA and the like. The biodegradable block includes polylactones such as PLGA, polycarbonate such as polytrimethylene carbonates, polyamino acids, polyamino acids with amino acid sequences that can be broken down by enzymes such as protease or collagenase, hyaluronidase and the like; proteins like albumin, fibrinogen, collagen, gelatin, keratin, cellulose derivatives like cellulose sulphate and the like and their blends, copolymers and the like. The most preferred compositions are based on polyethylene glycol, polyethylene oxide and their random or block copolymers such as Pluronic® or Tetronic®, Reverse Pluronic and the like. The preferred biodegradable blocks are polylactones, polyhydroxyacids, polytrimethylene carbonates, polypeptides, polyamino acids, PEG-polylactones, poly(glycerol sebacate) and the like. PEG based derivatives also include but not limited to succinates, glutarates, adipates, sebacates, citrates, tartarates, and aspartates and the like and their copolymers or blends. Preferred compositions undergo biodegradation under physiological conditions found in the human or animal body and the biodegradation products are non-toxic that can be safely removed by the body. The polymerizable groups are acrylates, methacrylates, acrylamides, methacrylamide, fumarates, maleates, itaconates, and the like or their combinations in any proportions. Most preferred polymerizable groups have the ability to polymerize rapidly preferably within 30 minutes, even more preferably within 10 minutes and most preferably within 5 minutes. The preferred polymerizable groups include but not limited to: acrylic acid and methacrylic acid derivatives such as acrylates, methacrylates, acrylamide and methacrylamide and the like. FIG. 3A shows molecular structure of PEG based biodegradable macromonomer as described in FIG. 2A wherein PEG 35000 Daltons is a central core (201) which is extended with polylactate oligomer with a molecular weight of around 600 Daltons (203) and is terminated with acrylate end group (202). EXAMPLE 1A teaches one illustrative method to synthesize such a macromonomer. Part 1 uses PEG 35000 Daltons to initiate polymerization of dI-lactide. The ratio of lactide to PEG is used to control the degree of polymerization and stannous octoate is used as a polymerization catalyst. The hydroxyl end group of the resultant polymer is reacted with acryloyl chloride to produce acrylate terminated PEG-lactate-acrylate copolymer (EXAMPLE 1A, Part 2). Similarly, Jeffamine™ (molecular weight around 1900 Daltons) is reacted with dI-lactide (lactide to Jeffamine weight ratio approximately 2:1) and then terminated with acrylate end groups (JALA). Many macromonomers have been synthesized and characterized and several PEG based biodegradable and biostable macromonomers were evaluated for their ability to polymerize and crosslink in various organic solvents, preferably biocompatible water miscible organic solvents that can be safely injected in human or animal body (EXAMPLE 1). Only under certain conditions, macromonomers can be effectively polymerized and crosslinked to form OSGs. If those effective conditions are not met, then polymerization and crosslinking cannot be achieved. Certain conditions in which polymerization and crosslinking could not be achieved to form a crosslinked gel and these conditions include but not limited to: insufficient macromonomer concentration (generally below 2 percent), inability to form self-assembly in the organic solvent, inefficient free radical initiator, low initiator concentration, low initiator efficiency in initiating polymerization, presence of polymerization inhibitors such as oxygen or inhibitors added to improve shelf life, low polymerizibility of unsaturated groups in the macromonomer, insufficient light intensity and inappropriate wavelength (in case of photoinitiator), use of macromonomers with only one unsaturated group and the like. Under the conditions we evaluated, many compositions can be effectively polymerized and crosslinked with and without the presence of non-crosslinked biodegradable polymers in many organic solvents. Those skilled in the art of free radical polymerization chemistry understand that proper effective polymerization conditions can be determined for each macromonomer if such conditions exist. In using solvent-based free radical based polymerizations, care is taken to minimize the side reactions such as chain transfer reactions. Solvents that are prone to chain transfer reactions are not preferred in free radical based polymerization. Halogenated solvents such as chloroform, dichloromethane are generally not preferred due to their ability to covalently link to the crosslinked structure. Also, non-crosslinked biodegradable polymers such as polypeptide with thiol groups are generally not preferred because thiols groups are known to undergo chain transfer reactions. Several non-limiting effective crosslinking conditions are provided in Table 1 and these can be used along with other examples and cited literature as a guide to select appropriate effective polymerization conditions for macromonomer compositions disclosed in this invention.

TABLE 1
EFFECTIVE CROSSLINKING CONDITIONS FOR MAKING COMPOSITE PHOTOCROSSLINKED GELS
IN VARIOUS ORGANIC SOLVENTS WITH BIODEGRADABLE POLYMERS PHYSICALLY ENTRAPPED
IN THE PHOTOCROSSLINKED NETWORK. THE NON-CROSSLINKED BIODEGRADABLE POLYMER
(NBP) IS PRECIPITATED INSIDE THE CROSSLINKED POLYMER TO ENCAPSULATE A DRUG.

| | CENTRAL | | | | | |
|---|---|---|---|---|---|---|
| | CORE# (201) | | | | | |
| | MOLECULAR | MONOMER | | NBP | NBP | |
| | WEIGHT | CONC. | NBP | MOL. WT. | CON. | |
| MACROMONOMER | (G/MOL) | (%) | TYPE. | (G/MOL) | (%) | SOLVENT |
| PEG 3K urethane acrylate | 3000 | 30% | PLGA 50:50 | 45K-55K | 20% | DCM |
| (PEG3KUA) | | | | | | |
| PEG 3K urethane acrylate | 3000 | 40% | PLGA 50:50 | 45K-55K | 20% | DCM |
| (PEG3KUA) | | | | | | |
| PEG 6K urethane acrylate | 6000 | 10% | PLGA 50:50 | 45K-55K | 20% | DCM |
| (PEG6KUA) | | | | | | |
| PEG 6K urethane acrylate | 6000 | 20% | PLGA 50:50 | 45K-55K | 20% | DCM |
| (PEG6KUA) | | | | | | |
| PEG 10K urethane acrylate | 10000 | 40% | PLGA 50:50 | 45K-55K | 20% | DCM |
| (PEG10KUA) | | | | | | |
| PEG 10K urethane acrylate | 10000 | 30% | PLGA 50:50 | 45K-55K | 20% | DCM |
| (PEG10KUA) | | | | | | |
| PEG 10K urethane acrylate | 10000 | 10% | PLGA 50:50 | 45K-55K | 20% | DCM |
| (PEG10KUA) | | | | | | |
| PEG 10K urethane acrylate | 10000 | 10% | Jeffamine lactide. | 5900 | 20% | DCM |
| (PEG10KUA) | | | | | | |
| PEG 10K urethane acrylate | 10000 | 10% | PLGA 50:50 | 10K-15K | 10% | DCM |
| (PEG10KUA) | | | | | | |
| PEG 10K urethane acrylate | 10000 | 15% | PLGA 50:50 | 10K-15K | 10% | DCM |
| (PEG10KUA) | | | | | | |
| PEG 10K urethane acrylate | 10000 | 20% | PLGA 50:50 | 10K-15K | 10% | DCM |
| (PEG10KUA) | | | | | | |
| PEG 10K urethane acrylate | 10000 | 10% | PLGA 50:50 | 10K-15K | 20% | DCM |
| (PEG10KUA) | | | | | | |
| PEG 10K urethane acrylate | 10000 | 10% | PLGA 50:50 | 45K-55K | 20% | DMSO |
| (PEG10KUA) | | | | | | |
| PEG 10K urethane acrylate | 10000 | 10% | PLGA 50:50 | 45K-55K | 25% | DMSO |
| (PEG10KUA) | | | | | | |
| PEG 10K urethane acrylate | 10000 | 10% | PLGA 50:50 | 45K-55K | 30% | DMSO |
| (PEG10KUA) | | | | | | |
| Jeffamine- lactide urethane | PEG 1900 and | 10% | PLGA 50:50 | 45K-55K | 20% | DCM |
| acrylate (JL2KUA) | PLA4000 | | | | | |
| Jeffamine- lactide urethane | PEG 1900 and | 10% | PLGA 50:50 | 45K-55K | 20% | DMSO |
| acrylate (JL2KUA) | PLA4000 | | | | | |
| Jeffamine- lactide urethane | PEG 1900 and | 10% | PLGA 50:50 | 45K-55K | 25% | DMSO |
| acrylate (JL2KUA) | PLA4000 | | | | | |
| Jeffamine- lactide urethane | PEG 1900 and | 10% | PLGA 50:50 | 45K-55K | 30% | DMSO |
| acrylate (JL2KUA) | PLA4000 | | | | | |
| Pluronic F127 urethane | 12500 | 10% | PLGA 50:50 | 45K-55K | 20% | DMSO |
| acrylate (F127UA) | | | | | | |
| Pluronic F127 urethane | 12500 | 10% | PLGA 50:50 | 45K-55K | 25% | DMSO |
| acrylate (F127UA) | | | | | | |
| Pluronic F127 urethane | 12500 | 10% | PLGA 50:50 | 45K-55K | 30% | DMSO |
| acrylate (F127UA) | | | | | | |
| PEG 20K urethane acrylate | 20000 | 5% | PLGA 50:50 | 10K-15K | 20% | DCM |
| (PEG20KUA) | | | | | | |
| PEG 20K urethane acrylate | 20000 | 10% | PLGA 50:50 | 45K-55K | 20% | DMSO |
| (PEG20KUA) | | | | | | |
| PEG 20K urethane acrylate | 20000 | 10% | PLGA 50:50 | 10K-15K | 10% | DMSO |
| (PEG20KUA) | | | | | | |
| PEG 20K urethane acrylate | 20000 | 10% | PLGA 50:50 | 10K-15K | 20% | DMSO |
| (PEG20KUA) | | | | | | |
| PEG 20K urethane acrylate | 20000 | 10% | PLGA 50:50 | 10K-15K | 25% | DMSO |
| (PEG20KUA) | | | | | | |
| PEG 20K urethane acrylate | 20000 | 10% | PLGA 50:50 | 45K-55K | 20% | DMSO |
| (PEG20KUA) | | | | | | |
| PEG 20K urethane acrylate | 20000 | 10% | PLGA 50:50 | 10K-15K | 10% | DCM |
| (PEG20KUA) | | | | | | |
| PEG 20K urethane acrylate | 20000 | 10% | PLGA 50:50 | 10K-15K | 15% | DCM |
| (PEG20KUA) | | | | | | |
| PEG 20K urethane acrylate | 20000 | 10% | PLGA 50:50 | 10K-15K | 20% | DCM |
| (PEG20KUA) | | | | | | |
| PEG 20K urethane acrylate | 20000 | 10% | PLGA 50:50 | 45K-55K | 20% | DCM |
| (PEG20KUA) | | | | | | |

* Photoinitiator 2-hydroxy-4′-(2-hydroxyethoxy-2-methylpropiophenone, concentration around 0.2% relative to total solvent volume; exposed to 360 nm long UV light up to five minutes.
#Central core (201) is PEG in most cases. DCM is for dichloromethane, DMSO is for dimethyl sulfoxide and DW is for distilled water. 45K is 45000 and 10K is 10000. NBP is for non-crosslinked biodegradable polymer. Many compositions polymerized under 120 seconds.

Some compositions we tested gelled much faster if a small amount of comonomer such as n-vinyl pyrrolidone, acrylic acid, acrylamide, n-vinyl caprolactam and the like is used during OSG preparation. This invention is limited to those effective conditions which lead to effective polymerization and crosslinking of macromonomers. Generally, in the preferred embodiment, each composition must comprise at least a portion of macromonomer, preferably 2 percent to 99 percent but most preferably 5 to 90 percent, with two or more polymerizable bonds. A combination of macromonomers with at least one polymerizable bond may also be used, however, such mixture must have an effective amount of macromonomers with two or more polymerizable bonds for effective polymerization and crosslinking. The crosslinked density and/or molecular permeability is generally controlled by using a greater number of unsaturated polymerizable groups per molecule. Macromonomers with 2, 3, 4, 5, 6, 7, 8, 9 and more polymerizable double bonds per molecule produce gels with increased crosslinked density respectively. As the number of polymerizable groups increases in the macromonomer, it generally results in crosslinked polymer with higher crosslinked density provided molecular weight and other reaction conditions are held constant. In general, the higher crosslinked density results in low molecular permeability of the crosslinked network. A number of polymerizable bonds and PEG with a different molecular weight in the macromonomer in desirable proportion is used to obtain crosslinked materials with desired crosslinked density and molecular permeability. The biodegradation time of a crosslinked network is also controlled by the type of biodegradable blocks used and the crosslink density of the network. Generally, biodegradable blocks with

polyglycolate ester have a short degradation time (3 days to 40 days), with polylactate block degradation time varies from few weeks to several months and polycaprolactone blocks degradation time varies from few months to few years. However, copolymers of polyglycolate, polylactate, polycaprolactone and other polylactones and trimethylene carbonates in desirable proportions can have biodegradation time in between few days to few years. EXAMPLE 1 teaches the synthesis of several illustrative macromonomers. Preferred macromonomers with different PEG molecular weights, different biodegradable blocks and a different number of polymerizable groups per molecule are synthesized. Polymerization or copolymerization of one or more macromonomers can lead to crosslinked networks with different physical and chemical properties which include but are not limited to: organic solvent gels with various crosslinking density, degradation time, molecular permeability, mechanical properties. EXAMPLE 1 also teaches synthesis and crosslinking of macromonomers made from natural polymers such as gelatin, collagen, hyaluronic acid, cellulose derivatives and the like. The side functional groups such as hydroxy, acid, amine, thiol and the like in these natural biocompatible and biodegradable polymers are covalently attached with polymerizable groups such as acrylate or methacrylate and are polymerized in organic solvents like DMSO. Polymerization and crosslinking of some of these polymers/macromolecules derivatives result in to crosslinked materials that degrade by enzymatic pathway upon implantation. Some illustrative effective polymerization and crosslinking conditions are also provided in EXAMPLE 1. EXAMPLE 2 provides additional illustrative examples of organic solvent gels with entrapped non-crosslinked biodegradable polymers (NBP) like PLGA. We have taken advantage of polymerizibility in the organic solvent to form composite biodegradable polymers. In one illustrative embodiment, 50.0 mg PEG35KUA macromonomer is dissolved in 0.5 ml warm n-methyl pyrrolidinone followed by the addition of 10 microliters of initiator stock solution (ISS). 50 microliters of macromonomer solution is then exposed to long UV light (wavelength 360 nm). The solution forms a soft gel within 300 seconds indicating that the macromonomer can be effectively polymerized in organic solvent NMP. 0.3 ml of NMP macromonomer solution as above is mixed with 60 mg of polylactide-co-polyglycolide (PLGA, 50:50) copolymer, molecular weight between 10000 to 15000 Daltons, until complete dissolution. The organic (NMP) solution comprises macromonomer, photoinitiator and PLGA polymer as an exemplary non-crosslinked biodegradable polymer. 50 μl of NMP solution is poured into a silicone mold cavity and exposed to UV light to form a soft organogel in the mold cavity . The organogel has PLGA entrapped into a crosslinked PEG35KUA network. The gel is washed with water to remove NMP which results in precipitation of PLGA within the crosslinked gel.

In the illustrative embodiments, we have used three different molecular weight of PLGA, 10000 -15000 Daltons, 45000-50000 Daltons and 100000-150000 Daltons to entrap in the crosslinked gel resulting composite materials with different chemical and physical properties associated with the different molecular weights. We also used PEG-PLA block copolymer as one of the illustrative non-crosslinked water soluble biodegradable polymers (Jeffamine-lactate copolymer, JAL or JALA, EXAMPLE 1) with thermoreversible gelation property. This polymer has thermoreversible gelation properties when used at a 20-35 percent concentration range in water. The molecular weight of the non-crosslinked biodegradable polymer in the composite material may range from 1000 to 2 million, preferably 1500 to 200000 Daltons and even more preferably 2000 to 150000 Daltons. The preferred biodegradable polymer used is hydrophobic or substantially hydrophobic. It can also be hydrophilic with solubility greater than 1 g/100 g of solvent preferably greater than 5 g/100 g of solvent. The preferred NBP are synthetic polyesters or polycarbonates that may be endcapped with acetate ester, triiodobenzoic acid derivatives such as Iodixanol or fluorescent compound derivatives such as fluorescein sodium salt or eosin or any other biocompatible end-capping compound. The end-capping of groups prevents participation in the crosslinking process and optionally also provides additional functionality such as radio-opaque property or fluorescence or color. End groups also prevent the initiation of unwanted hydrolysis of the non-crosslinked biodegradable polymer such as PLGA during storage and processing. Synthetic biodegradable polymers with end groups such as hydroxyl groups, carboxylic acid groups and the like can also be used but are generally not preferred. The non-crosslinked biodegradable polymer (NBP) may be linear, branched or dendrimer in nature. Branched or highly branched or dendrimer are preferred because such polymers entangle with the crosslinked network and are slow in diffusing out during the drug infusion process. The non-crosslinked biodegradable polymer may be solid, semisolid, wax type, neat liquid or low melting solid (melting point below 70 degree C.) or physically crosslinked gel. The non-crosslinked biodegradable polymer may also have additional properties like thermoreversible gelation or pH-sensitive gelation and the like or may be present as a neat liquid or low melting solids. The non-crosslinked biodegradable polymer may be a polymer or macromolecule. The polymer may also be a random copolymer, block copolymer, dendrimer and homopolymer. The preferred biodegradable polymer include but not limited to: synthetic hydrophobic biodegradable polymers; polymers, dendrimers, copolymers or oligomers of glycolide, dI-lactide, D-lactide, L-lactide, caprolactone, poly(glycerol sebacate), dioxanone and trimethylene carbonate; biodegradable polyurethanes; biodegradable polyurethanes made by block copolymers of degradable polylactone such as polycaprolactone and polycarbonate such as poly(hexamethylene carbonate); tyrosine-derived polycarbonates, tyrosine-derived polyarylates; polyamides; polyesters such as polyester made from glycerol and sebacic acid ; polypeptides; polyhydroxyacids; polylactic acid; polyglycolic acid; polyanhydrides; and polylactones. Poly(dI-lactide-co-glycolide, 50:50); poly(dI-lactide-co-glycolide, 65:35); poly(dI-lactide-co-glycolide, 75:25), poly(dI-lactide-co-glycolide, 85:15), poly(dI-lactide-co-ε-caprolactone, 25:75), poly(dI-lactide-co-ε-caprolactone, 80:20) are preferred due to their commercial availability. Non-cross linked biodegradable polymers also include polyhydroxyalkanoates, which are polyesters produced by microorganisms including and not limited to poly(3-hydroxybutyrate), 3-hydroxyvalerate, 4-hydroxybutyrate, 3-hydroxyhexanoate, 3-hydroxyoctanoate. Hydrophilic polymers or their derivatives which are soluble in organic solvents, which include, but are not limited to, polyethylene glycol-polyhydroxy acid or polyethylene glycol-polylactone copolymers (PEG-PL copolymers); polyvinyl alcohol-co-polylactone copolymers; and derivatives of cellulose; collagen or modified collagen derivatives; gelatin; albumin; hyaluronic acid, alginate, chitosan and dextran. The percent of non-crosslinked biodegradable polymer (NBP) in the composite polymer ranges from five percent to 95 percent (relative to total composite polymer weight), preferably 7 to 90 percent and even more preferably 10 to 60 percent. The weight percent of PEG in the preferred composite particle ranges from 5 to 90 percent, preferably 10-80 percent. The drug loading in the composite microparticle ranges from 0.1 to 70 percent, preferably 1 percent to 60 percent and most preferably 5 to 50 percent relative to total weight of the composite material. The drug-loaded in NBP of the composite ranges from 3 to 50 percent relative to NBP weight. Drugs may be added before the effective crosslinking process or can be infused via a solvent diffusion process. The drug is released in a sustained manner by diffusion and/or biodegradation/bioerosion from the composite materials. Drugs may be released between 1 day to 2 years, preferably 2 days to one year from the composite material. The preferred drug release profile is zero order in nature or close to zero order in nature. Drugs or bioactive compounds that have thiol or halogenated groups or functional groups that are prone to chain transfer reactions can be encapsulated via diffusion process rather than during the polymerization process to avoid effects of crosslinking conditions. Other chemical reactions such as Michael addition reactions that can occur can also be avoided/minimized by choosing proper reaction conditions when using free radical based polymerizations.

Organic solvent crosslinked gels with NBP can also be formed using condensation polymerization reactions. EXAMPLE 3 provides some illustrative embodiments for making organic solvent gels using condensation polymerization reactions. Preferred networks are formed using precursor compositions described schematically in FIG. 2D and 2E. The preferred compositions in 2D and 2E have a central core extended with biodegradable blocks and terminated with 2 or more functional reactive groups such as electrophilic groups (204). The other precursor shown in FIG. 2E also has a central core (201) extended with (203) biodegradable block and terminated with functional reactive groups such as nucleophilic groups (205). The total number of functional reactive groups on D and E (groups in 204+205) must be greater than or equal five to form an effective crosslinked network if reacted in molar equivalent quantities or substantially in molar equivalent quantities and under effective crosslinking conditions. A detailed description of 201 and 203 blocks was provided in an earlier section. Functional groups can be at the terminal end as in standard PEG or they can be at the side branch such as amine, thiol, hydroxy, carboxylic acid groups on proteins like albumin, gelatin or collagen. When functional groups such as 204 and 205 react with each other under effective reaction conditions, it leads to the formation of a crosslinked network. When electrophilic groups are isocyanates, and nucleophilic groups are amines or anilines, then crosslinked bond comprises urea; when electrophilic groups are acyl halide, and nucleophilic groups are alcohol or phenol, then covalent bonds formed are ester; when electrophilic groups are epoxides, and nucleophilic groups are thiols, then covalent bonds formed are thio-ethers; when electrophilic groups are epoxides, and nucleophilic groups are alcohols or phenols, then covalent bonds formed are ethers; when electrophilic groups are acyl nitriles, and nucleophilic groups are alcohol or phenol, then covalent bonds are of ester; when electrophilic groups are acyl halide, and nucleophilic groups are amine or aniline and the covalent bonds are of carboxamides; when electrophilic groups are aldehyde or ketones, and nucleophilic groups are hydrazines, then covalent bonds formed are of hydrazones; when electrophilic groups are hydrazides, and nucleophilic groups are carboxylic acids, then covalent bonds formed are of hydrazines; when electrophilic groups are isothiocyanates, and nucleophilic groups are amines or aniline, then covalent bonds formed are of thioureas; when electrophilic groups are alkyl halide, and nucleophilic groups are amine or aniline, then covalent bonds formed are of alkyl amines; when electrophilic groups are aldehyde or ketone, and nucleophilic groups are hydroxyl-amines, then covalent bonds formed are of oximes; when electrophilic groups are alkyl halides, and nucleophilic groups are thiols, then covalent bonds formed are of thioethers; when electrophilic groups are alkyl halide, and nucleophilic groups are carboxylic acid, then covalent bonds formed are of esters; when electrophilic groups are aldehyde, and nucleophilic groups are amine or aniline, then covalent bonds formed are of imines; when electrophilic groups are activated esters, the nucleophiles can be either amines or anilines and covalent bonds formed are of carboxamides; when electrophilic groups are acyl nitrile, and nucleophilic groups are aniline or amine, then covalent bonds formed are of carboxamides. The most preferred nucleophilic groups include but are not limited to: primary amine, thiol, hydroxyl and the like. Most preferred electrophilic groups include but are not limited to: succinimidyl carbonate, succinimidyl glutarate, succinimidyl adipate, succinimidyl sebacate, succinimidyl acetate, succinimidyl succinamide, acrylate, methacrylate, maleimide, epoxy, aldehyde, isocyanate, thioisocyanate, acid chloride, acid anhydride, vinyl sulfone. In some cases, if the reactive groups are not sufficiently reactive under mild reaction conditions (room temperature, atmospheric pressure and low concentration) then an activated derivative of the reactive group is used. For example, the reaction between the carboxylic acid group and hydroxy or primary amine group does not occur under mild conditions. In such situations, a catalyst that promotes the reaction under mild conditions is used. A zero-length catalyst such as carbodiimide based catalysts like N,N′-Dicyclohexylcarbodiimide (DCC) or 1-Ethyl-3-(3-dimethylaminopropyl)carbodiimide (EDC) that promote esterification or amide formation in presence of tertiary amines and catalyst such as n-hydroxysuccinimide or n-hydroxysulfosuccinimide and the like may also be used. Enzymes such as lipase that can work under mild conditions in organic solvents or in buffered aqueous solutions and can promote crosslinking/esterification (acid and alcohol) or transesterification may also be used. Enzymes that promote the reaction between an acid and primary amine are preferred. Preferred crosslinking reactions occur under mild conditions between activated acids, isocyanate or epoxy groups as preferred electrophilic groups. Amine, thiol or hydroxyl groups are preferred nucleophilic groups. Reactive groups that do not form side reaction products such as carbon dioxide, water, n-hydroxysuccinimide and the like are preferred in some applications. Reactions between epoxy and amine, isocyanate and amine, acrylate or methacrylate and thiol and the like that do not form side products are preferred. In the preferred embodiments, one of the two or both precursors (FIG. 2D or E) could be polymeric or macromonomeric in nature. The molecular weight of polymeric precursors ranges from 2000 to 3 million Daltons. Both the precursors could be polymeric in nature and the polymer could be linear, branched, with 3, 4, 5, 6, 7, 8, 9, 10, 11, 12, 13, 14, 15, 16, 20, 24 or more branches or could also be dendrimer. The other precursors could be a small molecular compound with molecular weight starting from 200 to 2000 Daltons. A combination of one high molecular weight precursor (from 2000 to 3 million Daltons) and one low molecular precursor (up to 5000 Daltons) is most preferred. Either of the precursors or both shown in 2D or 2E can have biodegradable sections to form a biodegradable crosslinked network. The precursors must be reacted under effective reaction conditions to form a crosslinked network. Such conditions include but not limited to: use of molar equivalent quantities of precursors that can lead to effective condensation polymerization and crosslinking, time, temperature, pressure, use of catalyst/s and cocatalyst/s in a desired quantity, solvents, the reactivity of functional groups under the conditions used, and the like. Those skilled in the art understand that effective crosslinking conditions must be determined experimentally for a given composition before using it as described in this invention. Under effective crosslinking conditions, the precursor that reacts within 24 hours, preferably within 1 hour, most preferably within 10 minutes to form a crosslinked network is preferred. Some illustrative conditions and compositions are provided in this invention, but those skilled in the art understand that many variations can be made to those compositions and reaction conditions to obtain effective crosslinking and such compositions are considered as a part of this invention. FIG. 4 shows a crosslinked network that is formed by reacting PEG-glutarate-NHS ester (model precursor with NHS ester as an electrophilic group) and PEG-tetramine (model precursor with amine as a nucleophilic group). PEG in the PEG-glutarate-NHS ester and PEG in the tetramine can be considered as the central core (FIG. 2, 201). The two glutarate ester bonds in the PEG-glutarate-NHS ester is a biodegradable section (FIG. 2, 203) and two NHS esters (FIG. 2, 204) can serve as electrophilic reactive groups. The four amines in the PEG tetramine serve as nucleophilic groups (FIG. 2, 205). When PEG-glutarate-NHS ester and PEG tetramine are reacted in molar equivalent concentration (equal number of NHS and amine groups) in organic solvents such as toluene or tetrahydrofuran, the reaction leads to condensation polymerization (total reactive groups are 6, four amines and two NHS) which is greater than 5 and therefore leads to the formation of the crosslinked network. FIG. 4 shows a partial schematic representation of illustrative chemical reactions involved in making composite biodegradable polymers using condensation polymerization reaction. PEG based precursors such as PEG-glutarate-NHS ester, PEG tetramine and a non-crosslinked hydrophobic polymer such as PLGA with non-reactive end groups, organic solvent and optionally a drug/visualization agent with non-reactive (towards precursors) chemical functional groups are mixed in an organic solvent like THF to form a homogeneous solution. The mixture is then poured into mold cavities before effective crosslinking and gelation to provide a desired shape to the precursor solution. Under effective crosslinking conditions, the precursors in the mold cavity undergo effective condensation polymerization and crosslinking to form organic solvent gel microparticles in which hydrophobic polymer PLGA remains entrapped in the crosslinked polymer. The gel particles which take the shape of the mold cavity are removed from the mold. The gel particles may be optionally washed with organic solvents to remove unwanted crosslinking reaction products such as n-hydroxysuccinimide and low molecular weight unreacted precursors. The washing conditions (solvent type and amount, incubation time, temperature, pressure and the like) are chosen such that a substantial amount of NBP in the crosslinked polymer is retained in the crosslinked polymer. The solvent is removed and the PLGA in the particle is precipitated “in situ” inside the crosslinked microparticle. The precipitated particle encapsulates the drug which is then released via diffusion and/or erosion of the biodegradable polymer and crosslinked gel. In some cases, if a biocompatible water miscible solvent such as NMP or DMSO is used for drug infusion, the solvent swollen microparticles can be directly injected in the body or tissue. After injection in the tissue, the solvent is dispersed in the tissue, precipitating the PLGA in the composite microparticles. The precipitated PLGA encapsulates the drug and provides sustained drug delivery in the tissue. EXAMPLE 3 shows several illustrative embodiments of crosslinked composite materials made using condensation polymerization reaction and entrapping NBP such as PLGA. Those skilled in the art will recognize that many changes can be made to prepare a crosslinked composite network with different properties such as made with different reactive groups, different crosslinked densities, different degradation times, different PEG molecular weights, different degradable blocks, different drug loadings, different non-crosslinked biodegradable polymers and the like. In another embodiment, a hydroxyl-terminated polycaprolactone (3 branched) is made by reaction of trimethylolpropane triol and caprolactone using stannous octoate as a catalyst. This hydroxyl terminated polymer is then reacted with PEG-glutarate with acid terminated groups using an organic solvent to form an OSG. The reaction between carboxylic acid and hydroxyl group (used in molar equivalent concentrations) is catalyzed by DCC and tertiary amine to produce a crosslinked network. The polycaprolactone and glutarate ester both provide biodegradability to the crosslinked network. The crosslinked density of the network is controlled by the molecular weight of precursors especially of central core 201, a number of functional reactive groups involved in polymerization (total reactive groups 5, 6, 7, 8, 9, 10, 11, 12, 13, 14, 15, 16 or higher) may be used. Higher density crosslinked networks generally have lower molecular permeability. Functional polymers like gelatin or collagen or synthetic polypeptide polymers with side chains having functional groups like hydroxyl, primary amine, carboxylic acid and thiol that may be crosslinked using a crosslinker like glutaraldehyde, hexamethylene diisocyanate, PEG terminated with epoxy or n-hydroxysuccinimide groups and the like. The functional groups may also be reacted with itself (interchain crosslinking) when exposed with effective crosslinking conditions such as exposure to catalysts like EDC and DCC in presence of tertiary amine and cocatalysts like n-hydroxysuccinimide. Gelatin can be crosslinked (intermolecular crosslinking) using DCC or EDC in presence of n-hydroxysuccinimide as cocatalyst. Crosslinked structures produced by condensation reaction are biodegradable when biodegradable bonds/sections present between the crosslinks. The nature of biodegradable bonds and crosslinked density controls in vivo degradation time of the crosslinked gels. For example, PEG based crosslinked gels with succinate, glutarate, adipate and sebacate will have longer in vivo degradation time respectively. PEG based crosslinked gels with succinate as a biodegradable section or bonds will degrade within 3 to 20 days, with glutarate will degrade within 15 to 60 days, adipate and sebacate gels will take even longer degradation time (assuming that crosslinked density is same for comparison purpose). By controlling the crosslink density and biodegradability of ester bonds, crosslinked gels with biodegradation time from a few days to 2 years may be formulated and used. Different NBP polymers preferably with non-reactive end groups (end-capped) with different molecular weights and chemical composition can be incorporated during composite preparation. For a detailed description of NBP, please refer to the specification regarding composite materials prepared using free radical polymerization. In one illustrative embodiment, the non-crosslinked biodegradable polymer used in making composite materials has additional properties such as thermosensitive gelation or pH sensitive gelation. In one illustrative embodiment (EXAMPLE 3D), Jeffamine-lactide copolymer (JAL) with hydroxyl end groups is used as a model thermosensitive biodegradable polymer. The copolymer displays a thermoreversible gel property in aqueous solutions when subjected to body temperature (37 degree C. at concentration 10-30 percent). This copolymer is entrapped in the crosslinked network formed by the reaction of PEG10K4ARM tetramine PEG10K4ARM glutarate NHS. The network may be formed in cold aqueous solution (0-10 degree C.) such as PBS where JAL is soluble or in organic solvents such as DMSO. It is understood that many types of biodegradable thermosensitive polymers can be incorporated to make composite materials and these include but not limited to are: Pluronic or PEO-PPO copolymers; reverse Pluronic/s or Tetronic/s; polyacrylamides such as poly-isopropyl acrylamide and their copolymers; gelatin (various grades); chitosan based compositions and its derivatives, cellulose derivatives, various PEG-polylactone copolymers, PEG-PLA, PEG-PLGA, PEG-polyhydroxy copolymers, and the like. The composite as described above in the form of microparticles or microspheres is especially useful for injectable drug delivery systems because the crosslinked network provides additional mechanical reinforcement to the thermosensitive material.

In another illustrative embodiment, a neat liquid or low melting solid (solid with a melting point less than 56 degree C.) is used as a non-crosslinked biodegradable polymer in making composite materials or microparticles. In one illustrative example (EXAMPLE 3D) sucrose acetate isobutyrate (SAB) is used as an illustrative biodegradable non-polymeric liquid carrier. The SAB is liquid at ambient or body temperature and is known to be a biocompatible biodegradable drug carrier. SAB is mixed prior to crosslinking of PEG10K4ARM tetramine and PEG10K4ARM glutarate NHS ester. Upon mixing of precursors, the crosslinked gel is formed by the reaction of PEG10K4ARM tetramine and PEG10K4ARM glutarate NHS ester. The SAB remains entrapped in the polymer. Both polymeric and non-polymeric liquid carriers may be included in the crosslinked polymers which include but not limited to: biocompatible organic solvents like DMSO, PEG, PEG endcapped with different functional groups, polymeric liquid like low molecular weight PLGA, ethyl lactate, vitamin E or its derivatives, vitamin E acetate, liquid polylactone or polyhydroxy polymers or copolymers or copolymers, PEG-polylactone copolymers, PEO-PPO-PEO polylactone copolymers, sucrose acetate, natural or synthetic biodegradable oils and fats, fatty acids, fatty alcohols, oleic acid and its derivatives and the like.

The composite materials disclosed in this invention can be used in many biomedical applications. The organogels can be used to make medical implants preferably biodegradable implants with drugs. Organogel implants with cylindrical shape is most preferred in many medical application. The preferred diameter of the implant may range from 0.5 microns to 50 mm, preferably 0.8 microns to 20 mm, most preferably 1 microns to 10 mm. In another application (EXAMPLE 7), an organic solution of precursors is used to coat expandable devices such as various types of stents and stent grafts (coronary stents, peripheral vascular stents, carotid stents and the like) and vena cava filters. The stents may be coated with biostable composite or biodegradable composite and the coating may also contain drugs, preferably anti-restenosis drugs like paclitaxel, everolimus and the like. In one illustrative example, a Bard E-Luminexx stent (product code ZBM06040, 6 mm diameter and 40 mm length) is spray coated with THF solution macromonomer 35KL5A2, photoinitiator, PLGA (50:50 molecular weight 15000-30000 Daltons and bupivacaine base. After polymerization and crosslinking with 360 nm light exposure, solvent is removed by air drying and then under vacuum. The PLGA precipitates inside the crosslinked polymer coating entrapping the drug which is released in a sustained manner over a period of time.

The organic solution of precursors is used to coat biostable and biodegradable sutures to provide the sutures with antimicrobial or other therapeutic properties. Some of the crosslinked compositions reported in this invention are elastomeric in nature and can be potentially useful in coating application. The elastomeric composition can be hydrophobic or hydrophilic. The OSG based composite material compositions can be especially useful on PGA and gut suture based suture threads because these materials can tolerate exposure to commonly used organic solvents such as THF, dichloromethane, acetone and the like. Such compositions will also be useful for coating on medical devices such as tissue based surgical patch or hernia patch. In one illustrative embodiment, high molecular weight PLGA polymer, macromonomer (35KL5A2 that forms elastomeric crosslinked polymer) photoinitiator and drug are dissolved in the organic solvent like DMSO or THF and extruded from an electrospinning apparatus to form fibers or nanofibers. The fibers may be further processed using a process such as weaving, twisting or knitting if needed. The nanofibers are then exposed to light to crosslink the 35KL5A2 to form a composite material. Crosslinking can be done before or after weaving, twisting or knitting.

Some composite biodegradable hydrogel materials comprising hydrophobic NBP reduce their tendency to absorb and swell water upon implantation. Such materials may be used to control excessive swelling after implantation. In one illustrative embodiment, PEG10Kurethane diacrylate and PEG10Kurethane diacrylate with PLGA based organic solvent gels are prepared and the gels with PLGA absorbs 40-60 percent less water than its control (PEG10Kurethane diacrylate without PLGA). A similar observation is noted for F127urethane acrylate-based gels. These and other data indicated that PLGA or the presence of hydrophobic biodegradable polymer in the composite materials absorb significantly less water than its counterparts. The amount of water can be controlled by properties of the polymer such as type of hydrophobic polymer (polyglycolate or polylactate polymer and the like), molecular weight of polymer, percent crystallinity, the nature of end groups, and its concentration in the composite material and the like.

The composite materials described in this invention can be especially useful for making microneedle array based devices for drug delivery. In one illustrative embodiment (EXAMPLE 6), a drug delivery microneedle array is fabricated from a composite material comprising crosslinked polymer and non-crosslinked biodegradable polymer (NBP). In the preferred embodiment, the array is made from an organic solvent gel. Briefly, a silicone rubber based Mpatch™ Microneedle array template mold is procured from Micropoint Technologies Pte Ltd. (Singapore). The mold had the following characteristics: 20 mm diameter and 4 mm height. 10 by 10 microneedle array holes, 700 microns cavity height (square pyramid shaped cavities) with 200 by 200 microns base, 500 microns pitch, the distance between each needle is 500 microns (center to center). PEG35KUA macromonomer was used as an exemplary precursor and polylactide-co-polyglycolide (PLGA, 50:50) copolymer with a molecular weight between 10000 to 15000 Daltons as an illustrative non-crosslinked biodegradable polymer. The precursors along with NBP, photoinitiator and optionally with a drug are dissolved in DMSO (an illustrative organic solvent) to make a homogeneous solution. The mold cavities are filled with the macromonomer solution, centrifuged for uniform filling and for removal of dissolved gases and then exposed to long UV light (360 nm) up to 5 minutes to form organic solvent gel crosslinked materials wherein the crosslinked PEG35KUA and PLGA is swollen in organic solvent DMSO. The solvent was removed by air/vacuum drying and the formed needles are attached to pressure sensitive adhesive tape (needle base attached to adhesive tape) and lifted from the mold. The composite microneedle array thus formed has a sharp edge for piercing on the distal end for tissue penetration and the proximal side is attached to the adhesive tape. The array comprises biodegradable crosslinked material with PLGA polymer entrapped in the crosslinked material wherein PLGA is precipitated inside the needle due to solvent removal (FIG. 6E). The PLGA and crosslinked network either alone or with combination provide sustained drug delivery properties. The high molecular weight nature of crosslinked material along with PLGA (preferably medium to high molecular weight) provides mechanical strength to the array which helps in skin/tissue insertion. This method also eliminates the use of injection molding and other polymer processing methods that need high temperatures/pressures in array fabrication. Reactive monomers may be replaced with organic solvents if needed. The needles may be loaded with drug/s and/or visualization agent/s prior to polymerization/crosslinking or after the array is made. In one embodiment, a neat liquid precursor without solvent (poly(glycerol sebacate)-methacrylate) is used to make an array as described above. The array device (a combination of array needles attached to the adhesive tape) is pressed onto the skin to insert the sharp needles in the skin completely and the adhesive tape is detached from the skin leaving the needle array inside the skin for therapeutic use. For additional description of array needles (needle shape, size, number of needles and the like) and the device, please refer to US Patent Application 20190046479 and related art, cited references therein, cited herein for reference only. The visualization agent such as colored/fluorescent compound in the array, preferably at the base of the array can assist in visualization of the array during implantation. In another embodiment, PEG1OKUA and PLGA (50:50, 10000-15000 Daltons) is dissolved in dichloromethane along with a photoinitiator. The solution is filled in the Mpatch™ Microneedle array template mold and centrifuged for 5 minutes, excess solution is wiped off from the mold surface and then exposed to long UV light for 5 minutes. The solution is converted into the gel with PLGA. The solvent is removed by air/vacuum drying and the array is removed from the mold. Alternatively, the polymerization can be done in solid state, in a frozen state or in the solution state as described elsewhere in this invention. In another embodiment, a control microneedle array is made using the same method as above except without PLGA and used as a control. The difference between control and composite material is used to optimize the type of NBP used in the array for controlled drug delivery application. FIG. 6E shows a section of a photograph of a microneedle array prepared according to one of the illustrative embodiments. The array needles are made using composite materials comprising crosslinked PEG based polymer and PLGA as a non-crosslinked biodegradable polymer entrapped in the needle material and is precipitated in situ inside the particle. The pyramidal based sharp needles and their opaqueness is due to the presence of precipitated PLGA can be clearly seen. Prior to solvent drying the needles shown in the photograph are semi-transparent in the organic solvent gel state. Upon solvent removal, the needle becomes opaque due to the precipitation of PLGA inside the needle. In some embodiments, the casting can also be done to make two layered microneedles wherein the base of microneedle has colored or fluorescent compound as a visualization agent and the rest of the needle has drug loaded composition (similar to schematically shown in FIG. 7E). The method of making layered microparticles is discussed in a separate section. When the colored microneedle array is inserted in the skin tissue, its base can be visualized with an unaided or aided human eye due to the colored/fluorescent nature of the needle base. Solid-state or frozen-state polymerization techniques disclosed in this invention can also be used to make a microneedle array. The crosslinked material is then attached to the adhesive backup material to make an implantable array device. In another embodiment, condensation polymerization precursors comprising a non-crosslinked biodegradable polymer and organic solvent or aqueous solution with optionally a drug/visualization agent are filled in the silicone mold array cavities as above and held in the mold until effective condensation polymerization and crosslinking of the precursors via a chemical reaction between nucleophilic and electrophilic groups of the precursors is achieved. The organogel needle comprising crosslinked material and the non-crosslinked biodegradable polymer is removed and the solvent is removed by drying or solvent exchange. The composite microneedle array formed has crosslinked biodegradable material comprising non-crosslinked biodegradable polymer precipitated inside the needle body. Many examples of condensation polymerization are described in this invention. Microneedle arrays also can be made using two-photon polymerization techniques or 3-D stereolithography techniques using composite materials described in this invention. Stereolithography is a well-known method for making 3 dimensional objects via photopolymerization process. In this process, a 3 dimensional object is first designed using computer aided design software such as Solidworks®. The 3 dimensional object is then broken into several 2 dimensional slices using a computer program. One slice of the object is scanned using a UV or visible light laser beam on the liquid photopolymerizable resin. The scanning/contact of the laser beam with the photopolymerizable resin almost instantaneously polymerizes and solidifies the resin. The object is created by scanning successive slices on top of each other to make a 3 dimensional object. The depth of laser beam is controlled by photoinitiator concentration as well as special additives designed to reduce laser beam penetration without affecting photopolymerization reaction. In projection stereolithography (ANYCUBIC Photon S 3D Printer, model Photon S, an illustrative 3D projection printer) instead of scanning a laser beam, an entire 2 dimensional image is projected on the liquid resin or precursor macromonomer solution with photoinitiator and drug. A digital light processing chip (DLP) is used to make an image of the high intensity light which is then projected on the liquid resin. Successive projection of several 2 dimensional images creates a 3 dimensional object. Projection stereolithography is believed to reduce the time required to make 3 dimensional objects significantly. The projections stereolithography is especially useful to microdevices such as microneedle arrays and ophthalmic/punctal implants described in this invention. The compositions described in this invention can be used make microneedle arrays, ophthalmic implants and other devices disclosed in this invention. Those skilled in the art can recognize that modifications can be made to those examples to make a biodegradable composite array comprising biodegradable crosslinked materials.

In one illustrative embodiment, an implantable array with a large amount of rapidly water dissolvable material such sugar or sodium chloride along with drug is made. The rapidly dissolvable cargo (salt and drug) from the array is designed to provide quick release of drug in the tissue and the crosslinked biodegradable polymer optionally with NBP as an additive provides improved mechanical properties needed for skin insertion without breakage. Rapid dissolution and mechanical properties functions are separated in this array device. Briefly, the precursor such as biodegradable macromonomer along with photoinitiator and organic solvent are mixed with drug and inorganic water soluble salt such as sodium chloride to form a suspension. The preferred size of the salt particles is smaller than 100 microns, preferably smaller than 20 microns even more preferably smaller than the needle sharpest point/edge used for skin insertion. The suspension is effectively polymerized in the microcavity array mold as described in Example 6B and solvent is removed. The inorganic salt entrapped in the needle dissolves quickly in the tissue upon insertion. The amount of salt in the array varies from 5 percent to 500 percent, preferably 10 to 200 percent relative to the weight of the macromonomer used. Sodium chloride is used as an illustrative additive for rapid dissolution. Other biocompatible additives such as other inorganic or organic salts, sugars and polymers can also be added and these include but not limited to sodium iodide, sodium bicarbonate, citric acid or its salts, magnesium chloride, calcium sulphate, calcium carbonate, maltose, galactose, sucrose, mannitol, trehalose, dextrin, xylitol, hyaluronic acid, dextran, polyvinyl pyrrolidone, polyvinyl alcohols, cellulose derivatives such as carboxymethyl cellulose, carboxymethylpropyl cellulose, cellulose sulphate, gelatin, collagen, fibrinogen and the like. In some illustrative embodiments, aqueous solutions are used instead of organic solvent to make rapidly dissolvable cargo in the needle. In some embodiments, aqueous precursor solution can be polymerized in presence of drug/visualization encapsulated microspheres or microparticles to make microneedle array devices. The microneedle array needles have crosslinked biodegradable polymers with drug encapsulated microspheres. Upon insertion in the skin tissue, the microspheres provide sustained drug release from 24 hours to several months depending on the drug encapsulated microsphere composition used. The use of drug encapsulated particles is especially useful if the drug cannot tolerate crosslinking conditions such as ultraviolet or visible light exposure and highly reactive functional groups in the condensation polymerization precursor. The encapsulation shields the drug/visualization agent from precursor polymerization harmful effects. Preferred size of the drug encapsulated microspheres is smaller than 100 microns, preferably smaller than 20 microns even more preferably smaller than the needle point/edge used for skin insertion/penetration. The drug loading in the microspheres may range from 1 percent to 40 percent, preferably 5 percent to 30 percent relative to the microencapsulation matrix/polymer used. Preferably the drug loaded microspheres used have a biodegradable polymer as an encapsulation matrix. The preferred biodegradable polymers used in microspheres are polymers or copolymers or blends of polyesters such as polyhydroxy acid or polylactones. The most preferred biodegradable polymer is PLGA.

In another embodiment, a solid state or frozen state polymerization is used for making microneedle arrays. Briefly, the precursor along with photoinitiator is mixed with an organic solvent or water based solution, degassed and poured in the array mold. The mold is then cooled to freeze the precursor solution and irradiated with light to effectively polymerize and crosslink the precursor. The crosslinked composition is removed from the mold and processed to make a microneedle array device. In some illustrative embodiments, a precursor layer without drug is polymerized on top of the needle which provides a base layer for the array device (FIG. 7G, 713). The base layer improves handling of the array after fabrication. The backup layer may also be reinforced with fibers such cotton, polyester, nylon or silk fibers. The fibers used could be knitted, woven or twisted. In one illustrative embodiment, a cheesecloth Grade 50 is used as a reinforcement of the backup layer. The cloth is applied on top of array mold, precursor solution is applied and polymerized. The base layer of the device is attached to a pressure adhesive tape which can help in attachment and removal of the device from the skin tissue. The methods and compositions for making multilayered compositions disclosed in this invention can also be used to make multilayered needles (FIG. 7E). In another embodiment, dry precursors along with drugs are made first by removal of solvent and then the dry precursor powder is pressed in the mold to form a needle shape. The molded green needle is effectively polymerized and crosslinked in solid state or dry state to improve its mechanical properties and then used in microneedle array formation.

Some of the compositions of organogels described in this invention are elastomeric in nature. In one illustrative embodiment (Example 18), PEG35UA macromonomer along with PLGA and photoinitiator is photopolymerized in DMSO to produce an elastomeric hydrogel. FIG. 6F shows an exemplary elastic composite organogel comprising crosslinked PEG based polymer with PLGA as non-crosslinked biodegradable polymer swollen in organic solvent (DMSO). The organogel is substantially transparent in nature. FIG. 6G shows implant 6F in stretched condition without breakage showing its elastic nature. FIG. 6H shows implant 6F in stretched condition with solvent partially removed and the PLGA in the implant is precipitated in the stretched implant locking its stretched shape. The precipitated polymer makes the implant 6H opaque. The illustrative organogel implant is not a loose gel but a gel that has well defined shape and size which can be manipulated without breaking. The organogel implant is elastomeric in nature and can be stretched to approximately doubling its size without breakage. The solvent is removed from the swollen stretched gel to precipitate the PLGA in the gel (FIG. 6H) and lock its stretched shape. The same implant can be loaded with drug/visualization agent/s during polymerization or after polymerization via solvent diffusion as discussed in this invention. The above embodiment is for illustration only. Many OSG and composite compositions disclosed in this invention are elastomeric in nature. Such compositions can be stretched from its original length to 10 percent or more, preferably 20 percent without breaking. Some hydrogel/organogel crosslinked compositions comprising PEG with a molecular weight greater than 10000 g/mole, preferably greater than 35000 g/mole, even more preferably 100000 g/mole can be stretched from 10 to 400 percent of its original length. The elastomeric organogel compositions disclosed herein may be biostable or biodegradable.

Composite Microparticles

The composite materials described above can be formed into microparticles or microspheres suitable for injectable drug delivery systems and other medical and non-medical applications. FIG. 1 shows a partial schematic representation of a method for making composite drug and/or visualization agent loaded biodegradable composite microparticles. The composite particles can have various 3-dimensional shapes which include but are not limited to: cylinders, cubes, cuboids, cones, spheres, rectangular prisms, triangular prisms, hexagonal prisms, square pyramids, rectangular pyramid, triangular pyramid, hexagonal pyramid, torus and the like. Spherical or cylindrical shapes are preferred. The shapes could be symmetrical, non-symmetrical or irregular or their combination in any proportion. FIGS. 1, 5, 6, 7, 11, 12 and 15 show various ways where composite particles could be made using methods described in this invention. The size/volume of the composite particle may range from 1.5 ml to 0.5 picoliters, most preferably from 0.1 ml to 1 picoliter, even most preferably around 10000 picolitres to 5 picolitres. Preferred particles can have average diameters ranging from 0.5 microns to 3000 microns, and height from 50 mm to 1 micron. In some compositions, a mixture of particles with various sizes and/or shapes are used. This mixture is created either intentionally by mixing particles of various sizes in any proportion or it may be the result of a manufacturing process parameters used in making such particles.

The composite gel microparticles may be optionally exposed to drug solution in organic solvents or aqueous solutions under the effective diffusion conditions wherein drug can diffuse inside the crosslinked microparticles and be entrapped by the biodegradable polymer such as PLGA inside the particle. This step may be useful if the drug cannot tolerate crosslinking conditions such as exposure to high intensity light or the drug has functional groups that interfere in condensation polymerization reactions involving electrophilic and nucleophilic groups. Diffusion conditions are chosen such that a substantial amount of non-crosslinked biodegradable polymers (NBP, PLGA) cannot diffuse out of the crosslinked network during the drug loading process. The solvent is removed and a non-crosslinked biodegradable polymer is solidified/precipitated inside the crosslinked polymer structure entrapping the drug in the precipitated and/or crosslinked microparticle. The microparticle with precipitated polymer inside the crosslinked gel is schematically shown as FIG. 1D and FIG. 7A. The entrapped drug is released in a sustained manner from the solidified PLGA polymer and crosslinked polymer upon implantation in the body. Both the crosslinked polymer as well as non-crosslinked biodegradable polymer control the release profile of the drug.

EXAMPLE 5 shows illustrative embodiments wherein coacervation and microfluidic apparatus is used to make composite microparticles/microspheres. Droplets can also be formed by suspending or emulsifying the organic solution composition using a medium such as silicone oil, vegetable oil, mineral oil, aqueous solution and the like in which precursor composition is insoluble. The formed emulsion/suspension droplets can be effectively polymerized by the thermal or free radical initiator as discussed before. Condensation

polymerization precursor solution/s are divided into two or three or more separate solutions comprising precursors, solvents, catalysts/cocatalysts and the like and are stored and fed into the apparatus separately. In one illustrative embodiment, precursor solutions are fed via separate syringes, mixed and then converted into droplets and then effectively crosslinked in droplet form to make the crosslinked gel particle. One syringe may comprise a protein solution or PEG amine (PEG10K4ARM amine as an example) solution in an aqueous buffer and the other may comprise protein crosslinker solution (PEG-glutarate NHS ester as an example) in an aqueous buffer. Another solution may comprise a macromonomer solution and the other solution may comprise a photoinitiator solution. Alternatively, photoinitiator and macromonomer solutions are premixed and used as one single solution only. Both syringes can also be held in the infusion pump apparatus to infuse solutions at a controlled rate or maybe manually pushed to infuse the solution with the desired rate. The coacervation apparatus comprises a “tube in tube” like structure wherein the outside tube is used to flow non-aqueous inert fluid-like mineral oil or silicone oil via an inlet at the desired flow rate. The inner tube is connected to the mixing chamber where precursor solutions are mixed and held for a short duration of time (substantially below the gel time of the mixed composition) and then pushed through the inner tube at the desired flow rate. Upon exiting the inner tube, the liquid droplets comprising all the components of crosslinkable composition in a non-crosslinked state are formed in the outer tube with inert liquid flowing at the desired rate. The size of the droplet is generally determined by variables such as flow rate of mobile phase (inert fluid-like mineral oil in the outer tube), precursor solution flow rate, the diameter of the inlet precursor solution outlets, surface chemistry of the tube surface, temperature, pressure inert fluid viscosity and precursor chemical composition and the like. The solution droplets are preferably separated by a separate phase such as the oil phase and the distance of separation between each droplet is controlled by variables as described above. The separated droplets travel distance (L) until effective crosslinking in the droplet is achieved. The travel distance L will be different for each crosslinking composition chemistry and effective polymerization conditions. The effective crosslinking conditions must be determined experimentally for each composition. The crosslinked droplets or microspheres (are collected at the other end of the tube in a suitable medium such as PBS or mineral oil and are separated and stored until future use. A thermal jacket may be used outside the outer tube to heat/cool the droplets and to assist in an effective crosslinking reaction. Electric potential may be applied between the collection apparatus and droplet formation apparatus to assist in droplet formation and control its size and shape. The tip of the apparatus where droplets leave can be vibrated at the desired frequency using ultrasonic vibrators to quickly discharge droplets from the tube surface and also to control their size. Macromonomer solutions with photoinitiator can also be used to form precursor droplets and then exposed to high intensity laser light to effectively polymerize and/or photocrosslinking the precursor.

In one illustrative embodiment (EXAMPLE 5), a macromonomer such as 35KUA and PLGA is illustrative NBP along with UV photoinitiator are dissolved in an exemplary organic solvent like dichloromethane to form a homogeneous solution. The solution is then subjected to droplet formation using a modified coacervation apparatus and each droplet is subjected to UV irradiation to effectively polymerize and crosslink while in the droplet form. The polymerized droplets are collected as organogel particles. Due to polymerization, crosslinking and gelation in organic solvent dichloromethane, the organogel droplet cannot fuse or break thus locking the shape of the droplet. This ability of organic solvent gel formation via the crosslinking process to lock the shape helps to prevent unwanted solution droplet agglomeration/breakage which is normally associated with liquid droplets in conventional PLGA microsphere preparation (EXAMPLE 11-1).

In another illustrative embodiment, an in-house fabricated silicone rubber microfluidic chip containing 0.29 mm fluid mobile phase dispensing path is used to flow one percent polyvinyl alcohol solution as a mobile phase. This path is connected to the dichloromethane solution of PLGA and PEG 35KUA with a photoinitiator as above via a 26-gauge stainless steel needle connected at a 90 degrees angle. Polyvinyl alcohol solution is filled in the 50 ml syringe and is connected to the syringe pump. The solution is dispensed at 50 ml per hour through the fluid chip path. The PLGA and PEG 35KUA solution as above is filled in the 10 ml syringe and is dispensed via a 26 gauge needle in the silicone chip path at 5 ml per hour. Approximate 100-150 micron size PLGA monomer solution droplets are formed in PVA solution and are collected in a petri dish which is continuously exposed to long UV light. The collecting dish is moved manually so that each droplet coming out of the fluid chip is collected on a new surface on the petri dish. This way droplets are exposed to light without any shadow effect which facilitates effective photopolymerization and crosslinking. The exposed droplets undergo quick effective polymerization (preferably under 300 seconds, most preferably under 60 seconds) and crosslinking. The crosslinking and organogel formation prevents the fusion of liquid droplets. Control droplets/microspheres with PEG 35KUA but without PLGA as above in the dichloromethane are also prepared in the same manner. The control microspheres containing crosslinked PEG 35KUA but no PLGA are substantially transparent in nature to the human eye while microspheres with PLGA are substantially opaque in nature after partial evaporation of dichloromethane. The composite microspheres prepared have substantially uniform size distribution. In another modification of the above example, the macromonomer is first dissolved in an illustrative aprotic solvent such as dimethyl sulfoxide (DMSO) instead of dichloromethane and the mobile phase is hexane or mineral oil-hexane mixture (80:20) is used. The solution is fed to a microfluidic chip to form uniform size droplets. The droplets are then collected in liquid nitrogen to freeze/quench the droplets. The use of DMSO as the aprotic solvent is for illustration only. Other organic solvents like dimethylacetamide, n-methyl pyrrolidinone and the like also can be used as long as insoluble mobile phase is used and monomer has the ability to form effective polymerization and crosslinking in those solvents. The liquid nitrogen is evaporated and the mixture in frozen/solid-state is exposed to long UV light to effectively crosslink the macromonomer in frozen state. Care is taken to expose all microspheres from all sides while in the frozen state for effective crosslinking before attaining ambient temperature. FIG. 6 shows photographs of the composite microspheres prepared using methods described in this invention (EXAMPLE 5). FIG. 6A shows composite organic solvent gel microspheres prepared from photocrosslinked PEG macromonomer and PLGA (molecular weight 10000-15000 Daltons, endcapped) that are swollen in solvent dichloromethane (size around 150 microns). The microspheres were prepared using the coacervation/microfluidic chip method. The crosslinked polymer nature of the droplet does not permit the droplets to merge or coalesce to form bigger droplets/particles even though the particle comprises solvent dichloromethane and PLGA polymer. FIG. 6B shows a photograph of the same particles as in A wherein solvent dichloromethane is substantially removed by evaporation and PLGA in the gel particles is almost completely precipitated “in situ” inside the microspheres making them substantially opaque in nature. FIG. 6C shows photograph composite microspheres wherein solvent dichloromethane was completely removed by evaporation and then incubated in water. Crosslinked PEG Hydrogel swells in water (substantially transparent) and PLGA in the microspheres is seen as opaque particles within the swollen PEG hydrogel. DMSO is a preferred aprotic solvent because of its desirable melting point (19 degree C.), its ability to dissolve a variety of drugs/visualization agents and non-crosslinkable biodegradable polymers (NBP), its water solubility, biocompatibility, and its ability to crosslink PEG based macromonomers in a frozen state in presence of NBP. Other water soluble biocompatible described in this invention can also be used. Another advantage of this invention is that organogel microspheres swollen in the biocompatible solvents like DMSO, NMP, PEG ethers and the like is used without substantial or complete solvent removal as an injectable organogel pharmaceutical composition. Such compositions are first suspended in the biocompatible fluid carrier/medium without dissolution and then injected in the body. Preferably such suspension is prepared just prior to injection. The injectable composition is preferably prepared and injected within 6 hours, preferably within 1 hour or preferably within 15 minutes prior to injection. The shorter time is preferred to reduce the amount of drug diffused out in the injection medium and reduce burst effect of the drug. Total weight of particles suspended in the medium may range from 2 percent to 98 percent, preferably 10 percent to 90 percent relative to total weight of the composition. Preferred carriers include but not limited to are: water for injection, PBS, glycerol, PEG or PEG endcapped ethers or their derivatives and their mixtures in any proportion. The suspended particles are injected in the body preferably in the muscle tissue for local or systemic effect. The injection medium and solvent in the microparticles disperses in the tissue and drug entrapped in the microparticles is released in a sustained manner. The most preferred organogel injectable particle composition use biodegradable polymer or thermosensitive or pH sensitive gel or liquid carrier for controlled release of drug.

In another embodiment (EXAMPLE 5), a microfluidic droplet generator is used to prepare precursor solution/s droplets of desired size and shape. The path of droplet travel in the chip is controlled by the chip design so that the precursor gets sufficient time in the droplet to crosslink. The commercial microchip generators can produce droplets of various sizes depending on factors like the chip design, fluid/capillary path dimensions, capillary/fluid path surface modification, flow rates of solutions and the like. Composite particles generated by fluid microchips are generally uniform in shape and the use of crosslinking helps to minimize the droplet agglomeration in the chip.

FIG. 5 shows a partial schematic representation of a method for creating composite microparticles of various shapes comprising a biodegradable crosslinked polymer and biodegradable hydrophobic polymer made using organic gelling compositions described in this invention. FIG. 5A1 shows an illustrative aluminum based mold with 300 microns diameter cylindrical holes and FIG. 5A2 shows gelatin based dissolvable hydrogel mold with 50 microns size cylindrical cavities. 501 shows mold material body with top and bottom surfaces and 502 shows cubical, cylindrical and hexagonal shaped cavities in the 501 body. FIG. 5B shows 502 mold cavities filled with precursors of uncrosslinked hydrogel or organic gelling compositions (503) described in this invention which may comprise non-crosslinked biodegradable polymer/carrier such as PLGA. The precursor optionally may also comprise drug, cells, visualization agent and porogen. The organic gel composition is effectively crosslinked under effective crosslinking conditions and the crosslinked composition (504) takes the shape of the mold cavity. The crosslinked microparticles (504) of various shapes are removed from the mold, isolated, optionally washed to remove crosslinking reaction byproducts and other undesirable components (C). The crosslinked hydrogel/ organic solvent gel microparticles are stored for future processing and use. In some embodiments, the precursor solution with drug/s or cells is frozen or dried in the mold. The frozen/dry shaped particles are taken out of the mold cavity and then crosslinked in a frozen/dry state to form a crosslinked product.

EXAMPLE 4 provides illustrative embodiments wherein silicone, hydrogel and other molds of desired sizes and shapes are used to make organic solvent gels or microparticles. One illustrative embodiment, silicone rubber mold with 2 cm by 2 cm mold with 20 circular cavities (350 microns diameter and 500 microns length) is cast from a stainless steel mold. Briefly, a reverse mold of the proposed mold is made using a CNC machine. Silicone rubber is cast into this mold to make a mold of desired cavities. PEG35KUA macromonomer as an exemplary precursor and polylactide-co-polyglycolide (PLGA, 50:50 copolymer, molecular weight 10000 to 15000 Daltons) as illustrative NBP and 0.5 ml illustrative organic solvent dichloromethane are mixed along with photoinitiator. The precursor solution is then poured into the mold cavities, excess solution is wiped off and the solution is exposed to long UV light for 5 minutes. The solution converts into a soft organic solvent gel in the mold and takes the shape of the mold cavity. The solvent is removed by air drying followed by vacuum drying. The microcylinders are removed from the mold by stretching the mold (10-1000 percent) and shaking. The recovered cylindrical microparticles are stored in the freezer under nitrogen until use. In another modification of the above example, dichloromethane is replaced with DMSO and the solution with the mold is centrifuged for uniform cavity filling prior to light exposure. The crosslinked composite microcylinders are removed by incubation in water and mechanical stretching. Yet, in another example, a 500 microns thick stainless steel plate or aluminum plate is laser drilled to create 300 microns diameter holes in an array format (30 by 30 holes, 2 mm pitch). The holes are used to cast precursor solutions to make 300 micron diameter and 500 micron height microcylinders. It is understood that mold cavities of different shapes, sizes and volumes can be used to make a variety of microparticles with different shapes, sizes and volumes. The mold materials are generally inert towards the precursor compositions. FIG. 11 shows one illustrative process to make spherical shaped bilayered particles (FLOW DIAGRAM 4). FIG. 11G shows a semispherical (half-spherical) shaped transparent or semi-transparent mold cavity (1111). The cavity 1111 is filled with precursor solution 1112 (H). I show a second precursor solution (1113) is filled in a second mold. Solutions 1112 and 1113 are then reversibly gelled and/or frozen to form solids (1115 and 1114). The mold H and I are aligned and kept on top of each other such that open surfaces of 1112 and 1113 touch each other and the resultant shape is spherical or substantially spherical in nature (J). Precursor solutions are exposed to light to effectively polymerize and crosslink both solutions without substantial mixing. The precursor may be in the frozen state, liquid state or physically crosslinked gel state or combination in any proportion thereof prior to crosslinking. H shows polymerized unibody spherical particles with two layers 1116 and 1117 wherein 1116 is crosslinked 1112 and 1117 is crosslinked 1113. The same process may be adopted to make spherical particles with hollow cavities. In this case, the mold cavity surface (1111) is coated with a desired thickness and then frozen. The frozen surfaces are then fused with other frozen coated composition via effective photopolymerization and/or crosslinking to make spheres that are hollow inside but have two layers of compositions.

In another embodiment, a microparticle making kit containing silicone rubber mold, casting gel, razor blades and instructions on making beads is purchased from AKINA, Inc., Lafayette, Ind., USA. The silicone rubber mold purchased had 20 micron size cylindrical columns protruding from its surface. 20 g of proprietary hydrogel polymer provided by the supplier is dissolved in 300 g of distilled water and 450 ml of ethanol in a 1000 ml bottle. The polymer solution is then placed on top of the silicone mold to produce a negative imprinted image of the silicone mold having 20 micron diameter cylindrical cavities. A photopolymerizable precursor solution comprising NBP in water-insoluble organic solvents like dichloromethane or toluene and the like is then added in the hydrogel mold cavities and excess solution wiped off. The precursor is then crosslinked by exposure to UV light and the crosslinked particles are recovered after dissolving the mold in hot water. Silicone mold is commercially available in 6, 10, 20 and 50 micron sizes from AKINA, Inc and can be used to make cylindrical particles of diameter 6, 10, 20 and 50 microns respectively. Other microparticles of different shapes and sizes may be made using custom or commercially available molds as described in this invention.

In another embodiment, a one inch section of a 300-800 micron internal diameter silicone tubing or glass capillary tubing is cut. The tube is filled with UV light based photo crosslinkable precursor composition (ensuring no entrapped air bubbles) using a syringe and long needle. The filled tube is then exposed to long UV light for 2-5 minutes to crosslink the precursor in the tube. Using a razor blade or histology microtome machine, 0.5 to 1 mm sections of the tube along with its contents are cut and are added to 100 ml PBS solution and vortexed. The crosslinked hydrogel cylinders are separated to produce hydrogel cylinders. In some cases, a soft plunger is used to eject the hydrogel from the tube. In another variation of this embodiment, the cast hydrogel is first removed from the tube by applying nitrogen gas pressure from one side and collecting the hydrogel cast micro-rods from the other side. The crosslinked gel is then dried and cut using a microtome machine to a desired length (10 to 500 microns). In another variation, 1 ml of 20 percent bovine albumin and 1 ml of PEG10K4ARM glutamate NHS crosslinker (50 mg/ml in PBS) are mixed and the precursor solution is added in the tube before gelling or crosslinking. The crosslinked composition is separated from the tube and cut to the desired length using a microtome machine.

Some of the composite OSG precursor compositions disclosed in this invention can be photopolymerized and crosslinked using UV or visible light. This property can be used to make microparticles of desired shape and size using standard lithographic methods known in the semiconductor microprocessor manufacturing art, screen printing art and the like. A sheet of an inert substrate such as glass, metal, silicon wafer, ceramic or polymer with the uniform flat planar surface is provided. The surface of the sheet is preferably spin coated with a solution of photocrosslinkable precursor comprising non-crosslinked biodegradable polymer (NBP) and a photoinitiator to obtain a coating of desired thickness “t”. The high molecular weight of NBP and its film forming properties helps in forming a uniform film. Optionally, the crosslinkable composition may also contain a drug and/or visualization agent. The solution may also be frozen or dried if it is capable of polymerization in the solid state or in the frozen state. Additional layers may be coated on top of the first layer if multilayered particles are desired. The coated surface B is then exposed to a UV or visible light via a photomask for a desired period of time until effective polymerization of light exposed coated composition is achieved. The photomask has circular shaped transparent areas (diameter “d”) such through which light can pass and other areas where light cannot pass in a desirable pattern and size. No polymerization in areas under the dark area due to the absence of light. Alternatively, an electron beam may be used to scan only areas where crosslinking is desired. The light or electron beam exposed areas of the coating undergo effective photopolymerization and crosslinking under effective polymerization conditions. The unpolymerized area of the coating is removed via precursor solvent washing, leaving behind the crosslinked polymer particle with diameter “d” and height/thickness “t” . The particles are separated from the surface to produce cylindrical microparticles with diameter “d” and height “t”. If one, two, three or more layers of uncrosslinked compositions that are polymerizable under solid state conditions are coated on top first layer and processed in the same manner as the first layer, the resultant particles obtained will have two, three or four or more layers, with each layer having different thickness, crosslinked polymer and drug/visualization agent. Masks with transparent areas with different shapes such as rectangle, square, triangle, pentagon, hexagon, heptagon, octagon, 3 star, 4 stars, circular shape with hubs and spokes and the like may be used to prepare microparticles with different shapes and sizes. Photomask may be purchased from commercial vendors known in the semiconductor industry. In one embodiment, a mask with 300 micron diameter clear circles in 25 by 25 array format is custom made by CAD/Art Services Inc., Bandon, Oreg. and used as described above. A PEG1OK urethane acrylate (20 percent), PLGA 45000 to 55000 Daltons (20 percent and 2,2-dimethoxy-2-phenylacetophenone as photoinitiator (0.1 percent) and Iodixanol (10 percent) are mixed in DMSO to form a homogeneous solution and the solution is spin coated on a glass slide to form 50-100 micron thick film. An aluminum sheet with 300 micron size holes separated by 1 mm distance is kept on the slide and the PEG1OK urethane acrylate is exposed for 5 minutes to 360 nm light passing through the holes in the aluminum mask. The light polymerizes and crosslinks in the exposed area of the mask. The exposed film is dipped in dichloromethane for 10-30 minutes to remove uncrosslinked unexposed monomers from the glass slide. The glass slide has 300 micron diameter size microcylinders separated by 1 mm with a thickness/height of 50-100 micron (coating thickness). The microcylinders are removed from the surface, dried under vacuum to remove solvent and stored until use. Those skilled in the art can understand that by changing the precursor type, type of non-crosslinked biodegradable polymer used, type of mask used, and other process conditions, microparticles with different crosslinked density, non-crosslinked biodegradable polymer type with different shapes and size can be produced.

Drugs in the composite microparticles may be added during the particle preparation step before crosslinking and polymerization. One of the novel features of composite particles described in this invention is that the drug can also be loaded under certain conditions generally referred to as “effective diffusion conditions” after the microparticles have been prepared. This way, only acceptable microparticles with suitable size, size distribution, surface characteristics, biocompatibility, biodegradation time and the like with desirable attributes are used. Such particles may be produced and stored in large quantities under GMP conditions. Drugs may be loaded in the pharmaceutical manufacturing conditions or may be loaded in the clinical setting or in the operating room just prior to injection. The composite microparticles can be considered as empty or partially filled micro-buckets/containers where drugs can be loaded/encapsulated inside the micro-bucket/container using a solvent diffusion process. Molecular permeability and other diffusion conditions of the composite particle acts as a door/lid of the micro-bucket wherein the drug can go inside the bucket. Once the drug is diffused inside the microparticle, then biodegradable polymer can be precipitated/solidified in situ and the drug is locked inside the microparticles. The locked drug inside the precipitated polymer can be released in a sustained manner via diffusion and/or bioerosion mechanism or combination thereof. In one illustrative embodiment, PEG-PLA macromonomer crosslinked material and non-crosslinked PLGA is used to form a composite material or microparticle. A composite microparticle (microsphere) made from photopolymerized PEG10K-lactate 5-tetraacrylate crosslinked and PLGA (molecular weight 50000 to 100000 g/mole) as a non-crosslinked biodegradable polymer entrapped in the photopolymerized gel is used to make a composite particle. The illustrative drug is a small molecular weight drug rifampin that is soluble in dichloromethane. The molecular permeability of this gel is below 40000 Daltons therefore the entrapped polymer cannot escape from the crosslinked gel without gel degradation. The molecular weight of rifampin is 822.94 g/mol is substantially below the molecular permeability of the crosslinked gel. The composite microparticle is incubated in a 10 percent rifampin solution in DCM for 5 to 60 minutes at room temperature. The dichloromethane absorbs and swells the composite particle and it also locally dissolves PLGA in the composite particle matrix. The drug diffuses inside the microparticle. Removal of solvent by drying/evaporation removes the solvent and precipitates the PLGA within the composite matrix microparticle. The infused drug remains entrapped in the crosslinked gel as well as in the PLGA. In another embodiment, same microparticle as above is used but rifampin is replaced with lodixanol (20 percent concentration in DMSO), an illustrative x-ray contrast agent (molecular weight 1550.19 g/mole) and dichloromethane is replaced with DMSO, incubation time is extended to 12 hours. The solvent DMSO is removed by vacuum drying or by exchange with dimethyl ether. The lodixanol encapsulated composite particles show good x-ray visibility.

In another embodiment, PLGA is entrapped in PEG based crosslinked hydrogel prepared by condensation polymerization. In the non-limiting illustrative embodiment, crosslinked hydrogel from four arm PEG10K-Glutarate NHS ester and PEG diamine molecular weight 10000 is made with PLGA (PLGA 50:50 molecular weight 45000-55000, PLGA, ester endcapped). Percent of PLGA in the composite material is 20 percent. PLGA being endcapped with ester moieties does not have functional groups that take part in crosslinking of PEG10K-Glutarate NHS ester and PEG diamine (molecular weight 10000). Upon effective crosslinking of PEG10K-Glutarate NHS ester and PEG diamine, in organic solvent DMSO, the crosslinked network in a swollen state is formed. PLGA remains entrapped in the crosslinked hydrogel and remains soluble in the free DMSO in the swollen hydrogel. The solvent is removed or PLGA is selectively precipitated by exposing/incubating the swollen gel in excess methanol. Methanol being non-solvent for PLGA, it precipitates PLGA in the gel and also removes DMSO. The methanol is removed and the microparticle has crosslinked gel with precipitated PLGA in the microparticle. In another embodiment, the incubation of composite swollen gel is done in DMSO comprising lodixanol, an illustrative visualization agent. Upon diffusion, the solvent is removed and the solidified microparticle has lodixanol encapsulated in the crosslinked gel as well as PLGA.

Many polymers are slower to diffuse in and out of the crosslinked gel than small molecular weight drug and visualization agents (molecular weight below 2000 Daltons) and therefore incubation time can be used as one of the independent controlling variables for drug/visualization infusion/diffusion inside the composite materials. Organic solvent soluble drugs having a molecular weight less than 2000 g/mole can be quickly infused in a short incubation time before non-crosslinked biodegradable polymer present in the composite particle diffuses out. The incubation time may vary from 2 minutes to 2 weeks, preferably 5 minutes to 72 hours and most preferably 10 minutes to 48 hours. When incubating in the solvent for the diffusion of a drug, care is taken so that the dissolved biodegradable polymer that is entrapped during the polymerization and crosslinking process remains substantially entrapped in the composite material. This is achieved by controlling the molecular permeability of the crosslinked network that is formed. Other variables that slow down the diffusion of biodegradable polymer from the swollen polymer include but not limited to: nature of hydrophobic sections inside the crosslinked network (length of the PLA biodegradable block or polypropylene oxide block covalently linked inside the crosslinked network), incubation time (high molecular weight polymers are slow to diffuse out and may require substantial time to diffuse out), the crosslinked density of the crosslinked network, the molecular weight of NBP that is entrapped, branched nature of NBP, nature of the solvent, the combination of solvent and non-solvent mixture for NBP and the like. Many synthetic polymers do not have a single molecular weight structure and generally comprise a mixture of polymers with different molecular weights (molecular weight distribution). Some low molecular weight fractions of the non-crosslinked biodegradable polymer may diffuse out during the infusion and drug loading step. Generally, a substantial amount of the biodegradable polymer relative to its original amount should remain inside the composite polymer microparticle after incubation in a solvent, preferably at least 50 percent and most preferably at least 70 percent should remain inside the composite microparticle. The crosslinked polymer in the composite material can be made by chain growth polymerization or condensation polymerization. In chain growth polymerization free radical polymerization preferably photopolymerization is even more preferred.

The preferred biodegradation time of a crosslinked network may be 3 days to 3 years, preferably 5 days to 2 years and even more preferably 7 days to 1.5 years. The polymers may degrade by hydrolysis or enzymatic pathway or combination thereof. The molecular weight of biodegradable blocks (203) in the crosslinked network may range from 300 to 60000, preferably 500 to 20000 g/mol. The preferred molecular permeability of crosslinked gels may range from 2000 Daltons to 200000 g/mol. The non-crosslinked biodegradable polymer (NBP) entrapped in the composite polymer may be physically entrapped or covalently linked in the composite polymer. In the preferred embodiments, the biodegradable polymer is physically entrapped (no covalent linking to the crosslinked network). The molecular weight of the non-crosslinked biodegradable polymer may range from 2000 to 2 million, preferably 1500 to 300000. The preferred biodegradable polymer is hydrophobic or substantially hydrophobic and soluble in commonly used organic solvents. For hydrogel compositions, the preferred NBP can be hydrophilic or hydrophobic. The NBP is preferred to be a synthetic polymer, even more preferably a copolymer of PEG or PPG and polylactone or polycarbonate. The NBP may be endcapped with acetate ester, triiodobenzoic acid derivative or fluorescent compound derivatives such as derivatives of fluorescein or eosin or any other biocompatible endcapping compound. The end groups generally prevent participation in the crosslinking process. End groups also prevent the initiation of unwanted hydrolysis of the NBP during storage and processing. Biodegradable polymers can have end groups such as hydroxyl groups, carboxylic acid groups and the like provided they do not interfere in crosslinking reaction or affect the biodegradation and other useful properties. The non-crosslinked biodegradable polymer may be linear, branched or dendrimer in nature. Branched or highly branched or dendrimer may be preferred because such polymers may entangle with the crosslinked network and may be slow to diffuse out during the drug infusion process. The non-crosslinked biodegradable polymer may be solid, semisolid, wax type, gel, neat liquid or low melting solid (melting point below 70° C.) in the composite particle. The non-crosslinked biodegradable polymer may also have additional properties such as thermoreversible gelation or pH sensitive gelation or other physical gelation properties. The description of preferred NBP is given in the earlier section. The weight of the drug relative to the total microparticle weight loaded in the composite microparticle may range from 0.1 percent to 60 percent, preferably 1 percent to 50 percent and more preferably 3 to 40 percent. Drugs may be added before the effective crosslinking process or after crosslinking via solvent infusion as described before. Drugs may be released in a sustained manner by diffusion and/or biodegradation/bioerosion from the composite materials. Drugs may release within 1 day to 2 years, preferably 2 days to 1 year from the composite material. In one illustrative embodiment (EXAMPLE 9), PEG10K acrylate macromonomer and PLGA (50:50, molecular weight 45000 to 55000 g/mole are used to make microspheres (PEG10K-PLGA microspheres) using modified microfluidic chip apparatus as described previously. The microspheres were incubated in rifampin solution for 5 minutes. The solvent was removed by air drying followed by vacuum drying. The samples were loaded in a dialysis bag and rifampin release was monitored for 5 days. FIG. 9 shows a controlled release profile of rifampin from composite microspheres comprising PEG based crosslinked polymer with in situ precipitated PLGA polymer. The rifampin entrapped in precipitated PLGA is released in a controlled manner for over 4 days. Drug release from crosslinked polymer network in the composite microparticle will depend on many factors including but not limited to: crosslink density, molecular permeability of the network, the biodegradation rate of the network, biodegradation pathway such as enzymatic or hydrolysis of the crosslinked network, length or molecular weight of inert organic soluble block (201) like PEG, length of the biodegradable block (202), hydrophobicity of the inert and biodegradable block and the like. The sustained release from NBP polymer may depend on many factors including but not limited to: NBP molecular weight and its molecular weight distribution, type of terminal end groups, number of branches, copolymer composition and its arrangement, size and shape of the precipitated particle in the composite, percent crystallinity of the polymer, drug solubility in the polymer and its interaction with polymer, rate of solvent removal or precipitation in the composite, the porosity of the precipitated NBP, type of solvent used in composite preparation, residual solvent left after drying or lyophilization and the like. Those skilled in the art can understand that the variables described above may be used to achieve the desired rate of release with minimum burst release. In the preferred embodiment, the crosslinked network in the composite particle has 2-90 percent, preferably 5 to 80 percent PEG relative to composite particle weight. The crosslinked network based on PEG is hydrophilic and can hydrate under in vivo conditions and provide a protein adsorption resistant surface which may be useful in improving the biocompatibility of the composite microparticle. In some cases, the crosslinked network may also serve a coating that can control the rate of drug release (rate limiting function). Infusion conditions (time, temperature, concentration) that can cause unwanted degradation or chemical reactions/interactions with composite matrix and biodegradable polymer are highly undesirable. Prolonged exposure to acidic and basic aqueous buffers can cause unwanted hydrolysis/degradation of composite material are generally avoided. In some embodiments, the NBP used in the composite microparticle may have properties like thermoreversible gelation property, pH sensitive gelation property or may be present as a neat liquid carrier.

Table 2 shows the difference between conventional drug encapsulated microparticles (Example 11-1) and drug loaded composite microparticles described in this invention (Example 5).

TABLE 2
DIFFERENCE BETWEEN COMPOSITE DRUG LOADED MICROPARTICLES DESCRIBED IN THIS
INVENTION AND CONVENTIONAL BIODEGRADABLE DRUG LOADED MICROPARTICLES.

| COMPOSITE MICROPARTICLES. | |
|---|---|
| PARTICLES COMPRISING CROSSLINKED | CONVENTIONAL MICROPARTICLES. |
| BIODEGRADABLE POLYMER AND NON- | PARTICLES COMPRISING NON-CROSSLINKED |
| CROSSLINKED BIODEGRADABLE POLYMER SUCH | BIODEGRADABLE POLYMER SUCH AS PLGA |
| AS PLGA WITH DRUG. | WITH DRUG. |
| Combination of crosslinked biodegradable polymer and | Only use NBP such as PLGA to make microspheres. |
| non-crosslinked biodegradable polymer (NBP) wherein | |
| NBP is physically entrapped in the crosslinked polymer. | |
| NBP is precipitated in situ inside the particle and is not | |
| covalently linked to the crosslinked biodegradable | |
| polymer. | |
| Particles can be prepared, suspended in a biocompatible | Cannot be suspended in organic solvent such as |
| organic solvent such as DMSO and injected in the tissue | DMSO for injection in the body. Most commonly used |
| without dissolution. The crosslinked nature of the particle | polymers like PLGA dissolve in organic solvents like |
| prevents dissolution in organic solvents. | DMSO. |
| Post loading of small molecular weight drugs is generally | Generally, not possible. Exposure to an organic |
| possible after the microparticles are formed. | solvent will generally dissolve the carrier polymer like |
| | PLGA. |
| Composite nature can potentially provide fracture | May be susceptible to fracture especially low |
| resistance because NBP is reinforced by a crosslinked | molecular weight polymers like PLGA during |
| polymer network. | processing and storage. |
| A combination of crosslinked density and bioerosion | The absence of crosslinked polymers limits the ability |
| property of a non-crosslinked biodegradable polymer is | to manage the desired drug release profile. |
| used to manage the desired drug release profile. | |
| Biodegradation depends on the biodegradation of | Biodegradation depends on the biodegradation of |
| crosslinked materials as well as non-crosslinked | biodegradable polymers such as PLGA. |
| biodegradable polymers such as PLGA. | |
| PEG based crosslinked materials used in some | No crosslinked polymer is used. |
| preferred compositions can provide improved | |
| biocompatibility after implantation due to the presence of | |
| PEG. | |
| The fast crosslinking process has the potential to reduce | Solution droplet agglomeration and splitting during |
| solution droplet agglomeration which may assist in | preparation has the potential to obtain broad size |
| uniform particle size distribution. | distribution. |
| NBP in the composite microparticles can be neat liquid | Cannot make solid microspheres (at body |
| or thermosensitive gel or pH sensitive gel while | temperature) from liquid carriers. |
| crosslinked polymer holds the microparticle or | |
| microsphere shape during the injection. | |

Thermoreversible gels have been used for sustained drug delivery (Review by L. Kloud et al.). Many physically crosslinked gels or thermoreversible gels such as gels made from Pluronic F127 or Tetronic 908 aqueous solutions have poor mechanical properties. Such gels typically cannot withstand mild to moderate shear force and generally cannot be fabricated and suspended as injectable microspheres for sustained drug delivery. In some composite microparticle compositions, the NBP used has thermosensitive or pH sensitive gelation properties. NBP can also be a neat liquid. Composite particles described herein can be suspended in an injectable medium such as PBS or saline and then injected in the body for local or systemic drug delivery. The NBP inside the composite particle is present as a thermosensitive gel, pH sensitive gel or as a neat liquid during the injection step. Thermosensitive gel and pH sensitive gel may be formed in situ inside the particle after the injection. This invention discloses two preferred methods to make microparticles comprising physically crosslinked gels or neat liquid carriers. In the first method, the Pluronic or other thermosensitive materials are used as NBP. NBP and precursor solutions such as macromonomer solutions are converted into microdroplets and then droplets are crosslinked to form composite microparticles. The concentration of NBP in the polymer is maintained such that the thermosensitive polymer can form a physically crosslinked gel when exposed to the aqueous solution such as physiological conditions present in the body (aqueous solution with pH 7.4, temperature around 37° C.). In the second method, the crosslinked composite particle is prepared with the desired size and shape without the NBP and then the particle is infused with thermosensitive, pH sensitive or liquid carrier material using solvent diffusion methods described in this invention. In some embodiments, the crosslinked particle is intentionally made porous and the porous space is then filled with thermosensitive, pH sensitive or liquid carrier material.

EXAMPLE 3 shows illustrative examples wherein thermosensitive gels or liquid carrier bases have been encapsulated or entrapped in the composite crosslinked material/particle. The crosslinked materials could be made using free radical polymerization of macromonomers or condensation polymerization of precursors, preferably PEG based precursors. In one illustrative example, PEG35KUA macromonomer, Jeffamine lactide copolymer (final concentration 25-35 percent) as illustrative thermosensitive polymer and photoinitiator are dissolved in the organic solvent. A small droplet of this solution is exposed to long UV light to crosslink the macromonomer. The crosslinked solution particles (organogel) are separated and the solvent is removed. The dried crosslinked particles have Jeffamine lactide copolymer physically entrapped/caged in the particle. The composites particles are suspended in PBS solution at 37° C. wherein Jeffamine lactide converts to a thermosensitive gel entrapped in the crosslinked hydrogel. This gel can be converted into a liquid solution at 0-10 degree C. if desired. The Jeffamine lactide gel is considered as a physically crosslinked thermosensitive gel that is entrapped in a crosslinked PEG35KUA macromonomer microparticle. In another modification of the above example, Pluronic F127 (final concentration 30 percent) is substituted in place of Jeffamine lactide. In another example, a macromonomer collagen solution in dilute hydrochloric acid, or acetic acid and photoinitiator is used in place of Jeffamine lactide copolymer to make a composite material. The composite material, when exposed to physiological conditions such as PBS solution pH 7.4, forms a collagen gel within the crosslinked macromonomer hydrogel material. The change of acidic conditions to basic pH leads to collagen gel formation (pH sensitive gel). The collagen gel may be further crosslinked by exposing the composite particle with crosslinker solution such as 0.2 percent glutaraldehyde in PBS (pH 7.2) or PEG10K4ARM glutarate NHS ester in PBS (0.5 percent, pH 7.2). The illustrative example of Jeffamine lactide uses organic solvent. However crosslinking reactions can also be carried out in aqueous solution preferably buffered solutions of macromonomers can also be used. When using an aqueous solution, temperature wherein the thermosensitive polymer is soluble in water must be used. In the case of Pluronic F127 and Jeffamine lactide, the preferred temperature aqueous solution is between 0-10 degree C. is used. In the case of gelatin, 50-70 degree C. is used. Those skilled in the art of thermosensitive polymer gelation know that experimental conditions such as concentration, pH and temperature of many thermosensitive and pH sensitive polymers are known in literature and such conditions must be used to form a composite material as described in this invention. In another illustrative example, the Pluronic and Jeffamine lactide gels are entrapped in the crosslinked gels that are formed by the condensation polymerization reaction. A drug can be entrapped along with thermosensitive or pH sensitive gels. Many thermosensitive polymers such as Jeffamine-lactide, PEG-polyhydroxy copolymers, Pluronics have a low molecular weight (1500 to 20000 g/mole) and such polymer can also be infused in the crosslinked particles by infusion methods as described in this invention for infusion of drugs. Generally, thermosensitive polymer solution in water or organic solvent is made first and then microparticles are incubated for a sufficient period of time to infuse/diffuse in the microparticle. The incubation time could be a few minutes to 2-3 weeks, preferably a few hours to 7 days. The crosslinked density of the crosslinked polymer must be sufficiently high to enable the diffusion of a thermosensitive polymer inside the particle. In the illustrative embodiment, thermosensitive polymers with molecular weight less than 60000 g/mole, even more preferably greater than 40000 g/mole should permeate through the crosslinked polymer. This infusion method is preferred if the crosslinked particle is porous in nature. The thermosensitive polymer can be filled partially or completely in the porous space of the particle. FIG. 8 shows a partial schematic representation of methods to make drug loaded microparticles or microspheres. FLOW DIAGRAM 1 shows methods to infuse drug and biodegradable carriers in crosslinked microspheres/microparticles. A schematically shows a crosslinked microsphere preferably crosslinked biodegradable microsphere. The particle A is then exposed to a homogeneous solution of drug and biodegradable carrier in an organic solvent. The organic solvent used is capable of swelling the crosslinked microsphere A but incapable of dissolving A. The particle is incubated in the drug solution for sufficient time until the desired amount of drug and the biodegradable carrier is infused inside the particle (B). Particle B is optionally washed with solvent to remove surface bound drug and carrier without substantial loss of drug and/or carrier. The biodegradable carrier used can be solid, semisolid, gel or liquid at room temperature or body temperature (37 degree C.). The solvent is removed from the swollen particle evaporation or lyophilization of B and drug and biodegradable carriers remain entrapped in the microsphere (C). The drug loaded particles are sterilized (D), suspended in an injectable medium like PBS solution (pH 7.4) and then injected into the body for local or systemic drug delivery.

In one illustrative embodiment microcylinders loaded with Rifampin as a model drug and Jeffamine lactide as an exemplary thermosensitive drug carrier were prepared and evaluated for controlled release properties. PEG1OK acrylate macromonomer is polymerized and crosslinked inside a silicone rubber or stainless steel mold to form microcylinders (EXAMPLE 8). The dried microcylinders thus prepared were used for drug loading experiments. The dried microcylinders were incubated in Jeffamine lactide and rifampin solution in dichloromethane for 30 minutes and dried to remove the solvent (referred to as sample microcylinders). Another set of microcylinders were incubated in rifampin solution only without Jeffamine lactide as a carrier (referred to as Control 1). Microcylinders incubated in dichloromethane without rifampin and Jeffamine lactide are used as Control. The incubated particles were dried to remove solvent and then transferred to the dialysis bag and incubated in 10 ml PBS for 1 minute to wash loosely bound rifampin. All the samples were then incubated in a fresh 10 ml PBS solution at 37 degree C. At each time point, all PBS solutions were removed and fresh 10 ml PBS was added. The rifampin concentration was monitored for all three samples for 120 hours at several time points. The concentration of Rifampin was monitored by measuring absorbance at 474 nm using UV-VIS spectrophotometer. Average and standard deviation was reported for each time point. FIG. 10B shows a controlled release profile of rifampin from composite microcylinders. As expected, control microspheres do not show the release of rifampin. Control 1 microspheres with no NBP show a fast release in 50 hours and samples with Jeffamine lactide as a NBP and thermosensitive polymer show a controlled release up to 120 hours. The thermosensitive Jeffamine lactide gel inside the composite microcylinders is significantly effective in slowing down the controlled release of rifampin from the microcylinders as compared to the control 1 sample. Control 1 is also an example of drug crystals that are formed inside PEG based hydrogel microspheres (discussed separately elsewhere in the specification, FLOW DIAGRAM 2) which dissolve slowly and provide a release up to 50 hours. Depending on the drug solubility under physiological conditions (pH 7.4), the release will be shorter or longer.

Some polymers such as gelatin or some PEO-polylactone copolymers undergo gelation when injected as a hot solution (less than 65 degree C., preferably less than 50 degree C.) and cooled to body temperature (0-40 degree C.) may also be used. Many other types of thermosensitive polymers are known in the art. Among these biodegradable or bio-dissolvable polymers (polymers that dissolve in the human body and are removed safely from the body without harmful effect) are preferred. The thermosensitive polymers that can be used include but not limited to are: Pluronic or PEO-PPO copolymers; reverse Pluronics; polyalkyl acrylamides such as polyisopropyl acrylamide and their copolymers; gelatin (various grades); chitosan based compositions and its derivatives, cellulose derivatives, various PEG-polylactone copolymers, PEG-PLA, PEG-PLHA, PEG-polyhydroxy copolymers, and the like or combinations thereof. U.S. Pat. Nos. 6,004,573 and 7,740,877, US patent application 20140256617 and references therein, cited herein for reference only, disclose thermosensitive gel compositions. Such compositions can also be used to make composite particles as described in this invention. The release profile of the drug will depend on the crosslinked density of the hydrogel microsphere and the type of thermosensitive polymer used. The thermosensitive compositions described herein can deliver a variety of drugs including protein drugs. The drug may be microencapsulated in a biodegradable matrix for better control over the release profile. A detailed list of drugs is given in the definition section of this document. Up to 0.1 percent to 30 percent drug may be loaded (relative to composite particle weight) in the thermosensitive composition. Actual loading will depend upon the type of drug used, drug solubility, type of thermosensitive polymer used and the like. Visualization agents such as coloring or medical imaging agents may be added to the thermosensitive composition to assist in the delivery of the composition and to follow its degradation after implantation. U.S. Pat. No. 7,790,141, cited herein for reference only, discloses radio-opaque compositions and such compositions may be added and used for local delivery as described before.

This invention also discloses compositions and methods wherein the composite microparticles/materials use neat liquids as drug carriers. The biodegradable crosslinked particles may be infused with the liquid carrier (FIG. 8, FLOW DIAGRAM 1) or maybe encapsulated during the precursor crosslinking process. The biodegradable crosslinked particles may be made using free radical polymerization of biodegradable macromonomers or may be made via condensation polymerization of precursors as described in this invention. The liquid carrier infused composite microparticles are prepared and then suspended in a suitable injectable medium such as PBS wherein the liquid carrier used is insoluble in the medium and then injected into the human or animal body for sustained release of a drug. The liquid carrier may be oil or other polymeric or non-polymeric liquid. The carrier liquid may be water soluble (solubility 1 g/100 g water) or water insoluble (solubility less than 1 g/100 g water). The liquid carrier is substantially liquid at room temperature or at around body temperature (37 degree C.) or it could be a low melting polymer or non-polymer with a melting point below 60 degree C. The biocompatible liquid carriers may be hydrophobic or hydrophilic. Preferred biocompatible liquid carriers are hydrophobic in nature. The preferred liquid carriers that can be used include and not limited to: sucrose acetate isobutyrate, vitamin E and its derivatives; fatty acids like oleic acids and its derivatives; fatty alcohols; amino acids with fatty acid side chain, liquid non-ionic surfactants like polysorbate, Tween® 40 or Tween® 80; polymers like liquid polylactones, liquid polyhydroxyacids, liquid PEG-polylactone copolymers, PEO-PPO-polylactone copolymers, liquid polylactide, liquid polylactide and glycolide copolymers, liquid polytrimethylene carbonate, liquid polyorthocarbonates, and its copolymers or combinations thereof and the like are preferred. The low melting polymers may be copolymers of polyalkylene oxide-polyhydroxy acid, preferably PEG-hydroxyacid copolymer. PEG-hydroxyacid copolymer may be AB or BAB or ABA type block polymer. Biodegradable liquids are most preferred. The biodegradable liquids or low melting polymers used in this invention may last in the body from 3 h to one year, preferably from 12 h to 180 days, even more preferably from 24 h to 90 days. The drug loading in liquid carriers may range from 0.01 percent to 50 percent, most preferably 0.1 percent to 40 percent, even more preferably from 1 to 30 percent.

In one illustrative embodiment (EXAMPLE 3), a composite of free radical crosslinked hydrogel and biodegradable liquid carrier is prepared and used. PEG35KUA macromonomer, sucrose acetate isobutyrate are dissolved in ethanol along with photoinitiator to form a precursor solution. 50 μl of this solution is then exposed to long UV light in a circular mold. The solution forms a firm disk shaped organogel within 300 seconds. The solvent is removed from the crosslinked gel and is dried under vacuum with entrapped sucrose acetate isobutyrate entrapped in the gel that serves as a liquid carrier in the biodegradable crosslinked gel. Using a similar manner, vitamin E acetate is used in place of sucrose acetate isobutyrate and the solvent is changed from ethanol to dichloromethane. In another embodiment, an illustrative biodegradable liquid polymer (polycaprolactone triol average molecular weight around 900 g/mole) is used as a liquid carrier. In another embodiment, PEG-polylactate copolymer that is liquid at 30 degree C. is used as a liquid carrier. In another illustrative embodiment, PEG10K4 ARM tetramine and PEG10K4ARM glutarate NHS ester and sucrose acetate isobutyrate are used to form crosslinked biodegradable hydrogel particles using dichloromethane as an organic solvent. The entrapped sucrose acetate isobutyrate is used as a carrier for drugs. As discussed before, liquid carriers also can be infused into preformed biodegradable microparticles or microspheres via solvent diffusion. The method is similar to methods used for the infusion of drugs in the composite materials using organic solvents as discussed previously.

In another illustrative embodiment (EXAMPLE 8B) rifampin as an exemplary drug and vitamin E as an illustrative liquid carrier were dissolved in tetrahydrofuran. Dry PEG1OK microcylinders were then incubated in Rifampin and Vitamin E solution for 5 minutes. The solvent was removed by air drying followed by vacuum drying. The samples were loaded in a dialysis bag and rifampin release was monitored for 5 days as mentioned previously. FIG. 10A shows a controlled release profile of rifampin from composite microspheres comprising PEG based crosslinked polymer with vitamin E as a liquid drug carrier. Vitamin E helps to release the drug in a controlled manner for 5 days. In some embodiments, porous microparticles are used and pores in the microparticles are then filled using the liquid carrier. The porous hydrogel is exposed to a biodegradable polymer solution (liquid polycaprolactone or PEG-polylactide copolymer) with the drug. After complete infusion of polymer solution in the porous space, the solvent is removed leaving behind the crosslinked hydrogel infused with liquid biodegradable polymer with or without the drug.

Many drugs have poor water solubility but high potency. Paclitaxel is a well-known anticancer drug that has low water solubility (less than 0.1 μg/mL). However, it has better solubility in organic solvents like DMSO (5 mg/ml) and ethanol (1.5 mg/ml). Composite biodegradable microspheres with or without NBP prepared as above may be loaded with water insoluble drugs such as paclitaxel using an organic solvent solution. The solvent is removed and the drug is precipitated/crystallized inside the microspheres. The drug loaded microspheres are then suspended in an injection medium like PBS and injected inside the body. The crystallized drug slowly dissolves under physiological conditions providing local and systemic controlled drug delivery. The drug crystals are also protected by the crosslinked hydrogel network for creating immune or other foreign body response during their controlled drug delivery. FIG. 8, FLOW DIAGRAM 2 shows a method to prepare drug loaded biodegradable hydrogel microspheres wherein the drug is substantially insoluble in water. E schematically shows a crosslinked microsphere preferably crosslinked biodegradable hydrogel microsphere. The microsphere E is exposed to a homogeneous solution of the drug in an organic solvent. The organic solvent is capable of swelling crosslinked microsphere E but incapable of dissolving E and the organic solvent has higher or substantially higher solubility of the drug as compared to water. The particle E is incubated in the drug solution for sufficient time until the desired amount of drug is infused inside the particle E (F). Particle F is optionally washed with solvent/water to remove surface bound drug without substantial loss of drug from F. The solvent is removed from the swollen particle by evaporation or lyophilization or by drug's non-solvent exchange which precipitates/crystalizes drug particles inside E (G). The drug loaded particles are then sterilized (H), suspended in an injectable medium like PBS solution (pH 7.4) and then injected into the body for local or systemic drug delivery. The crystallized drug within the particle body slowly dissolves under physiological conditions and provides a systemic or local therapeutic effect. In one illustrative embodiment, 2 mg paclitaxel is dissolved in 4 ml dimethyl sulfoxide or ethanol. 20 mg of dry crosslinked PEG1OK macromonomer based microspheres without NBP as a carrier is prepared as described in this invention (EXAMPLE 8, 9). The microspheres are then incubated in a paclitaxel solution for 60 minutes. The solvent is removed by vacuum drying or by incubating in 100 ml water for 30 minutes. The incubation in water or vacuum drying removes the solvent and precipitates the drug inside the PEG10K microspheres. The loose drug on the surface of the microspheres is removed by washing with water. Finally, the PEG1OK microspheres with paclitaxel are lyophilized. The lyophilized microspheres are sterilized using ethylene oxide and then suspended in 1 ml PBS. The suspension can be injected in the body for controlled drug delivery. The precipitated drug crystals inside the microsphere slowly dissolve providing controlled drug delivery. Using a similar procedure as above, bupivacaine free base and dexamethasone (both water insoluble drugs, solubility less than 2 g/100 g) are precipitated inside the microspheres. In another variation of the above method, biodegradable microspheres made from gelatin or microspheres made by condensation polymerizations of PEG derivatives are used instead of PEG1OK microspheres or microparticles. The drugs described above may also be added prior to polymerization and/or crosslinking, especially in organic solvents as discussed in the previous example and then the solvent is removed to precipitate or crystallize the drug.

Solid State Polymerization of Macromonomers and their Applications.

We have discovered that many macromonomers preferably biodegradable macromonomers depicted in FIG. 2 such as gelatin methacrylate, PEG35KL5A, PEG20KUA, JALA, F127LA and the like reported in this invention can be polymerized preferably photopolymerized in solid state or in frozen solution state (FSP) under certain effective conditions. We have used this discovery to make microparticles with encapsulated cells and drug/s, multilayered particles, sheet or fiber like materials with different compositions with controlled distribution and the like. This discovery can also be used for medical, food, cosmetics, agriculture, chemical processing and other industrial applications. EXAMPLE 10 provides some illustrative examples of solid state polymerization and their illustrative applications without any limitation. In one illustrative embodiment, PEG35K-LACTATE-5-diacrylate (35KL5A), an illustrative biodegradable macromonomer, is dissolved in PBS along with photoinitiator Irgacure and its effective polymerization is tested in a liquid solution state at ambient temperature. The solution can be effectively polymerized in a liquid solution state in a short period of time when exposed to long UV light. A small amount of the same solution is first frozen at −20° C. on a glass slide inside the freezer and then exposed to the frozen solution to 360 nm light for 10 minutes in the frozen state. The exposed solution is then warmed to room temperature and incubated in water for 6 hours. The solution is converted into a crosslinked gel that is swelled in the water without dissolution indicating effective polymerization and crosslinking in the solid state (frozen state). A control sample that is frozen but not exposed to light dissolves completely in water upon incubation indicating no crosslinking. The same experiment is repeated with additional macromonomer concentrations in PBS and found to crosslink in the solid state. Using a similar procedure as above, PEG20K urethane diacrylate (PEG20KUA) is dissolved in 0.5 ml DMSO (an illustrative organic solvent) and then mixed with 10 μl of Irgacure 2959 photoinitiator stock solution (ISS). One drop of the solution is exposed to long UV light (365 nm light with intensity 26,500 μW/cm²). The solution forms a gel within 60 seconds of light exposure indicating effective crosslinking of the precursor in liquid/solution state. 50 μl of the same precursor solution is then transferred on a glass slide and is frozen to 0 to −20° C. The frozen solution is then exposed to light for 60 seconds. The exposed solution is warmed to room temperature. The frozen and exposed solid is then incubated in the water showing formation of crosslinked gel without dissolution indicating the formation of effective solid state polymerization (frozen solution state) within 60 seconds. Using a similar procedure as above, 10 percent solution of PEG20KUA in dimethyl sulfoxide

DMSO and in polyethylene glycol dimethyl ether (molecular weight 600) is tested for effective polymerization in the frozen solid state. Effective polymerization is observed for both the solvents in liquid as well as frozen state which leads to the formation of a crosslinked gel. DMSO based formulation as above is also tested with PLGA polymer (molecular weight 125000-150000, 20 percent w/v) as an illustrative non-crosslinked biodegradable polymer (NBP) additive. The PLGA solution is also polymerized in solution and in a frozen state forming a composite material. NBP such as PLGA provides the ability to encapsulate drug/s as well as other useful properties. High molecular weight PLGA has a film forming ability which helps in providing improved mechanical stability to the crosslinked composition and film can be formed before the crosslinking initiation step in the precursor stage. Using a similar procedure as above, gelatin methacrylate (EXAMPLE 1B) is photopolymerized and crosslinked in solution and as well as in frozen state using PBS (pH 7.4) as a solvent. We tested three polymerization systems with different organic solvents as well as with aqueous buffers which freeze around −40 to 40° C. for effective polymerization and crosslinking in frozen state. The preferred temperature range for a frozen solution is preferably below ambient temperature and may range from −270 to 60° C., preferably from −192 to 40° C. Liquid gases such as liquid helium and liquid nitrogen are preferred cooling media to make frozen samples. The preferred solvents or their mixtures in any proportion include but not limited to: organic solvents such as DMSO, n-methyl 2-pyrrolidinone, benzyl alcohol, benzyl benzoate, polyethylene glycol or its derivatives, dioxane, water or aqueous buffers like PBS, triethanolamine, acetate, HEPES, TRIS with pH 6 to 8 and the like. Precursor solvents and solvent mixtures that have freezing point in between −80 to 60° C., preferably −30 to 45 degree C. are preferred. Osmotically balanced aqueous solutions that freeze around zero ° C. are preferred for live cell encapsulation applications. Solvents that are biocompatible and biodegradable are most preferred in biomedical applications. Preferred compositions crosslink in solid or frozen state via thermal or photopolymerization with 24 hours, preferably within 12 hours, even more preferably with 2 hours and most preferably within 30 minutes.

In one exemplary embodiment, 1 g of PEG35K-LACTATE-5-acrylate diacrylate (35KL5A) is dissolved in 9 g ethyl acetate. 1 ml of this solution is mixed with 10 mg of Irgacure 2959 photoinitiator and optionally with 20 microliters of vinyl pyrrolidinone as comonomer. 20 μl of the solution is then exposed to 360 nm long UV light (Black-Ray UV lamp, 360 nm light, 10000 mW/cm2 intensity) for 5 minutes. The liquid solution is converted into soft gel indicating effective polymerization and crosslinking in ethyl acetate in a short period of time. Another 50 μl monomer solution is added on top of a glass slide and the solvent is evaporated for 30 minutes at ambient temperature (around 30° C.). The substantially dry composition without solvent is then exposed to a Black-Ray UV lamp (360 nm light, 10000 mW/cm2 intensity) light for 10 minutes in the dry state. The exposed solid is then incubated in the water at room temperature for 2 h. The exposed solid did not dissolve in water but formed a crosslinked water swollen hydrogel indicating crosslinking of the composition in a dry solid state without the solvent. Control macromonomer in the sample that is dried but not exposed to light dissolved in water and did not form a crosslinked hydrogel. In another experiment, 5 droplets of the ethyl acetate macromonomer solution prepared as above are deposited on 5 glass coverslips (about 100 μl solution each slide) and weighed. The ethyl acetate is evaporated to a different degree of dryness by controlling drying time. The drying is monitored gravimetrically by weighing before and after drying. One sample is completely dried in a vacuum for 3 hours until no change in weight is observed. The solvent range in the droplet varied from 80 to zero percent in all samples tested. The partially dried (solvent amount 40-95 percent), and substantially dried (solvent amount 5 to 40 percent) and completely dried samples (solvent amount 5 to 0.001 or less percent) are exposed to UV light. All samples effectively crosslinked in partially, substantially and completely dry state. Using a similar procedure as above, PEG20K urethane diacrylate (PEG20KUA) is dissolved in toluene and then mixed with 10 μl of Irgacure 2959 photoinitiator stock solution). One drop of the solution is exposed to long UV light (365 nm light with intensity 26500 μW/cm²). The solution is converted to gel within 40 seconds of light exposure indicating effective crosslinking of the precursor in the solution state. Another 50 μl of the same solution is transferred on a glass slide and allowed to evaporate in a fuming hood for 30 minutes at room temperature. The partially dried composition is then exposed to light for 60 seconds. The exposed sample is then incubated in water and formed a crosslinked soft gel indicating effective solid state polymerization. Using a similar procedure as above, a 10 percent solution of PEG20KUA in dichloromethane is tested for effective polymerization in solution state and in a dry state. Effective polymerization is observed in a liquid solution state as well as a dry state. DMSO based formulation as above is also tested with illustrative NBP like PLGA (MW 125-150K, 20 percent) as an additive. The NBP/PLGA composition is also polymerized in solution and in a dry state as above. NBP such as PLGA provides the ability to encapsulate the drug and release the encapsulated drug in a controlled manner. Many NBP such as PLGA also have the ability to form film/fiber. This ability of film/fiber forming abilities can help to cast the precursor in a film or fiber state and can improve the mechanical properties of the crosslinked composition.

Organic solvents preferably commonly used organic solvents such as acetone, THF, ethanol, methanol, methyl ethyl ketone, dichloromethane, chloroform, toluene, ethyl acetate, ethyl lactate and the like may be used to prepare precursors in a dry state or frozen state. Volatile solvents such as THF, dichloromethane, chloroform and the like with boiling point ranging from −40° C. to 110° C. are most preferred. Solvents with low flammability are also preferred. Biocompatible solvents like water or aqueous buffers, DMSO, NMP, PEG dimethyl ether and the like are most preferred for biomedical applications. Generally, the precursor and other components are solubilized in the preferred solvent at a concentration of 0.01 to 70 percent, preferably 5 to 30 percent. If desired, a substrate is coated with solutions by dip coating or spray coating or spin coating and the like. The solution may also be filled in a mold cavity of the desired size and shape. The solvent is then removed by air drying, vacuum drying, sublimation/lyophilization and the like to obtain a dry state. The dry state may be partially dry, substantially dry or completely dry.

Alternatively, the solution may be frozen. The freezing can be done in a controlled manner (at a desired cooling rate of 0.1 to 30 degree per minute) or it may be quenched in cooling baths or in liquefied gases like liquid nitrogen or liquid helium or any other known methods in cryogenic engineering. Water or aqueous solution buffers like PBS buffer (pH around 7), acetate buffer pH around 4, triethanolamine buffer (pH around 7) and the like may be used for preparing dry state or frozen precursors. Buffers that provide physiological conditions such as PBS buffer pH 7.4 are preferred for applications involving live cells. To control the shape of frozen solid, one or more polymeric or non-polymeric additives may be added that can help to maintain the shape of frozen solid preferably without mechanical damage during handling and arrangement prior to crosslinking. The additive added should not interfere with the crosslinking process. Preferably the additive added is biocompatible if used for biomedical application. The additive may be small molecules such as sugar or it may be a polymer/macromolecule such as polyethylene glycol or its derivative, biodegradable or biostable polymers and its derivatives, cellulose and its derivatives such as hydroxymethyl cellulose, dextran, hyaluronic acid, collagen, gelatin, polyvinyl alcohol, polyvinyl pyrrolidone and the like. Inorganic fillers such as magnesium carbonate, barium sulfate, calcium carbonate, iron oxide, aluminum oxide and the like may also be used. The amount of additives may range from 0.001 percent to 20 percent, preferably 0.005 percent to 15 percent relative to the total mass of the frozen particle.

The precursor composition in the frozen or dry state is then exposed to electromagnetic radiation such as visible or UV light for a sufficient amount of time and at the desired wavelength and intensity to initiate effective solid state polymerization of the precursor. Biostable and biodegradable macromonomers that provide biostable or biodegradable crosslinked polymer as described in the previous section and FIG. 2 can be used. Biodegradable monomers or macromonomers with different biodegradable blocks and molecular weight, with additives like NBP can be effectively polymerized in a dry state or frozen state if effective polymerization conditions are provided. In some instances, dry macromonomer powder particles are formed upon drying. Monomers and macromonomers that photopolymerize in solid state or frozen state are preferred. The light used in photopolymerization includes but is not limited to ultraviolet, visible, or gamma ray or electron beam. UV and visible light and its corresponding initiating systems reported in this invention and known in the art are preferred. The intensity of light used for polymerization may range from 10000 mW/cm2 to 10 W/cm² or higher. Laser light sources are most preferred. Monomer compositions that gel or crosslink under two hours, preferably under 30 minutes and most preferably under 10 minutes are preferred. Many polymerizable macromonomer compositions are known in the art as well as macromonomers disclosed in this invention. Effective solid state polymerization conditions may be experimentally found for a given macromonomer system if such conditions exist. PEG based biodegradable and biostable composition macromonomers are known in the art as well as reported in this invention can be used in solid state polymerization. Macromonomers are generally described in FIG. 2A and B with different biodegradable blocks, central core like PEG or its copolymers, different types of polymerizable groups and number of polymerizable groups per macromonomer (greater than 2) can be effectively polymerized in the frozen state if effective polymerization conditions are provided. As discussed before, effective free radical polymerization depends on many factors including the type of polymerizable groups, the number of polymerizable groups and its polymerizibility, the molecular mass of macromonomer, macromonomer concentration, initiator concentration and its efficiency to initiate polymerization, organic solvents or their mixtures used and their ability to chain transfer, aqueous solutions or buffered solutions, pH of the aqueous solution, catalyst and cocatalysts used, impurities that inhibit polymerization, temperature, pressure, presence of oxygen in the atmosphere, wavelength and intensity of light used and the like. The illustrative examples provide effective polymerization conditions for exemplary compositions. Those skilled in the art of free radical polymer chemistry can understand that effective frozen state or dry state polymerization conditions can be found for each of the macromonomer compositions if such conditions exist. The preferred compositions that crosslink in solid/frozen state are biodegradable in nature.

The preferred crosslinked compositions polymerized in solid state or frozen state as described herein may be organic solvent gels or hydrogel in nature and preferably comprise PEG or its copolymers. The PEG molecular weight may be further limited to 400 to 35000 Daltons for biomedical implant applications. In many embodiments, before using photopolymerization and crosslinking in a dry/frozen or solution state, the ability of the precursor to effectively crosslink is generally experimentally determined first in liquid or solution state. Only those compositions that can be crosslinked under effective conditions in the solid state or in the frozen state must be used. This invention provides many exemplary compositions and methods that can be crosslinked under effective conditions. Those can be used as a guide to developing other crosslinking compositions for other monomers as described in this invention.

In some embodiments, precursors optionally with NBP can be subjected to standard extrusion methods or electrospinning methods that may be used to form fibers/filaments or fiber-like structures without crosslinking. The formed fibers or extruded structures which can be can be then polymerized and crosslinked in the solid state (partially dry state or completely dry state or frozen state). Compositions that can be extruded below 100 degree C., preferably below 70 degree and even more preferably below 65 degree are most preferred. Lower temperature use reduces premature crosslinking of precursors in the composition. The extruded fibers are cooled, chopped into the desired size and then irradiated with light to crosslink in the solid state. Alternatively, chopped particles may be incubated first in the initiator solution to infuse the initiator and then crosslinked in the swollen state using light. Solutions of NBP and precursors may also be extruded in a non-solvent or in a coagulation bath and coagulated fibers are chopped and crosslinked with light. Alternatively, the extruded precursors may also be irradiated and crosslinked first and then cut to the desired size. The extruded fibers or filaments with the same or different compositions may be knitted, woven, twisted to obtain woven/knitted/twisted structures. The woven/knitted/twisted structures may be exposed to light to crosslink via photopolymerization to lock/fuse the fibers/filaments in the woven/knitted/twisted form. The woven/knitted/twisted structures thus formed could be used for a variety of commercial applications.

Solid state polymerization (SSP) of macromonomers can be used in many applications. In one application SSP is used to make microparticles of the desired shape and size. In one illustrative embodiment, PEG20K urethane diacrylate (PEG20KUA, 10 percent) and PLGA (MW 125-150K, 20 percent) and Irgacure (0.1 percent) solution in DMSO is applied on a glass slide in a 1 by 1 cm square area and dried under vacuum to produce a substantially dry film. The PLGA and macromonomer formed a fine coating on the glass plate. This film is covered with a photomask (10 by 10 array pattern, 1 mm pitch and with 0.3 mm clear circular area for light passage) and is exposed to UV light (sample held at 2.5 cm from the lamp, 365 nm light with intensity 26500 μW/cm^{2) for} 2 min to effectively polymerize the precursor in dry state. The array plate is removed and the coating is then washed with a DMSO containing a photoinitiator to remove the unpolymerized coating. If needed, the washed composition may be further exposed to long UV light for additional crosslinking. The array pattern showing 300 microns diameter crosslinked polymer gel microcylinders is clearly seen on the plate. Crosslinking and gelation take place in the exposed area in the solid state. The crosslinked cylindrical particles are removed from the glass plate and stored until use. The PLGA in the particles may be infused with the drug for controlled release or may be added during precursor state if the drug can tolerate the crosslinking conditions. The thickness of the dry film and mask pattern is used to prepare microparticles of various shapes and sizes. Alternatively, the coated film can be exposed to an electron beam with sufficient energy to effectively crosslink the film in the solid state with the desired pattern.

FIG. 15 shows a partial schematic representation of a method for making drug or live cell encapsulated microparticles using frozen or dried precursors. In this method, the ability of the precursor solution to undergo crosslinking/polymerization in a solid state or frozen state is used for microencapsulation of drug/s or cells. Precursor/s compositions capable of undergoing effective polymerization in solid state or in the frozen condition are mixed with drug/live cells and the mixture is given the desired shape such as microdroplets. The mixture is then cooled/solidified in a controlled way to preserve the desired shape of the droplet and to maintain the viability of live cells if used. Generally, the programmable cooler is used to cool at 1 degree per minute until −70 to −90° C. and then final freezing in liquid nitrogen at −192° C. for better cryopreservation of cells. If necessary, a cryopreservative such as DMSO or glycerol (concentration around 10 percent relative to total solution weight or cell culture medium weight) may be added to improve cell viability during freezing operation. Commercial cryopreservative agents like CTS™ Synth-a-Freeze™ Medium, Synth-a-Freeze™ Cryopreservation Medium, Recovery™ Cell Culture Freezing Medium and the like may also be used as cryopreservative agents. The agent added should not interfere in the solid state polymerization and crosslinking process. The frozen mixture is then irradiated with visible or long UV light, UV or deep UV light or electron beam, preferably long UV or visible light to initiate effective polymerization and crosslinking in solid state or frozen state. The crosslinked particles are then warmed to room temperature or ambient temperature or stored in a frozen state until further use. In one illustrative application, solid state polymerization is used to encapsulate live cells preferably mammalian cells. Chinese hamster ovary live cells are suspended in PEG20K urethane diacrylate (PEG20KUA) in PBS (pH 7.4) precursor solution comprising Irgacure 2959 photoinitiator (0.1 percent) and 10 percent DMSO as an exemplary cryopreservative agent. A small amount of precursor suspension is removed to measure the viability of cells using live/dead assay to ensure at least 80 percent of cells are viable. The suspension is then added in silicone mold with 100 cylindrical mold cavities separated by 2 mm (cavity dimension 330 microns diameter and 500 microns height). Each cavity is filled with cell suspension, excess solution is wiped off and the mold is frozen at 0 degree C. and then to −192° C. using ice/liquid nitrogen (controlled cooling to maintain substantial cell viability). The cryopreservative agent used enables cells to withstand the freezing and thawing process. The frozen particles are thawed at −10° C. and exposed to long UV light (Black-Ray UV lamp, 360 nm light, 10000 mW/cm2 intensity, for 5 minutes) to initiate polymerization and crosslinking in the solid frozen state. The mold is warmed to room temperature and the crosslinked gel microcylinders are removed from the mold and suspended in the culture medium. The viability of cells is checked after encapsulation. Cells generally tolerate the crosslinking process without substantial viability loss (less than 30 percent loss in viability). Since cells are frozen during the crosslinking process, it is hypothesized that they can better withstand the encapsulation process (polymerization and crosslinking process) conditions without viability loss. In some cases, the cell suspension in monomer solution is frozen around zero degree C. and then exposed to light to effectively polymerize and crosslink in the frozen state. The crosslinked cells are then frozen in a controlled manner and stored in liquid nitrogen until use. In another modification as above, a drug solution/suspension, comprising macromonomer and photoinitiator in an organic solvent or in aqueous solution is sprayed using a sprayer to produce microdroplets (around 10 to 1000 microns in diameter) and the droplets are collected in the cryogenic bath. Cryogenic baths known in the art can be used for cooling. A cryogenic medium comprising water ice or solid carbon dioxide is preferred. Cryogenic baths comprising liquid gases like liquid nitrogen or liquid helium are most preferred. The frozen droplets are separated and warmed to −10 to 15 degree C. while maintaining a frozen state and exposed to UV/visible light as above for up to 10 minutes for effective polymerization and crosslinking (all droplets are exposed without shadowing effect). The crosslinked microspheres with encapsulated cells or drugs are then suspended in a suitable medium like culture medium and used. In the preferred embodiment, the particles are separated from each other by a fluid such as inert gas or oil before exposing to light. The separation distance is chosen such that it effectively prevents joining two particles during crosslinking. Preferred separation is 0.01 mm to 100 mm, preferably 0.1mm to 10 mm.

FIG. 15 shows a partial schematic representation of a method to make drug/live cell encapsulated microparticles/microspheres using frozen or dry precursor solutions. A photopolymerizable precursor solution is injected via sprayer (1501) to form microdroplets (1502) in the air. The sprayer 1501 has a tube in tube like arrangement wherein an outer tube (1503) is used to carry pressurized air or an inert gas like nitrogen or argon. The inner tube (1504) of the sprayer is used for transportation of precursor solution (1505) such as photopolymerizable macromonomer solution in water or organic solvents with a photoinitiator. The precursor solution may comprise a drug and/or live cells depending on the solvent used. Both the solution and gas exit from a small sprayer nozzle (1506) forming the solution droplets (1502) in the air. Optionally, the droplet emitting nozzle tip may be vibrated ultrasonically or other mechanisms to control droplet size. In some cases, the sprayer may be replaced with a droplet generating apparatus (1510) which generates picoliter to microliter size droplets (1511) of the desired volume. The droplets are then collected in a freezing medium (1507) preferably in liquid gases like liquid nitrogen or liquid helium which converts the liquid droplets into frozen microparticles/microspheres. Electric potential between droplet emitting tip and droplet collection beaker may be used as an additional tool to control droplet size. Upon evaporation of liquid gas, the frozen droplets (1508) are exposed to UV/visible light to initiate effective polymerization and crosslinking of precursor solution in the frozen state. The crosslinked microspheres (1509) are stored in a frozen state or at ambient temperature until further use. For living cells microfluidic apparatus may be used in place of a sprayer and cooling rates may be controlled to maintain substantial cell viability. In some embodiments, the frozen microspheres of desirable compositions may be arranged in a desirable 2D or 3D pattern and then exposed to light to lock the frozen state arrangement to make a unibody 2D or 3D material. Yet in another modification as above, a cell suspension is replaced with bupivacaine hydrochloride (as a model drug, final concentration 10 percent w/w), shaped into frozen microdroplets as above and polymerized and crosslinked by light exposure to form drug encapsulated microspheres. It is understood that Chinese hamster ovary cells are used only for illustration and other types of cells, preferably mammalian cells and most preferably therapeutic cells like therapeutic stem cells may also be used. Cellular elements like cellular components or fragments, enzymes, bacteria, viruses, fungi, algae, DNA, RNA, mRNA and genes and their combination in any proportion may also be used in solid state polymerization and encapsulation as discussed above. The same technique can also be used to entrap live cells in the tissue engineering scaffolds where organs or organ parts can be grown in a laboratory environment. Macromonomers that provide biodegradable crosslinked polymers are preferred in tissue engineering applications. Macromonomers comprising polycarbonates, gelatin, collagen, hyaluronic acid, fibrinogen, PEG, PEG-PPO, PEG-PLA , PEG-polylactone, and the like or their blends or copolymers are preferred for tissue engineering application. Alternatively, droplets may be made using many types of 3D printer like devices known in tissue engineering or 3D printer art. The 3D printed particles are frozen and then exposed to light for encapsulation. In one exemplary embodiment, frozen shapes with LEGO® like features/shapes are used to mechanically bond with each other and then effectively crosslinked to prepare mechanically as well as covalently bonded crosslinked materials. In one illustrative embodiment, bead-like frozen structures with hollow cavities are made first. The hollow space in the bead could be used to thread polyethylene vinyl acetate copolymer or PLGA fiber or filament with the drug in the beads. Bupivacaine hydrochloride is used as an illustrative drug. Other drugs may also be used using methods and compositions described in this invention

Multilayered Crosslinked Compositions

In this invention, we disclose many methods and compositions to obtain two or more layered unibody particles or microparticles or objects wherein each layer is covalently linked with other layer/s and each layer can have either the same or different chemical composition. Each layer also can have different physical or chemical properties. Also disclosed are sheet-like materials and other 3-dimensional unibody materials whose compositions are made from submicron to several millimeter size distant blocks of precursors. Each block is covalently linked with another block to form a unibody composite material. The arrangement of different blocks, their shape, size and their location in the sheet or 3-dimensional object is predetermined before making the material. The resultant novel composite materials have unique combinations of properties such as density or refractive index gradients/patterns, a unique combination of elastomeric and rigid materials and the like. In the preferred mode, submicron to mm size shapes of precursors with the same or different compositions are arranged to make the desired pattern and then crosslinked within and between the precursors to produce a composite material. Shapes of precursors also can be arranged such that they can be mechanically interlocked before crosslinking. Such arrangement produces mechanically interlocked and covalently bonded crosslinked materials. Shapes of precursors also can be arranged such that they can have the desired amount of empty space or cavities of a predetermined shape. Upon crosslinking, the resultant crosslinked composite material produced is mechanically interlocked and is also covalently bonded and can also have the desired amount of porosity whose shape is also controlled. Various illustrative embodiments teach several methods to make two layered unibody materials. Those methods and compositions can be applied to make multilayered 2-dimensional or 3-dimensional materials. In preferred embodiments, the precursors that polymerize by free radical or condensation polymerization mechanism as described in this invention are used. Free radical or chain growth polymerizable precursors are most preferred. The use of organic solvents and aqueous solutions and their combinations in any proportion is used to polymerize and crosslink precursors to make two or more layered materials.

FIG. 7 shows a partial schematic representation of composite and/or multilayered materials described in this invention. FIG. 7 A, B, C show preferred composite and layered particles of various types, shapes and sizes. The spherical shaped composite material A (microsphere) comprises crosslinked material (701), preferably biodegradable crosslinked material with a non-crosslinked biodegradable polymer (702) wherein the biodegradable polymer 702 is precipitated in situ inside the crosslinked material 701. B is the same as A except in a microneedle shape or form. C is similar to A in the form of a coating on a medical device. FIG. D shows multilayered particles wherein layers 703, 704 and 705 are completely encapsulated in a separate encapsulating material layer (706). The layers 703, 704 and 705 present as separate layers in the 706 encapsulating layer and are not covalently bonded. FIG. E shows the microneedle of a microneedle array device with two layers, a base layer (707) and microneedle layer 708 and each layer may be loaded with a visualization agent and/or drug. FIG. F1 shows a single cylindrical unibody particle with two layers (709, 710) wherein layers 709 and 710 are covalently linked. FIG. F1A shows a microscopic image of two layered unibody particles (diameter around 300 microns, length around 500 microns) similar to F1 wherein the blue layer is a crosslinked PEG-based macromonomer hydrogel layer with blue colorant additive and the transparent layer is a crosslinked gelatin methacrylate hydrogel and both layers are covalently linked to form a single unbody microparticle. FIG. F2 shows a unibody three-layered particle with 709, 710 and 711 as unique layers that are covalently linked with each other wherein 712 shows interface between two layers. F3 is similar to F1 except in the form of torus or ring shape with two layers. F4 shows spherical particles with two layers. H1 shows a sphere/disk shaped (715) particle that is covalently attached to the film or sheet surface (716) to form a unibody material. H2 is similar to H1 wherein 715 is attached to the luminal surface of a tubular body. H3 is similar to H1 wherein 715 is embedded/encapsulated in a hydrogel or polymer sheet/film (717). H4 is a cubical particle (718) with an embedded spherical particle (715). 715 may be physically entrapped or covalently linked to 717 or 718. Each layer in all particles described above may have a different composition or encapsulant or physical/chemical property.

In one illustrative embodiment, a silicone mold cavity is used to make layered microparticles. FIG. 17 shows a schematic representation of a method for making biostable or biodegradable multilayered microparticles. FIG. 17A schematically shows a cylindrical mold cavity (1701). FIG. 17B shows the schematic mold cavity 1701 that is partially filled with a precursor composition (1702). The illustrative 1702 precursor composition may comprise a biodegradable polymer and visualization agent such as colored or fluorescent compound. The composition in 1702 is optionally frozen or reversibly gelled or dried to prevent mixing with an additional layer of crosslinkable precursor composition to be added as a second layer. In FIG. C, a second layer precursor composition (1703) is added. The illustrative 1703 precursor composition may comprise a biodegradable polymer and drug. Both layers (1702 and 1703) are optionally frozen or reversibly gelled to add a third layer of crosslinkable precursor. In FIG. D, a third layer precursor composition (1704) is added. The illustrative 1704 precursor composition may comprise a biodegradable polymer and radio-opaque compound. All layers may be partially or completely liquefied by warming to room temperature if frozen or converted into liquid/solution state if reversibly gelled and then effective crosslinking is initiated before components in each layer substantially diffuse into other layers. The crosslinked three layered unibody particles are removed from the mold with preserved multilayered structure (E). In some embodiments, the crosslinking may be initiated when some or all the layers are partially or completely dried, frozen to a solid state, in physical gel form or liquid state or combination thereof in any proportion. The crosslinking of precursors occurs in all layers producing layered microparticles with distant layers wherein each layer may comprise different composition or has different chemical/physical properties. All layers in particle E are not separable and are fused with each other and combinations of all layers exist as one single unibody particle/body. In some embodiments glass capillaries, channels of a microfluidic device (preferably made using glass or inert materials), silicone or other inert material tubing or mold cavities are used to cast precursor solutions into multilayered particles. Depending on the precursor, mold material may be hydrophobic, hydrophilic, hydrogel or organogel. Mold materials may be stainless steel, glass, silicone rubber, or any other suitable inert material that is compatible with the precursor components. Glass, metal or silicone rubber based tubes or capillaries may be obtained from commercial sources and the surface of the tube may be modified/coated with coatings to manipulate surface tension, ease of particle removal and the like. Biocompatible coatings like silicone or mineral oil, vitamin E may be used to lubricate easy implant removal. Hydrophilic or hydrophobic coatings may be covalently bonded or physically coated to adjust surface tension.

In one illustrative embodiment, a freezing solution technique is used to make multilayered particles. The freezing enables it to maintain a separate phase while adding precursor components before crosslinking. A silicone rubber mold with 100 cubical cavities in a 10 by 10 array format separated by a 2 mm distance is prepared. In a 15 ml glass vial, PEG35KUA macromonomer solution in DMSO comprising PLGA 10000-15000 Daltons, Iodixanol as a model radio-opaque agent and 2-hydroxy-4′-(2-hydroxyethoxy-2-methylpropiophenone as long UV photoinitiator is prepared. Similarly, another solution as above is made wherein lodixanol is replaced with bupivacaine hydrochloride (drug) as a model drug. lodixanol solution is added to each of the mold cavities and the mold is frozen in liquid nitrogen. The frozen mold is taken out and the remaining space in the mold cavity is filled with bupivacaine hydrochloride solution. The mold is then exposed to long UV light (360 nm) for 5 minutes to polymerize both the precursor compositions. During polymerization, the precursor may be in the frozen state, frozen state and liquid state or liquid state. If both components are in the liquid state, the polymerization is done quickly before substantial mixing of two liquid components. The polymerized unibody particles are taken out and DMSO is removed by vacuum drying. Removal of solvent precipitates the PLGA in the crosslinked matrix and encapsulates the drug and lodixanol in the crosslinked matrix as well as in PLGA. The single particle thus formed has two zones/layers in unibody structure wherein one of the zones comprises lodixanol as a model radio-opaque agent and the other zone comprises bupivacaine hydrochloride as a model local anesthetic drug. Both components are encapsulated PLGA matrix as well as crosslinked PEG based polymers. In another embodiment, one or more liquid precursors are used in a mold to produce a two or more layered unibody material. The mold used has removable inert material spacer/s to make cavities of desired size and shape. The liquid precursors are added to each cavity and the removable spacer is removed. The liquid precursors in the cavities come in contact with each other and are then exposed to light to quickly effectively polymerize and crosslink before substantially mixing of precursors to produce a unibody object.

In another exemplary embodiment as above, a physically crosslinked gel such as a thermosensitive gel is used to make multilayered particles. F127LA macromonomer at 25 per concentration in water or PBS (pH 7.4) can be crosslinked in a solution state or in a gel state (around 37 degree to 50 degree C.). F127LA macromonomer, lodixanol and UV photoinitiator Irgacure 2959 are mixed at 0-10 degree C. to form a homogeneous solution. Another cold solution is made wherein iodixanol is replaced with albumin as a model protein drug. Cold F127LA-lodixanol solution is added in each cavity of the silicone mold as above and the mold temperature is increased to 40 degree C. to cause physical crosslinking or reversible thermal gelation of F127LA macromonomer solution. While in the gel state, the second F127LA/albumin cold solution is added in the remaining space of the cavity. Excess solution is wiped off from the mold surface and the solution is immediately exposed to 360 nm UV light to initiate photopolymerization in both layers. During polymerization the precursor may be in the gel state, gel and liquid state or liquid state depending on the temperature used. If both components are in the liquid state, the polymerization is done quickly without substantial mixing of two liquid components. The polymerization of both solutions leads to single gel unibody particles with two layers with one layer comprising F127LA-lodixanol and the other layer comprising crosslinked F127LA-albumin. Other precursor macromonomers that have pH sensitive or thermosensitive gelation properties can be used similarly. These include derivatives of Pluronic, Tetronics, peg-polylactone copolymer, PEG-PPG copolymers, gelatin, collagen, chitosan, cellulose derivatives and the like. In another embodiment, Jeffamine lactide acrylate (JAL2UA) is used as another illustrative biodegradable thermosensitive macromonomer to make two layered particles as described above.

In another illustrative embodiment (EXAMPLE 12A-3), a solvent evaporation technique is used to fill the mold cavities. Briefly, a precursor comprising water or organic solvent is added to completely fill the mold cavity. The solvent is partially evaporated or removed to make room/space in the cavity for a second precursor solution. Solvent evaporation by air or vacuum drying, sublimation or lyophilization and the like may be used to create the additional space. The second precursor solution is then added in the newly created space by solvent evaporation and both the solutions are effectively polymerized and crosslinked to prepare a bilayered or two layer unibody implant as discussed before. Depending on the amount of solvent used in the first composition and the amount of solvent evaporated, the space created for the second composition can be varied. If necessary, the steps as outlined above may be repeated to obtain additional layers.

FIG. 12 shows a partial schematic representation of a method for making drug or live cell encapsulated multilayered microparticles using transparent glass capillary. In this method (FLOW DIAGRAM 5), a transparent glass capillary tube or mold cavity (1201) is inserted with a piston (1202) which can be moved along the length of the capillary tube to suck/pull a controlled amount of the desired precursor solution (FIG. A). FIG. 12B shows the glass capillary described in FIG. 12A inserted in a photopolymerizable precursor solution A reservoir (1203). The piston is moved upward to create a controlled amount of space in 1201 by pulling out a measured amount of precursor solution A in the capillary (1204). Depending on the composition used, capillary action (surface tension) may also be used to drive a measured amount of solution in the capillary without the use of a piston. The solution on the external walls of capillary 1201 is wiped/cleaned to prevent contamination. The assembly is then transferred to photopolymerizable precursor solution B (reservoir C, 1205). The piston is moved upwards and a measured/controlled amount of precursor solution B is then pulled/sucked in the glass capillary, schematically shown as 1206. The assembly is then taken out, excess solution B is wiped out from external walls of the capillary (D) and then exposed to UV or visible light to induce effective photopolymerization and crosslinking of the precursor solutions A and B. The effectively polymerized precursor solutions A and B are schematically shown as 1208 and 1207 respectively (E). The piston is then pushed downwards to inject a polymerized microcylinder particle (F) from the capillary which exists as a single unibody particle but having two distinct layers 1208 and 1207 within a single unibody structure.

In one illustrative embodiment, bilayer unibody material is made from a single macromonomer but using an additive/filler to change the properties of the other layer. In one illustrative embodiment, PEG35KUA macromonomer is dissolved in PBS along with a photoinitiator and magnesium carbonate is added as an illustrative visualization aid or as a filler to form a suspension. Similarly, another solution is prepared the same as above but without the filler magnesium carbonate. Macromonomer solution without filler is pulled inside a transparent glass pipette capillary tube followed by a solution with magnesium carbonate forming two solution layers inside the pipette tube. The layers are then exposed to light to quickly polymerize and crosslink both the layers. The crosslinked material is injected out and has two layers fused/merged with each other forming a unibody material. One layer is transparent (without filler) and the other layer is opaque (with magnesium carbonate). In one modification of the above embodiment, solutions of both the precursors are alternately loaded in the pipette and then crosslinked to prepare cylindrical unibody objects with many alternating layers of transparent and opaque layers. A similar experiment is carried out in glass capillaries plates with 10 micron diameter and 400 micron height) where the composition is first completely filled in the plate and then the solvent is partially removed by air/vacuum drying and then the second composition is filled in the newly created space in the capillaries and then crosslinked using long UV light. The crosslinked unibody is removed from the mold. If needed the length can be further reduced by cutting the particles. Glass capillary tubes with diameters ranging from 50 microns to 2000 microns may be used to create cylindrical multilayered objects. Even smaller size capillaries or mold cavities may be made using hollow fiber tubes, microfluidic channel devices and the like. Microfluidic channels of a microfluidic device can have many shapes such as rectangular shaped channels, cylindrical tubular channels and the like and such channels may be used to prepare particles with other 3 dimensional shapes such as rectangular prism shaped multilayered particles. In the illustrative embodiment, one macromonomer composition is used to make bilayer particles by adding filler material. Instead of filler magnesium carbonate, other additives that may be used include but not limited to: magnetic particles, colorant, radio-opaque agent, drug encapsulated microparticles and the like that give different physical properties to the second layer. In some embodiments, the ability of macromonomers to polymerize and crosslink in two different solvents is used to make multilayered particles. Preferably solvents used are commonly used organic solvents or water based buffered solutions. EXAMPLE 1 to 3 discloses various illustrative examples of precursors that polymerize in aqueous and organic solvents. The precursors disclosed in this invention and other precursors known in the art can be used in making illustrative multilayered particles. In another modification of the above example, the macromonomer solution without filler is made in DMSO instead of PBS and used to make multilayered particles. The particles thus formed have one layer as hydrogel formed in an aqueous medium with magnesium carbonate as a filler and another layer as an organogel formed in organic solvent DMSO. In another embodiment, the PEG20KUA macromonomer is dissolved in distilled water along with initiator stock solution. Separately, another solution is made comprising PEG20KUA in dissolved DMSO along with photoinitiator and PLGA (125-150K) as a model non-biodegradable polymer (NBP) additive. Both solutions are filled sequentially in a glass capillary with 0.5 mm diameter and exposed to long UV light. The polymerized unibody microcylindrical particle is taken out from the capillary. The bilayer particle is then immersed in coumarin (a model drug and fluorescent dye) solution in DMSO for one minute to diffuse coumarin in the particle and then washed with water two times to remove excess coumarin. The coumarin preferentially accumulates in the organogel PLGA layer as compared to the non-PLGA hydrogel layer of the particles. The illustrative PLGA polymer used is water-insoluble but soluble in an organic solvent such as DMSO. Thus, crosslinking in the organic solvent is useful in entrapping non-water-soluble additives like PLGA in one of the layers. In another embodiment, PEG20KUA macromonomer is dissolved DMSO along with UV photoinitiator Irgacure 2959. Separately, another solution/suspension is made comprising PEG20KUA which is dissolved in distilled water, photoinitiator and magnetic ferrite powder as filler. Both solutions are filled in a 0.5 mm diameter glass capillary in sequence and exposed to long UV light. The polymerized unibody microcylindrical particle is taken out from the capillary. The bilayer particle is then tested for magnetism using a small laboratory magnet. The ferrite portion attaches to the magnet indicating the magnetic property of the bilayer particle. A similar bilayer particle is made using iron oxide powder instead of ferrite powder; it also showed magnetic properties to the layer. In another variation of the same experiment, the UV exposure and crosslinking are done in a glass capillary in presence of the magnetic field. The poles of magnetic particles in the precursor solution are pre-aligned in the magnetic field and then effective crosslinking is initiated using long UV light to lock the particle's magnetic oriented position in the magnetic field. The magnetic particles in the layer are entrapped with a unified orientation. In another modification of the current example, PEG20KUA macromonomer and lipase as an illustrative enzyme are dissolved in TRIS buffer (lipase concentration 1 mg/ml, 100 mM TRIS and 1 mM EDTA, pH 8) which is then used in place of DMSO solution and bilayer particles are made as described above with ferrite particle. The bilayer particles thus produced have magnetic particles in one layer and another layer has enzyme lipase encapsulated in the crosslinked matrix. Examples 12D1-12D28 show the preferred combination of various layers in a multilayered particle.

In one illustrative embodiment, a two layered magnetic particle is made using a procedure similar to described above. One layer has biostable crosslinked material with illustrative enzyme lipase in one layer and another layer has biostable crosslinked material with magnetic particles. Thus, a combination of magnetic properties and enzymatic/catalytic activity of the layers are kept separate which may be useful in many industrial applications including but not limited to applications in the food, pharmaceutical, paper, detergent and biofuel industry. Lipase enzyme is used as an illustrative catalyst and other chemical/biochemical catalysts that can be used include but not limited to are: trypsin, amylase, glucose isomerase, papain, pectinase protease, lipase, amylase, cellulase, xylanase, nitrile hydratase, transaminase, monoamine oxidase, penicillin acylase, and the like. PEG or PEG copolymer based crosslinked materials are especially useful for enzymes/catalysts that are active in organic solutions. Excellent swellability of PEG based materials in aqueous and organic solutions and their ability to control the molecular permeability can be used to design many enzyme based multilayered particles. Multilayered or two-layer particles with one layer having magnetic particles and the other layer comprising one or more live mammalian cells or genetically modified bacteria or yeast are embedded. Such particles may be used to manufacture bioactive compounds made by mammalian cells or bacteria or yeast. The permeability of hydrogels keeps the cells or bacteria alive when incubated in the desired medium which enables them to produce desired bioactive compounds. The magnetic property can help the particles to align the particles in a desired fashion in a bioreactor and also it may be used to separate the cells or bacteria from the reaction medium.

In some embodiments, the precursor solution/s are injected in the glass tubing or microfluidic chip path using a syringe pump or other liquid infusion/injection device. The amount and sequence of precursor solution injection are precisely controlled by the infusion pumps with attached valves to the pump so that the desired sequence and amount of precursor solutions are infused in the microfluidic chip or glass capillary tube. The infused components are then polymerized and crosslinked as described before to produce unibody multilayered particles. Glass capillaries with different diameters and lengths may be purchased from glass manufacturers or laboratory equipment suppliers. The transparent nature of glass is useful for precursors that can be photopolymerized using UV or visible light. Glass capillary plates with well-defined length and diameter are available from commercial sources (Hamamatsu Corporation, Photonics Division, Bridgewater, NJ, product J5022-11 with 10 micron diameter and 400 micron height) may also be used. In some embodiments, glass slides with laser drilled holes of the desired diameter and slide thickness may also be used as molds. Glass slides with desired hole sizes can be custom made from commercial vendors such as Potomac Photonics, Baltimore MD. The methods described above are used to make cylindrical single or multilayered particles with diameters ranging from 1-2000 microns and with lengths from one micron to 50 mm. In some embodiments, while using glass capillaries, a larger than the expected length of the bilayered particle is obtained. In such cases, the desired length is achieved by subjecting the particle to sectioning/cutting using a microtome machine to obtain the desired length. Before sectioning, the particle may be embedded in cutting media like wax or in gelatin gel or maybe be frozen or lyophilized to provide mechanical support during the cutting operation and then cut.

In some embodiments, a combination of the precursor solution in the organic solvent and aqueous solution are used to make multilayered microparticles. Precursor solutions made in different organic solvents may also be used to make multilayered particles. The solubility of precursors in organic solvent and aqueous solutions, preferably buffered aqueous solutions gives more flexibility to dissolve many types of precursors/monomers. The solubility is also useful to add other components such as drugs, visualization agents, fillers, reinforcing agents, non-crosslinked biodegradable polymers (NBP) and the like which may have a solubility in a different organic solvent or aqueous solutions. Many useful and commercially available non-crosslinked biodegradable polymers and biostable polymers such as polylactones like PLGA are generally only soluble in organic solvents and therefore use of organic solvents may be necessary in such cases. Many combinations of organic solvents in making unibody multilayered microparticles can be used and only those solvent combinations that provide unibody implants are preferred. Some illustrative embodiments demonstrated the use of organic solvent combinations in preparing multilayered unibody implants. Those skilled in the art can recognize that many combinations of organic solvents and aqueous buffered solutions can be used. Due to the vast array of organic solvents available and their combinations, such solvents are considered as part of this invention. Preferred combinations of organic and aqueous solutions include but not limited to: dimethyl sulfoxide (DMSO) and water or water based buffers like PBS buffer pH 7.4; ethyl acetate and DMSO; PEG dimethyl ether molecular weight 600 and ethyl acetate, PEG molecular weight 200 to 2000 and ethyl acetate and the like. In some experiments, solvent combinations like dichloromethane-water, ethyl acetate-water, toluene-water formed non-unibody crosslinked gels or particles under effective crosslinking conditions (each layer is polymerized separately without merging/fusing to other layers; polymerization occurs within layers but not between layers). Such gels may need to be further encapsulated into another encapsulated material, preferably crosslinked material to form a unibody particle (FIG. 7D). It is generally observed/hypothesized that miscible solvents combinations tend to provide unibody particles and immiscible solvents pairs tend to provide non-unibody particles when used with free radical polymerizable macromonomers, however, this invention is not limited to this hypothesis. A particular precursor/s solvent combination and other crosslinking conditions may be tested/evaluated for unibody particle formation before using it in the desired application. Many examples of successful unibody formations are provided in this invention and those skilled in the art can understand that modifications can be made to those compositions and experimental conditions to obtain desired multilayered particles.

In some embodiments, two or more molds are used to make multilayered particles. FIG. 11 shows a partial schematic representation of methods for making multilayered microparticles/implants using two or more molds. FLOW DIAGRAM 3 describes a method of using two or more molds to make cylindrical two layered particles and FLOW DIAGRAM 4 describes a modified version of FLOW DIAGRAM 3 to make spherical two layered particles. FIGS. A and B schematically show molds used for casting of multilayered particles using methods and compositions described in this invention. A shows a rectangular body (1101) made out of silicone rubber or gelatin hydrogel or other desirable mold material and the mold body has one or more microcavities (1102) created for the casting of crosslinkable precursor microparticles. Mold A also has 4 cylindrical columns or guideposts (1103) used for alignment and holding the second mold B. Mold B is similar to A and has a base body (1104) with a top surface (1104T) and bottom surface (1104B) and mold cavities (1105) that are open on both surfaces (1104T and 1104B). B also has additional 4 holes (1106, open on 1104T and 1104B surfaces) on each corner whose diameter is slightly bigger than the diameter of guideposts on A but the center of guideposts on A (1103) and center of corner holes on B (1106) are aligned when placed on top of each other. The size, shape and arrangement of cavities on molds A and B are preferred to be identical. FIG. C shows mold cavities on mold A filled with precursor solutions such as macromonomer solution with photoinitiator (1107) and excess solution is wiped off.

Optionally, the precursor solution may be frozen or thermoreversibly gelled to reduce diffusion of components within each layer. The mold B is then inserted in mold A via guideposts (1103) through the corner holes (1106) aligning the mold cavities of A and B (FIG. D). The mold cavities of B are then filled with a second photocrosslinkable precursor solution (1108) which may be different from the first one (1107). The excess solution is wiped off. First or both solutions may be partially or completely liquified and the mold cavity is exposed to UV/visible light to induce effective photopolymerization and crosslinking of precursor 1107 and 1108 compositions. The polymerization and crosslinking occur in both molds forming a singular crosslinked unibody implant (F). The crosslinked gels are removed from the mold. The particle thus produced has a single body or unibody (F) with two layers of (1109 and 1110) crosslinked compositions.

In some embodiments, a sealing gasket may be needed between the two molds to prevent leakage of low viscosity precursor solution outside the molds. Generally, quick polymerization and crosslinking of the precursor solution before leaking is used to avoid leakage. Additional layers may also be added by the use of additional molds similar to mold shown in FIG. 11B provided mold cavities are aligned and leakage of precursor solution is prevented. In some embodiments, the ability of some compositions to form a crosslinked polymer in a solid state or frozen solution state or thermoreversible gel state is used. Compositions that can be polymerized in a frozen state or liquid state or a combination thereof are preferred. Precursor solutions that can be frozen between −40 to 50° C. are most preferred. All mold cavities are filled, excess solution is wiped off. The molds are then aligned in a frozen state. If solid state polymerization is used then precursor surfaces must be in physical contact with other precursors to form a unibody implant. One, both or all frozen solutions may be liquified just before crosslinking or exposure to light. The crosslinking is then initiated. This method helps to reduce or eliminate leakage issues associated with the low viscosity precursor solution. The process shown in FLOW DIAGRAM 4 is used to make spherical shaped bilayered particles. G shows a semispherical (half-spherical) shaped transparent or semi-transparent mold cavity (1111). The cavity 1111 is filled with precursor solution 1112 (H). I show a second precursor solution (1113) filled in the second mold. Solutions 1112 and 1113 are reversibly gelled and/or frozen to form gels/solids (1115 and 1114). The mold H and I are aligned and kept on top of each other such that open surfaces of 1112 and 1113 touch each other and the resultant shape is spherical in nature (J). Precursor solutions are exposed to light to effectively polymerize and crosslink both solutions without substantial mixing. The precursor may be in the frozen state, liquid state or physically crosslinked gel state or combination thereof. H shows polymerized unibody spherical particles with two layers 1116 and 1117 wherein 1116 is crosslinked 1112 and 1117 is crosslinked 1113.

In some embodiments, a photolithographic method is used to make multilayered particles. In one illustrative embodiment, a method similar to described in EXAMPLE12-3 is used. Instead of using one layer as described in the example, two layers of precursors are used on top of each other. Both layers may have different compositions but can crosslink and form unibody composition. The layers are exposed to UV light via photomask or electron beam scanning and polymerized. Upon removing unpolymerized mass via precursor solvent washing, multilayered particles are collected. Only those compositions that can be effectively polymerized in all layers in solid state or frozen state or liquid state can be used in this method.

In some embodiments, two frozen precursor particle compositions are prepared first. The frozen particles are crosslinked together to form a unibody bilayered particle (FIG. 13H). Using a similar technique three or more particles are joined to make a multilayered particle (FIG. 13I). In one illustrative embodiment, a sheet-like material with alternate blocks of gelatin methacrylate with and without magnesium carbonate is polymerized in a checkered fashion as described above. A 10 percent solution of gelatin methacrylate in distilled water along with Irgacure 2959 (0.1%) as a photoinitiator. The prepared solution is divided into two parts. To one part magnesium carbonate (about 10 percent) as a model visualization agent that induces opacity to the crosslinked gels is added. The other solution did not have filler particles. Magnesium carbonate filler may be substituted with drug encapsulated microparticles as a visualization agent as well as a drug delivery agent. The two solutions are then filled into 2 mm by 2 mm by 2 mm silicone rubber mold cavities and frozen. The frozen cubes of each part are arranged in a 4 by 4 alternate checkered pattern (total 16 cubes, an arrangement similar to shown in FIG. 14E in cold frozen condition) in another silicone rubber mold. The arrangement is tight and at least one surface of the cube is in physical contact with the other cube in a frozen state. The cubes are then optionally warmed to room temperature until the melting of gelatin methacrylate on the surface of the cube just initiates and is then exposed to long UV light (360 nm) for 5 minutes. The exposed cubes undergo photopolymerization and crosslinking in solution state and/or frozen state forming a unibody structure with zones of gelatin methacrylate filled with magnesium carbonate (opaque gel) and without magnesium carbonate (clear gel). The crosslinked structure is incubated in water for 20 minutes at ambient temperature to test the formation of the crosslinked and unibody structure. Upon incubation in water, none of the cubes are dissolved indicating effective polymerization and crosslinking and all cubes were fused with each other indicating unibody formation (FIG. 14E1 and 14E2). Using a similar method, composite sheet materials with alternate blocks of hard crosslinked materials (crosslinked PEG2KUA) and soft crosslinked materials PEG1OOKUA or PEG35KUA) materials are made. The precursors used in the above examples are for illustration only. Many monomers and macromonomers are known in the art and some of them have been described in this invention.

In FIG. 12, FLOW DIAGRAM 6 shows a modification of the FLOW DIAGRAM 5 method wherein cold precursor microparticles/microspheres are used to form layered composite unibody material. A needle connector portion of a syringe is cut off leaving behind a syringe barrel and plunger. E shows a glass syringe barrel (1210) with a glass syringe plunger (1211). The syringe is cooled in the freezer at −20 degree C. The syringe plunger is moved down to create a space in the barrel (1212). This space is then filled with cold frozen microspheres of precursor solution (1213) with a photoinitiator with an ability to effectively polymerize in a frozen state such as gelatin methacrylate solution in PBS (GM). After filling space 1212 completely, the plunger is pulled down again to create additional space in the barrel. The additional space is then filled with a second layer of cold frozen microspheres/microparticles (1214) of different or same precursor solution with photoinitiator with an ability to effectively polymerize in the frozen state such as PEG1OKUM in PBS. The frozen microspheres in both the layers are exposed to light until effective polymerization of precursor microspheres in the syringe barrel. The effective polymerization produces two layered unibody objects comprising polymerized crosslinked precursors (I, 1215 and 1216). The polymerized bilayer unibody object comprising layers of 1215 and 1216 is pushed out of the syringe (J) and stored until use. In another variation of this method, two frozen microspheres precursors (1213 and 1214) are mixed using equal volume and then filled into the empty space of a barrel (1212) and exposed to light to effectively polymerize both the precursors. This yields a composite of crosslinked unibody material wherein precursors 1213 and 1214 are covalently linked with each other within the crosslinked unibody structure. The crosslinking takes place within and between microspheres forming a composite material and 1213 and 1214 are randomly distributed in the unibody structure.

EXAMPLE 11 and 12 teach illustrative embodiments for making multilayered particles. In the preferred embodiments, the particles are made by using precursors of crosslinked biostable and biodegradable compositions. The precursors are polymerized by free radical polymerization or by condensation polymerization mechanisms to make such particles. Many illustrative embodiments use photocrosslinkable compositions that are polymerized in the solution state, neat liquid state, solid state or frozen state or combination thereof. EXAMPLE 11 teaches methods for making multilayered biostable or biodegradable hydrogel/organic solvent gel compositions. In one illustrative embodiment, a PLGA polymer is used to make rifampin encapsulated microparticles/microspheres in the first step. In the exemplary embodiment, rifampin encapsulated microspheres and bupivacaine encapsulated microspheres size ranging between 1 to 70 microns are made first using the standard emulsion based method known in the art. Microspheres without the drug Rifampin are also made and are used as control. The control microspheres could also be stained to obtain colored PLGA microspheres without drugs. The rifampin, bupivacaine or control microparticles as mentioned above are then used to make multilayered crosslinked microparticles. Many methods are known in the art to make drug/visualization agent encapsulated microspheres using biodegradable polymers such as PLGA as an encapsulation matrix for controlled drug delivery application. Such methods may be preferentially used. Rifampin and bupivacaine loaded PLGA encapsulated microspheres are made separately. The microspheres are then suspended in PBS solution of PEG35KUA macromonomer along with photoinitiator. The suspension with rifampin is first loaded in a glass capillary (500 micron ID) followed by suspension in bupivacaine are loaded as two distant precursor solution layers (layer height around 2 mm) and then exposed to long UV light for effective photocrosslinking and polymerization. Since both the layers have the same macromonomer, polymerization and crosslinking result into a single cylindrical unibody hydrogel particle with two distant layers each layer containing bupivacaine and rifampin respectively and both drugs are present in an encapsulated form in the unibody crosslinked PEG35KUA hydrogel matrix. The particles may be cut from both ends using a microtome machine to obtain the desired length if needed. The drugs are encapsulated in the PLGA matrix for the desired controlled release profile. Rifampin also serves as a visualization agent as well as a drug. In another configuration, a three layered hydrogel particle with around 20 percent of the volume occupied by microspheres containing radio-opaque agent, around 10 percent volume occupied by microspheres with a colored or fluorescent agent and remaining volume (around 70 percent) occupied by microspheres containing drug/s is made. In another example, the crosslinked PEG35KUA macromonomer matrix is replaced with a crosslinked biodegradable matrix obtained from condensation polymerization of PEG10K4 ARM tetramine and of PEG10K4ARM glutarate NHS ester in PBS. The precursors and encapsulated microspheres are mixed to form a suspension and then loaded in the glass capillary as two different solution layers before crosslinking of precursors. The condensation polymerization and crosslinking are completed in the capillary forming a bilayered matrix with microspheres embedded in the crosslinked gel. In another variation of this embodiment, one layer has no microspheres in the crosslinked matrix and the other layer has microspheres such as PLGA with bupivacaine or rifampin and or their mixture in any proportion are used. EXAMPLE 12D provides illustrative precursor solution combinations to make a variety of preferred multilayered particles with different properties/composition in each layer. The drug/microspheres microparticles or microspheres used in making multilayered particles have an average size ranging from 0.2 microns to 2000 microns, preferably 0.5 microns to 1000 microns. The drug content of such microparticles may range from 0.1 percent to 40 percent, preferably 0.5 to 30 percent. The preferred drugs are antimicrobial, antibiotic, local anesthetic, anti-inflammatory and anticancer drugs. The total weight percent of the microsphere relative to the particle weight may range from 1 percent to 300 percent, preferably 1 percent to 100 percent. Preferably biodegradable particles are made from synthetic or natural biodegradable polymers. A list of preferred biodegradable polymers is disclosed elsewhere in the invention. Synthetic biodegradable copolymers and polymers of polylactones and polycarbonates are most preferred. The particles may also have visualization agents encapsulated in the particles. The concentration of visualization agents may range from 0.05 percent to 60 percent preferably 0.1 percent to 50 percent. In some embodiments, inorganic biocompatible particles such as magnesium carbonate, calcium carbonate and other biocompatible and biodegradable salts may be used as particles. In the preferred embodiment, one layer has drugs encapsulated in microspheres and the other layer has microparticles with visualization agents. Those skilled in the art can understand that many permutations and combinations of these compositions and methods disclosed herein may be used to obtain different types of multilayered particles and such combinations are considered as a part of this invention. FIG. 14 shows microscopic photographs of the multilayered unibody materials prepared using methods and compositions described in this invention. FIG. A shows cylindrical shaped two layered unibody particles wherein one layer has only crosslinked polymer and the other layer has the same crosslinked polymer with a non-crosslinked biodegradable polymer (NBP). About the bottom, two thirds part of the particle is a semitransparent organic solvent gel (1401) made from crosslinked PEG20KUA with no NBP. The opaque portion (top one third, 1402) is a composite material comprising crosslinked PEG20KUA with in situ precipitated NBP (PLGA polymer). FIG. B is similar to A except the PLGA portion of the layer is loaded with coumarin as a model drug with fluorescent properties (1403). The green fluorescence of coumarin encapsulated in NBP under long UV light (1403) shows a bilayered structure with sharp boundaries between two layers. FIG. C shows a unibody two layered crosslinked cylindrical implant made using PEG20KUA macromonomer (1404) and gelatin methacrylate (1405). This particle is then treated with fluorescein and lodixanol using EDC and n-hydroxysuccinimide which covalently bonds gelatin to fluorescein and lodixanol but not to crosslinked PEG2OUA. The crosslinked gelatin portion (1405) shows green fluorescence due to covalently bonded fluorescein and radio-opaque properties due to covalent bonding of iodinated compound like lodixanol and the crosslinked PEG20KUA macromonomer shows no fluorescence or radio-opaque properties due to lack of covalent sites (functional groups) to bind fluorescein and lodixanol. 1406 shows a sharp boundary between 1404 and 1405 showing a bilayered structure in a unibody mass wherein 1404 is a synthetic hydrogel and 1405 is a natural polymer hydrogel. FIG. C is also an example of a bilayer unibody particle wherein one layer (1404) is a biostable hydrogel and the other layer (1405) is a biodegradable hydrogel that degrades by an enzymatic pathway. FIG. D shows a microscopic image of two layer unibody magnetic microparticles wherein 1407 shows non-magnetic crosslinked PEG20KUA layer with no magnetic particles and 1408 shows magnetic layer with crosslinked PEG20KUA loaded with iron oxide. FIG. E1 shows the unibody gelatin hydrogel unibody sheet with alternate bands of clear and opaque hydrogel in 4 by 4 format. 1409 shows bands of crosslinked gelatin methacrylate with no magnesium carbonate (transparent gel) and 1410 shows bands of crosslinked gelatin methacrylate with magnesium carbonate (opaque gel). E2 is the same as E1 wherein E1 was incubated in warm water for 20 minutes. The non-dissolution of crosslinked gelatin hydrogel in the body and slight swelling of E1 shows an effective crosslinking process. The transparent and opaque gel parts also remain attached in the desired pattern and do not separate from each other indicating covalent bonding of cubes and their unibody structure. F shows two microcylinders (1412 and 1413) encapsulated in a hydrogel matrix (1411). The 1412 microcylindrical part comprises fluorescent particles encapsulated in a hydrogel matrix. The 1413 microcylindrical part comprises a hydrogel matrix wherein PLGA is precipitated in situ inside the hydrogel. 1412 and 1413 are made separately first and then encapsulated in the 1411 matrix.

Bilayered or multilayered implants can be used in many medical applications including controlled drug delivery applications most preferably ophthalmic drug delivery. A cylindrical shape of the two or multilayered implant is preferred. The preferred diameter of the two or multilayered implant may range from 0.5 microns to 50 mm, preferably 0.8 microns to 20 mm, most preferably 1 microns to 10 mm. The interface or boundary between any two layers (712) may be generally very in size typically in the range of 10 percent to 0.0001 percent of total volume and may have both the components present (drug and visualization agent in this case). The relative volume of each layer in the particle may occupy 1 to 99 percent of the total volume, preferably 5 to 95 percent, and most preferably 10 to 90 percent of the volume. In a bilayer configuration, one layer may occupy 50 percent of the volume and the other layer may occupy 50 percent of the volume; one layer may occupy 60 percent of the volume and the other layer may occupy 40 percent of the volume; one layer may occupy 70 percent of the volume and the other layer may occupy 30 percent of the volume; one layer may occupy 80 percent of the volume and the other layer may occupy 20 percent of the volume; one layer may occupy 90 percent of the volume and the other layer may occupy 10 percent of the volume. In three or four layered particles, each layer may occupy equal volume or may occupy unequal (not equal) volume. In a cylindrical particle, each layer may occupy 1 to 99 percent total length or height, preferably 5 to 95 percent, and most preferably 10 to 90 percent of the length or height. The drug and/or visualization agent present in each layer have a concentration of 0.01 percent to 50 percent relative to total particle weight, preferably 1 to 40 percent. In some embodiments, one drug or visualization agent from one layer can diffuse into other layer/s via interface layer 712. To prevent such diffusion, it is preferred that drug or visualization agents are in a separate phase or present in an encapsulated form. The concentration range of the visualization agent is similar to the drug concentration range. The diffusion can also be prevented by adding a barrier between two layers. Generally, in the preferred embodiments, drug and visualization mostly stay in each layer and do not substantially migrate or diffuse into another layer. In the preferred embodiments, drug and visualization agents are microencapsulated to prevent diffusion into other layers.

In one combination (EXAMPLE 12D11) an aqueous solution of macromonomer PEG35KL5A and gelatin methacrylate in PBS are photocrosslinked in a glass capillary tube to make bilayer unibody gel particle whose one layer is made using synthetic crosslinked PEG based precursor such as PEG35KL5A macromonomer and other layer made using photocrosslinked natural polymer derivative like gelatin methacrylate. The crosslinked PEG35KL5A degrades via hydrolysis of polylactate bonds and crosslinked gelatin degrades via enzymatic hydrolysis of amino acid sequences in the gelatin polymer chain when implanted in the human body. Their combination in a single unibody particle/implant provides a unique combination where degradation mechanisms and chemical compositions are vastly different. The gelatin used in this illustrative embodiment is also considered as an example of illustrative functional polymer with reactive functional groups like carboxylic acid, hydroxy, amine, and the like. These functional groups may be further used to covalently link useful compounds like drug/s, antibodies such as cancer tumor antibodies for targeting cancer tumors, radio-opaque agents like Iodixanol or colored or fluorescent compounds like fluorescein, catalysts like enzymes and the like. In one illustrative example, the gelatin-PEG35KL5A layered particle as above is incubated with n-hydroxysuccinimide (NHS), N-(3-Dimethylaminopropyl)-N′-ethylcarbodiimide (EDC), sodium fluorescein and iodixanol in DMSO. The EDC with help of NHS covalently binds fluorescein and iodixanol to the gelatin matrix layer of the particle thus making it fluorescent and radio-opaque. In another embodiment a multilayered particle with functional reactive groups is made. (EXAMPLE 12D16). A monomer with reactive functional groups such as glycidyl or carboxylic acid is added during the polymerization and crosslinking step to produce a synthetic crosslinked polymer with reactive functional side groups. The functional groups in the layer can be used for the covalent bonding of enzymes and other chemical entities. The functional groups that can be incorporated may be electrophilic or nucleophilic. Functional groups that can be incorporated include but are not limited to: epoxide, carboxylic acid, hydroxyl, carboxylic acid, activated acids like -n-hydroxysuccinimide ester, amine, aldehyde, isocyanate and the like. Monomers with functional reactive groups or their mixtures in any proportion can be added during the precursor crosslinking/polymerization step in the desired amount to obtain a layer in the particle with the desired amount of functional groups which can then be used in chemical reaction via functional groups.

In another embodiment, a monomer with alkyl side chains such as stearyl methacrylate or lauryl methacrylate acid is added during the polymerization and crosslinking step to produce a synthetic crosslinked polymer with an alkyl side chain (alkyl chain with 18 carbon atoms, 018, when using stearyl methacrylate or 12 carbon, 012 when using lauryl methacrylate and the like). Layers with one or two or more alkyl chains containing 6 to 22 carbon atoms (C6 to C22 alkyl chains) can be made depending on the intended use. Particles with multiple layers wherein each layer contains different types of the alkyl chain is envisioned for chemical separation use. The amount of alkyl content can be controlled by adding different amounts of monomers during precursor crosslinking steps. A 4 layered particle with each layer containing C8, 010, C12, C18 alkyl chains can be made and such particles may be packed in a chromatography column and used in qualitative and quantitative separations. Such particles may have an additional magnetic layer to help in packing the particles in the column wherein magnetic property is used in alignment and packing.

In one illustrative embodiment (EXAMPLE 12D9), particles having layers with different drug release profiles are made. Generally, fast releasing the drug for implantable application means: delivering the drugs within 7 days, preferably within 3 days and most preferably within one day. Slow release means: releasing the drug within 3 months to 2 years, preferably within 3 to 6 months and most preferably within 7 days to 3 months. Dexamethasone or bupivacaine hydrochloride is used as an illustrative example. Dexamethasone or bupivacaine hydrochloride unencapsulated form (fast release) and encapsulated form (slow release) is added in different layers of the particle (EXAMPLE 12D9). Many water soluble (solubility above 5 percent in PBS, pH 7.4) drugs can be used as fast releasing drugs in the hydrogel portion of one of the layers. The other layers may use the same drug in the microencapsulated form to obtain a controlled/slow-release version of the drug. In some embodiments, crosslinked density is used to control the drug release rate. Composite materials disclosed in this invention may be used to obtain different release profiles of many drugs. Artisans skilled in the art of controlled release know that variables like, type of biodegradable polymer used, its hydrophobicity and percent crystallinity, drug loading, particle size, encapsulated and non-encapsulated form of drug, degradation mechanism (erosion, enzymatic degradation), crosslinking density, electrostatic/ionic interactions and molecular permeability of crosslinked network and the like can be used to obtain a desired fast or slow release profile for each layer.

In one illustrative embodiment, one layer has biostable crosslinked material and another layer is made using biodegradable crosslinked material. In one embodiment, hexamethylene methacrylate in DMSO is crosslinked in presence of polymethyl methacrylate as a non-crosslinked biostable polymer in a biostable crosslinked polymer layer and the other layer has gelatin methacrylate crosslinked polymer in distilled water as biodegradable crosslinked polymer. Many combinations of biostable and biodegradable polymers described in this invention can be used.

In one illustrative embodiment, one layer comprises a cationic or basic polymer and the other layer comprises an anionic or acidic polymer. A combination of basic and acidic polymer in the same layer has the potential to form ionic interactions with themselves and separating them into distinct layers in a unibody article can be useful in controlled drug delivery and other applications. In one embodiment, an illustrative basic polymer polyallylamine which has amine groups as basic groups in the side chain is used. In another layer, an acidic polymer like polyacrylic acid with carboxylic acid or sulfonic acid side groups is used. The acidic and basic groups may be present in an ionized form or present as a salt form. Amine groups may be present as a hydrochloric acid salt form. The acid group may be present as sodium or potassium salt. The ionization may range from 1 to 99 percent. The use of polyallylamine as a basic polymer and polyacrylic acid as an acidic polymer is for illustration only. Other basic or acidic polymers known in the art can also be used. The basic polymers that can be used but are not limited to are: polylysine, chitosan, poly2-aminoethyl methacrylate and the like. The acidic polymers that can be used but are not limited to are: hyaluronic acid, polymethacrylicacid, polyaspartic acid, hydrolyzed Gantrez™ copolymers which contain alternating units of methylvinylether and maleic anhydride and the like. The ionic polymers as described above may be useful in controlled release of drugs with ionic groups.

In one illustrative embodiment, one layer has crosslinked material with the drug (an anticancer drug) and another layer has a targeting molecule such as an antibody against a specific cancer tumor. When such a particle is injected into the bloodstream or in the body, the targeting molecule will locate the disease site, attach to the site and then deliver the drug at the site locally. Many markers can target diseased tissue and such markers can be added to one of the layers of the multilayered particle. Other layers can be used to release the drug in a sustained manner once the particle reaches targeted diseased tissue such as cancerous tissue.

In another embodiment, layers with different crosslinking densities are made. The physical and chemical properties that depend on the crosslinking density of the network will be varied in the multilayered materials. Mechanical properties like modulus of materials, its molecular permeability, hardness, elastomeric nature and the like are dependent on crosslinking density. These and other properties could be varied from layer to layer in the multilayered materials described in this invention. For example, one layer could be hard and the other layer could be soft and elastomeric. In one illustrative embodiment, PEG2KUA macromonomer in DMSO and PEG100KUA macromonomer in distilled water are injected/filled sequentially in a glass capillary tube and then quickly polymerized and crosslinked to form a unibody multilayered implant with alternate sequences of high crosslinked density material (PEG2KUA) and low crosslinked density material (PEG100KUA). The low molecular weight of PEG (2000 Daltons) used in PEG2KUA and its higher molar concentration provides higher crosslinking density as compared to crosslinked PEG100KUA (PEG molecular weight 100000 Daltons). In addition, filler materials such as carbon black, fumed silica and other materials known fillers in the rubber and polymer materials art as well as a variety of non-crosslinked biostable and biodegradable polymers in a desirable proportion (1 percent to 3000 percent relative to the weight of precursor) may be added to further modify properties of the inventive layered compositions. The PEG2KUA and PEG100KUA and their sequence are used for illustration only. In another embodiment, the crosslinked density is varied by using tetra acrylate or octa acrylate precursors. The four or eight polymerizable bonds in the precursor provide higher crosslinking density than diacrylate or macromonomer with two polymerizable bonds. Those skilled in the art can understand that many variations can be made in the precursor composition before crosslinking and precursor compositions can be made to obtain desired properties of the multilayered materials. In another embodiment, a precursor comprising polydimethylsiloxane is used as an elastomeric layer and another layer comprising hexamethylene methacrylate in DMSO is crosslinked in presence of polymethyl methacrylate as non-crosslinked polymer hard material. A sheet-like multilayered material comprising blocks of elastomeric and hard materials arranged in a desired pattern can be used in many industrial and medical applications.

In some embodiments mechanical interlocking and/or covalent bonding is used to bind two layers in a multilayered object or particle. Frozen particles with Lego-like mechanical bonding features are first made from precursor solutions. The frozen solids are then mechanically joined together using the interlocking features. The interlocked particles are then exposed to light to effectively crosslink and form unibody objects. The formed crosslinked object has a combination of covalently linked parts and also mechanically interlocked parts. FIG. 16 shows a partial schematic representation of composite materials made using mechanically and covalently bonded composite materials. A1, A2 and A3 show unibody composite materials made using methods and compositions described in this invention. 1601 is a hard crosslinked material and 1602 is a soft elastomeric crosslinked material that serves as a bridge between 1601 material. 1603 is a portion of 1602 that is mechanically embedded as well as covalently locked into 1701 material. A2 is similar to A1 wherein the 1602 bridge has a curvature connecting 1601 hard material. When A2 is stretched, the curvature of 1702 provides space to expand the material. A3 shows chain-like arrangement between 1601 and 1602 materials.

Ability to form multilayered structures described in this invention can also be used to form glass, ceramic, polymer and metal-based compositions comprising multiple layers via a sintering process. In one illustrative example, a bilayered particle-containing soda lime glass particles in one layer and borosilicate glass particles in another layer is made via photocrosslinking of macromonomer. The layered particle thus prepared is dehydrated and then subjected to a sintering process by heating around 400° C. in air. At this temperature, the encapsulating matrix is substantially burned away leaving behind the bilayered particle with fused particles of soda lime glass in one layer and borosilicate glass in another layer. The glass is annealed to relieve thermal and other processing stresses in the body. PEG based macromonomer is used as an example only and those skilled in the art know that other precursor materials that provide minimum residue at the sintering steps can also be used. The materials used in the particle must be able to fuse (soda lime glass and borosilicate glass) with each other to form a single unibody structure. Those skilled in the art can understand that many combinations of various materials may be used as long as sintered materials provide unibody solid particles with two more layers.

In one illustrative embodiment (EXAMPLE 12D12) one layer has a drug and the other layer has RFID tag to encode information about the device or drug formulation. The RFID tag may be substituted with other electronic or chemical, biochemical and biological sensors for a given application.

In another illustrative embodiment (EXAMPLE 13-1) multilayered rod like structure with hard and soft materials is made. PEG2KUA and PEG1OOKUA macromonomer solutions along with photoinitiator are loaded in a 500 micron glass capillary in alternate sequence 3 times and then irradiated with light to effectively polymerize and crosslink the monomers. The crosslinked structure forms a 500 micron diameter unibody rod like structure with alternate bands of crosslinked PEG1OOKUA and PEG2KUA. PEG2KUA is relatively rigid compared to highly flexible and elastomeric crosslinked PEG1OOKUA material. The combination of alternate soft and hard bands are produced by the use of these two crosslinked macromonomers. Using a similar procedure as above, PEG1OKUA and PEG35KUA are photocrosslinked to make 3 and 5 segment rod particles. By changing different types of precursors or macromonomers reported in this invention or from the prior art, a variety of rod-like structures can be made. In another variation of this example, a sheet-like structure is made. FIG. 13 shows a partial schematic representation of a method to make unibody materials with diverse compositions in the desired 2 or 3-dimensional patterns. A photopolymerizable precursor (P1) solution with a photoinitiator is first filled into a mold and then frozen/dried to form a cubical or hexagonal prism shaped solid (A). B shows another precursor composition (P2) with a photoinitiator in a frozen cubical shape or hexagonal prism shape. A and B generally have the same shape and dimensions. A and B are arranged in a checked pattern in a frozen state (C) preferably in another mold so that at least one of the surfaces of A and B touch each other. C1 shows a hexagonal prism shaped arrangement of precursor P1 and P2 wherein one side of the prism touches with another side. A micromanipulator tool may be used for smaller sized frozen particles to achieve a desired pattern or arrangement in the frozen state. Compositions P1 and P2 may be warmed to liquefy (partially or completely) without substantial mixing. The compositions P1 and P2 in C are then exposed to light to effectively crosslink P1 and P2. The polymerization takes place within and between P1 and p2. P1 and P2 may be completely melted or partially melted or maybe in a frozen solid-state or combination thereof before crosslinking. The effective polymerization leads to the formation of a unibody structure where all cubes are covalently linked to each other to form a single unibody structure (D). The physical and chemical properties associated with crosslinked P1 and P2 are dispersed in the body of D in the desired pattern. E is similar to D wherein some cubes are intentionally removed or from the frozen arrangement or not placed during the arrangement prior to crosslinking leaving behind the void or porous space (E1) in the crosslinked structure. FIG. H shows a bilayered unibody particle made by the fusion of A and B via photopolymerization and crosslinking as described above. FIG. I shows a unibody three layered particle (ABA) made similar to H. FIG. J shows a unibody concentric disk like design in which inner circle/disk (J1) and outer circle/disk (J2) are covalently linked to form a two layered unibody composite material. FIG. J can be considered as a contact lens wherein J1 could be a contact lens part that provides refractive properties and an outer layer (I2) provides drug delivery or other useful function/property. FIG. F shows cylindrical or spherical shaped frozen/dried precursor solids/solutions with two different frozen compositions (F1 and F2) arranged in the desired pattern and then crosslinked to preserve the pattern arrangement and produce a unibody porous structure. The empty space between the spheres (F3) may be used to create the desired amount of porous space in the crosslinked unibody structure by controlling frozen particle size, shape and pattern. FIG. G shows diamond shaped frozen particles with compositions G1 and G2 arranged to create a hexagonal shaped cavity (G3a) and triangular cavity (G3b). The arrangement is locked in place by photocrosslinking of the G1 and G2 to create a crosslinked unibody structure with hexagonal and triangular shaped cavities/porous space in the unibody material.

In one illustrative embodiment, a 4 ml 10 percent solution of gelatin methacrylate in distilled water is made and Irgacure 2959 is added as a photoinitiator (0.1%). The prepared solution is divided into two parts. One part is mixed with magnesium carbonate (about 10 percent) as a model visualization agent or drug encapsulated microparticles and the other part is used without magnesium carbonate. The gelatin methacrylate solution and gelatin methacrylate suspension with magnesium carbonate are filled into 2 mm by 2 mm by 2 mm silicone rubber mold and frozen. The frozen solutions are removed from the mold. The frozen gelatin methacrylate and gelatin methacrylate with magnesium carbonate cubes are arranged in 4 by 4 checkered alternate fashion (total 16 cubes, an arrangement similar to shown in FIG. 13C) in the cold condition in another silicone rubber mold. The arrangement is tight and at least one surface of the cube is in physical contact with the other cube in a frozen state. The cubes are then optionally warmed to room temperature until partial melting of gelatin methacrylate on the surface of the cube is obtained and then exposed to long UV light (360 nm) for 5 minutes. The exposed cubes undergo photopolymerization and crosslinking in solution state and frozen state forming a unibody structure with zones of gelatin methacrylate filled with and without magnesium carbonate. The presence of magnesium carbonate makes the gelatin methacrylate opaque. The crosslinked structure is incubated in water for 20 minutes at ambient temperature to test the formation of the crosslinked and unibody structure. Upon incubation in water, none of the cubes dissolved in water indicating effective polymerization and crosslinking and formation of unibody structure. The cubes also have not formed individually and cannot be separated from the unibody mass. The single body mass of crosslinked sheet like gel has alternative bands of clear (crosslinked gelatin methacrylate with no magnesium carbonate) and opaque (crosslinked gelatin methacrylate with magnesium carbonate) bands/zones in a unibody structure. FIG. 14E shows an illustrative unibody gelatin hydrogel sheet with alternate bands of clear and opaque hydrogel in a 4 by 4 format made using the method described herein. 1409 shows bands of crosslinked gelatin methacrylate with no magnesium carbonate (transparent gel) and 1410 shows bands of crosslinked gelatin methacrylate with magnesium carbonate (opaque gel). If zero number is assigned to clear gel and one number is assigned to opaque gels, then the pattern of 1010 in the first row, 0101 in the second row, 1010 in the third row and 0101 in the fourth row is encoded. Such information can be made machine-readable and processed to generate a numerical code that can be used as an identifier for assessing information in a database. The clear and opaque pattern used herein is for example only. Fluorescence of fluorescent compounds or color of colored compounds as well their location in a sheet matrix.

FIG. 13 shows a partial schematic representation of steps involved in the preparation of tissue engineering scaffold with or without cells prepared according to methods and compositions described in this invention. Live cell suspension and precursors such as macromonomer solution along with photoinitiator are mixed and frozen to the desired shape such as cubical shape. FIG. 13A schematically shows a live mammalian type X inside a frozen precursor in the form of a cube or hexagonal prism. FIG. 13B is similar to A but has a different cell type (type Y, Live) inside a frozen precursor solution. Frozen particles A and B are arranged in two or two three-dimensional patterns such that at least one surface of frozen particles is in contact with each other (C). The frozen particles are then crosslinked by exposing to visible light or long UV light so that each frozen particle is covalently linked with another particle without substantially losing cell viability (D). In some cases, intentional porosity (E1) may be created in the crosslinked structure to provide access to the tissue culture medium (E). D schematically shows a 3-dimensional construct made as described above. D and E show live encapsulated cells in the crosslinked scaffold. Commercially available injector devices like SonoPlot Microplotter II from SonoPlot Inc, Middleton, WI which can deposit/inject 5 microns to 200 microns liquid droplets with a droplet volume greater than 0.6 picolitres. The device can deposit discrete droplets as well as a continuous line of deposited liquids. Such deposits may also be used to make composite materials and particles described in this invention. In the preferred embodiment, liquid droplets with a variety of precursor compositions in the desired size are deposited first using one or more SonoPlot Microplotter I device and then frozen or dried to remove the solvent. FIG. 15 schematically shows a device like the SonoPlot Microplotter (1511) that is injecting precursor droplets (1511) which are collected in cryogenic baths like liquid nitrogen to make frozen precursor microdroplets. The solid frozen/dry particles are arranged in a desired pattern and composition and then effectively crosslinked to freeze the patterned arrangement to make a unibody structure.

Materials with shapes like circular shapes (cylindrical shaped) could be used to create porous materials. The arrangement of frozen materials like cylindrical rods or microspheres before crosslinking leaves gaps or empty space between them due to the nature of their shapes (FIG. 13F, Space F3). These gaps (FIG. 13F3) are embedded in the crosslinked unibody structure to form a porous unibody structure. In some embodiments, frozen cubes have been intentionally removed from the stack prior to crosslinking. Alternatively, the frozen cubes are not filled/added in certain spaces during the arrangement of frozen cubes to make the gaps or porosity. The gap created by the removal of uncrosslinked frozen cubes leaves porosity in the crosslinked structure. FIG. 13E shows porous crosslinked solid created by removal of 4 cubes from the crosslinked matrix prior to crosslinking. By controlling the shape and size of such structures, porosity or shape/geometry of porosity in unibody structure could be introduced and controlled for many biomedical and optical applications. For non-porous structures, symmetrical shapes like a cube or hexagonal prism shapes are preferred. For porous structures, shapes like cylindrical or non-symmetrical structures are preferred.

One illustrative embodiment uses a two dimensional arrangement of frozen cubes to make a unibody structure like a sheet, however, two or more sheets could be stacked on top of each other prior to

crosslinking to make a 3 dimensional material before crosslinking. Various combinations of materials described in examples 10 to 13 can be used for the formation of multilayered structures with encapsulated entities and these include but not limited to: drugs, cells, sensors, enzymes, proteins, antibodies, and the like. In some embodiments, bilayer precursor cubes are made first and one of the layers is magnetic and the other layer comprises live cell/s. The magnetic property of the frozen cube is then used to arrange the magnetic cubes in the desired pattern and then photocrosslinked as described above. This way small size frozen cubes with cells or drugs could be arranged precisely using magnetic property and electromagnetic mold surfaces. Laser and ultrasonic based tools such as tweezers may also be used to precisely lift/move/arrange particles in the desired pattern. Automated machines with robotic arms can also arrange small size particles in a precise manner for the desired pattern. Alternatively, cubes with different compositions/physical properties may be cast in different molds similar to described in FIG. 13, stacked on a two dimensional surface separately and then aligned and placed to create the desired pattern and then crosslinked to create a single unibody structure. The live-cell sheet was made using methods as described above where cubes containing one or two or three or more cells could be used to make organs using tissue engineering methods known in the art. Similarly, cells such as pancreatic islets (islets of Langerhans) could be arranged in the desired pattern and entrapped in the stacked sheets as described above to make an artificial pancreas. Live mammalian cells such as Chinese hamster ovary (CHO, epithelial cell line derived from the ovary of the Chinese hamster) cells, Hybridoma cells and the like may be encapsulated in sheet or rod like or other shapes can also be used to make important cell derived therapeutic proteins/drugs like Humira (Adalimumab), Sovaldi (Remicade), Rituxan (Rituximab), Enbrel (Etanercept) and the like. Multilayered particles with Chinese hamster ovary cells, Hybridoma cells and the like in one layer and magnetic particles in other layers may be manipulated using magnetic fields in a bioreactor. This manipulation may be useful in the separation of cells from the bioreactor, their arrangement in contact with cell culture medium and the like.

In structural or engineering material development applications, elastomeric and hard materials could be arranged in the desired manner using the methods described above to create unique composite materials with anisotropic and/or symmetric properties.

Polyacrylamide unibody hydrogels could be made using the methods described in this invention wherein bands of different crosslinking densities can be introduced in the gel. Such gels could be useful in gel electrophoresis analysis. Briefly, acrylamide solutions with different amounts of acrylamide and/or crosslinker N, N′-methylenebisacrylamide along with free radical initiator or photoinitiator are made and frozen in the mold in the form of strips. The frozen solutions are then arranged in the desired fashion and crosslinked as above to produce unibody gel with different crosslinking densities in the same unibody structure. Alternatively, the acrylamide solutions may be separated using a removable spacer as described earlier. The solutions are added in the mold with spacer and the spacer is removed and the solutions are quickly polymerized before substantial diffusion of acrylamide or N, N′-methylenebisacrylamide in other layers. The effective polymerization and crosslinking is done before 5 minutes, preferably before 2 minutes and even more preferably before 1 minute. Lower temperature or frozen or dry monomers may also be used as described before to reduce the mixing of different solutions. A high intensity laser light preferably laser light is used to accelerate the polymerization and crosslinking.

Several properties/materials can be added to each layer of the multilayered unibody particles described in this invention. Properties/materials that can be given or added to each layer include but not limited to: drugs, therapeutic cells, enzymes, visualization agent, biodegradable nature of the layer, duration of biodegradation in the body, drug release rate, crosslinking density of the composition, solvent swelling properties, modulus of the material, magnetic property, encapsulated drug/visualization particles, propellants, explosives, thermosensitive gels, liquid carrier for drugs, solvents, sensors, optical transparency, radio-opaque property, ultrasonic image visualization property, fluorescence, color, luminescence, conductivity, magnetic resonance imaging property, porosity, density, and the like. Several non-limiting examples of preferred combinations of property/compositions provided below:

The preferred crosslinked multilayer composition comprises:

1) At least one layer is a hydrogel.

2) At least one layer that is a biodegradable hydrogel.

3) At least one layer that degrades by enzymes.

4) At least one layer that degrades by hydrolysis.

5) At least one layer that is a biostable hydrogel.

6) At least one layer that is an organic solvent gel.

7) At least one layer as a biostable organic solvent gel.

8) At least one layer that is magnetic.

9) At least one layer that is electrically conducting.

10) At least one layer that is made using free radical polymerization.

11) At least one layer that is made using photopolymerization.

12) At least one layer that is made using condensation polymerization.

13) At least one layer that is porous.

14) At least one layer that comprises a drug or visualization agent.

15) At least one layer that comprises a drug or visualization agent that is microencapsulated.

16) At least one layer that comprises a drug or visualization agent that is microencapsulated in a biodegradable polymer.

17) At least one layer that comprises a sensor.

18) At least one layer that comprises an RFID chip.

19) At least one layer that comprises a non-crosslinked biodegradable polymer.

20) At least one layer that comprises a non-crosslinked thermosensitive or pH sensitive polymer.

21) At least one layer that comprises a liquid carrier.

22) At least one layer that comprises a non-crosslinked biostable polymer.

23) At least one layer that comprises functional reactive groups.

24) At least one layer that comprises a filler.

25) At least one layer that is optically transparent or semi-transparent.

The preferred combination of two or more layers in a crosslinked multilayered object:

1) One layer is a hydrogel and the other layer organic solvent gel.

2) One layer has a drug with a molecular weight less than 2000 g/mole and the other layer has a drug with a molecular weight greater or equal to 2000 g/mole.

3) One layer has a drug with a molecular weight less than 2000 g/mole and the other layer has protein or biologic drug.

4) One layer has a drug that is released in a controlled manner within 7 days and the other layer has a that is released in a controlled manner within one year.

5) One layer comprises a crosslinked polymer with high crosslinked density. The average molecular weight between the crosslink is less than 10000 g/mole. The other layer comprises a crosslinked polymer with low crosslinked density. The average molecular weight between the crosslink is greater than 10000 g/mole.

6) One layer comprises a crosslinked polymer with molecular permeability for the molecules with less than 25000 g/mole and the other layer has molecular permeability for the molecules greater than 25000 g/mole.

7) One layer comprises a crosslinked polymer with a drug or visualization agent that is microencapsulated and the other layer has a drug or visualization that is not microencapsulated.

8) One layer comprises a crosslinked polymer with a drug and the other layer comprises a crosslinked polymer with a visualization agent.

9) One layer comprises a synthetic crosslinked polymer and the other layer comprises a natural crosslinked polymer.

10) One layer is hydrophobic and the other layer is hydrophilic.

11) One layer is magnetic and the other layer has a drug, visualization agent, enzyme, bacteria virus, enzyme, fungi or algae.

12) One layer comprises a crosslinked polymer that absorbs and swells less than 10 percent water relative to total weight and the other layer comprises a crosslinked polymer that absorbs more than 10 percent water relative to the total weight.

13) At least two layers bonded by mechanical interlocking and covalent bonding.

14) One layer comprises a crosslinked polymer that is elastomeric and the other layer is non-elastomeric.

15) One layer comprises a crosslinked polymer that is transparent or semitransparent and the other layer has a crosslinked polymer that is opaque.

16) One layer comprises a crosslinked polymer that has a refractive index around 1.5 and the other has refractive index 1 percent higher or lower than the first layer.

17) One layer comprises a crosslinked polymer that has high oxygen permeability and the other has low oxygen permeability.

18) One layer comprises a crosslinked polymer that has a density around 1 g/100 g and the other layer has a density 5 percent higher or lower than the first layer.

19) One layer comprises crosslinked and biostable non-crosslinked polymer and the other has a different biostable non-crosslinked polymer than the first layer.

20) One layer comprises crosslinked and biostable thermoplastic elastomers and the other has a different biostable thermoplastic elastomer than the first layer.

21) One layer comprises a crosslinked polymer and biostable thermoplastic elastomer and the other has a different biostable thermoplastic elastomer than the first layer.One layer comprises a crosslinked polymer comprising polydimethyl polysiloxane and the other layer has crosslinked polymer comprising polyethyleneoxide.

Applications of the Inventive Materials

Wound Management and Controlled Drug Delivery Applications.

The sheet-like materials with alternate bands of transparent and opaque regions shown in FIG. 14E can be modified to make wound dressing and other biomaterials for controlled drug delivery, wound dressing and other medical products. The transparent and opaque materials used in 14E are replaced with bands of materials that are known to be useful in managing wounds. Wound dressing hydrogel materials comprising collagen, cellulose derivatives like hydroxyethyl cellulose, polyethylene glycol or oxide, decellularized tissue, hyaluronic acid and the like are known in the wound dressing art. Such materials may be used to form composite unibody materials such as shown in FIG. 14E wherein gelatin material with magnesium carbonate used in making the material is replaced with crosslinked hyaluronic acid or crosslinked PEG based hydrogel described in this invention. This will produce sheet-like unibody materials with alternate bands of crosslinked gelatin methacrylate and hyaluronic acid or crosslinked PEG based hydrogel. In one exemplary embodiment, the opaque magnesium carbonate band in the composite material (FIG. 14E) is replaced with hydrogel comprising polydimethylsiloxane units. The inclusion of polydimethylsiloxane improves oxygen permeability to the wound. In another modification of the same concept, magnesium carbonate used in (EXAMPLE 13, FIG. 14E) is replaced with drug loaded microspheres such as bupivacaine or rifampin loaded PLGA microspheres to create sheet-like unibody material. This will create sheet-like material with alternate bands of crosslinked gelatin methacrylate and crosslinked gelatin methacrylate with bupivacaine or rifampin loaded PLGA microspheres. In another embodiment (EXAMPLE 16), a unibody composite hydrogel is produced that has 2 mm blocks of crosslinked PEG1OK hydrogel with hydroxypropyl methylcellulose or cellulose sulfate as an NBP, PEG1OK hydrogel with PLGA and bupivacaine base, gelatin methacrylate with sodium hyaluronate as NBP is arranged in a grid pattern. Bupivacaine in PEG10K-B block provides controlled release of bupivacaine base for the local anesthetic effect. Entrapped hydroxypropyl methylcellulose and hyaluronic acid in crosslinked hydrogel provide wound healing properties and water absorption properties needed for wound healing. Use of bupivacaine is used as an illustrative drug. Other drugs useful in wound management such as antibiotics, growth factors, antiinflammatory drugs and the like can also be used. A combination of wound healing agents, local anesthetic agents and antibiotics in any proportion is preferred for wound management application. In another embodiment, a two layer unibody sheet like material is made (EXAMPLE 16C) wherein one layer comprises polydimethylsiloxane for improved oxygen permeability and another layer comprises a standard hydrogel that provides a moist environment for wound healing. The preferred area of wound dressing materials may range from 0.5 square centimeters to 500 square centimeters, preferably 1 square centimeter to 100 square centimeters. The shape of the wound dressing may be any shape commonly found in commercial wound dressings however symmetrical shapes such as rectangular and circular shapes are preferred. The total thickness of the wound material sheet may range from 0.2 mm to 5 mm, most preferably 0.5 mm to 3 mm.

Ophthalmic Applications.

The methods and compositions described in this invention have many applications in the ophthalmic devices area. The wound dressing sheet like materials with multiple zones or bands in a grid pattern can be adopted to make novel ophthalmic devices which include but are not limited to corneal shields, corneal bandages, contact lens, punctal plugs, cornea tissue engineering scaffolds and the like. The Microneedle array disclosed in this invention can also be adopted for ophthalmic drug delivery. In this invention, we disclose a contact lens device with a unibody structure and have 2 or more layers in the device. The first layer is generally designed to provide correct refractive power or it may provide a transparent material layer with greater than 90 percent visible light transmission with no refractive power. The second layer or other additional layers preferably reside on the outer peripheral side of the contact lens. All the layers are covalently linked with each other to make the unibody contact lens. The second or additional layer provides additional properties such as the controlled release of a drug, fluorescence, color, electrical conductivity, different crosslinking density, oxygen permeability, optical properties, and the like. In addition, we also disclose compositions and methods to provide contact lenses with controlled porosity. The controlled porosity in the lens structure is expected to improve the oxygen permeability of the lens without significantly affecting the mechanical properties of the lens. The refractive power of the contact lenses can be adjusted by creating a gradient refractive index (GRN) in the lens body.

Solid state polymerization of frozen monomer mixtures disclosed in this invention can be used to make standard contact lenses and multilayered contact lenses. The outer portion of the contact lens that does not cover the pupil of the eye (about 5 to 40 percent of total area) and therefore can be reinforced with knitted/woven fibers to improve tear resistance or mechanical properties. The outer portion of the contact lens with monomers and cotton or other woven fibers are made and frozen. The inner portion of the lens without woven fibers is also made separately and frozen. Both portions are joined and then exposed to light for effective polymerization and crosslinking to fuse/join both layers and form a unibody contact lens. The woven fibers may be loaded with drugs if desired for local ophthalmic drug delivery. Alternatively, the outer layer may be added with NBP like PLGA prior to polymerization and then loaded with a drug via infusion/diffusion process as described in this invention. In another illustrative embodiment (Example 14) a composite of free radically polymerizable monomers and crosslinked PVA based hydrogel is disclosed for contact lens and other medical applications. Monomers are crosslinked using a free radical polymerization and PVA is crosslinked using the freeze-thaw technique. Either crosslinking can be done first. In the preferred embodiment, PVA is used as an NBP in the crosslinked hydrogel followed by crosslinking of PVA using the freezing-thawing technique known in the art. In addition, a two layer unibody contact lens material is made for local ophthalmic drug delivery (Example 15). The outer layer of contact lens is added with jeffamine lactide as an illustrative NBP which can be loaded with ophthalmic drugs using solvent diffusion techniques as described in this invention. The monomers used in the manufacturing of contact lens can be polymerized in frozen state instead of solution state. The frozen state polymerization method disclosed in this invention creates porous hydrogel materials that help to improve oxygen permeability of the lens materials.

The methods and compositions described in this invention can be made to make ophthalmic drug delivery implants which can be inserted in many sections of the eye for controlled drug delivery. Bilayer or multilayer punctal implants, preferably biodegradable hydrogels based intracanalicular or punctal implants can be made using methods and composition described in this invention. FIG. 16 shows a partial schematic representation of composite materials made using mechanically and covalently bonded composite materials. It also shows preferred ophthalmic or punctal bilayered implants. FIG. 16B1 is a two layered unibody cylindrical punctal/medical implant wherein the top portion of the implant has microspheres with visualization agent (1604) and the bottom portion of the implant has drug encapsulated microspheres (1605). FIG. 16B2 shows bilayered (1606 and 1607) punctal implant comprising different drugs present in two different layers (1606 and 1607). Preferably drugs in 1606 and 1607 layers are microencapsulated in a biodegradable polymer. FIG. B3 shows a bilayered (1608 and 1609) punctal implant comprising the same drug in each layer but has a different controlled release rate/profile. The drug in the 1608 layer releases all the drug quickly in 0.1 to 7 days (fast release) and the drug in the 1609 layer releases the drug from 8 days to one year (slow release). B4 shows a bilayered (1610 and 1611) unibody implant wherein one of the layers (1610) has substantially more ability to absorb water and swell under physiological conditions (pH 7.4, 37 degree C.) than the other layer (1611). B41 shows the size and shape of the implant immediately after inserting in the punctal cavity. B42 shows B41 implant after absorption of water within 24-72 hours after implantation. The layer 1610S swells significantly more than the layer 1611S which helps to immobilize the implant in the punctal cavity and prevents its migration. One of the layers such as The 1610S may absorb and swell 10 percent more water than the layer 1611S. In the preferred range the swelling difference may be 10 to 300 percent range. FIG. B5 shows bilayered (1606 and 1607) punctal implant comprising a visualization agent such as fluorescent agent (1612) and ophthalmic drugs such as dexamethasone (1613). Preferably, drugs and visualization agents in 1612 and 1613 layers are microencapsulated in a biodegradable polymer like PLGA. FIG. B6 shows a trilayered implant wherein the implant has a swellable layer 1610S and two layers for controlled drug delivery. Preferred drugs are antiinflammatory or antibiotics. Layer 1614 may comprise dexamethasone and 1615may comprise moxifloxacin.

In one exemplary embodiment, a bilayered implant has fluorescent compounds such that fluorescein is covalently attached to the biodegradable matrix. One illustrative embodiment discloses a bilayer composition wherein one layer has functional groups that can be used for visualization agent attachment. In the illustrative embodiment a bilayer cylindrical hydrogel comprising crosslinked PEG based hydrogel and gelatin are disclosed. The gelatin portion of the implant is treated with fluorescein and EDC to covalently bond the fluorescein to the gelatin layer. The fluorescence of the implant helps to monitor its movement after implantation in the punctal cavity. For the punctal implant application, the length of the punctal implant may range from 500 microns to 2 mm and the diameter of the implant may range from 300 microns to 1500 microns. The drug release from the implant may range from 3 days to 18 months preferably 7 days to one year. Punctal implants described as above may also be used in the other area of the eye such as vitreous humour wherein the length of the implant can be up to 2 cm. Ophthalmic implants described above can be adopted to address ophthalmic diseases like age-related macular degeneration, cataract, diabetic retinopathy, glaucoma, ophthalmic infections, dry eye disease, allergic conjunctivitis and the like. The size of the implant as described above may change depending on the application. An additional list of ophthalmic drugs that can be incorporated in the inventive ophthalmic implants or microparticles described in this invention can be found in U.S. Pat. No. 8,409,606 cited herein for reference only.

Clinical Diagnostics

FIG. 16C shows a unibody hydrogel grid like sheet similar to shown in FIG. 14E. The sheet can be used for clinical diagnostics of diseases like virus detection, cancer biomarkers and other clinical indications. A typical clinical test involves an antigen for a specific clinical condition and an antibody specific to the antigen. The antigen forms a complex with detection antibody which is covalently linked through an enzyme or secondary antibody. The enzymatic activity is used to create a chromophore which under specific reaction conditions produces a colored substance, fluorescence or luminescence signal. This signal is used for qualitative or quantitative analysis. The illustrative sheet has 10 columns and 4 rows produced using a similar method as described in this invention. At position R1C1, an antigen specific to the disease to be detected is infused in the hydrogel matrix during the effective polymerization and crosslinking process. This location serves as a positive control. Briefly, frozen cubes of antigen, calibration chemicals and blank cubes are arranged in a rectangular sheet format and then crosslinked to produce a unibody of sheet wherein all the frozen compositions are linked via covalent bonding. The location of the antigen, sample and calibration in the sheet also can be used for analysis. R1C2 and R1C8 spaces are intentionally left blank to aid in visual analysis. R1C3, R1C4, R1C5, R1C6 and R1C7 have known concentrations of antigen and it can be used for internal calibration purposes. R1C9 and R1C10 contain antibodies specific to the antigen being analyzed in duplicate. Row two is a duplicate of R1. When the grid is exposed to blood or serum or other liquids to be analyzed, the antigen in the sample interact with the antibodies in the R1C9 and R2C10 and upon subsequent processing with enzyme and other specific test related reagents produces a response such as color, fluorescence or luminescence which can be used for diagnostics purpose. Using the calibration curve generated by R3 to R7 signal, quantitative analysis also can be made. The number of rows and columns used herein is for demonstration only. Several rows can be added to the sheet for other types of clinical analysis. For example, the grid-like gel can be used to analyze the Influenza (flu) virus and SARS-CoV-2 virus from a single sheet of material. Similarly, several cancer biomarkers can be analyzed simultaneously using the general method described above. One single sheet can be used for clinical diagnosis of one, two, 3, 4, 5 up to 100 or more clinical indications.

The microneedle array disclosed in this invention and related applications can be used for collecting tissue fluids for clinical diagnostics application. A preferred crosslinked PEG based hydrogel microneedle array is made with water soluble NBP such as hyaluronic acid or cellulose derivatives according to compositions and methods disclosed in this invention. The preferred crosslinked materials used in the array has molecular permeability up to 100000 g/mole, preferably up to 60000 g/mole. The molecular permeability enables to infuse/diffuse low molecular weight components present in the tissue fluid in the crosslinked material for clinical diagnostics use. The preferred microneedle array used is substantially dry and has preferably 20 to 80 percent porosity. The porosity helps to rapidly absorb tissue fluids. The microneedle crosslinked materials used may be hydrophilic or hydrophobic, hydrophilic or hydrogel based materials are preferred. The microneedles materials may be biodegradable or biostable. Preferred materials are biodegradable. The preferred biodegradation time is less than 6 months, preferably less than 30 days. Preferred microneedles are colored or fluorescent. The dry microneedle array is inserted in the skin tissue. The array needles are kept in contact with the tissue for 1 minute to 600 minutes preferably with 2 minutes to 120 minutes. During this time, the array needle absorbs and swells with the surrounding tissue fluid. The high molecular permeability and porosity helps to absorb/infuse tissue fluid components with molecular weight less than 100000 g/mole. The array is removed from the tissue and the absorbed tissue fluids are analyzed for relevant clinical indications. C1inical Indications include but not limited to analysis of: glucose, urea , sodium or potassium, lipids, monosodium urate and the like. The analysis can be done on the entire array or on the individual needle. The analysis of individual needle is preferred.

PERSONAL HYGIENE PRODUCTS The compositions and methods to make crosslinked hydrogels disclosed in this invention can be easily adapted to make personal hygiene products such as sanitary napkins, adult diapers, baby diapers and the like. The ability to polymerize in a solid/frozen state and with predefined shapes and controlled porosity can be exploited to make superabsorbent hydrogels for personal hygiene products. In one illustrative embodiment, a mixture of PEG1OOKUA, acrylic acid and sodium acrylate in water along with UV photoinitiator is sprayed into a cryogenic bath or liquid nitrogen to make frozen microspheres of size 10-2000 microns. The microspheres are poured into a 10×5×2 mm mold cavity in a frozen state. The microspheres are arranged in a closely packed arrangement wherein microspheres touch with each other similar to shown in FIG. 13F. One or more layers are formed depending on the application desired. The arranged microspheres are then exposed to intensity UV/visible light or electron beam to effectively polymerize and crosslink the microspheres in a frozen state. Upon polymerization, the microspheres form a unibody object wherein polymerization and crosslinking takes place within the frozen microspheres as well as between the microspheres. Crosslinked mass is washed with water to remove unpolymerized monomer and soluble fragments and dried. The frozen water inside the frozen droplets creates micropores within frozen microspheres which enables high water retention. Upon freezing, free water in the composition forms ice crystals inside the frozen structure. The ice formed cannot participate in the polymerization and crosslinking process upon irradiation and thus act as a filler. Macromonomer surrounding the frozen ice crystals polymerizes in the frozen state excluding ice. Upon warming to room or ambient temperature and drying, or upon lyophilization, ice crystals/water are removed from the crosslinked materials producing porosity in the crosslinked material. Variables like freezing conditions employed, additives added, amount of water and the like can be used to create porosity in the crosslinked structures. The amount of water or other solvents used during polymerization may be varied to adjust/control porosity of hydrogels. The use of crosslinkers such as PEG1OOKUA prevents dissolution and makes the crosslinked material elastomeric. The space between microspheres during frozen state unibody provides a path for body fluids such as urine to quickly spread throughout the unibody material. This path can be adjusted in many ways using precursor particle size, shape and arrangement as discussed in this invention. FLOW DIAGRAM 6 described in FIG. 12 can also be used to make unibody crosslinked objects made using precursors described above. It is preferred that the amount of sodium acrylate in the mixture of acrylic acid and sodium acrylate is above 75 percent. EXAMPLE 19 provides illustrative super absorbing hydrogel made using acrylic acid sodium acrylate microspheres. Using other teachings in these patents, an efficient super absorbent hydrogel comprising sodium or potassium acrylate can be obtained for personal hygiene use. The desired size, shape and arrangement of the absorbent materials used for personal hygiene products can be printed using 3 D printing techniques known in the art or described in this invention. A desired monomer mixture preferably a monomer mixture comprising sodium or potassium acrylate along with a photoinitiator and crosslinker is used as a starting mixture. The liquid mixture is then used exposed layer by layer to obtain a desired shape and size which are then washed and dried. Several materials can be printed at the same time. The printed materials are then used to make personal hygiene products. The frozen compositions comprising sodium acrylate are arranged such that desire amount of porosity between the frozen structure (FIG. 13E, FIG. 13F, FIG. 13G) is created and then crosslinked in solid state. Arrangement of water absorbing elements of the personal hygiene products can be arranged such as shown in FIG. 7H1, H2, H3. The porous structures with desired microfluidic channels geometry/map and structures shown in FIG. 13E, FIG. 13F, FIG. 13G for absorbing biological fluids can also be printed using 3D printer as discussed before.

Oral Drug Delivery

Many drugs are delivered by oral route and the compositions and methods described in this invention can be adapted for oral drug delivery. FIG. 18 shows a schematic representation of methods for making oral drug delivery systems. FLOW DIAGRAM 7 shows a method to make multilayered (4) layered oral drug delivery pharmaceutical tablets. FIG. A represents a circular mold cavity and B represents a removable spacer or divider which can be placed inside cavity A to make two or more cavities/compartments. C shows a cavity A with spacer B with 4 equally divided compartments/cavities. C is then filled with a precursor solution comprising a drug or visualization agent or sensors such as RFID tag or other biochemical sensors. Compartments in C are then filled with 4 different types of precursor compositions (1801, 1802, 1803 and 1804) that have the capacity to form unibody material upon effective polymerization and/or crosslinking. E is the same as D except spacer B is removed and the precursor may be in liquid, solid or frozen state or combination thereof. E is subjected to effective crosslinking conditions such as exposure to UV light to crosslink and fuse all 4 layers to form a unibody circular disk shaped tablet that can be swallowed (F) for oral drug therapy. F shows 1801P, 1802P, 1803P and 1804P as crosslinked layers of a unibody material made from precursors 1801 to 1804 respectively. F may be further processed such as lyophilized or dehydrated and then coated with taste masking agents and the like to make an oral pharmaceutical tablet composition. FLOW DIAGRAM 8 shows a method to make oral drug delivery pharmaceutical capsules using hard or soft shell oral drug delivery capsule shells. G and H schematically shows two halves/parts of standard gelatin or HPMC based hard/soft capsule shells (commercial capsule sizes vary from 000 to 4) that are commonly used in the pharmaceutical industry. The cavities in the shell are filled with the same or different types of precursor compositions (I,1805; J,1806). Preferably the compositions have the capacity to form unibody material upon effective crosslinking. The precursor compositions used are inert towards the capsule material and do not affect capsules structural integrity and other properties. 1805 and 1806 comprises drug or visualization agent or sensor such as RFID tag or other biochemical sensor. Precursors in I and J are subjected to effective precursor crosslinking conditions to form crosslinked compositions (1805C and 1806C). The shells are then fused to form the oral capsule. Alternatively, the precursor can be frozen/dehydrated first and shells are fused to form a capsule and crosslinking is initiated to form a unibody two layered body inside the capsule. N shows standard gelatin or HPMC oral capsule (1808) that is enclosed with two or multilayered drug delivery particles (1809) as described in this invention.

In one illustrative embodiment (EXAMPLE 20), a 4 compartment oral tablet is made comprising two drugs in a unibody crosslinked material. One compartment is filled with magnetic particles to enable control over the passage of tablets in the abdominal cavity. Two compartments are used for illustrative oral drug diclofenac sodium. One of the two is a slow release composition due to the presence of an illustrative NBP polymer which helps to slow down the release of diclofenac sodium from the crosslinked gel. The other one without NBP is a fast releasing composition of diclofenac sodium. The fourth layer comprises different oral drug furosemide. The 4 compartments used herein are for illustration only. 2, 3, 4, 5, 6, 7, 8, 9 or more layers could be used in making a multilayered tablet. Two to five layers of tablets are most preferred. In the illustrative embodiment, all the layers are in one plane but additional layers could be placed on top of each other as described in this invention. The liquid, solid or frozen state polymerization or their combination in any proportion can be used to encapsulate the drug and form a unibody tablet as described in this invention. A removable barrier is used to make compartments of desirable size and shape. The thickness of the removable barrier, its shape and its arrangement in the mold cavity can be used to create a desirable pattern of precursor solutions prior to removal and crosslinking. Removable barriers can be used to make bands of one or more crosslinked materials with varying crosslinking densities. The tablet size can vary depending on the drug potency and target animal or human. Tablet sizes and shapes adopted by the current pharmaceutical industry could be used. The unibody object formed has one layer with magnetic particles to provide magnetic property to the object. Two layers have diclofenac sodium as an illustrative drug. One of the layers comprising diclofenac sodium has no NBP and is used for quick/fast release of drugs from the tablet. The other layer comprising diclofenac sodium has hydroxypropylmethylcellulose as an illustrative NBP polymer to slow the release of diclofenac sodium upon ingestion. A non-crosslinked polymer additive such as hydroxypropylmethyl cellulose is used herein for example only. Other NBP like additives that can be added to slow the release include many natural and synthetic macromolecules/polymers such as gelatin, polyacrylic acid, chitosan, poly(methyl vinyl ether-alt-maleic anhydride), poly(allylamine), PVA, PEO and the like. In another embodiment, commercially available empty gelatin or HPMA (vegetarian capsule) capsules are used to fill the precursor solution with drugs. Precursors and crosslinked hydrogels and organogels as described in this invention can be used as a carrier for drugs. An empty capsule cavity is filled with the precursor composition. Components of precursors and solvents are chosen such that they do not damage the capsule material and maintain its structural integrity. If water based precursor compositions are used, fast polymerization, solid state/frozen state polymerization is used before water can damage the structural integrity of the capsule. After polymerization, the crosslinked composition may be dried or lyophilized to remove the solvent. Two halves of the capsules are joined to make a sealed capsule ready for ingestion and oral drug delivery. Each half can have the same or different crosslinked composition and drug. Please refer to the discussion of multilayered implants which also can be applied herein to make multilayered oral tablet or capsule. Some preferred organogel compositions described in this invention can be used to make oral tablets and capsules. Many oral drugs and/or its derivatives/salts are soluble in organic solvent and thus are suitable for encapsulating in organogel compositions described in this invention. Some preferred oral tablets or capsules comprising organogel composition can be used without drying/lyophilization provided organic solvent used is a non-solvent for the capsule material and is water soluble and biocompatible. The non-solvent used must be inert towards the capsule material and should not substantially affect the physical and chemical properties of the capsule. Water miscible organic solvent in organogels are preferred. The list of preferred organic solvents is described in the earlier section. In one illustrative example, polyethylene glycol methyl ether is used as an illustrative organic solvent. Upon ingestion and after the dissolution of the gelatin shell material, the polyethylene glycol methyl ether is quickly dispersed in the gastric fluid helping the quick release of the drug without going through the solid to the solution phase. Particles with two or more layers of particles with drugs have been disclosed in earlier sections. Such particles may be enclosed in the capsule for oral drug delivery (FIG. 18N). Commercially available empty gelatin or HPMA based hard and soft capsules in sizes ranging from 000 to 4 can be used. The size of the capsule will depend on the size of the particle being enclosed. Other custom sized capsules and commercially available capsule materials also can be used. In another embodiment a 3 layer oral tablet where each layer has a cholesterol sequestering property has been disclosed. In some embodiments, the drugs may be loaded in the capsule or oral tablet via solvent diffusion technique. The preferred wavelength for making an oral capsule/tablet is a long UV or visible light, especially visible laser light with wavelength 390 to 800 is most preferred.

A pharmaceutical oral capsule such as shown in FIG. 18M can be used as a quick dissolvable tablet. The crosslinked composition polymerized in the tablet may be organogel or hydrogel with the drug or bioactive compound. The solvent used in organogel is preferably biocompatible and water soluble. Optionally liquid or solid carriers in the organogel or hydrogel for the drug can be used to quickly disperse the drug into the abdominal cavity upon ingestion. The solid or liquid carrier include but not limited to: editable oil, canola oil, glycerol, PEG and its derivatives with molecular weight 400 to 1000, sugars such as maltose, fructose and the like. The preferred solid carrier is sugar or inorganic salt such as sodium chloride.

In another embodiment, hydrogel microparticle/microspheres comprising psyllium husk as an active agent/non-crosslinkable biodegradable polymer is made. Precursors such as gelatin methacrylate and PEG35KUA were used as illustrative compounds to make the psyllium husk comprising microspheres. The encapsulation is believed to prevent the agglomeration of psyllium husk fibers forming a block of hydrogels. It is believed that the encapsulation matrix also prevents interaction with the active bacteria present in the digestive system which is believed to cause flatulence. The semipermeable encapsulation matrix of PEG based crosslinked matrix allows the psyllium husk matrix to retain its ability to interact with bile acid products and lower the serum cholesterol.

The following non-limiting examples are intended to illustrate the inventive concepts disclosed in this document. Those skilled in the art will appreciate that modifications can be made to these examples, drawings, illustrations, specifications and claims, which are intended to fall within the scope of the present invention.

### Materials and Methods

Eosin Y, ethyl eosin, acrylic acid n-hydroxysuccinimide ester, polyethylene glycol, polyethylene oxide and polypropylene oxide block copolymers, 2-Hydroxy-1-[4-(2-hydroxyethoxy)phenyl]-2-methyl-1-propanone (Irgacure 2959), n-hydroxysuccinimide, were purchased from Sigma-Aldrich. Multifunctional hydroxyl and amine-terminated polyethylene glycols were purchased from Dow Chemicals, BASF, Huntsman, Texaco, Creative PEG Works. Disuccinimidyl glutarate (DSG), sulfosuccinimidyl suberate (DSS) and N-hydroxysulfosuccinimide were purchased from Pierce or Sigma-Aldrich. PEG based monofunctional, difunctional, trifunctional, tetra-functional and octa-functional NHS esters and other derivatives were sourced from commercial sources such as Creative PEG Works, Winston Salem, N.C., USA; Jenkem Technology USA, Allen, Tex., USA.; BOC Sciences, Shirley, N.Y. USA; Laysan Bio, Inc. Arab, Ala.; NOF America, Corporation, White Plains N.Y. USA and Sigma Aldrich, USA. They may also be synthesized by procedures described in illustrative embodiments reported in this invention or using methods known in the art. Trilysine acetate salt (Cat No 402495) was purchased from Bachem Americas. Lipase (Type VII, Cat No L1754) is obtained from Sigma. Various monomers are obtained from commercial sources like Sigma Aldrich, Polysciences, Sartomer USA, Exton Pa., and Gelest Morrisville, Pa., and Scientific Polymer Products, Ontario N.Y. Many crosslinked products are generally added/filled with inorganic fillers such as silica (HI-SILO silica product from PPG) to improve mechanical and other physical properties. In addition to fillers, crosslinked compositions may comprise other additives such as antioxidants, stabilizers, coloring pigments and the like. Many reagents, solvents can be purchased from commercial sources such as, by way of example, and not limitation, Polysciences, Fluka, ICN, Sigma-Aldrich and the like. SYLGARD™ 184 Silicone Elastomer Kit is purchased from local suppliers to cast silicone rubber molds. Most of the reagents/solvents are purified/dried using standard laboratory procedures. Small laboratory equipment and medical supplies can be purchased from Fisher or Cole-Parmer.

Cell culture experiments are performed using a standard mammalian tissue culture laboratory or microbiology laboratory capable of handling and growing mammalian and human cell cultures. Unless mentioned, Live cells, blood or plasma or serum related experiments are done in a sterile manner. Commercial sources like Sigma-Aldrich, Gibco, VWR, Thermofisher and the like are used to obtain cell culture medium. American Type Culture Collection (ATCC) and other commercial vendors are used to obtain mammalian cells. A clean room or sterile hoods are generally used to conduct the live cell culture experiments and to maintain sterility.

Long UV light lamps (model UV-300, Wenzhou Aurora Technology Company Ltd., Wenzhou, China, 365 nm light with intensity around 26,500 μW/cm² when held 1 inch above the surface) or Black-Ray UV lamp, (360 nm light filter, about 10000 mW/cm2 intensity when held about 1 inch above the surface) is used. Argon laser emitting at 513 nm is used for visible light polymerization initiated by Eosin. Solid state laser sources emitting at 520 nm, 532 nm , 445 nm, 488 nm with total power output from 0.05 Watts to 25 Watts can be purchased from Laser Lab Source, Bozeman, Mont. 59718 and other commercial sources. The size, shape and distribution of microparticles or microspheres can be assessed by laboratory microscope with varying magnification power or with scanning electron microscopes. The distribution of various sizes in a mixture is assessed by a particle size analyzer.

Molecular weight is determined by gel permeation chromatography (GPC); or NMR (proton) or mass spectrophotometry.

Biocompatibility is assessed by several USP tests recommended by the US FDA. People skilled in the art know that commercial laboratories are available which perform routine biocompatibility tests for their clients. These tests include implantation and subsequent histological testing for immunogenicity, inflammation, drug release and degradation studies.

In vitro degradation of the polymers/gels is monitored gravimetrically at 37 degree C., in aqueous buffered medium such as, by way of example, and not limitation, phosphate buffered saline (pH 7.4). In vivo biocompatibility and degradation life times are assessed after subcutaneous implantation. The implant is surgically inserted into the animal body. The degradation of the implant over time is monitored gravimetrically or by chemical analysis. The biocompatibility of the implant is assessed by standard histological techniques.

Chemical analysis such as, by way of example, and not limitation, structure determination is done using nuclear magnetic resonance (proton and carbon-13), Raman spectroscopy, x-ray diffraction and infrared spectroscopy.

High-pressure liquid chromatography or UV-visible spectrophotometry is used to determine drug elution profiles.

Thermal characterization such as, by way of example, and not limitation, melting point, shrink temperature and glass transition temperature are done by differential scanning calorimetric analysis. The aqueous solution properties such as, by way of example, and not limitation, self-assembly, micelle formation and gel formation are determined by fluorescence spectroscopy, UV-visible spectroscopy and laser light scattering instruments. Drug release studies are conducted in PBS under sink conditions at 37 degrees C. and the drug elution is monitored by HPLC or UV-VIS spectrophotometer.

### EXAMPLE 1

Macromonomer synthesis.

Biodegradable macromonomer synthesis.

### Example 1A

Synthesis of PEG based biodegradable macromonomer with polymerizable endgroups.

Part 1: Synthesis of polyethylene glycol lactate copolymer.

50 ml Pyrex pressure sealing tube is flame dried and cooled under argon. 30 g of PEG 35000 (PEG with molecular weight 35000 g/mole), 1.234 g of dI-lactide and 30 mg of stannous octoate are charged into the tube. The tube is then connected to a vacuum line followed by argon gas and sealed under argon. The tube is then immersed in a silicone oil bath maintained at 140 degrees C. The reaction is carried out for 16 h at 140 degree C. The polymer from the tube is recovered by breaking the Pyrex tube. The polymer is then dissolved in 100 ml chloroform or toluene and precipitated in 2,000 ml cold hexane or ether. The precipitated polymer (35KL5) is recovered by filtration and dried under vacuum for 1 day at 60 degrees C. It then is immediately used in the next reaction below.

Part 2: End-capping of 35KL5 with polymerizable or crosslinkable group

15 g of polyethylene glycol lactate polymer (35KL5) prepared above is dissolved in 250 ml dry toluene. About 50 ml of toluene is distilled out under an argon atmosphere to remove traces of water from the reaction mixture. The solution is cooled to room temperature, 0.33 g of triethylamine and 0.3 g acryloyl chloride are added. The reaction mixture is then warmed to 50-60 degree C. and stirred for 30 minutes at 50-60 degree C.; cooled to room temperature and filtered. The product (macromonomer) is precipitated by adding the filtrate to 2,000 ml cold ether. The precipitated macromonomer (PEG35K-LACTATE-5-diacrylate; 35KL5A or PEG35KL5A) is recovered by filtration. It is then dried under a vacuum for 12 h at 50 degree C. The macromonomer synthesized has PEG 35000 Daltons as a central block that is extended with polylactide as a biodegradable block (five lactide units on both sides of PEG chain) and terminated with an acrylate polymerizable group. The polylactate group is between acrylate and PEG.

Using a similar procedure as above, 30 g of PEG 35000 (PEG with molecular weight 35000 g/mole or Daltons) is reacted with 2.468 g of dI-lactide to form a PEG35K-LACTATE-10 (35KL10) copolymer which was then endcapped with acrylate group to form PEG35K-LACTATE-10-diacrylate (35KL10A). Similarly, 30 g of PEG 35000 is reacted with 24.68 g of dI-lactide to form a PEG35K-LACTATE-10 (35KL100) copolymer; and 30 g of PEG 35000 is reacted with 61.71 g of dI-lactide to form a PEG35K-LACTATE-250 (35KL250) copolymer. 35KL100 and 35KL250 were reacted with acryloyl chloride to form diacrylate derivatives (35KL100A and 35KL250A respectively). As the percentage of lactide in the copolymer is increased, water solubility of the copolymer at room temperature (25 degree C.) is decreased but remains substantially unaffected in organic solvent like dimethyl sulfoxide (DMSO). PEG-polylactone or PEG-polycarbonate copolymers prepared as above with solubility less than 10 g /100 g in water at 25 degree C. but with solubility greater than 10 g/100 g in an organic solvent (DMSO) are preferred in many embodiments involving crosslinked organic solvent gels. The solubility in water will depend on the PEG molecular weight used and the ratio of PEG to lactone or carbonate cyclic monomer used in the polymer synthesis.

Using a similar procedure as above, 30 g PEG 10000 (PEG molecular weight 10000 g/mole, four branches, one terminal hydroxyl group per PEG branch, tetrafunctional) is reacted with 8.460 g dl lactide in the first part and 1.668 g acryloyl chloride and 1.882 g triethylamine in the second part. The macromonomer (PEG4L5A or PEG10K4LA) formed has four acrylate groups per molecule which upon polymerization and crosslinking produces crosslinked hydrogels that have higher crosslinking density than its bifunctional but the same molecular weight counterpart produced using a procedure as above. All cyclic lactone polymerizations reported herein are catalyzed by stannous octoate.

In another modification as above, 30 g PEG 20000 is reacted with 1.234 g dl lactide in the first part and 0.501 g acryloyl chloride and 0.565 g triethylamine in the second part to produce PEG 20K-lactate-acrylate macromonomer (20KL5A or PEG20KL5A).

In another modification as above, 30 g PEG 10000 is reacted with 1.234 g dl lactide in the first part to produce PEG-lactide copolymer (10KL5) and 0.33 g of triethylamine and 0.3 g acryloyl chloride in the second part to produce PEG 10K-lactate-acrylate (10KL5A or PEG1OKL5A) macromonomer.

In another modification as above, 10 g Pluronic F127 is reacted with 1.200 g dI-lactide in the first part to produce Pluronic F127-lactate and 0.297 g acryloyl chloride and 0.335 g triethylamine in the second part to produce Pluronic F127-lactate-acrylate (F127LA) macromonomer. This macromonomer displays thermosensitive gelation at 20-40 percent in distilled water or PBS (pH 7.4) around 37 degree C. Briefly 2 g of the macromonomer F127LA is dissolved in 8 g cold distilled water at 0 to 10 degree C. in a glass test tube. The solution is warmed to 37 degree C. in a water bath where it forms a gel (physical gelation) which can be converted back into solution upon cooling to 0 to 10 degree C. (thermoreversible gelation). This macromonomer solution can be crosslinked using a photoinitiator and light in gel form (around 37 degree C.) as well as in solution form (around 0 to 10 degree C.). This property of gelation in solution and physical gel form is used in making multilayered gels described in some embodiments.

In another embodiment, in a 250 ml flask 1.005 g Jeffamine (CAS 65605-36-9) was dissolved in 100 ml toluene. 20-30 ml toluene was distilled out under a nitrogen atmosphere. The solution was cooled to RT and 2.011 gm DI-lactide and 0.1 ml stannous octoate were added sequentially in the solution under nitrogen at RT. The solution mixture was refluxed for 4 hours under a nitrogen atmosphere and cooled and then poured in ice cold hexane to precipitate Jeffamine lactide copolymer. Hexane was removed by decantation and the rest of the solvent is removed by air drying. Finally, the product was dried under vacuum for 24 h and kept in a desiccator until use. The Jeffamine lacide thus produced is then reacted with acryloyl chloride and triethylamine to produce acrylate terminated Jeffamine lactide (JALA). This PEG based biodegradable macromonomer displays thermosensitive gelation at 25 to 40 percent (w/v) in distilled water and at around 37 degree C. This solution can also be crosslinked in gel form (around 37 degree C.) as well as in solution form (around 0-10 degree C.).

In another modification as above, 30 g PEG 20000 is reacted with 0.720 g dl lactide and 0.590 g glycolide to make PEG20K-PLGA copolymer in the first part and 0.497 g acryloyl chloride and 0.561 g triethylamine in the second part to produce PEG20K-PLGA-diacrylate macromonomer. In this macromonomer, the biodegradable segment/block is a copolymer of lactide and glycolide.

In another modification as above, 30 g PEG 20000 is reacted with 0.570 g caprolactone and 0.580 g glycolide to make PEG20K-PCLGA copolymer in the first part and 0.497 g acryloyl chloride and 0.561 g triethylamine in the second part to produce PEG20K-PCLGA -diacrylate macromonomer. This macromonomer has polycaprolactone and polyglycolide copolymer as biodegradable blocks.

In another modification as above, 2 g 1,6-hexanediol is reacted with 1.44 g dI-lactide and 0.1.160 g glycolide to make PLGA copolymer in the first part and 2 g PLGA copolymer is then reacted with 0.337 g acryloyl chloride and 0.380 g triethylamine in the second part to produce hydrophobic organic solvent soluble PLGA -acrylate macromonomer. This hydrophobic PLGA macromonomer is insoluble in water and soluble only in an organic solvent like THF and is a neat liquid at ambient temperature.

In many embodiments, PEG molecular weight is kept between 400 to 35000 g/mole and weight ratio of PEG in PEG-polylactone copolymer is kept below 50 percent preferably below 30 percent, so that water solubility of the resultant polymer is reduced to less than 10 g/100 g preferably 5 g /100 g of water at 25 degree C.

By changing PEG molecular weight (diol or polyol), the number of acrylate groups per PEG and length of polylactones or polycarbonates as indicated above, crosslinked networks with differing degradation times and molecular permeability can be made which can be used in a variety of drug delivery and cell encapsulation applications. Macromonomers with biodegradable blocks of polymer and copolymers of lactate, caprolactone, glycolate, trimethylene carbonate, dioxanone and other biodegradable polyester blocks, their copolymers provides crosslinked gels with different degradation times. By changing the nature of biodegradable blocks, their length and crosslinking density, crosslinked hydrogels with degradation times from days to several months can be obtained. Macromonomer comprising with 10 to 60 percent PEG and 90 to 40 percent biodegradable polymers are preferred for polymerization in organic solvents. Macromonomers with water solubility of 10 g/100 g water at 25 degree C. or lower solubility are preferred for polymerization in organic solvent and drug delivery applications.

Several macromonomers synthesized as above can also be effectively polymerized and crosslinked. Some illustrative example are given below: Effective crosslinking of biodegradable macromonomers using free radical polymerization initiators.

### Example 1A-1

Long UV light initiated polymerization in the organic solvent.

In 100 ml beaker 3 g of PEG35K-LACTATE-5-acrylate diacrylate (35KL5A) prepared as above is dissolved in 9 g DMSO. 1 ml of this solution is mixed with 10 mg 2-hydroxy-4′-(2-hydroxyethoxy)-2-methylpropiophenone (Irgacure 2959) photoinitiator and optionally with 20 microliters of n-vinyl pyrrolidone as comonomer. 20 μl of the solution is then exposed to 360 nm long UV light (Black-Ray UV lamp, 360 nm light, and 10000 mW/cm2 intensity) for 5 minutes. The liquid solution is converted into soft gel indicating effective polymerization and crosslinking. In some embodiments, 300 mg of 2-Hydroxy-4′-(2-hydroxyethoxy)-2-methylpropiophenone is dissolved in 0.7 g of n-vinyl pyrrolidone to prepare a stock solution (ISS) and this solution is added to macromonomer solution giving final initiator concentration ranging from 0.05 percent to 5 percent but typically at 0.1 to 0.5 percent range.

In another modification of the above example, DMSO is replaced with PBS pH (7.4). The hydrogel produced is biodegradable due to the presence of polylactate groups in the crosslinked network.

### Example 1A-2

Visible light initiated polymerization.

In a 100 ml beaker 3 g of PEG35K-LACTATE-diacrylate (35KL5A) prepared as above is dissolved in 9 g PBS to form a macromonomer solution. In another 10 ml glass vial, 200 mg of eosin Y is dissolved in 700 mg of n-vinyl pyrrolidone to prepare eosin Y stock solution. In one ml of macromonomer solution, 3 microliters of eosin Y stock solution, 50 microliters of 5 Molar triethanolamine in PBS are added to PEG-LACTATE-5-acrylate solution and the solution is covered with aluminum foil to prevent premature polymerization by ambient light. 20 μl of the solution is exposed to high intensity 514 nm light (argon laser) or visible flood light, intensity 10-100 mW per centimeter square to initiate photopolymerization and crosslinking of macromonomer. The liquid solution is converted to solid soft gel in 5 minutes of exposure. The conversion of a solution to a soft gel indicated effective polymerization and crosslinking. Photopolymerization of eosin-triethanolamine initiating systems depends on many factors that will affect gel time of the precursor solution. Eosin concentration may vary from 5 micromolar to 0.5 mM, triethanolamine concentration may vary from 5 mM to 0.1 M, vinyl pyrrolidone concentration varies from 0.001 percent to 0.1 percent, Laser power at 514 nm wavelength varies from 120 mW to 2 W, macromonomer concentration varies from 10 to 40 percent.

In another modification, lithium phenyl-2,4,6-trimethylbenzoylphosphinate (LPTP) photoinitiator is used as UV and visible light initiator (sigma catalog 900889). This initiator is known to be cytocompatibile and initiates at 360 nm in long UV range as well as 400 nm in the visible range (B. D. Fairbanks et al., Biomaterials, Volume 30(35) Page 6702, 2009). Briefly, the initiator LPTP is dissolved in PBS at 0.1 percent concentration. The monomer PEG35K-LACTATE-5-acrylate diacrylate is added to the initiator solution to make a 10 percent final monomer concentration. 30 μl of the solution is exposed to long UV light (360 nm) and visible light (400 nm) using UV lamp (Wenzhou Aurora Technology Company Ltd., Wenzhou, China, model UV-300, 365 nm light and visible white light) with minimum intensity 26,500 μW/cm for 6 minutes to form a crosslinked gel.

### Example 1B

Macromonomer with polymerizable groups attached to side groups of linear polymers.

Crosslinked polymer networks that degrade via enzymatic degradation mechanisms in vivo.

### Example 1B-1 Gelatin modification with methacrylate polymerizable group.

5.04 g gelatin is dissolved in 25.0 ml of carbonate/bicarbonate buffer, pH 9.0. To this solution, 0.5 ml methacrylic anhydride (0.1 ml per gram gelatin) is added dropwise with constant stirring. The reaction mixture is kept at 40 degree C. The reaction product is checked after one hour for its gelation. 500 μl of product is taken out in a vial and 2 μl of photo-initiator solution ISS is added to it. About 100 μl solution is filled into a silicone mold cavity (diameter 3 mm and depth 1 mm) and exposed to UV light. It is noticed that the gel forms within 30 seconds of UV light exposure. The reaction is cooled to room temperature. The product is precipitated by the addition of acetone to the reaction mixture and is washed and separated using acetone and dried under vacuum. In some cases, the gelatin methacrylate product is purified by dialysis with 10000 molecular weight cutoff membranes. The dialyzed solution is lyophilized to recover the product. 1 g of gelatin macromonomer as above is dissolved in 9 ml PBS. Separately 300 mg Irgacure 2959 is dissolved in 700 mg of n-vinyl pyrrolidone to produce an initiator stock solution (ISS). 3 μl of above solution (ISS) is added to 1 ml of gelatin solution and the solution is filled in silicone rubber mold with cylindrical cavities as above and exposed to 360 nm Long UV light (Black-Ray UV lamp, 360 nm light, 10000 mW/cm2 intensity) for 5 minutes. The crosslinked gelatin microcylinders are removed. The crosslinked gelatin undergoes enzymatic gelation upon implantation in the body.

### Example 1C

Hyaluronic acid modification with methacrylate polymerizable group.

1 g of sodium hyaluronate is dissolved in PBS pH 7.4. 10 ml of this solution is added with 0.5 ml glycidyl methacrylate, 0.5 ml triethylamine and 0.1 g tetrabutylamine hydrobromide. The reaction is carried for 48 hours at room temperature with gentle stirring. The methacrylate modified derivative is precipitated in 100 times excess cold methanol. The precipitated polymer is dried under vacuum and one ml 2 percent of the modified polymer solution is mixed with 6 μl of Irgacure solution in n-vinyl pyrrolidone (ISS) and photopolymerized in silicone rubber cylindrical cavities using 360 nm light for 1 to 10 minutes. The resultant gel microcylinders degrade via an enzymatic pathway. The hydroxyl and carboxylic and groups in the crosslinked hyaluronic acid gel can be used in grafting or cyclic lactones or carbonates.

Using a similar procedure as above, 1 gram of hydroxypropyl methyl cellulose (Hypromellose, 220890SH-100SR) is modified to obtain methacrylate ester derivative.

For additional examples of macromonomers, biodegradable crosslinked compositions and free radical polymerization initiating systems please refer to U.S. Pat. Nos. 5,410,016, 5,573,934, 6,201,065, 6,566,406, 9,023,379, 6,387,977, 9,789,073, and cited art therein; cited herein for reference only.

### EXAMPLE 2

Effective free radical polymerization conditions for macromonomers

Organic solvent gels made by free radical polymerization.

Composite crosslinked organic solvent gels (organogels) or hydrogels compositions.

Composite materials comprising crosslinked polymer and non-crosslinked biodegradable polymers.

### Example 2A

Formation of composite materials comprising crosslinked biodegradable or biostable macromonomer and non-crosslinked organic solvent soluble biostable/biodegradable polymer by free radical polymerization.

Synthesis of free radical polymerizable macromonomer (PEG urethane acrylate) as a precursor or macromonomer.

In a 250 ml flask, 5.0 g of polyethylene glycol 35000 (PEG 35000 Daltons molecular weight) is dissolved in 140 ml of toluene from which 20-30 ml of toluene is distilled out. The solution is cooled and is equipped with a stirrer and nitrogen inlet. While stirring 80 μl of hexamethylene diisocyanate was added followed by one drop of dibutyltin dilaurate was added in the reaction flask and the solution was refluxed for 2 hrs. The reaction mixture was cooled to room temperature and 58 μl of 2-hydroxyethyl acrylate was added. The reaction mixture is refluxed for 2 hours, cooled at room temperature. The solution is then poured in 150 ml of cold hexane for precipitation of the product. The product, PEG35000-urethane acrylate (PEG35KUA) is filtered and is washed with 50 ml of cold hexane and dried under vacuum and stored in a dark colored bottle in the refrigerator until use.

Using a similar procedure as above, PEG 2000 Daltons, PEG 3000 Daltons, PEG 6000 Daltons, PEG 10000 Daltons, PEG 20000 Daltons, PEG 100000 Daltons, Pluronic F127, Tetronic 908 and Reverse Pluronic 25R2 were used to make PEG 2000 urethane acrylate (PEG2KUA), PEG 3000 urethane acrylate (PEG3KUA), PEG 6000 urethane acrylate (PEG6KUA), PEG 10000 urethane acrylate (PEG1OKUA), PEG 20000 urethane acrylate (PEG 20KUA), PEG 100000 urethane acrylate (PEG100KUA), Pluronic F127 urethane acrylate (F127UA), Tetronic 908 urethane acrylate (T908UA), Pluronic 25R2 urethane acrylate (25R2UA) macromonomers respectively.

### Example 2A-1

Effective Photopolymerization of PEG35KUA in aqueous solution.

Polymerization and effective crosslinking in aqueous solution, PBS (pH 7.4).

300.1 mg of 2-hydroxy-4′-(2-hydroxyethoxy-2-methylpropiophenone) is first dissolved in 1 ml of DMSO (initiator stock solution, ISS-1). 50.0 mg of PEG35KUA macromonomer is dissolved in 0.5 ml PBS and 10.0 μl ISS-1 is added. After mixing, 50 microliters of the above solution is poured into a circular mold cavity and exposed to long UV light (360 nm light). A soft gel is formed under 3 minutes of exposure indicating effective polymerization and crosslinking.

### Example 2A-2

Polymerization and effective crosslinking in organic solvents.

Polymerization in n-methyl pyrrolidinone (NMP).

50.0 mg of PEG35KUA macromonomer is dissolved in 0.5 ml of warm n-methyl pyrrolidinone followed by the addition of 2 microliters of initiator stock solution (ISS). 50 microliters of NMP solution is exposed to long UV light. The solution forms a soft gel within 300 seconds indicating effective crosslinking. 0.3 ml of NMP macromonomer solution with photoinitiator as above is mixed with 60 mg of polylactide-co-polyglycolide (PLGA, 50:50) copolymer, molecular weight 10000 to 15000 Daltons, until complete dissolution. 50 μl of the above solution is poured into a mold cavity and exposed to UV light as above to form a soft gel. The gel has PLGA entrapped into a crosslinked PEG35KUA network. The gel is washed with water to remove NMP which results in precipitation of PLGA within the crosslinked gel.

### Example 2A-3

Effective polymerization in dimethyl sulfoxide (DMSO)

50.0 mg of PEG35KUA macromonomer is dissolved in 0.5 ml of warm DMSO. 250.4 mg of 2-Hydroxy-4′-(2-hydroxyethoxy)-2-methylpropiophenone is dissolved in 0.5 ml of vinyl pyrrolidone to prepare an initiator stock solution (ISS-2). 10 microliters of ISS-2 is added to the macromonomer solution. 50 microliters of DMSO solution is then exposed to long UV light. The solution forms a firm gel within 300 seconds. The same solution as above is mixed with 20 mg of bupivacaine base (an illustrative water insoluble drug) and photopolymerized. The solvent is removed by vacuum drying or exposure to water to precipitate or crystalize the drug in situ inside the organic solvent gel. The crystalized/precipitated drug is released from the gel by the dissolution of crystals upon implantation.

0.3 ml of DMSO macromonomer solution with ISS-2 as above is mixed with 60 mg of polylactide-co-polyglycolide (PLGA, 50:50 copolymer, molecular weight 30000 to 50000 Daltons) until complete dissolution. 50 μl of the above solution is poured into the mold cavity and exposed to UV light for 5 minutes as above to form a soft gel. The gel has PLGA entrapped into a crosslinked PEG35KUA network. The gel is washed with methanol which results in precipitation of PLGA within the crosslinked gel and it also removes DMSO and initiator fragments. In another modification as above, PLGA is replaced with PLGA having a molecular weight between 100000 to 150000 Daltons. In another modification, the DMSO macromonomer solution with PLGA is mixed with 6 mg of bupivacaine base (an illustrative drug) prior to photopolymerization. The bupivacaine solution is poured into mold and photopolymerized as above in a mold to form a disk. The disk has crosslinked PEG hydrogel with PLGA and drug entrapped in the gel. The composite gel is washed with water wherein PLGA is insoluble and the drug has low water solubility. Water removes DMSO and precipitates the PLGA entrapping bupivacaine in the precipitated PLGA as well in the crosslinked macromonomer.

### Example 2A-4

Polymerization in polyethylene glycol dimethyl ether as an organic solvent, molecular weight 550 Daltons (PEGDME).

50.0 mg of PEG35KUA macromonomer is dissolved in 0.5 ml of warm polyethylene glycol dimethyl ether (molecular weight 550 Daltons, PEGDME) as a solvent followed by addition of 10 microliters of initiator stock solution (ISS-2). 50 microliters of PEGDME solution is exposed to long UV light in a circular mold cavity. The solution forms a firm gel within 300 seconds.

Using a similar method as above, dichloromethane, acetone, tetrahydrofuran (THF), methanol, ethanol, chloroform, toluene were tested for free radical polymerization. All solvents tested produced effectively polymerized crosslinked soft or firm organic solvent gels when used under effective polymerization conditions.

### Example 2A-5

Composite of free radically crosslinked hydrogel and a biodegradable liquid carrier.

50.0 mg of PEG35KUA macromonomer and 50 mg of sucrose acetate isobutyrate are dissolved in 0.5 g ethanol followed by the addition of 10 microliters of initiator stock solution (ISS-2). 50 microliters of macromonomer and sucrose acetate isobutyrate solution is exposed to long UV light in a circular mold. The solution forms a firm gel within 300 seconds. The solvent is removed from the crosslinked gel under vacuum with entrapped sucrose acetate isobutyrate as a liquid carrier in the biodegradable crosslinked gel. Using the same method as above, vitamin E acetate is used in place of sucrose acetate isobutyrate and the solvent is changed from ethanol to dichloromethane. In another embodiment, an illustrative biodegradable liquid polymer (polycaprolactone triol average molecular weight 900 g/mole) is used as a liquid carrier. In another embodiment, PEG-polylactone copolymer that is liquid at 30 degree C. is used as a liquid carrier.

In some embodiments, after removing the solvent, the crosslinked polymer network remains swollen in the liquid carrier forming an organogel implant material.

### Example 2A-6

Composite of biodegradable free radically and covalently crosslinked hydrogel and physically crosslinked hydrogel.

50.0 mg of PEG35KUA macromonomer and 150 mg of Jeffamine lactide (JAL, an illustrative biodegradable polymer with thermosensitive gelation properties) are dissolved in 0.5 ml of dichloromethane followed by addition of 10 microliters of initiator stock solution (ISS). 50 microliters of macromonomer and Jeffamine lactide solution is exposed to long UV light in a circular mold. The solution forms a firm gel within 300 seconds. The solvent is removed from the crosslinked gel under vacuum. The composite hydrogel with entrapped thermosensitive polymer (Jeffamine lactide) is exposed to warm PBS at 37 degrees for 48 h. The Jeffamine lactide forms a thermoreversible gel inside the crosslinked PEG35KUA macromonomer gel when exposed to water at 37 degree C. and at 15-30 percent concentration. Both the crosslinked hydrogel and Jeffamine lactide gel can be used alone or in combination for drug delivery application. In another modification as above, the JAL is substituted with Pluronic® F127 (commercially available thermosensitive polymer) at a concentration of 30 percent to produce Pluronic F127 encapsulated composite hydrogels. In another modification of the above example, aqueous solution of PBS is used in place of organic solvent dichloromethane. The components are dissolved in PBS at 0-10 degree C. and then crosslinked with light to produce crosslinked hydrogel with thermosensitive polymer. At around 37 degree C., the thermosensitive polymer is present as a thermosensitive physically crosslinked hydrogel in the photocrosslinked hydrogel.

In some organogel preparations, care must be taken to dissipate the heat of polymerization liberated during polymerization and crosslinking process. This is especially more important when using neat monomer liquids or precursors used as a reactive solvent. This may be done by many methods known in the art including but not limited to: limiting the amount of material used, using cryogenic baths and the use of specialized heat transfer devices to remove heat from the reaction medium.

### EXAMPLE 3

Crosslinked compositions made by condensation polymerization.

Synthesis, polymerization and crosslinking of biodegradable water/organic solvent soluble precursor/s based on PEG.

### Example 3A

Precursors soluble in organic and water based solvents

Preparation of crosslinked synthetic biodegradable hydrogels made by precursors comprising electrophilic and nucleophilic groups.

Preparation and crosslinking of biodegradable hydrogels prepared from PEG based precursor/s.

Part 1: Conversion of PEG hydroxyl groups into carboxylic groups.

100 g branched polyethylene glycol (molecular weight 10000 Daltons, 4 arm; PEG10K4ARM) is dried at 60 degree C. overnight under vacuum prior to use. In a 1000 ml flask, 30 g PEG10K4ARM is dissolved in 500 ml of toluene. 200 ml of toluene is distilled out to remove trace amounts of water in PEG and the solution is cooled to room temperature. 6.84 g glutaric anhydride and 1.6 g dimethylamino pyridine (DMAP) are added to the flask and the mixture is refluxed for 12 hours under nitrogen atmosphere and additional 100-200 ml toluene is distilled off. The solution is cooled and the PEG10K4ARM glutarate with 4 acid end groups is isolated by precipitating the product in 2,000 ml of dry, ice cold ether. The product is recovered by filtration and dried under vacuum at 60 degree C. and used immediately in subsequent carboxyl group activation reaction.

Part 2: Activation of PEG10K4ARM glutarate with acid end group using n-hydroxysuccinimide.

In a 500 ml reaction flask, 30 g of PEG10K4ARM glutarate as prepared in Part 1 is dissolved in 300 ml of dry methylene chloride or dimethylformamide or THF. To this solution, 1.52 g of n-hydroxysuccinimide, 1.6 g of 4-Dimethylaminopyridine (DMAP) and 2.7 g of 1,3-dicyclohexyl carbodiimide (DCC) are added. The reaction mixture is cooled to 0 degree C. using an ice bath and stirred overnight under a nitrogen atmosphere. The reaction byproducts, dicyclohexylurea, are removed by filtration. The filtrate is evaporated under vacuum and the residue obtained is redissolved in 100 ml of toluene with warming and stirring. The toluene solution is precipitated in 2,000 ml of cold ether. The precipitated PEG10K4ARM glutarate-NHS ester is filtered and dried under a vacuum at 55 degree C. until a constant weight is achieved and stored under nitrogen at −20 degree C. until use. PEG10K4ARM glutarate NHS ester can also be purchased from Laysan Bio, Inc. Arab, AL. PEG with four branches serves here as the central block (core) that is extended with glutarate ester and then terminated with electrophilic n-hydroxysuccinimide (NHS) as end groups. PEG based monofunctional, difunctional, trifunctional, tetrafunctional, octa functional NHS esters similar to synthesized as above can be purchased from commercial sources such as Creative PEG Works, Winston Salem, N.C., USA; Jenkem Technology USA, Allen, Tex., USA.; BOC Sciences, Shirley, N.Y. USA; Laysan Bio, Inc. Arab, Ala.; NOF America, Corporation, White Plains N.Y. USA and Sigma Aldrich, USA. The same suppliers can also provide or make custom made derivatives of PEG with various molecular weights and branching with other electrophilic and nucleophilic terminal end groups and with various degradable esters.

Part 3: Effective polymerization and crosslinking of precursors via condensation polymerization. In a 15 ml glass vial, 1 g of PEG10K4 ARM tetramine is dissolved in 9 ml of PBS (pH 7.4). In another 15 ml glass vial, 1 g of PEG10K4ARM glutarate NHS ester prepared as above is dissolved in 9 ml of PBS (pH 7.4). The freshly made solutions are immediately used in crosslinking reactions (PEG10K4ARM glutarate NHS ester undergoes hydrolysis in PBS if not used immediately). 10 microliters each of NHS ester and amine is mixed on a glass plate and observed for gel formation. The gel forms with 5 minutes of mixing. In some cases, 1-10 microliters of 10 percent triethanolamine in PBS is added to accelerate the crosslinking reaction. The crosslinking reaction is pH sensitive and pH is carefully controlled (pH range 6 to 8) to achieve crosslinking time within 10 seconds to 10 minutes. The formed gel is soft and elastic indicating effective crosslinking. In another method, a dual syringe (Product code 6B23-3ml×3ml, 1:1 Ratio, with 2mm×8 Element mixer tip) from Plas-Pak Industries, Inc., Norwich, Conn., USA is used. The 2 ml of PEG10K4ARM tetramine (10 percent) is loaded in one syringe. The other syringe is loaded with 2 ml of PEG10K4ARM glutarate NHS ester (10 percent). 20 μl of the solution is injected (equal volumes of each component producing molar equivalent quantities of precursors) are dispensed via mixer and the tip (The residence time in the mixer and tip must be less than gel time of the crosslinking composition) and observed for gelation. The gel forms within 10 minutes. It is generally preferred to use equal or substantially equal numbers of electrophilic and nucleophilic groups during crosslinking reactions to achieve faster gelation and achieve high molecular weights. In a variation of the above example, PEG10K4ARM glutarate NHS ester is replaced with PEG 10K4ARM succinate NHS ester or PEG10K4ARM adipate NHS ester or PEG10K4ARM suberate NHS esters (purchased from commercial sources or synthesized using similar procedure as described above) and crosslinked with PEG tetramine in molar equivalent quantities as mentioned before. A combination of these degradable esters may be used to obtain the desired biodegradation rate. The variation in hydrolysis rates of esters in crosslinking agents (succinate, glutarate, adipate, suberate) gives different degradation rates for the crosslinked gels. The in vivo degradation rates may vary from a few days to several months. The crosslinking density is controlled by varying the total number of reactive groups in the precursors (total of electrophilic and nucleophilic groups) and may typically contain 5, 6, 7, 8, 9,10, 11,12, 13, 14, 15, 16 or more reactive groups. In general, higher number of total reactive groups in the precursors produce higher crosslinking density or lower molecular permeability or tighter network. Crosslinking density or molecular permeability is also controlled by molecular weight or molecular chain length distance between reactive groups. Generally, a shorter chain length distance between reactive groups leads to tighter or denser networks. PEG molecular weight and its branching can be used to control crosslinking density. Low molecular weight and/or highly branched PEG generally give highly dense crosslinking networks which generally have low molecular permeability. PEG molecular weight can be used to control crosslinked density. Crosslinked materials with high crosslinking density will generally take a longer time to degrade. The overall degradation rate is controlled by the degradable ester used in the network, its concentration in the network as well as its crosslinking density.

1 g of PEG10K4 ARM tetramine is dissolved in 9 ml of DMSO in a 15 ml glass vial. In another 15 ml glass vial, 1 g of PEG10K4ARM glutarate NHS ester prepared as above is dissolved in 9 ml of DMSO. 1 ml of each solution is filled into separate syringes and injected using a dual syringe injector. The solutions are mixed in equal volume and then immediately poured into silicone cylindrical cavity mold or Mpatch based microneedle mold as above. The filing is done prior to gelation. The reaction is continued in the mold until complete crosslinking in DMSO is achieved to form an organic solvent gel. The organic solvent gel is removed from the mold and stored until use. In another example, 0.2 g of PLGA (molecular weight 15000-30000 Daltons, endcapped with acetate group) and 1 g of PEG10K4 ARM tetramine is mixed with 9 ml of DMSO in a 15 ml glass vial. This solution is then used to crosslink with PEG10K4ARM glutarate NHS ester as above. The gel formed has PLGA entrapped in the organic solvent gel composition which could be precipitated in the particle after removal of solvent.

In another modification of the above example, 1 g of Trilysine acetate salt is dissolved in 9 ml of DMSO in a 15 ml glass vial. In another 15 ml glass vial, 1 g of PEG10K4ARM glutarate NHS ester prepared as above is dissolved in 9 ml of DMSO. Both solutions are mixed in equimolar quantities to form an organogel in DMSO. The same reaction also can be done in aqueous buffered solutions (pH 6 to 7.4). Depending on the final pH used, the gel time of the crosslinked gel changes. Compositions with gel time few seconds to several minutes can be obtained by varying the pH of the reaction medium.

### Example 3B

102.1 mg of glycerol ethoxylate (Molecular weight 1000), 0.2 ml of Toluene, 19 μl of glutaryl chloride and 42.0 μl of Triethylamine are mixed well and filled in mold cavities. After 30 minutes, sticky organic solvent gels are observed in the mold cavities. In another example, 37.5 mg of glycerol ethoxylate, 0.1 ml of Toluene, 9 μl of glutaryl chloride are mixed and this mixture is poured into the mold cavity and then 33 μl of triethylamine is added and mixed. Delayed addition of triethylamine helps to delay the gelation which helps to give sufficient workup time to fill the cavities in the mold before crosslinking and gelation. The gel is removed from the mold and washed with water to remove triethylamine hydrochloride and other unreacted products. In another embodiment as above, 102.1 mg of glycerol ethoxylate and 50 mg of PLGA (molecular weight, acetate endcapped) is reacted with glutaryl chloride as above to prepare an organic solvent gel with PLGA entrapped in the gel. The stoichiometry between electrophilic and nucleophilic groups is varied until gelation time between 2 to 10 minutes. Equivalent or very close to equivalent stoichiometry between electrophilic and nucleophilic groups in the precursors is most preferred for effective polymerization and crosslinking.

### Example 3C

Precursors soluble in organic solvents and produce organic solvent gels upon crosslinking.

Preparation of crosslinked enzymatically degradable organic solvent gels.

Crosslinking of linear polymers with side functional groups like gelatin or hyaluronic acid using hexamethylene diisocyanate.

In a 15 ml glass vial, 1 g of gelatin dissolved in 20 ml of dry DMSO. The mixture is warmed to 60 degree until dissolution and held at 45 degree C. until use in crosslinking reaction. In another 15 ml glass vial, 0.1 g of hexamethylene diisocyanate (HMDI) is dissolved in dry 10 ml of DMSO. 100 μl of gelatin is mixed with 20 μl of HMDI solution are mixed and immediately mixed in silicone mold cylindrical cavities. The mixture crosslinks in mold cavities in 2-24 hours to form a composite of crosslinked gelatin organic solvent gels. The gel implants are removed from the mold, washed with water to remove the solvent and lyophilized to form crosslinked gelatin implants. The implants form biodegradable hydrogels. In one variation, PLGA (0.1 g, 50:50, molecular weight 10000 to 15000 Daltons, ester endcapped) is mixed with gelatin before crosslinking. The crosslinked organic gel formed had PLGA as an illustrative NBP entrapped in the gel.

### Example 3D

Formation of composite materials comprising crosslinked biodegradable polymer/macromolecules formed by condensation polymerization and non-crosslinked organic solvent soluble biodegradable polymer.

Composite materials wherein crosslinked materials are created by precursors comprising electrophilic and nucleophilic groups wherein the total number of electrophilic and nucleophilic groups in the precursor is greater than or equal to 5.

### Example 3D-1

Composite hydrogels comprising PEG and non-crosslinked biodegradable polymer in an organic solvent.

In a 15 ml glass vial, 1 g of PEG10K4ARM tetramine and 1 g of PLGA molecular weight 50000-60000 Daltons, (ester endcapped), is dissolved in 5 ml of dichloromethane. In a 15 ml glass vial, 1 g of PEG10K4ARM glutarate NHS ester prepared as above is dissolved in 5 ml of dichloromethane. 20 μl of each solution as above and 10 μl of 10 percent triethylamine solution in dichloromethane are mixed in a test tube. The crosslinking is completed at room temperature without loss of solvent. The material is optionally washed twice with 1 ml of dichloromethane for 10 minutes to remove reaction products and unreacted precursors. Solvent is removed by air drying/vacuum drying. The crosslinked PEG based biodegradable hydrogel comprising PLGA composite material is produced. The PLGA end groups are ester endcapped (no electrophilic or electrophilic groups that can react with end groups) to inhibit participation in the crosslinking reaction. The glutarate in the crosslinked polymer and lactate and glycolate ester in PLGA undergo hydrolysis upon implantation in the human or animal body providing biodegradability. The drug such as rifampin can be loaded in a crosslinked network and/or entrapped PLGA by incubation in drug solution and removing the solvent as discussed before. Solvent removal precipitates the PLGA inside the crosslinked material and encapsulates the drug with the precipitated polymer. The permeability of the crosslinked network permits diffusion of rifampin in the PLGA but prevents PLGA from diffusing out of the crosslinked network. The drug can be incorporated before crosslinking occurs provided it does not comprise reactive groups capable of reacting with precursors under effective crosslinking conditions. Using a similar procedure as above, 10 g of PEG10000-glutarate ester (linear bifunctional) and 5 g of PEG10K4ARM tetramine are reacted in presence of ester endcapped PLGA (50:50) molecular weight 10000-15000 g/mol to produce crosslinked gel microparticles.

In a 15 ml glass vial, 1 g of PEG10K4ARM tetramine and 1 g of PLGA molecular weight 50000-60000 Daltons, (ester endcapped), is dissolved in 5 ml of dichloromethane. In a 15 ml glass vial, 1 g of PEG10K4ARM succinate NHS ester prepared as above is dissolved in 5 ml of dichloromethane. 1 ml of each solution is mixed and allowed to react to form a composite polymer network with succinate as degradable blocks. Using a similar procedure as above, 1 g of PEG20K4ARM tetramine (PEG 20K tetramine, 4 arm) is reacted with 0.5 g PEG10K4ARM glutarate-epoxide in presence of PLGA in dichloromethane at room temperature (20 degree C.). The resultant network does not produce side products due to the ring opening reaction of epoxy with the amine group. A crosslinking reaction between isocyanate and alcohol or amine is another example wherein no side products are produced due to crosslinking reactions. PEG20K4ARM tetramine and PEG10K4ARM glutarate-isocyanate is reacted in equimolar quantities in dry tetrahydrofuran (THF) at room temperature in presence of ester endcapped PLGA (50:50) molecular weight 10000-15000 g/mol to produce degradable polyurethane/polyurea.

### Example 3D-2

Composite hydrogels comprising crosslinked PEG based hydrogels and non-crosslinked biodegradable polymer in aqueous environment.

Composite hydrogels comprising crosslinked PEG based hydrogels and non-crosslinked biodegradable thermosensitive polymers.

In a 15 ml glass vial, 1 g of Jeffamine-lactide copolymer (JAL) with hydroxyl end groups, is dissolved in 3 ml of cold PBS solution. To this solution 1 g of PEG10K4ARM tetramine is added and shaken until complete dissolution. In a 15 ml glass vial, 1 g of PEG10K4ARM glutarate NHS ester synthesized as described in previous example or commercially purchased is dissolved in 3 ml of PBS. 100 microliters of each of the above solutions is mixed to form a crosslinked polymer at 0 to 10 degree C. with addition of one drop of triethanolamine solution (10 percent in PBS) as a catalyst. The crosslinked gel has Jeffamine-lactide copolymer (JAL) as water soluble PEG based polylactate copolymer. This copolymer also displays a thermoreversible gel property when subjected to body temperature (37 degree C. at concentration 10-30 percent). The drug can be incorporated before (provided the drug does not have a functional group capable of reacting with amine or NHS ester group) crosslinking as described before or after crosslinking by solvent incubation and diffusion. The crosslinked polymer also provides structural support to the thermoreversible gel of Jeffamine-lactide copolymer (JAL) thus improving its handling during implantation and/or injection. If composite material as above is made in the microsphere or microparticle (size less than 1000 microns) form, then the composite microspheres materials can be injected in the body for local or systemic drug delivery using a standard needle and syringe. In another modification as above, the JAL is substituted with Pluronic F127 (a well-known commercially available thermosensitive polymer) at a concentration of 30 percent to produce Pluronic F127 encapsulated composite hydrogels.

### Example 3D-3

Composite hydrogels comprising crosslinked PEG based hydrogels and liquid carriers.

Composite hydrogels comprising crosslinked PEG based hydrogels and sucrose acetate isobutyrate as liquid carriers.

In a 15 ml glass vial, 1 g of PEG10K4 ARM tetramine and 1 g of sucrose acetate isobutyrate are dissolved in 5 ml of dichloromethane. In a 15 ml glass vial, 1 g of PEG10K4ARM glutarate NHS ester prepared as above is dissolved in 5 ml of dichloromethane. 20 μl of each solution as above and 10 μl of 10 percent triethylamine solution in dichloromethane are mixed. The crosslinking is completed at room temperature without loss of solvent. After crosslinking and gel formation, the solvent is removed by air drying followed by vacuum drying. The resulting crosslinked material is a PEG based biodegradable crosslinked hydrogel in which sucrose acetate isobutyrate is entrapped as a liquid carrier.

If a hydrophobic drug is added during the crosslinking reaction (provided the drug does not take part in the crosslinking reaction), then the drug can be encapsulated in a liquid carrier which can be released in a sustained manner. If composite material as above is made in the microsphere or microparticle (size less than 1000 microns) form, then the composite microspheres materials can be injected into the body for local or systemic drug delivery using a standard needle and syringe.

In another modification as above, sucrose acetate isobutyrate is replaced with polycaprolactone molecular weight 2000 Daltons as a model liquid biodegradable polymer.

### Example 3D-4

Composite hydrogels prepared from linear functional polymers with functional groups that are crosslinked using a crosslinker.

Composite hydrogels prepared from gelatin as an exemplary linear functional polymer with functional groups that are crosslinked using hexamethylene diisocyanate.

Encapsulation of soluble synthetic biodegradable polymer in crosslinked gelatin via organic solvent gel.

In a 15 ml glass vial, 1 g of gelatin and 1 g of PLGA molecular weight 10000-15000 Daltons, ester endcapped, are mixed with 20 ml of dry DMSO. The mixture is warmed to 60 degree until dissolution and held at 45 degree C. until use in crosslinking reaction. In another 15 ml glass vial, 0.1 g of hexamethylene diisocyanate (HMDI) is dissolved in dry 10 ml of DMSO. 100 μl of gelatin and PLGA solutions at 45 degree is mixed with 20 μl of HMDI solution for crosslinking gelatin with HDMI. The mixture crosslinks in 2-12 hours to form a composite of crosslinked gelatin and PLGA. The composite material is washed with warm water followed by washing with dichloromethane and then dried. The endcapping of PLGA groups prevents PLGA from reacting with diisocyanate and thus stays as PLGA material in the composite material. In a 5 ml glass vial, 50 mg PLGA (50:50, molecular weight 10000-15000 Daltons) is dissolved in 0.25 ml DMSO to make a clear solution. In another 5 ml glass vial, 60 mg Gelatin and 1.0 ml of DMSO are mixed and warmed to 60 degree C. until a clear solution is obtained. 50 microliters of PLGA solution and 50 microliters of gelatin solution are transferred into another 5 ml glass vial and then 5 μl hexamethylene diisocyanate is added as a crosslinker. The mixture forms a soft organic solvent gel in 3-12 hours. The composite organic solvent gel thus produced has crosslinked gelatin and PLGA as non-crosslinked biodegradable material entrapped in the crosslinked gel. In this case, the reactive groups are on the side chain of gelatin and hexamethylene diisocyanate is used as a crosslinker.

For additional examples of crosslinked polymers made via condensation polymerization, please refer to U.S. Pat. Nos. 9498557, 7009034, 6201065 and references cited therein, cited herein for reference only.

### EXAMPLE 4

Preparation of composite microparticles by casting from molds.

Use of elastomeric mold to form microparticles of desired shape and size.

A 2 cm by 2 cm silicone rubber mold with 20 circular cavities (350 microns diameter and 500 microns length) is prepared separately from a stainless steel reverse mold. Silicone rubber is cast into this reverse stainless steel mold made using a CNC machine to make a mold of desired cavities. 50.0 mg of PEG35KUA macromonomer and 100 mg of polylactide-co-polyglycolide (PLGA, 50:50 copolymer, molecular weight 10000 to 15000 Daltons) and 0.5 ml of dichloromethane are mixed in 5 ml glass vial. After complete dissolution, 10 microliters of initiator stock solution (ISS) is added and vortexed. The precursor solution is then filled in the mold and is then exposed to long UV light for 5 minutes. The solution converts into a soft gel in the mold. The solvent is removed by air drying followed by vacuum drying. The microcylinders are removed from the mold by stretching (50-200 percent) and shaking the mold. The recovered cylindrical particles and stored in the freezer under nitrogen until use.

In another modification of the above example, the DMSO is used in place of dichloromethane and the mold filled with the is centrifuged for uniform cavity filling and removing dissolved gases prior to light exposure. The crosslinked composite microcylinders are removed by incubation in water and mechanical stretching. Yet in another example, a 500 microns thick stainless steel plate is laser drilled to create 300 microns diameter holes in an array format (30 by 30 holes, 2 mm pitch). This stainless steel plate is used for casting microcylinders with 300 microns diameter and 500 microns height. The molded gels are removed using pressurized jet air stream or using a blunt syringe needle.

A microparticle making kit containing silicone rubber mold, casting gel, razor blades and instructions on making beads is purchased from AKINA, INC., Lafayette, Ind., USA. The silicone rubber (polydimethylsiloxane) has 20 micron size cylindrical columns protruding from its surface. 20 g of proprietary hydrogel polymer provided by the supplier is dissolved in 300 g of distilled water and 450 ml of ethanol in a 1000 ml bottle. The hydrogel polymer solution is placed on top of silicone mold to produce a negative imprinted image having 20 micron diameter and 20 micron height size cylindrical cavities. 1 g of macromonomer PEG35KUA, 20 mg Irgacure 2959, 50 microliters of n-vinylpyrrolidone and 9 g of dichloromethane are mixed. 1 ml of this mixture is added on the hydrogel mold and excess solution is wiped off. The mold cavities are filled with a precursor solution. The solution in the cavity is exposed to Long UV light (Black-Ray UV lamp, 360 nm light, 10000 mW/cm2 intensity) for 5 minutes. The mold along with its crosslinked PEG gels particles added to 100 ml of warm water to dissolve the mold. The crosslinked hydrogel pieces are separated by centrifugation or filtration process and then lyophilized. Silicone mold is available in 6, 10, 20 and 50 micron sizes from AKINA, Inc and can be used to prepare the same size particles as described above. Other microparticles of different shapes and sizes may be custom made using similar methods as described herein.

In another variation of this embodiment, a one inch section of a 300-800 micron silicone tubing or glass capillary tubing is cut. The tube is filled without any air bubbles with UV light based photo crosslinkable composition as described above, using a syringe and long needle. The filled tube is then exposed to long UV light for 2-5 minutes to crosslink the precursor in the tube. 0.5 to 1 mm sections of the tube along with its contents are cut and are added to 100 ml of PBS solution and vortexed. The crosslinked hydrogel cylinders are separated to produce hydrogel cylinders. The soft plunger is also used in some cases to eject the hydrogel from the tube. In another variation of this embodiment, the cast hydrogel is first removed from the tube by applying nitrogen gas pressure from one side and collecting the hydrogel cast micro-rods from the other side. The crosslinked gel is then dried and cut using a microtome machine to the desired length (5 to 500 microns). In another variation, 1 ml of 20 percent bovine albumin and 1 ml of PEG10K4ARM glutamate NHS crosslinker (50 mg/ml in PBS) are mixed and the solution is added in the tube before gelling or crosslinking. The crosslinked albumin composition is separated and then cut to the desired length (500 microns). In another variation, the crosslinked composition is then stretched up to 50 to 200 percent of its length and then dried/lyophilized. The dried/lyophilized composition is removed from the tube and is then cut into the desired size/length for future use.

### EXAMPLE 5

Preparation of composite biodegradable microspheres

### Example 5-1-1

Composite microspheres from crosslinked biodegradable hydrogel and non-crosslinked organic solvent soluble biodegradable polymer by free radical polymerization.

Use of microfluidic chips for making drug encapsulated composite microparticles.

Droplet generating microfluidic chips and related accessories are obtained from microfluidic ChipShop GmbH, Stockholmer Deutschl, Germany. Accessories like Male Mini Luer fluid connectors, the green version (catalog number 09-0541-0331-09); Male Mini Luer fluid connectors, the opaque version (catalog number 09-0538-0331-09); Male Mini Luer plugs, the red version (catalog number 09-0551-0334-09); silicone tube, ID: 0.5 mm (catalog number 09-0802-0000-00); PTFE tube, ID: 0.5 mm (catalog number 09-0803-0000-00), droplet generator chip, (catalog number 13-1002-0162-03, polycarbonate material); orange frame (15-4001-0000-12). T-piece for tubing and the like are obtained prior to the experiment. A droplet generator chip is inserted in the handling orange frame. The central entrance for the aqueous phase (macromonomer solution along with visible light initiating components like eosin/triethanolamine photoinitiator as described previously) is connected via an opaque Mini Luer connector, silicone sleeve, a PTFE tube, and 50 ml glass syringe on the syringe pump. The mineral oil is connected to the chip via Mini Luer connectors, silicone sleeves, PTFE tube, the splitting T-piece, the pump tube and 100 ml syringe on the syringe pump. Before connection, the mineral oil and aqueous macromonomer solution with photoinitiator (eosin and triethanolamine) are primed to remove any air bubbles. The macromonomer solution composition must gel within 3-300 second exposure of appropriate light (green light wavelength 512 nm and high intensity). The mineral oil is dispensed first and then an aqueous solution is introduced. The flow rates of mineral oil and monomer solution are varied until a continuous stream of red precursor solution droplets of the desired size are formed in the receiving channel. If needed the droplets can be observed via a high speed video camera and the size of the droplet captured by the camera is used to adjust the flow rate and other variables to obtain the desired droplet size. The droplets must travel about 3-300 seconds prior to reaching the receiving chamber. The red solution droplets are irradiated with green laser light or white flood light to polymerize the droplets while traveling to the collection chamber (total light exposure time about 1 minute). Alternatively, the droplets are collected in a dish containing mineral oil and exposed to the green light for effective polymerization and crosslinking. The polymerized droplets are collected, isolated and stored until further use. In another modification of the above embodiment, live cells are added to the monomer solution and the polymerized droplets with live cells are collected in the tissue culture medium. The entire experiment with cells/drugs is carried out in a sterile manner. In another modification, the droplets with drugs or cells are collected without light exposure in liquid nitrogen where they get frozen immediately. The liquid nitrogen is evaporated and the frozen droplets are exposed to green light (around −10 to zero degree C. before melting) and the crosslinked droplets are stored for future use.

### Example 5-1-2

Preparation of composite microspheres using in house fabricated silicone microfluidic chips.

500 mg PEG 10 KUA macromonomer, 1.0 g PLGA (1:1, molecular weight 10000-15000 g/mole), 100 mg Bupivacaine base and 5.0 ml dichloromethane were mixed in 20 ml glass vial. 100 microliter initiator solution (300 mg 2-hydroxy-4′-(2-hydroxyethoxy-2-methylpropiophenone dissolved in 3.0 ml vinyl pyrrolidinone) was added to macromonomer solution. The macromonomer solution was filled in a 5 ml syringe which was connected to a 26 G syringe needle and housed in a syringe pump. The 26 G syringe needle tip was inserted in a molded silicone microfluidic chip with a single channel having a diameter of 0.2 mm. One percent 20 ml Polyvinyl alcohol (PVA) solution is filled in a 20 ml syringe and is loaded in another syringe pump. The syringe was connected to a microfluidic chip via silicone tube and 26G needle. An SS capillary with 0.2 mm id was connected to the other end of the fluid path to receive the droplets form and to collect in a Petri dish comprising PVA solution. The apparatus was flushed with a PVA solution and the flow rate of PVA solution was maintained at 50 ml per hour. Macromonomer solution is injected into the fluid chip at 90 degree angle at 5 ml per hour. The macromonomer solution droplets formed were collected in a

Petri dish containing a PVA solution. The Petri dish was moved manually while receiving the droplets so that each droplet will not come in contact with existing collected droplets. The Petri dish was also exposed to a UV lamp (Wenzhou Aurora Technology Company Ltd., Wenzhou, China, model UV-300, 365 nm light and visible white light) with minimum intensity 26,500 μW/cm, 3 inch distance from the Petri dish surface) while collecting droplets. The polymerized macromonomer solution droplets did not agglomerate as a result of photopolymerization and crosslinking and gave substantial uniform size droplets. The flow rates of both solution and microfluidic channel diameter were varied to obtain transparent microspheres 600 to 800 micron size diameter polymerized organic solvent gel microspheres. The polymerized microspheres obtained have PLGA dissolved in dichloromethane. The collected microspheres were kept at room temperature to remove the solvent. Upon airdrying, the PLGA in the solvent precipitates inside the microsphere making the microspheres opaque. The PLGA remains entrapped in the crosslinked macromonomer. Using a similar procedure as above, crosslinked composite microspheres comprising PEG35KUA (10 percent) and PLGA (20 percent, 1:1, molecular weight 10000-15000 g/mole) in dichloromethane were made. In another experiment, PEG1OKUA macromonomer was replaced with PEG1OKL5A as biodegradable macromonomer to produce composite microspheres comprising biodegradable crosslinked PEG1OKL5A polymer and PLGA.

In another alternative embodiment, an in house fabricated silicone rubber microfluidic chip containing 0.29 mm fluid mobile phase dispensing path is used to flow one percent polyvinyl alcohol solution as a mobile phase. This path is connected to the dichloromethane solution comprising PLGA and PEG35KUA with long UV light photoinitiator solution(ISS) as above via a 26-gauge stainless steel needle connected at 90 degrees angle to the mobile phase path. Polyvinyl alcohol PVA solution was filled in a 50 ml syringe which is connected to the syringe pump. The PVA solution was dispensed at 50 ml per hour through the fluid chip path. The PLGA and PEG 35KUA with long UV photoinitiator solution as above was filled in the 10 ml syringe and is dispensed via a 26 gauge needle in the silicone chip path at 5 ml per hour. PLGA monomer droplets with an approximate size of 150 microns are formed which are collected in a petri dish exposed to long UV light. The collecting dish was moved manually so that each droplet coming out of the fluid chip is collected on a new surface. The exposed droplets undergo immediate effective polymerization (within 60 seconds) and crosslinking which prevents the fusion of liquid droplets. Control droplets/microspheres with PEG35KUA and without PLGA as above in the dichloromethane were also prepared. The control microspheres containing crosslinked PEG 35KUA but no PLGA were substantially transparent in nature to the human eye while microspheres with PLGA were substantially opaque in nature after partial evaporation of dichloromethane. The composite microspheres prepared had uniform size distribution. In another modification of the above example, the monomer was dissolved in DMSO instead of dichloromethane and the mobile phase was hexane or mineral oil-hexane mixture (80:20) and the droplets are collected in liquid nitrogen. The liquid nitrogen was evaporated and the mixture was warmed and exposed in a frozen state to long UV light to effectively crosslink the macromonomer. Care was taken to expose all microspheres before attaining ambient temperature. The crosslinked microspheres had PLGA entrapped in the PEG 35KUA crosslinked gel. In some embodiments, frozen macromonomer solution droplets thus prepared are stored in frozen condition without crosslinking. These droplets are then used to form multilayered objects as described in this invention.

In another modification of the above embodiment, standard plain PLGA microspheres with or without drugs were made using the same experimental setup as described above. The macromonomer solution with or without the drug in the above setup is replaced with a ten percent PLGA solution in dichloromethane with or without rifampin as a model drug. PVA solution was used as a mobile phase. The microspheres thus formed were collected in PVA solution (no need for UV light exposure), stirred overnight to remove solvent and then centrifuged and dried in a vacuum oven for 24 hours.

In another embodiment, microfluidic chips from Micronit Microfluidics Enschede, Netherlands are used. The Focused flow droplet generators (both small and large droplet generators, hydrophobic coating coated as well as uncoated), Fluidic Connect PRO Chip Holder with 4515 Inserts, Teflon connection kit, inline filters and tubing and connector accessory for syringe pumps are used to generate microspheres or microdroplets.

Many commercial vendors such as Dolomite Microfluidics, Royston, United Kingdom or Micronit Microfluidics, Enschede, Netherlands sell microfluidic chip kits specifically designed for preparing microdroplets and/or drug/encapsulated microspheres. Such kits can be purchased and used to make microspheres for a given drug/cell delivery application. Telos® System or Telos® Starter Kit from Dolomite Microfluidics enables to make drug encapsulated microspheres using microfluidic chip systems.

### EXAMPLE 6

### Example 6A

Preparation of microneedle drug delivery array device made using composite material.

Use of organic solvents and biodegradable macromonomers to make composite microneedle array. Silicone base MPatch™ Microneedle array template mold is procured from Micropoint Technologies Pte Ltd. (Singapore). The mold had the following characteristics: 20 mm diameter and 4 mm in height. 10 by 10 microneedle array holes, 700 microns cavity height (square pyramid shaped cavities) with 200 by 200 microns base, 500 microns pitch, the distance between each needle is 500 microns (center to center). 50.0 mg of PEG35KUA macromonomer and 100 mg of polylactide-co-polyglycolide (PLGA, 50:50) copolymer (molecular weight between 10000 to 15000 Daltons) are mixed with 0.5 ml of dry warm DMSO until complete dissolution. 10 microliter of 2-hydroxy-4′-(2-hydroxyethoxy-2-methylpropiophenone) stock solution (ISS) was added to the macromonomer solution. The mold cavities were filled with the macromonomer solution, centrifuged for uniform filling and then exposed to long UV light (360 nm). The solvent was removed and the formed needles are attached to pressure sensitive adhesive tape (distal end with needle base) and lifted from the mold. The composite microneedle array thus formed had a sharp needle on the distal end for tissue penetration and the proximal side is attached to the adhesive tape. The array comprised biodegradable crosslinked material and PLGA polymer entrapped in the crosslinked material. The array device can be used as a biodegradable microneedle array device for skin/tissue implantation. The PLGA and crosslinked network either alone or in combination can provide sustained drug delivery properties. The high molecular weight nature of crosslinked material along with PLGA as a reinforcing polymer provides mechanical strength to the array. This method eliminates the use of injection molding and other methods that require high temperature processing/exposure in array fabrication. The needles may be loaded with drug/s and/or visualization agent/s prior to polymerization/crosslinking or after the array is made as described in the earlier section. The visualization agent such as colored/fluorescent compound in the array, preferably at the base of the array can assist in visualization of the array during implantation. In another modification of the above embodiment, the precursor solution in DMSO is degassed and frozen first and then exposed to light to crosslink in the frozen state.

In another embodiment, 100 mg of PEG1OKUA or PEG35KLA and 50 mg of PLGA (50:50, 10000-15000 Daltons) and 5 mg of bupivacaine base is dissolved in 0.5 ml of dichloromethane and 10 μl of initiator solution (50 mg 2,2-Dimethoxy-2-phenyl acetophenone in 0.5 ml of Vinyl pyrrolidone) is added. The solution is filled in the Mpatch™ Microneedle array template mold and centrifuged for 5 minutes, excess solution is wiped off from the mold surface and then exposed to long UV light for 5 minutes. The solution is converted into the gel with PLGA dissolved in the solvent. The solvent is removed and the array is removed from the mold. In another embodiment, a control microneedle array is made using the same method as above except without PLGA. In another modification of the above embodiment, the mold cavity is filled with dichloromethane solution comprising macromonomer, PLGA, bupivacaine base and photoinitiator and the same solvent is allowed to evaporate creating a space in the mold as a result of evaporation. The newly created space by the evaporation is then filled with the same precursor formulation without bupivacaine base but with 10 percent magnesium carbonate suspended in the solution as a visualization agent. The composition is then exposed to light before substantial mixing of two solutions to polymerize and crosslink the composition. The microneedle array thus formed has a tip and some portion of the needle body comprises a drug and the base portion of the needle comprises a visualization agent (magnesium carbonate). If needed, the solutions may be frozen and then polymerized as disclosed in this invention. The base layer with visualization agent thickness in the needle may be controlled by the amount of solvent evaporation in the mold. The base layer with the visualization agent may occupy 5 to 50 percent of needle volume, preferably 10 to 40 percent of the volume. Other methods of multilayered particle formation discussed separately may be used.

A microneedle array is also made using precursors that form a crosslinked network using condensation/step growth copolymerization as discussed before. The reaction may be done in aqueous solutions or in organic solvent depending on the drug to be delivered. Silicone base MPatch™ Microneedle array template mold is used to make an illustrative biodegradable hydrogel array comprising PEG. 1 g of Trilysine acetate salt is dissolved in 9 ml of PBS in a 15 ml glass vial. In another 15 ml glass vial, 1 g of PEG10K4ARM glutarate NHS ester prepared as above is dissolved in 9 ml of PBS. Both solutions are mixed in equimolar quantities and gel time is adjusted by controlling the pH of the final mixture. The final pH upon mixing is varied between 6 to 7.4 to obtain a gel time between 2 to 5 minutes which gives sufficient work up time with the composition prior to crosslinking. The composition is filled in the MPatchTM mold cavities before gelation. After effective crosslinking and polymerization of precursors, the condensation/step growth polymerization crosslinked based hydrogel array is then removed from the mold and dried/lyophilized. If desired the drug/visualization agent is added in the precursor prior to crosslinking. The drug used may be in the microencapsulated form such as PLGA encapsulated microspheres. Sugars, NBP like HPMA, hyaluronic acid, inorganic fillers like magnesium carbonate, calcium sulfate, sodium chloride and the like may be also be added at a concentration of 0.5 percent to 90 percent relative to total weight to improve mechanical properties of the hydrogel array needles.

### Example 6B

Preparation of microneedle drug delivery array using crosslinked hydrogel with non-crosslinked biodegradable polymer or filler such as sodium chloride or sugar. Device made using organic solvent gel. 100 mg of PEG35KLA is dissolved in 0.5 ml of ethyl acetate and 2 μl of initiator solution ISS is added. To this solution 200 mg of sodium chloride (size less than 100 microns) as a model water soluble salt and 10 mg bupivacaine base is added as a model drug. The suspension is degassed using multiple freeze-thaw cycles in vacuum and liquid nitrogen as cooling medium. The suspension is then transferred to cavities of the Mpatch™ Microneedle array template mold. The suspension in the mold is exposed to long UV light for 5 minutes for effective polymerization and crosslinking. The solvent is removed by drying under air/vacuum and the crosslinked microneedles array is removed from the mold. The array needle comprises crosslinked biodegradable PEG35KLA polymer and sodium chloride as an inorganic water soluble biocompatible filler and drug. Upon insertion of array needles in the skin tissue, the filler and drug quickly disperses in the skin tissue in the producing rapid drug response. Other organic and inorganic biocompatible inorganic salts and organic compounds can be substituted with sodium chloride. In another modification, the solvent in the suspension with drug is substantially or completely removed and dry precursor is mixed with magnesium stearate as a binder (final concentration one percent relative to total mass). The mixture is added to a stainless steel microneedle array mold (similar to Mpatch™ Microneedle array template) and pressed using a hydraulic press at a pressure of 10 kg. The precursor micro-tablet is removed from mold and then exposed to long UV light to effectively crosslink the precursor in solid state or dry state. The crosslinked needles are then used for fabrication microneedle array to make a microneedle array drug delivery device.

### Example 6C

Use of aqueous solutions to make microneedle arrays.

Use off solutions comprising water soluble salts or fillers or non-crosslinked water soluble polymers.

In a 10 ml glass vial, 1 g of PEG35KLA, 2 g of maltose and 10 mg bupivacaine hydrochloride as model drug are dissolved in 5 ml PBS buffer (pH 7.4) and 15 μl of initiator stock solution ISS is added. The solution is degassed using freeze-thaw cycle in vacuum and liquid nitrogen as cooling medium. The solution is transferred in cavities of the Mpatch™ Microneedle array template mold. The solution in the mold is exposed to long UV light for effective polymerization and crosslinking. The crosslinked microneedles array are dried/lyophilized and then removed from the mold using a pressure sensitive adhesive tape. The array needle comprises crosslinked biodegradable PEG35KLA polymer and maltose as water soluble biocompatible filler and the water soluble drug. Upon skin insertion, the filler maltose and drug bupivacaine hydrochloride disperses in the skin tissue producing rapid drug response. In another modification of the same above example, maltose is replaced with a 0.5 g carboxymethyl cellulose as a non-crosslinked water soluble polymeric additive. The polymeric additive serves as a rapidly dissolving/swelling component and a mechanical reinforcement agent. The crosslinked PEG35KLA along with water soluble high molecular weight carboxymethyl cellulose additive improves mechanical properties of the needle.

### Example 6D

Microneedle array comprising drug encapsulated microspheres and crosslinked biodegradable hydrogels.

In a 10 ml glass vial, 1 g of PEG35KLA is dissolved in a 5 ml PBS buffer (pH 7.4) and 15 μl of initiator stock solution ISS is added. 2 g of bupivacaine encapsulated PLGA microspheres (10 percent drug loading, w/v PLGA 50:50, molecular weight 10000-15000 Daltons, size 10-50 microns) are added to PEG35KLA solution. The solution is degassed and transferred in cavities of the Mpatch™ Microneedle array template mold. The solution in the mold is exposed to long UV light for effective polymerization and crosslinking. The crosslinked microneedles array are dried/lyophilized and then removed from the mold using a pressure sensitive adhesive tape. The array needle comprises crosslinked biodegradable PEG35KLA polymer and PLGA encapsulated microspheres comprising bupivacaine as a model drug. Upon skin insertion of an array, bupivacaine is released from the encapsulated microspheres in a controlled manner.

### Example 6E

Use of aqueous frozen monomer solutions to make microneedle array devices. Solid state polymerization to make microneedle array devices.

In a 10 ml glass vial, 1 g of gelatin methacrylate, 2 g of maltose and 10 mg bupivacaine hydrochloride as model drugs are dissolved in a 10 ml PBS buffer (pH 7.4). The solution is added with 30 microliters of ISS initiator stock solution. Another gelatin methacrylate solution with photoinitiator is prepared the same as above but without the drug and maltose. A degassed solution with the drug is added in the cavities Mpatch™ microneedle array template and frozen to −10 degree C. A cold degassed solution without drug is then added on top of first frozen microneedle to make about 500 micron height layer on top of the frozen microneedles and then immediately exposed long UV light for 10 minutes. The solution and frozen microneedle array are effectively polymerized in frozen state-solution state. The device is lyophilized or dried and then removed from the mold. The polymerized microneedle device is similar to shown in FIG. 7G with two layers wherein a 500 micron thick crosslinked gelatin methacrylate layer without the drug and filler serves as a backing layer (FIG. 7, 713) to the drug comprising microneedle array (FIG. 7, 714). The attached crosslinked gelatin layer to the microneedles improves handling of the array during insertion. A pressure sensitive tape is then attached to the layer 714 and is used as a microneedle device. The device with its needle is inserted in the skin tissue and the maltose and drug are dispersed in the tissue producing therapeutic effects of the drug. Crosslinked gelatin being biodegradable can stay in the skin tissue which can be removed by enzymatic biodegradation process from the tissue if implanted. The device also can be removed from the skin tissue after 10 minutes to 24 hours when most of the drug is dispersed in the tissue. In another variation of above example, the cotton cheesecloth (#50 grade) is cut to a desired size and then applied on top of the mold (on top of frozen gelatin methacrylate array solution). Gelatin methacrylate solution is applied on top of cheesecloth to create a 500 micron thick layer and exposed to light and polymerized. The polymerized gelatin array with gelatin back layer (714) is obtained. The gelatin layer (714) in this case is reinforced with cotton fiber mesh. The reinforcement of the gelatin backing layer improves the mechanical properties of the device.

Alternatively, 3D printing technologies such as Continuous Liquid Interface Production (CLIP) technology known in the 3D printing art to print microneedle arrays using macromonomer compositions (biodegradable or biostable) described in this invention. The compositions can be printed in organic solvents or aqueous based solutions or as neat liquids or combination thereof as described in this invention. Microneedle arrays made using composite biodegradable materials described in this invention (combination of crosslinked PEG based crosslinked polymers and non-crosslinked polymers like PEG) are especially suitable for 3D printing technology.

### EXAMPLE 7

Preparation of coated medical devices using coated compositions described in this invention.

### Example 7A

Coated bioprosthetic tissue or surgical patch

2 cm by 2 cm bovine pericardial tissue or porcine submucosa is decellularized by incubation in a surfactant solution for 24 h. The decellularized tissue is then lyophilized to remove unbound water from the matrix. 500.0 mg of PEG20KUA macromonomer is dissolved in 5 ml warm THF followed by the addition of 100 microliters of initiator stock solution (ISS). 50 microliters of THF solution is exposed to long UV light. The solution forms a soft gel within 300 seconds. 3 ml of macromonomer solution as above is mixed with 600 mg of polylactide-co-polyglycolide (PLGA, 50:50) copolymer,(molecular weight between 10000 to 15000 Daltons) and 120 mg of bupivacaine base (20 percent relative to PLGA polymer weight, an illustrative drug) until complete dissolution. The lyophilized tissue is then dip coated in the drug solution and is then immediately irradiated with long UV light to initiate polymerization of PEG35KUA in THF. The polymerization and crosslinking forms a composite polymer coating on the tissue with entrapped PLGA. THF is removed by air drying and vacuum drying which precipitates the PLGA in the crosslinked polymer and entraps the drug in the PLGA coating and crosslinked polymer. The flexible crosslinked network enables good adhesion and elasticity to the coating while PLGA with encapsulated bupivacaine provides sustained drug release. Using a similar procedure as above, a precursor composition that polymerizes by condensation polymerization is used to make a composite material. In one illustrative embodiment, a coating that is made by condensation polymerization (Example 3D) comprising sucrose acetate as a liquid carrier is made along with bupivacaine base or hydrochloride as a local anesthetic agent. The drug/anesthetic coated biodegradable device is used as an adjunct to close surgical or non-surgical wounds (typical wound length greater than 1 cm). The coated device is inserted in the wound. The area of the device covers (30 percent to 300 percent, preferably 100 percent of the wound area) prior to closing the wound using surgical sutures. The wound closing sutures are threaded through the coated tissue device so that it remains in the wound site without substantial movement from the wound site and provide local drug delivery in the wound area. The device releases the drug for 1-10 days, preferably 2-5 days locally depending on the coating formulation used. Entire device is saturable and biodegradable and does not need to be removed after implantation. Local anesthetic provides local post-surgical pain relief until substantial wound healing. In another embodiment, local anesthetic in the coating in the device is replaced with one or two antibiotic or antiseptic drugs for local infection control and wound healing.

### Example 7B

Coated suture and microparticles prepared from them.

Plain twisted gut suture 4.0 size (150-200 microns diameter) is obtained from local medical suppliers. 500.0 mg of PEG20KUA, 10 mg of Irgacure 2959, 1000 mg of PLGA and 50 mg of bupivacaine base are mixed in 5 ml of THF until a homogeneous solution is obtained. About 30 cm size of suture thread is cut and is dip coated and then exposed to long UV light for 5 minutes while rotating. The solvent is removed by air drying and the process is repeated until a thickness of about 50 micron coating is obtained. The coated and uncoated sutures are evaluated for controlled release of bupivacaine release from the suture thread. The bupivacaine concentration is reported as mg of drug released per centimeter of suture thread.

In another modification as above, the suture is first covalently linked with fluorescein using EDC as a catalyst. The resultant fluorescent suture is then coated as described above. The coated fiber is then sliced at a 150 microns interval perpendicular to the fiber axis using a microtome machine. The cut particles have a diameter of 200-250 microns (200 microns thickness from fiber and 50 microns from the coating) and a height around 150 microns (cut length) to produce microcylinders. The microcylinders have fiber cores at the center along the axis that are fluorescent due to chemical modification as described above and composite material coating for controlled drug delivery application.

### Example 7C

Coated PTFE vascular graft

Expanded PTFE vascular grafts from CR bard (product code 20S06, 6 mm diameter x 20 cm length) are used. A two cm section of the graft is cut and spray coated with THF macromonomer solution with PLGA and bupivacaine base as above and then photopolymerized immediately with 360 nm light. Care is taken to ensure that the coating composition does not reach the luminal side of the graft and the coating is applied only on the outer surface.

### Example 7D

Coated expandable device (stent)

Bard E-Luminexx stent (product code ZBM06040, 6 mm diameter and 40 mm length) is spray coated with THF solution as above with macromonomer, photoinitiator, PLGA and bupivacaine base. After polymerization and crosslinking with 360 nm light exposure, the solvent is removed by air drying and then under vacuum. The drug entrapped in PLGA is released in a sustained manner over a period of time.

### EXAMPLE 8

Controlled drug delivery from composite microparticles comprising thermosensitive or pH sensitive polymers as a carrier.

### Example 8A

Controlled drug delivery from composite microparticles comprising Jeffamine lactide is used as a non-crosslinked organic solvent soluble illustrative biodegradable thermosensitive polymer.

Infusion of Jeffamine lactide and rifampin inside the microspheres via solvent diffusion.

Microcylinders loaded with Rifampin and Jeffamine Lactide as drug carriers.

100 mg of PEG1OKUA macromonomer was dissolved in dichloromethane to make a 10 percent solution. A 20 microliter with Initiator stock solution was added to the macromonomer solution and the mixture was applied on silicon mold with cylindrical cavities. The excess solution was wiped off and the solution was exposed to long UV light for 5 minutes. The gelled samples inside the mold cavities were taken out from the mold, washed with dichloromethane and dried in air and finally in a vacuum. The dried microcylinders were used for drug loading experiments. 310 mg of Jeffamine lactide solution was dissolved in 1.5 ml dichloromethane. One ml of this solution is added with 5 mg of rifampin drug (Rifampin and Jeffamine lactide solution, sample solution). Rifampin 5 mg was dissolved in 1 ml dichloromethane (Rifampin solution, control 1 solution). 40 mg of dry PEG1OKUA crosslinked microcylinders prepared as above were incubated in 0.5 ml of sample solution for 30 minutes. The incubated particles were dried to remove solvent and then transferred to the dialysis bag and incubated in 10 ml PBS for 1 minute to wash loosely bound rifampin. The sample particles thus prepared had Rifampin in the microcylinders along with Jeffamine lactide as a carrier and PEG1OK crosslinked as a matrix. Similarly, 40 mg dry PEG1OK crosslinked microcylinders were incubated and loaded with rifampin by incubating in control 1 solution. The control 1 particles thus prepared had Rifampin but no Jeffamine lactide. 40 mg of dry particles were incubated in dichloromethane solution for 30 minutes and dried. These were used as control samples with no rifampin and no Jeffamine lactide. All the samples were then incubated in a fresh 10 ml PBS solution at 37 degree C. At each time point, all PBS solutions were removed and fresh 10 ml PBS was added. The rifampin concentration was monitored for all three samples for 120 hours at several time points. The concentration of Rifampin was monitored by measuring absorbance at 474 nm using UV-VIS spectrophotometer. The experiment was done in duplicate. Average and standard deviation was reported for each time point.

### Example 8B

Controlled drug delivery from composite microparticles comprising liquid carriers.

Controlled drug delivery from composite microparticles comprising vitamin E as illustrative biodegradable liquid carriers.

Infusion of Vitamin E and rifampin inside the microparticles by solvent diffusion.

Release from composite microparticles comprising vitamin E as model liquid carrier.

3 mg rifampin and 242 mg of vitamin E were dissolved in 0.6 ml tetrahydrofuran. 20 mg PEG1OK microcylinders were soaked in 0.3 ml Rifampin and Vitamin E solution for 5 minutes. The solvent was removed by air drying followed by vacuum drying. The samples were loaded in a dialysis bag and rifampin release was monitored for 5 days as mentioned previously.

### EXAMPLE 9

### Example 9A

Controlled drug delivery from composite microparticles.

Controlled drug delivery from composite microspheres comprising PLGA as non-crosslinked organic solvent soluble illustrative biodegradable polymer.

PLGA was entrapped during polymerization and crosslinking.

1.003 g PEG1OK acrylate macromonomer was dissolved in 10.0 ml dichloromethane. To this solution, 200 microliter initiator solution was added (300 mg 2-hydroxy-4′-(2-hydroxyethoxy-2-methylpropiophenone in 3.0 ml vinyl pyrrolidinone). A 4 ml macromonomer solution was filled into a 5 ml syringe and it was then housed in a syringe pump. The syringe was connected to a microfluidic chip via fluorinated polymer tubing. 20 ml of 1 percent polyvinyl alcohol (PVA) solution was used as a mobile phase and housed into another syringe pump. Both solutions were infused (50 ml per hour for PVA solution and 5 ml per hour for PEG1OK acrylate solution) via a silicone microfluidic chip to create microdroplets which were then collected and exposed to UV light to crosslink the droplets. The solvent is removed by air drying followed by incubation in distilled water, then 3 times in methanol and finally vacuum dried. The product formed is referred to as PEG1OK microspheres. Using a similar procedure as above, composite microspheres comprising PLGA were prepared. Briefly, 501 mg of PEG1OK Acrylate macromonomer, 1.001 g of PLGA (50:50, molecular weight 45000 to 55000 g/mole were dissolved in 5 ml dichloromethane and 100 microliter of initiator solution (300 mg 2-hydroxy-4′-(2-hydroxyethoxy-2-methylpropiophenone in 3.0 ml vinyl pyrrolidone was added). The PLGA solution and PVA solutions were infused via a silicone microfluidic chip and the droplets were collected and photocrosslinked. The collected composite droplets were washed and dried and stored until use (referred as PEG1OK-PLGA microspheres).

20 mg rifampin was dissolved in 4 ml tetrahydrofuran. 20 mg of dry PEG1OK-PLGA microspheres were incubated in rifampin solution for 5 minutes. The solvent was removed by air drying followed by vacuum drying. The samples were loaded in a dialysis bag and rifampin release was monitored for 5 days as mentioned previously.

### Example 9B

Controlled drug delivery from microparticles wherein drug is crystalized/precipitated in situ inside the microparticle.

Paclitaxel loaded hydrogel microparticles or microspheres

2 mg paclitaxel is dissolved in 4 ml dimethyl sulfoxide. 20 mg of dry PEG1OKUA microspheres prepared as above microspheres are incubated in paclitaxel solution for 60 minutes. The solvent is removed by vacuum drying or by incubating in 100 ml water for 30 minutes. Paclitaxel has extremely low solubility in water (less than 1 percent). The incubation in water or vacuum drying removes the solvent and precipitates/crystalizes the drug inside the PEG1OKUA microspheres. The loose drug on the surface of the microspheres is removed by washing with water. Finally, the PEG1OKUA microspheres with paclitaxel are lyophilized. The lyophilized microspheres are sterilized using ethylene oxide and then suspended in 1 ml PBS. The suspension can be injected into the body for controlled drug delivery. The precipitated drug crystals inside the microsphere slowly dissolve providing controlled drug delivery.

Using a similar procedure as above, bupivacaine free base and dexamethasone (both water insoluble drugs, solubility less than 2 percent) are infused and then precipitated inside the microspheres. In another variation of the above method, biodegradable microspheres made from gelatin or microspheres made by condensation polymerizations of PEG derivatives are used as biodegradable microspheres instead of PEG1OKUA microspheres.

The drugs described above may also be added prior to polymerization and/or crosslinking, especially in organic solvents as discussed in the previous example and then the solvent is removed to precipitate or crystallize the drug.

### EXAMPLE 10

Solid State Polymerization of macromonomers.

Solid state polymerization of frozen solutions.

Solid state polymerization of biodegradable macromonomers that produce crosslinked biodegradable polymers.

Solid state polymerization of PEG based biodegradable macromonomers.

### Example 10-1

Solid state photopolymerization in frozen solution state:

In a 100 ml beaker 3 g of PEG35K-LACTATE-5-acrylate diacrylate (35KL5A) is dissolved in 9 g of PBS. To 1 ml of this solution 3 μl of initiator stock solution is added and vortexed. 20 μl of the solution is then exposed to 360 nm long UV light (Black-Ray UV lamp, 360 nm light, 10000 mW/cm2 intensity) for 5 minutes. The liquid solution converts into a soft gel indicating effective polymerization and crosslinking in the solution state. Another 50 μl of the monomer solution is first frozen at −20 degree C. on a glass slide in the refrigerator and then exposed to 360 nm light for 10 minutes in the frozen state. The exposed solution is then warmed to room temperature and incubated in water. The crosslinked gel swells slightly in the water without dissolution indicating effective polymerization and crosslinking in the solid frozen state. The crosslinked gel thus produced is biodegradable due to presence of lactate groups in the crosslinked polymer. A control solution that is frozen but not exposed to light is dissolved in water indicating that the exposure of UV light initiates polymerization and crosslinking in the solid/frozen state. The same experiments are repeated at 20 and 30 percent macromonomer concentration in PBS. In both cases, the solution photocrosslinked in solution and frozen/solid state.

Using a similar procedure as above, 50.2 mg of PEG20K urethane diacrylate (PEG20KUA) is dissolved in 0.5 ml DMSO and then mixed with 2 microliters of ISS stock solution. One drop of the solution is exposed to a Long UV light lamp (Wenzhou Aurora Technology Company Ltd., Wenzhou, China, model UV-300, 365 nm light with intensity 26500 μW/cm²). The solution formed a gel with 60 seconds of light exposure indicating effective crosslinking. 50 μl of the same solution is transferred on a glass slide, frozen to −20 degree C. which is then exposed to light for 60 seconds. The light exposed solution is warmed to room temperature ensuring no melting of the gel occurs. The gel is incubated in water wherein it maintains its size and shape indicating the formation of effective solid state polymerization. Using a similar procedure as above, 10 percent solution of PEG20KUA in PBS, polyethylene glycol dimethyl ether (molecular weight 600) is tested for effective polymerization in solution and in frozen state. Effective polymerization is observed in both cases. DMSO based formulation as above is also tested with PLGA polymer (molecular weight 125000-150000 Daltons, 20 percent) as a non-crosslinked polymer additive. The PLGA comprising composition is also polymerized in solution and frozen state. PLGA encapsulates the drug and its film forming ability provides mechanical stability to the crosslinked composition. In another variation of the above experiment, 1,4-dioxane is used as a solvent instead of DMSO and the polymerization is done at −10 to -5 degree under frozen conditions. The solvent is removed by lyophilization after effective polymerization and crosslinking.

### Example 10-2

Solid state polymerization in the dry state after partial or complete removal of the solvent

In a 100 ml beaker 3 g of PEG35K-LACTATE-5-acrylate diacrylate (35KL5A) is dissolved in 9 g of ethyl acetate or THF. 1 ml of this solution is mixed with 10 mg of Irgacure 2959 photoinitiator and optionally with 20 microliters of vinyl pyrrolidinone as comonomer. 20 μl of the solution is then exposed to 360 nm long UV light for 5 minutes. The liquid solution converts into a soft gel indicating effective polymerization and crosslinking. 50 μl monomer solution described in this example is added on top of a glass slide and the solvent is evaporated for 30 minutes at ambient temperature (around 30 degree C.). The substantially dry composition without solvent is then exposed to 360 nm light for 10 minutes in the dry state. The exposed solid is then incubated in water at room temperature. The solid did not dissolve in the water but formed a crosslinked gel in water indicating crosslinking of the composition in the dry state. Without polymerization and crosslinking, the dry composition should dissolve in the water without forming a gel. In another experiment, 5 solution droplets (about 100 mg each) are weighed on 5 glass slides. The solutions are then evaporated to different degrees of dryness and weighed. The samples with different amounts of dryness or solvent are then exposed to UV light wherein all samples were able to polymerize and crosslink upon exposure to UV light.

Using a similar procedure as above, 50.6 mg of PEG20K urethane diacrylate (PEG20KUA) is dissolved in 0.5 ml toluene and then mixed with 10 μl of Irgacure 2959 photoinitiator stock solution (50 mg in 0.5 ml n-vinyl pyrrolidone). One drop of the solution is exposed to long UV light (365 nm light with intensity 26,500 μW/cm²). The solution changes to a gel with 40 seconds of light exposure indicating effective crosslinking. Another 50 μl of the same solution is transferred on a glass slide and dried in a fuming hood for 30 minutes at room temperature. The dried composition is then exposed to light for 60 seconds. The exposed solution is incubated in water and forms a crosslinked soft gel indicating the formation of effective solid state polymerization. Using a similar procedure as above, the 10 percent solution of PEG20KUA in dichloromethane is tested for effective polymerization in solution and dry state. Effective polymerization is observed in liquid as well as in dry state. DMSO based formulation as above is also tested with PLGA polymer (MW 125-150K, 20 percent) as a non-crosslinked polymer additive. The PLGA composition is also polymerized in solution and dry state. PLGA provides the ability to encapsulate drugs and its film forming ability provides mechanical stability to the crosslinked composition. Film forming ability helps to form microparticles using the photolithographic method discussed elsewhere in the specification. Other monomers with different biodegradable blocks, molecular weight, with additives like biodegradable polymers can be effectively polymerized in the dry state if effective polymerization conditions are provided. In some instances, dry powder particles are formed upon drying and crosslinking has taken place in the particles separately.

### Example 10-3-1

Solid state polymerization to form microcylinders using photolithography.

PEG20K urethane diacrylate (PEG20KUA, 10 percent) and PLGA (MW 125-150K, 20 percent) and Irgacure (0.1 percent) solution in DMSO is applied in a 1 X 1 cm area on a glass slide and the applied solution is dried under vacuum. The PLGA and macromonomer form a fine coating on the glass plate. This film is covered with a photomask (10 by 10 array pattern with 0.3 mm clear circular area for light passage) and exposed to UV light for 2 min. The array plate is removed and the coating is washed with a DMSO containing photoinitiator to remove the unpolymerized coating. If needed, the washed composition may be further exposed to long UV light for additional crosslinking. The array pattern showing 300 microns diameter crosslinked polymer is clearly seen on the plate. Crosslinking and gelation take place in the exposed area in the solid state. The particles are removed from the glass plate and stored until use. The PLGA in the particles may be used for controlled drug release. Drugs may be added during the precursor state if the drug can tolerate the crosslinking conditions.

### Example 10-3-2

Solid state polymerization to form bilayered microcylinders using a photolithography technique.

1 g of gelatin methacrylate is dissolved in 9 ml water. 3 μl of ISS solution and one drop of red ink (used as model visualization agent) is added. This solution is coated uniformly on a glass plate and frozen in the refrigerator. Another solution is prepared as above wherein red ink is replaced by blue ink and this solution is coated on the first red frozen solution layer ensuring no dissolution under cold conditions. Both layers are frozen at −20 degree C. The frozen plate is taken out and placed on an ice bath and then exposed to UV light (360 nm) passing through an aluminum plate array with 10 by 10 three hundred microns size holes. The unexposed solution is washed with distilled water. The particles are removed from the glass plate and observed under a microscope for size, uniformity and bilayered structure. The unibody crosslinked gelatin methacrylate particles with bilayer structure (around 300 micron size diameter, cylindrical shape, blue and red layers) with a clear demarcation between blue and red layer is seen.

### Example 10-4

Solid State Polymerization to encapsulate drugs/enzymes or live cells.

Chinese hamster ovary (CHO) cells (supplied by ATCC, CHO-K1 (ATCC® CRL-9618) are thawed to 37 degree C. and transferred to a 75 centimeter square tissue culture flask containing 20 ml of ATCC formulated F-12K Medium with fetal bovine serum (final concentration of 10%). CHO cells are used as an illustrative mammalian cell line because it is widely used in the manufacturing of therapeutic protein drugs. The cells are incubated at 37° C. in a suitable incubator that provides 5% CO2 in the air atmosphere. The medium is changed daily. After 2-3 days and reaching full confluence, the cell culture medium is removed and cells are rinsed with 0.25% trypsin, 0.03% EDTA solution. An additional 1 to 2 mL of trypsin-EDTA solution is added and is incubated at 37 degree C. until the cells detach. The cells are centrifuged, the supernatant is removed and cells are resuspended in 5 ml cell culture media containing 10 percent DMSO as a cryopreservative agent. Separately, 100 mg of PEG20K urethane diacrylate (PEG20KUA) is dissolved in 0.5 ml PBS (pH 7.4) and then mixed with one μl of Irgacure 2959 photoinitiator stock solution (ISS). To this solution, 0.1 ml cell suspension is added and mixed. Small amount of suspension is checked for viability via live dead assay. The suspension is then added in silicone mold with 100 cylindrical mold cavities separated by 2 mm (cavity dimension 300 microns diameter and 500 microns height). Each cavity is filled with cell suspension, excess solution is wiped off and the mold is placed at 4 degree C. in refrigerator for 5 minutes and then in a programmable cooler to cool at 1 degree per minute until −70 to −90 degree C. and then finally in liquid nitrogen at −192 degree C. Many cell lines need controlled-cooling and cryopreservative to survive the freezing operation. The cryopreservative agent enables cells to withstand freezing and thawing steps. The frozen particles are thawed at −10 to 0 degree C. and exposed to long UV light (Black-Ray UV lamp, 360 nm light, 10000 mW/cm2 intensity, for 5 minutes) to initiate polymerization and crosslinking in solid state. The mold is warmed to room temperature and the crosslinked gel microcylinders are removed from the mold and suspended in the culture medium. The viability of cells inside the crosslinked gel is checked after encapsulation. Cell lines generally tolerate crosslinking processes without substantial viability loss (less than 30 percent loss).

In another modification as above, the cell suspension in the macromonomer as above is sprayed to produce microdroplets of around 100 to 1000 microns in diameter. The droplets are collected in liquid nitrogen for rapid freezing. The quick freezing has the potential to affect the viability of cells and tolerance for a particular cell line. Cell viability with quick freezing may be improved by using cryopreservatives. The frozen droplets are warmed to −10 degree C. and exposed to UV light for 5 minutes (all droplets are exposed without shadowing effect). The crosslinked microspheres with encapsulated cells are then suspended in a culture medium and checked for cell viability. Yet in another modification, 0.2 ml of the macromonomer cell suspension is loaded in a 1 ml syringe with a 32 gauge needle. The cell suspension is injected from the syringe and the droplets are collected in the liquid nitrogen where they immediately freeze. The frozen droplets are then exposed to long UV light in a frozen state at −10 degree C. for 5 minutes to polymerize and crosslink the macromonomer and entrap the cells. The encapsulated cells are then stored in a tissue culture medium until further use. Yet in another modification as above, the cell suspension is replaced with bupivacaine hydrochloride (as a model drug, final concentration 10 percent), shaped into frozen microdroplets as above and polymerized and crosslinked to form drug encapsulated microspheres.

In some compositions, an additive is specifically added to improve handling to maintain the desired frozen shape upon freezing. The additive also helps in handling frozen particles without significant damage or breakage during the arrangement of frozen particles. The additive may be polymeric/macromolecular or non-polymeric; hydrophobic or hydrophilic polymer; biostable polymer or biodegradable polymer or inorganic or organic filler. For polymeric additives, molecular weight may range from 10000 Daltons to 2 million Daltons. In the preferred mode, the additive and precursor are first dissolved in a common aqueous or organic solvent along with a photoinitiator to form a homogeneous solution. The number of additives may range from 0.01 percent to 80 percent relative to total solution weight, preferably 0.05 percent to 60 percent.

In some embodiments, the frozen precursor microspheres are stored with cells/drugs until use. The stored microspheres are then utilized in making bilayered or multilayered objects as described in this invention.

### EXAMPLE 11

Biodegradable hydrogels with multiple layers comprising microencapsulated microsphere particles.

### Example 11-1

Preparation of multilayered hydrogel with microparticles (single hydrogel particle with multiple layers/sections/zones comprising drug/visualization agent encapsulated microparticles)

Part A: Preparation of drug/visualization agent loaded microspheres using conventional process. Preparation of PLGA colored microspheres (stained)

0.5 g Poly(lactide-co-glycolide) copolymer (PLGA)(PURAC Biochem, Netherlands, PDLG 5002 polymer) is dissolved in 4.5 ml of ethyl acetate to make approximately 10 percent solution. In a 50 ml beaker, 5 ml of 1 percent polyvinyl alcohol (PVA) solution in distilled water (Sigma Aldrich, catalog number P8136, 30000-70000 g/mol, 87-90 percent hydrolyzed) and a magnetic stir bar are added. The solution is stirred and while stirring, 1 ml of PLGA solution is added dropwise. After complete addition, the solution is transferred to a 50 ml PP centrifuge tube and vortexed for 2 minutes. The drug suspension is then added to 40 ml of PVA solution and stirred vigorously overnight on a magnetic stirrer to remove ethyl acetate. Next day, PLGA suspension (approximately 35 ml) is transferred to a 50 ml polypropylene centrifuge tube. The suspension is centrifuged at 2500 rpm for 10 minutes. The supernatant is removed and the PLGA microsphere pellet is resuspended in 35 ml of distilled water and vortexed vigorously for 2 minutes, frozen and lyophilized. The microspheres are lyophilized and recovered as off white microspheres.

0.1 g of microspheres prepared as above are incubated in 5 ml tea leaves extract solution prepared in water. The tea-stained microspheres are separated from the solution, washed with distilled water and dried under vacuum. Similarly, 0.1 g of PLGA microspheres as above are exposed to a 1 percent solution of ethyl eosin prepared in ethyl alcohol to stain with fluorescent ethyl eosin. Similarly, Turmeric dissolved in ethyl alcohol is also used to obtain turmeric stained PLGA microspheres.

PLGA solution in ethyl acetate used as above is first mixed with 50 mg of rifampin until complete dissolution of the drug. This PLGA-rifampin solution is then used to make rifampin loaded PLGA microspheres as indicated above. The microspheres have mild red color due to the presence of rifampin which serves as a coloring agent as well as a model drug. Using a similar procedure as above, a 50 mg bupivacaine base is used to make bupivacaine encapsulated microparticles.

Part B: Preparation of multilayered biodegradable hydrogel microparticles with microencapsulated particles. 0.5 ml of 25 percent cold solution (0 to 10 degree C.) of JAL2UA in PBS (pH 7.4) is mixed with 200 mg of magnesium carbonate powder and, 10 μl of 2-hydroxy-4′-(2-hydroxyethoxy-2-methylpropiophenone) stock solution used as UV photoinitiator and mixed. Separately, 0.5 ml of 25 percent cold solution of JAL2UA in PBS (pH 7.4) is mixed with 100 mg of rifampin encapsulated microsphere powder and 10 μl of 2-hydroxy-4′-(2-hydroxyethoxy-2-methylpropiophenone) stock solution UV photoinitiator and mixed. Approximately 0.75 ml of cold suspension of JAL2UA-magnesium carbonate as above is added to a stainless steel rectangular mold with cavity size 5 cm by 5 cm by 1 mm to produce a 0.3 mm thick layer in the mold. The mold is warmed to 40 degree C. to form JAL2UA-magnesium carbonate thermosensitive gel. Cold suspension of JAL2UA-rifampin suspension is then added on top of the 0.3 mm thick layer obtained above. Both solutions are warmed to 40 degrees to form two different thermosensitive gel layers. Both gels are exposed to long UV light (2 minutes) via a photomask (300 micron diameter circles separated by 1 mm dark areas). The polymerization and crosslinking occur only through the exposed areas of the mask (300 microns diameter). The mold is cooled to liquefy the unpolymerized thermoreversible gel and then washed with PBS. The polymerized and crosslinked microcylinders have 300 microns diameter and 600 microns height. The microcylinders formed have two layers, the first layer (approximately 300 microns diameter and 300 microns height) comprises magnesium carbonate particles as visualization agent (opaque layer) and the second layer has rifampin loaded microspheres serving as drug-containing layer. The crosslinked JAL2UA serves as a biodegradable hydrogel encapsulating both layers.

In another variation, 300.1 mg of 2-hydroxy-4′-(2-hydroxyethoxy-2-methylpropiophenone) is first dissolved in 0.7 ml n-vinyl pyrrolidone. This solution is used as a long UV light initiator solution (initiator stock solution). In a 5 ml glass vial, 50.0 mg of PEG35KUA macromonomer is dissolved in 0.5 of ml PBS and 10.0 μl initiator stock solution (described above) is added. The precursor solution is further added to 50 mg of rifampin microencapsulated particles to form a suspension. In another vial, another precursor solution in PBS is made as above and is added to 20 mg of magnesium carbonate(opacity inducing agent or model visualization agent). A glass capillary 500 micron ID is attached with a pipette filler rubber bulb. First rifampin suspension is pulled in the capillary followed by magnesium carbonate suspension. Magnesium carbonate suspension has a higher density than rifampin suspension therefore, magnesium carbonate stays at the bottom without mixing with rifampin suspension. Since the concentration of monomers and initiators used is the same in both the layers, there is no diffusion of precursors/ingredients across the layers. Both the layers are exposed to 360 nm UV light until effective polymerization of both layers is achieved. The polymerized layers are taken out from the capillary as a single cylindrical unibody particle with two distant layers with the opaque layer containing magnesium carbonate and the transparent layer containing rifampin microencapsulated microspheres. In another variation of the same experiment, magnesium carbonate is substituted with bupivacaine encapsulated PLGA microspheres (10 percent drug loading, w/v PLGA 50:50, molecular weight 10000-15000 Daltons) to make bupivacaine and rifampin loaded bilayered microparticle. In both the examples listed above, a crosslinked hydrogel material is used as a base material to prepare multilayered particles and the layers comprise microencapsulated particles.

EXAMPLE 11-2-1 Preparation of multilayered hydrogel microparticles wherein separate layers are stacked and then encapsulated in a biodegradable hydrogel matrix.

### Example 11-2-1

Preparation of cylindrical gel particles.

In this example three layers of cylindrical particles are made with particles stacked on top of each other and then encapsulated in a hydrogel to make a single particle.

In a 100 ml beaker 3 g of PEG1OK-LACTATE-5-diacrylate (35KL5A) (preparation described earlier) is dissolved in 9 g of PBS. 1 ml of this solution is mixed with 3 μl of ISS solution and 30 mg of rifampin encapsulated PLGA particles. The suspension is poured into silicone rubber or stainless steel mold cavities. The mold has 300 micron of diameter and height as cavities. Excess solution is wiped off and the mold comprising the solution is exposed to 360 nm long UV light to crosslink the solution. The crosslinked hydrogels (300 microns diameter, 300 microns height) are removed. The hydrogel has yellowish/red shade due to rifampin PLGA particles suspended in the clear hydrogel. These hydrogel particles are labeled as A. Another set of hydrogels are prepared in the same manner wherein rifampin microspheres are replaced with magnesium carbonate as an opaque visualization agent. The microparticles are labeled as B. Third set of hydrogel microcylinders is prepared wherein rifampin encapsulated microspheres are replaced with bupivacaine in PLGA encapsulated microspheres. The microparticles are labeled as C.

### Example 11-2-2

Stacking and encapsulation of cylindrical gel particles with three layers.

1 ml of PEG10K-LACTATE-5-diacrylate in PBS as above is mixed with 3 of μl of initiator stock solution (ISS). A silicone mold with cavities having a size of 500 microns diameter and 1.2 mm height is used. A small amount of macromonomer solution is first added to fill about 5 percent of the mold cavity and the solution is frozen. On the top of the frozen solution, microparticles A, B, and C (described in EXAMPLE 11-2-1) are added sequentially. The remaining macromonomer mixture is poured into the mold cavity and filled completely. The hydrogel microparticles A, B and C are completely submerged in the precursor solution. The excess solution is wiped off and the solution is exposed to long UV light to initiate photopolymerization and crosslinking of PEG10K-LACTATE-5-diacrylate. The frozen solution is partially or completely melted before crosslinking. Upon effective conversion of solution into a gel, the hydrogel particles are removed from the mold. The hydrogel has ABC microcylinders embedded inside the crosslinked PEG10K-LACTATE-5-diacrylate hydrogel.

### Example 11-2-3

Stacking and encapsulation of cylindrical gel particles with four layers.

1 ml PEG1OKUA macromonomer solution (20 percent) in PBS along with UV photoinitiator and 1 g of magnesium carbonate stained with a fluorescent dye are mixed. The suspension is then loaded in a 0.55 mm glass capillary for a length of about 1.2 mm. The suspension is exposed to light and crosslinked. The crosslinked microcylinder is taken out and stored.

1 ml PEG1OKUA macromonomer solution in DMSO (20 percent) along with UV photoinitiator and 100 mg of PLGA (1:1, molecular weight 10000-15000 g/mole are mixed until complete dissolution of PLGA. The solution is then loaded in a 0.55 mm glass capillary for a length of about 1.2 mm and exposed to light and crosslinked. The crosslinked microcylinder is taken out, incubated in water to precipitate the polymer and solvent removal.

The internal lumen of a 0.7 mm glass tube/capillary was coated with mineral oil. A PEG35KUA macromonomer solution along with a photoinitiator in PBS is filled in the tube. Microcylinders with crosslinked PEG1OKUA with magnesium carbonate and PLGA are then pushed inside the capillary. The microcylinders are placed in such a way that about 1.4 mm of PEG35KUA macromonomer solution is either side of the microcylinders and the microcylinders are completely immersed in the PEG35KUA macromonomer solution. The entire assembly is then exposed to light to polymerize and crosslink PEG35KUA macromonomer solution. The crosslinked PEG35KUA macromonomer is removed from the mold. The 4 layer implant thus formed has a clear hydrogel on either side of the implant made out of crosslinked PEG35KUA macromonomer. The center of the implant has PEG1OKUA crosslinked microcylinders comprising fluorescent magnesium carbonate and PLGA.

Separately 0.55 mm diameter and about 1.3 mm length (available solution) was filled in glass capillary (0.6-0.7 mm) and then one fluorescent PEG 10K acrylate and MgCO3 micro cylinder (dia—0.55 mm) and one PEG 10K and PLGA 50:50 micro cylinder (dia 0.55 mm) were dipped in the solution and then the solution was exposed under UV light for about 5 minutes and then removed cylinder using a single plunger. Obtained layered gel was observed under a microscope in normal and UV light.

### EXAMPLE 12

Preparation of multilayered particles.

### Example 12A-1

Preparation using a single mold and using the frozen solution technique.

A silicone rubber mold with 100 cubical cavities in a 10 by 10 array format (cavity size 2000 microns length, 2000 microns width and 2000 microns height) separated by 2 mm distance is prepared. In a 15 ml glass vial, 500.0 mg of PEG35KUA macromonomer, 1 g of PLGA 10000-15000 Daltons and 250 mg of lodixanol are dissolved in 5 ml DMSO to obtain solution number 1. 15 microliters of 2-hydroxy-4′-(2-hydroxyethoxy-2-methylpropiophenone stock solution in n-vinylpyrrolidone (ISS) is added to the solution and is mixed thoroughly. In another 15 ml vial, solution as above is made wherein lodixanol is replaced with bupivacaine hydrochloride(drug) to obtain solution number 2. 2 μl of solution 1 is added in each cavity and the mold is frozen in liquid nitrogen. The frozen mold is taken out and the remaining space in the mold cavity is filled with solution number 2. The mold and solution warmed to room temperature and exposed to long UV light (360 nm) for 5 minutes to polymerize the solution. Care is taken to ensure that the polymerization is done prior to diffusion of drug and lodixanol to other layers. The polymerized particles from the mold are taken out and DMSO is removed by vacuum drying at 40 degree C. for 3 days or by exchanging with dichloromethane which is then removed by a combination of air drying and vacuum drying. Removal of solvent precipitates the PLGA in the crosslinked matrix and encapsulates the drug and lodixanol in the crosslinked matrix as well as in PLGA. The single particle formed has two zones/layers in unibody structure wherein one of the zones comprises lodixanol and the other zone comprises bupivacaine hydrochloride.

### Example 12A-2

Preparation of multilayered particles made using thermosensitive gelation property of the precursor.

In another embodiment as above, 0.25 g of F127LA, 0.75 g of PBS solution, 50 mg of lodixanol are mixed in a 5 ml glass vial at 0-10 degree C. with vigorous shaking. In another 5 ml glass vial, 0.25 g of F127LA, 0.75 g of PBS and 7.5 mg bovine serum albumin as a model drug are mixed I at 0-10 degree C. with vigorous shaking. To both the cold solutions 3 μl of 2-hydroxy-4′-(2-hydroxyethoxy-2-methylpropiophenone photoinitiator stock solution is added. 2 μl of F127LA/ lodixanol solution is added in each cavity of the silicone mold as above and the mold temperature is increased to 40 degree C. to cause thermal gelation of F127LA macromonomer solution. While in the gel state, the second F127LA/albumin cold solution is added in the remaining space of the cavity. Excess solution is wiped off from the mold surface and the solution is immediately exposed to 360 nm UV light to initiate photopolymerization in both layers. The polymerization of both solutions leads to single gel particles with two zones/layers with the radio-opaque zone consisting of lodixanol and the other zone containing F127LA/ albumin which provides sustained drug release properties unaffected by the presence of lodixanol. The particles are removed from the mold and stored until use.

### Example 12A-3

Preparation using a single mold with a solvent evaporation technique.

A 500 micron thick stainless steel plate is laser drilled to create 300 microns diameter holes in an array format (30 by 30 holes, 2 mm pitch). In a 15 ml glass vial, 500.0 mg of PEG35KUA macromonomer, 1 g of PLGA 10000-15000 Daltons and 100 mg of bupivacaine base and are dissolved in 5 ml of ethyl acetate. 15 microliters of 2-hydroxy-4′-(2-hydroxyethoxy-2-methylpropiophenone stock solution in NVP is added to the solution and is mixed thoroughly. In another 15 ml vial, 500.0 mg of PEG35KUA, PLGA 10000-15000 Daltons and 100 mg of lodixanol are dissolved in 5 ml DMSO followed by the addition of 15 microliters of ISS solution. The steel mold is kept on a soft silicone rubber sheet and ethyl acetate precursor solution is added to completely fill the mold cavities and excess solution is wiped off. The mold is weighed and solvent ethyl acetate is partially evaporated to create a space in the mold cavity. The evaporation is monitored by weighing the mold over a period of time so that about 10 percent of volume space is created in the mold cavity due to solvent evaporation. The precursor solution with lodixanol in DMSO is added in the cavity on top of the first solution until the cavity is filled completely. The excess solution is wiped off and the solution is exposed to long UV light for 5 minutes until effective polymerization and crosslinking. The unibody bilayered organic solvent gel comprises a bupivacaine base as a model drug in one layer and lodixanol as a model radio-opaque agent in another layer. The gels are removed from the mold. The particles are dried under vacuum and stored until further use. The dried particle comprises the drug (bupivacaine base) in one layer and radio-opaque agent (lodixanol) in another layer entrapped in crosslinked PEG35KUA macromonomer and PLGA. Ethyl acetate solution may be frozen first before adding DMSO solution to prevent mixing.

A similar experiment is carried out in the glass capillaries plates (Hamamatsu Corporation, Photonics Division, Bridgewater, N.J., product J5022-11 with 10 micron diameter and 400 micron height) where the composition is first completely filled in the plate and then the solvent is partially removed by air/vacuum drying and then the second composition is filled in the newly created space in the capillaries and then crosslinked using long UV light. The crosslinked unibody is removed from the mold. If needed the length can be further reduced by cutting the particles.

### Example 12B

Preparation of multilayered particles using multiple molds.

1 g of F127LA, 3 g of PBS solution, 300 mg of lodixanol are mixed in 15 ml glass vials at 0-10 degree C. with vigorous shaking. To this solution, 9 microliters of ISS solution is added and mixed. Separately 1 g of

F127LA, 3 g of PBS solution, 100 mg of albumin are mixed in 15 ml glass vial at 0-10 degree C. with vigorous shaking followed by the addition of 9 microliters of ISS solution. Two silicone rubber based molds are used to make multilayered particles (FIG. 11). Mold one is a 5 x 5 cm and 2 mm thick rectangular silicone mold that is custom made. The mold has 25 circular cavities with 1000 microns diameter and 500 microns depth in 5 by 5 matrix format separated by 2 mm. The 4 corners of the mold have 30 mm long 2.5 mm diameter stainless steel rods embedded which act as guideposts to align the second mold on top of the first mold. The center of the rod is at 45 degree angle from the corner of the mold at a distance of 5 mm (FIG. 11). The second silicone mold is also in the form of a sheet with the following properties. Dimension 5×5 cm, 1 mm thick. The sheet has circular holes 1000 micron in diameter in 5 by 5 matrix format. The 4 corners of the mold two have 2.8 mm diameter holes which act as an alignment aid to align cavities of both molds. The center of the corner hole is at 45 degree angle from the corner of mold two at a distance of 5 mm. When mold two is inserted on top of the first mold one via corner holes and guiding posts, the center of the cavity of mold 1 matches with the center of mold 2 holes and this arrangement increases the depth of mold one from 500 microns to 1500 microns (500 microns from mold one and 1000 microns from mold two). Mold one cavity is filled with cold F127LA/ lodixanol in PBS solution (0-5 degree C.). The temperature of the mold is raised to 40 degree C. with a hot air gun. The increase in temperature causes physical gelation of F127LA/Iodixanol solution. Mold two is then inserted on top of mold one via guiding rods on mold one and corner holes on mold 2 (FIG. 11). This aligns the center of holes in mold two with the center of cavities on mold one. F127LA/albumin cold solution is added on mold two cavities and excess solution is wiped off. The top mold is hard-pressed to prevent leakage between the molds. Optionally mold can be cooled to 0−10 degree C. to partially or completely melt both the compositions. The mold is then exposed to long UV light for 5 minutes before substantial mixing of both solutions. Polymerized circular two layered unibody particles are removed from the mold wherein the bottom part of each particle has lodixanol as a visualization agent and the top part has albumin as an illustrative protein drug.

### Example 12C

Multilayered particle using glass capillary.

Particles made using free radical polymerization in two different solvents

Free radical polymerizable precursor compositions that polymerize in water as well as in organic solvents are exploited to make multilayered particles. EXAMPLE 1 to 3 discloses various illustrative examples of precursors that polymerize in aqueous and organic solvents using condensation and free radical polymerization. Some of those precursors are used in making multilayered unibody particles. In a 5 ml glass vial, 50.0 mg of PEG35KUA macromonomer is dissolved in 0.5 ml of PBS and 2.0 microliters initiator stock solution (ISS) is added followed by adding 20 mg of magnesium carbonate as a visualization aid to form a suspension. In another vial, 50.0 mg of PEG35KUA macromonomer is dissolved in 0.5 ml dimethyl sulfoxide (DMSO) and 2 microliters initiator stock solution is added. A glass pipette with pipette filler is used to suction off controlled amounts of both precursor solutions each with length around 0.5 mm. Since the concentration of monomers and initiators used is the same in both layers, there is no diffusion across layers. Both layers are exposed to 360 nm UV light before substantial mixing to achieve effective polymerization of both layers. The polymerized layers are taken out from the capillary as a single cylindrical particle with two distant layers showing an opaque layer containing magnesium carbonate as an opacity inducing component and a transparent layer without magnesium carbonate. Even though polymerization has taken place in each layer, it forms a single unibody cylindrical mass. It is hypothesized (not necessarily bound by the hypothesis) that the partial solubility of PEG35KUA macromonomer in both layers enables covalently bonding of both layers at the water and DMSO interface. In another embodiment as above, a third precursor solution (same as the first layer solution with magnesium carbonate) is pulled upwards in the capillary after pulling the first two solutions described above. All three layers are then subjected to crosslinking by UV light forming a three layer single particle with a crosslinked layer swollen in DMSO (OSG gel) that is sandwiched between two hydrogel layers containing magnesium carbonate.

In a 5 ml glass vial, 50.2 mg of PEG20KUA macromonomer is dissolved in 0.5 ml of distilled water and a 2 μl initiator stock solution (ISS) is added. Separately, 100.6 mg of PEG20KUA is dissolved in 1.0 ml of DMSO and 3 μl of ISS solution is added. To this 100.3 mg of PLGA (125-150K) is added and mixed well until complete dissolution. Both solutions are filled sequentially in a glass capillary with 0.5 mm diameter and exposed to long UV light. The polymerized unibody microcylindrical particle is taken out from the capillary. The bilayer particle is then immersed in coumarin (a model drug and fluorescent dye) solution in DMSO for one minute to diffuse coumarin in the particle and then washed with water two times to remove excess coumarin. The coumarin preferentially accumulates in the PLGA layer as compared to the non-PLGA layer of the particles.

In a 5 ml glass vial, 50.6 mg of PEG20KUA macromonomer is dissolved in 0.5 ml of DMSO and a 2 μl initiator stock solution (ISS) is added. Separately, 50.8 mg of PEG20KUA is dissolved in 0.5 ml of distilled water, 2 microliters of ISS solution is added followed by approximately 100 mg of magnetic ferrite powder. Both solutions are filled in a 0.5 mm diameter glass capillary in sequence and exposed to long UV light. The polymerized unibody microcylindrical particle is taken out from the capillary. The bilayer particle is then tested for magnetism using a small laboratory magnet. The ferrite portion of the particles attaches to the magnet indicating the magnetic property of the bilayer particle. A similar bilayer particle is made using iron oxide powder synthesized as per the literature method (M. Helminger, et. al. Adv. Funct. Mater., volume 24, 3187-3196, 2014) and it also showed magnetic properties. In another variation of the same experiment, the UV exposure and crosslinking is done in a glass capillary in presence of the magnetic field. The poles of magnetic particles in the precursor solution pre-align in the magnetic field and then crosslinking is initiated using long UV light to lock the particle's position in the magnetic field. In another modification of the current example, PEG20KUA macromonomer and lipase enzyme are dissolved in TRIS buffer (lipase concentration 1 mg/ml, 100 mM TRIS and 1 mM EDTA, pH 8) which is then used in place of DMSO solution and bilayer particles are made as described above with ferrite particle. The bilayer particles thus produced have magnetic particles in one layer and another layer has lipase enzyme encapsulated in the crosslinked matrix.

### Example 12D

Multilayered particles with each layer having a different physical or chemical property, chemical composition or drug.

Following free radical polymerizable precursor macromonomer solutions as listed in Table 3 are prepared and used to make two or more layered unibody particles in the subsequent examples 12D1-12F. About 2 microliters of photoinitiator stock solution (ISS) per ml of macromonomer solution is used to obtain about 0.1 percent final photoinitiator concentration in all solutions listed below.

TABLE 3
ILLUSTRATIVE BILAYERED COMPOSITIONS

| | |
|---|---|
| Solution A: | 50.0 mg PEG35KL5A macromonomer is dissolved in 0.5 ml DMSO. |
| Solution B: | 50.0 mg PEG35KL5A macromonomer is dissolved in 0.5 ml ethyl acetate. |
| Solution C: | 50.0 mg PEG35KL5A macromonomer is dissolved in 0.5 ml dichloromethane. |
| Solution D: | 50.0 mg PEG35KL5A macromonomer is dissolved in 0.5 ml Toluene. |
| Solution E: | 50.0 mg PEG35KL5A macromonomer is dissolved in 0.5 ml PBS (pH 7.4) or distilled |
| | water. |
| Solution F: | 50.0 mg PEG35KUA macromonomer is dissolved in 0.5 ml PBS (pH 7.4) or distilled water. |
| Solution G: | 150.0 mg F127LA macromonomer is dissolved in 0.5 ml cold (0-10 degree C.) PBS and |
| | used as a cold solution. The solution turns into a gel when subjected to a temperature |
| | around 37 degree C. |
| Solution H: | Same as E with 10 mg of Iodixanol encapsulated PLGA microparticles (size 1-10 microns, |
| | 20 percent Iodixanol loading, PLGA 1:1, molecular weight 150000-200000 Daltons). |
| | Iodixanol is used as an illustrative radio-opaque contrast agent. |
| Solution I: | 50 mg of PEG-10K4arm-lactate-tetraacrylate (PEG4AL5) is dissolved in 0.5 ml ethyl |
| | acetate. |
| Solution J: | Same as B with addition of 50 mg PLGA (1:1, molecular weight 15000-30000 Daltons). |
| Solution K: | Same as E with addition of 0.5 mg of dexamethasone or bupivacaine hydrochloride. |
| Solution L: | Same as E with addition of 50 mg of dexamethasone or bupivacaine base encapsulated |
| | PLGA microparticles (size 1-10 microns, 10 percent drug loading, PLGA 1:1, molecular |
| | weight 15000-30000 Daltons). |
| Solution M: | 50.0 mg gelatin methacrylate macromonomer is dissolved in 0.5 ml PBS. |
| Solution N: | 50.0 mg gelatin methacrylate macromonomer is dissolved in 0.5 ml DMSO. |
| Solution O: | Same as solution E or F with the addition of one Radio-frequency identification chip |
| | (RFID) chip (Hitachi “Dust” RFID tag (128 bits read only memory, 38 digit number |
| | identification) suspended in the solution. |
| Solution P: | Same as solution E with the addition of 1 mg of Rituximab (anticancer antibody, Anti- |
| | CD20; Chimeric IgG, used for Non-Hodgkin's lymphoma). |
| Solution Q: | Same as E with the addition of 25 mg of rifampin encapsulated PLGA microparticles (size |
| | 1-10 microns, 10 percent drug loading, PLGA molecular weight 15000-30000 Daltons). |
| Solution R: | Same as A with the addition of 2 mg of paclitaxel. |
| Solution S: | Same as E with the addition of 5 mg of bovine serum albumin as a model protein drug. |
| Solution T: | Same ss E or F with the addition of 30 mg magnetic particles. |
| Solution U: | Same as E except with the addition of 5 mg of silver or gold particles. |
| Solution V: | Same as B with 500 mg of borosilicate glass particles. |
| Solution W: | Same as B with 500 mg of soda lime glass particles. |
| Solution X: | Same as E except with the addition of 0.5 mg of enzyme lipase as a model enzyme for |
| | chemical synthesis. |
| Solution Y: | Same as A except with 100 mg of glycidyl methacrylate is added as an illustrative |
| | functional monomer. |
| Solution Z: | Same as A except with 100 mg of lauryl methacrylate as a monomer with alkyl side chain. |
| Solution AA: | Same as E except with 5 mg of gelatin as water soluble crosslinkable polymer. |
| Solution BB: | Same as E with around 1 to 100000 live Chinese hamster ovary cells (CHO) with viability |
| | greater than 90 percent. |
| Solution CC: | Same as E except with 10 mg of diamond particle dust suspended in the solution. |
| Solution DD: | Same as B with 500 mg of calcium carbonate or magnesium carbonate or sodium |
| | carbonate or sodium bicarbonate. Preferably the particles are stained with dye. |
| Solution EE: | 200.0 mg 1,6-hexanediol diacrylate is dissolved in 0.3 ml methyl methacrylate. |
| Solution FF | 200.0 mg 1,6-hexanediol diacrylate is dissolved in 0.3 ml Methyl 2-chloroacrylate. |
| Solution GG | Same as B but with 100 mg of sodium chloride salt (size less than 100 microns). |
| Solution HH | Same as F but with 100 mg of psyllium husk. Polymerization is done quickly (less than 5 |
| | minutes) before psyllium absorbs and swells in water |
| Solution II | 150.0 mg F127UA macromonomer and 100 mg of Niacin is dissolved in 0.5 ml DMSO. |
| Solution JJ | Same as G or E but with 100 mg of polyallylamine. |

### Example 12D1

About One to five microliters of solution A and Solution B are pulled into a 500 micron diameter glass capillary tube and both solutions are then immediately exposed to 360 nm long UV light with an intensity of 26,500 μW/cm² for 5 minutes. Typically, gelation occurs in under 60 seconds. Both precursor liquid solutions convert into soft gel forming a single unibody gel particle. The crosslinked particles are ejected from the capillary by air pressure or mechanical plunger and stored until further use. The solvent ethyl acetate and DMSO are removed under vacuum forming dehydrated microparticles. All the other combinations described below are polymerized in a similar manner as above to produce 2 layered particles. Various preferred combinations of multilayered materials with unique properties are listed below.

### Example 12D2

Combination of B and E, Combination of C and E and Combination of D and E did not result in a single unibody particle and resulted into two separate gel particles where polymerization and crosslinking proceeds separately in each layer. This may be due to experimental conditions such as the immiscibility of solvents used. These crosslinked products may be stacked in any order and further encapsulated into crosslinked polymers such as crosslinked PEG35KL5A (FIG. 7D, 1006). Generally, each solvent combination is tested for the effective formation of a single multilayered unibody particle formation before using it to make multilayered particles.

### Example 12D3

Combination of A and E leads to the formation of single particles with zones/layers containing crosslinked PEG35KL5A macromonomer that is swollen in DMSO in one layer and in PBS in another layer. This combination results in two layered unibody particles comprising hydrogel and organogel.

### Example 12D4

Combination of L and S leads to the formation of a single particle with layers comprising crosslinked PEG35KL5A macromonomer. One layer has small molecular weight drug such as dexamethasone and the other layer has a protein drug such as albumin.

### Example 12D5

Combination of A and G: This combination produces bilayer unibody hydrogels wherein one layer is made using a macromonomer that has thermosensitive gelation property and other layer is made of using a macromonomer that has does not have thermosensitive gelation property. The crosslinked composition thus produced has one layer (F127LA) that expands on cooling in water due to its thermosensitive properties and other layer does not expand on cooling. This is also an example of a unibody particle with two layers with different thermal and water absorption properties for each layer.

### Example 12D6

Combination of A and I: This combination produces a multilayered product wherein crosslink density of the two layers are different from each other. The different crosslinking density produces different physical and chemical characteristics related to crosslinking density. The tetraacrylate monomer used in one layer (crosslinked PEG-10K4arm-lactate-tetraacrylate) and Diacrylate used in other layer (PEG35KL5A) provides the crosslinked materials with different crosslinking densities. The higher crosslinking density in the tetraacrylate layer is due to the lower molecular weight of macromonomer (higher molar concentration and lower molecular weight between acrylate groups) used and a greater number of polymerizable groups per molecule used during the crosslinking process.

### Example 12D7

Combination of J and A: This combination produces particles with two layers wherein one of the layers is a composite material comprising NBP as an additive. The composite materials comprises a non-crosslinked biodegradable polymer such as PLGA encapsulated in the organic solvent gel which may be precipitated in situ inside the layer forming composite material. The other layer is made using organic solvent gel or hydrogel without entrapped NBP polymer. The NBP may be loaded with a drug or visualization agent.

### Example 12D8

Combination of K and U: One layer comprises dexamethasone as a model drug and other layer comprises a model a model visualization agent. Silver and gold being radio-opaque in nature and therefore show prominently in x-ray imaging. The presence of metallic particles in sufficient quantities also makes the layer electrically conductive. This combination also results in multilayered particles wherein at least one of the layers is electrically conducting or is radio-opaque and the other layer has a drug.

### Example 12D9

Combination of K and L: This combination results in layered particles wherein each layer has a different drug release profile. One layer has biodegradable hydrogel comprising hydrogel with dexamethasone or bupivacaine hydrochloride microencapsulated particles and another layer has the same drugs without microencapsulation. This results in layers with different controlled drug release characteristics. One layer has a fast releasing drug (fast releasing due to non-encapsulation form of drug dexamethasone or bupivacaine hydrochloride) and the other layer has a slow releasing form of the drug (encapsulated form). Encapsulation of drugs slows drug release as compared to non-encapsulation form. The particle size of the particles used in the emulsion/suspension before crosslinking must be lower than the capillary internal diameter or mold cavity diameter, preferably 10 to 100000 percent lower, most preferably 20 to 10000 percent lower. In some experiments, 50, 250, 300 and 500 microns diameter glass capillaries are used.

### Example 12D10

Combination of L and Q leads to the formation of single particles with layers wherein each layer has different drugs present in the encapsulated form. Microencapsulated rifampin is used as a coloring agent as well as a drug in one layer and the other layer has drug dexamethasone in microencapsulated form.

### Example 12D11

Combination of A and M: One layer is made using a hydrogel that degrades via the enzymatic process in the body (crosslinked gelatin methacrylate). The other layer comprises a synthetic biodegradable hydrogel (crosslinked biodegradable polymer that degrades by hydrolysis process). This is also an example of two layered particles wherein at least one layer has composition that comprises functional groups capable of reacting with other chemical entities such as visualization reagent or drug. Crosslinked gelatin in one layer has reactive chemical/functional side groups such as amine, hydroxyl and carboxylic acid that can be used for chemical modification. One important modification is a grafting reaction. Functional groups in the gelatin layer are used in grafting reactions. Various polymers may be grafted to the gelatin layer, preferably grafted polymers are biodegradable polymers. About 1 g of dry gelatin comprising particles are incubated in a dry tetrahydrofuran solution comprising 20 percent dI-lactide and stannous octoate (0.1 percent) as a catalyst. The grafting reaction is performed at 60 degrees for 72 h under nitrogen atmosphere. The grafted particles are separated by filtration and dried under vacuum. The hydroxy, amine and carboxylic acid groups in gelatin initiate polymerization of dI-lactide forming polydI-lactide polymer side chains on the crosslinked gelatin polymer layer. The grafted polymer chains can be loaded with drug or visualization if desired using solvent diffusion techniques as disclosed in this invention. Other cyclic lactones and carbonates such as caprolactone, glycolide, trimethylene carbonate and the like may also be used in grafting reaction to produce polylactones or polycarbonates or their copolymers as biodegradable grafted polymers on gelatin layer. Gelatin functional groups can also be used to attach visualization agents like fluorescein or iodixanol.

### Example 12D12

Combination of K and O: one layer has the drug and the other layer has RFID tag to encode information about the device or drug. The RFID tag may be substituted with other microsensors such as pH sensors, temperature sensors and the like.

### Example 12D13

Combination of P and R: One layer has an anticancer drug like paclitaxel and other layer has illustrative anticancer antibodies that may be used as a homing tool or targeting tool for cancerous tumors.

### Example 12D14

Combination of A and U: One layer has biodegradable hydrogel and the other layer has a layer with gold or silver particles.

### Example 12D15

Combination of V and W: One layer comprises a crosslinked polymer with soda lime glass particles and the other layer comprises borosilicate particles. The layered particle thus prepared is dehydrated and subjected to a sintering process by heating at around 650 degree C. in air. At this temperature, the encapsulating matrix is substantially burned away leaving behind the bilayered particle with fused particles of soda lime glass in one layer and borosilicate glass in other layer. The particle is then annealed at around 450 degree C. overnight to remove process induced stress in the particles. This layered particle is a single particle with two types of glass fused with each other forming a unibody material.

### Example 12D16

Combination of F and Y: One layer comprises crosslinked hydrogel and the other layer comprises crosslinked polymer with reactive functional groups such as glycidyl groups. The glycidyl methacrylate comonomer used in the Y produces a crosslinked gel layer with glycidyl functional reactive side groups that can be used for covalent bonding of colored compositions or enzymes or other useful compounds. The glycidyl methacrylate can be substituted with other functional monomers and these include but not limited to: acrylic acid methacrylic acid for acid functional groups, 2-hydroxyethyl acrylate or methacrylates for hydroxy functional groups, 2-isocyanatoethyl methacrylate or acrylate for isocyanate functional groups, 2-aminoethyl acrylate or methacrylate with amine functional groups, methacrylaldehyde or methacrolein with aldehyde functional group, acrylic acid or methacrylic acid n-hydroxysuccinimide ester with n-hydroxysuccinimide ester as groups and the like.

### Example 12D17

Combination of F and Z: One layer with crosslinked hydrogel and the other layer of organic solvent gel comprising crosslinked polymer with lauryl as model alkyl side chain. The lauryl methacrylate comonomer used in the Z produces crosslinked gel layers with lauryl as chain (C12 alkyl chain). The lauryl methacrylate can be substituted with other alkyl chain containing monomers with C4 to C22 alkyl side chains and these include but not limited to: hexyl acrylate or methacrylate (C6 chain), octyl acrylate or methacrylate (C8 chain), decyl acrylate or methacrylate (C10 chain), stearyl acrylate or methacrylate (C18 chain).

### Example 12D18

Combination of R and T: One layer with crosslinked biodegradable organic solvent gel with paclitaxel as model drug and the other layer comprises magnetic particles. The presence of magnetic particles provides magnetic properties to the layer which can be useful in many applications.

### Example 12D19

Combination of Q and H: This combination provides a bilayered particle for controlled drug delivery wherein one layer has a drug and other layer has a visualization agent. Either drug or visualization agent or both are present in microencapsulated form such as microencapsulated PLGA microspheres. One layer has rifampin encapsulated microspheres and other layer has radio-opaque contrast agent encapsulated microspheres.

### Example 12D20

Combination of X and T: In this combination, one layer comprises crosslinked polymer or hydrogel comprising enzyme or biocatalyst and the other layer comprises magnetic particles. At Least one layer comprises a crosslinked polymer that is hydrophilic.

### Example 12D21

Combination of A and AA: One layer comprises crosslinked biodegradable PEG35KL5A polymer and the other layer has the same polymer as the first layer but also comprises gelatin as model water soluble NBP. The gelatin in the layer is physically entrapped in the crosslinked PEG35KL5A as NBP. The layered particle is then incubated in 0.2 percent glutaraldehyde solution to further crosslink gelatin with glutaraldehyde. The resultant particle has one layer with crosslinked PEG35KL5A and other layer with crosslinked PEG35KL5A and gelatin that is crosslinked with glutaraldehyde.

### Example 12D22

Combination of BB and T: One layer comprises crosslinked polymer or hydrogel with live CHO cells (around 1 to 100000 cells) and other layer comprises magnetic particles.

### Example 12D23

Combination of CC and T: One layer comprises crosslinked polymer or hydrogel with diamond dust particles which function as identification tags and other layer comprises magnetic particles.

### Example 12D24

Combination of 0 and T: One layer comprises crosslinked polymer or hydrogel with RFID tag and the other layer comprises magnetic particles.

### Example 12D25

Combination of DD and A: One layer is a crosslinked polymer or hydrogel and the other layer contains particles that are explosive in nature or particles that have the ability to provide propulsion to the particle. The layer with sodium bicarbonate can be irradiated with an infrared laser or visible light laser. The heat generated by laser light absorption decomposes sodium bicarbonate releasing carbon dioxide thereby providing kinetic energy to the microparticle/microsphere. Such particles can be used as micro bullets to penetrate the skin tissue. The other end of the particle preferably has a sharp end that facilitates easy penetration in the skin tissue. The laser energy and decomposition of sodium bicarbonate provides required kinetic energy such that the particle can penetrate in the tissue at the depth of 0.5 to 5 mm.

### Example 12D26

Combination of EE and FF: One layer is a crosslinked polymer comprising methyl methacrylate and other layer comprising methyl 2-chloroacrylate. Since one of the layers has methyl 2-chloroacrylate which has a higher refractive index than methyl methacrylate, the two layers have a different refractive index. Two dimensional and three dimensional multilayered materials with different refractive indexes have many applications as photonic materials if prepared using methods and compositions described in this invention. The refractive index may also be changed by adding non-crosslinked biodegradable or biostable as additives as described before.

### Example 12D27

Combination of GG and A: This combination produces particles with two layers wherein one layer is porous and the other layer is non-porous. The ethyl acetate layer comprises sodium chloride as a porogen entrapped in the layer. After polymerization and crosslinking, the composite material is incubated in water for 24 hours to leach out sodium chloride from the ethyl acetate layer. Removal of sodium chloride salt crystals creates porous space in the layer producing porous hydrogel. This combination produces layered crosslinked material wherein at least one of the layers is porous. The porosity introduced in one layer also lowers the density of the layer. Thus, two layered hydrogels produced as above have different densities. The porous layer has a much lower density than the other non-porous layer. By changing the porosity (amount of sodium chloride added), the density of the layer can be changed. The density difference also improves contrast in ultrasonic imaging of the material.

### Example 12D28

Combination of HH, II and JJ results in a trilayered unibody object with cholesterol sequestering properties. Combinations of ingredients that are known to reduce serum cholesterol upon oral ingestion are used in the formation of these multilayered particles. When precursor composition is crosslinked alone, it provides crosslinked PEG35KUA with encapsulated psyllium husk microparticles. The psyllium husk is known to reduce cholesterol upon ingestion. When the precursor composition II is crosslinked alone, it provides crosslinked PEGF127UA in organic solvent gel with Niacin as an illustrative cholesterol reducing drug. Niacin is known to reduce cholesterol. The hydrophobic block in Pluronic F127 is believed to provide controlled drug delivery of Niacin upon oral ingestion. Precursor composition JJ when crosslinked alone provides a polyamine polymer like polyallylamine entrapped in the crosslinked polymer. Upon effective crosslinking, polyallylamine is exposed to epichlorohydrin to crosslink the entrapped polyallylamine. The free amine groups in polyallylamine are acidified with hydrochloric acid to convert about 60 percent free amine to amine-hydrochloride salt. Semipermeability of crosslinked F127UA and its hydrophobic polypropylene block is believed to provide the ability to trap bile acids during digestive cavity transit. The crosslinked polyallylamine is also known to entrap bile acid upon oral ingestion. A combination of all three components in one single unibody object is expected to have an enhanced cholesterol reduction effect upon oral ingestion.

### Example 12E

Multilayered particles made using condensation polymerization and crosslinking.

### Example 12E-1

Multilayered particles comprising visualization agents.

In a 15 ml glass vial, 1 g of PEG10K4 ARM tetramine is dissolved in 9 ml of PBS (pH 7.4) and 100 mg of magnesium carbonate is added to the solution and vortexed to form a suspension. In a 15 ml glass vial, 1 g of PEG10K4ARM glutarate NHS ester (prepared as described in EXAMPLE 3 is dissolved in 9 ml of PBS (pH 7.4). 1 ml of each precursor is mixed in a 15 ml glass vial and immediately loaded (before gelation) into a glass capillary (500 micron diameter, 5 mm length, solution length 0.2 mm). The excess solution is wiped off. In another 15 ml glass vial, 1 g of PEG10K4 ARM tetramine is dissolved in 9 ml of DMSO. In another 15 ml glass vial, 1 g of PEG10K4ARM glutarate NHS ester is dissolved in 9 ml of DMSO. 1 ml of each precursor is mixed in a 15 ml glass vial and immediately loaded (before gelation) in the same capillary containing the earlier loaded solution (solution length 0.2 mm). Excess solution is wiped off from the glass surface and both solutions are allowed to crosslink in the capillary at room temperature without substantially mixing. Upon complete gelation, the single crosslinked gel particle is removed. This particle has two layers wherein one layer is transparent and is occupied by organic solvent gel (gel in DMSO) and the remaining other layer comprises a hydrogel. The hydrogel portion of the gel is opaque due to microparticles of magnesium carbonate. In another example as above, the magnesium carbonate is replaced with drug encapsulated microparticles for controlled delivery of drugs (Rifampin or bupivacaine base encapsulated microspheres). In another embodiment similar as above the precursor solutions are prepared in PBS. One precursor comprises PLGA microspheres with dexamethasone and the other precursor comprises rifampin encapsulated microspheres which serves as visualization agent (coloring agent) as well as drug. The prepared bilayered particles comprise two layers wherein one layer has drug and other layer has a visualization agent both encapsulated in a biodegradable polymer such as PLGA.

In another modification as above, DMSO solution above is added with non-crosslinked biodegradable polymer PLGA (10 percent, 50:50, molecular weight 15000-30000 Daltons, acetate endcapped) and forms a bilayer particle as above. This particle has an organic solvent gel layer with PLGA polymer entrapped in the organic solvent gel and hydrogel layer. The PLGA can be used for drug encapsulation as discussed in this invention. Other combinations of multilayered particles made using condensation polymerization such as particles with different degrees of crosslinking density, drug release profile, RFID and the like can be made as described above.

### Example 12F

Multilayered objects comprising hydrophobic and hydrophilic layers.

1 g of PEG35KUA macromonomer is dissolved in 9 ml distilled water. 30 microliters of ISS solution is added to the solution and the solution is degassed with nitrogen. Similarly, inhibitor free 1.5 g n-Lauryl acrylate, 0.5 g ethyleneglycol dimethacrylate and 8 g of DMSO are mixed until complete solution and 30 micrometers ISS solution is added. The solution is bubbled with nitrogen to remove dissolved oxygen. The solution is added in a glass petri dish and then frozen immediately to −10 degree C. Ice-cold solution PEG35KUA is then added on top of the frozen solution and then irradiated immediately with high intensity long UV light (360 nm) for 30 minutes. The cross linked unibody sheet is then washed with acetone to remove unreacted monomer and dried. The composite material thus obtained has a hydrophobic layer comprising n-lauryl acrylate and ethylene glycol dimethacrylate and other layer has PEG35KUA based crosslinked hydrogel. The composite material thus obtained can be potentially used for cleaning oil spills. The porous hydrophobic layer prepared by cryogenic photopolymerization process in the composite material is designed to absorb oil or other hydrophobic chemical spills. The density of such composite material is designed to be less than 1 g/ml so that the material can float on the water, especially sea water. The material may be covalently linked or added with a visualization agent to visually identify the materials visually during usage as oil spill cleaning material.

### EXAMPLE 13

Fabrication of multilayered rods and sheets

### Example 13-1

Fabrication of multilayered cylindrical rods with alternate sequences of elastomeric and non-elastomeric crosslinked material.

In a 10 ml glass vial, 500 mg of PEG2KUA macromonomer is dissolved in 5 ml of DMSO and a 15 μl initiator stock solution is added. Separately, 500 mg of PEG100KUA is dissolved in 5.0 ml of distilled water, 15 microliters of ISS is added. PEG2KUA solution is then loaded first in a 0.5 mm diameter glass capillary at a length of 1 mm followed by adding PEG100KUA solution at 1 mm length and this sequence is repeated 5 times. The entire solution in the glass capillary is then exposed to long 360 nm UV light to quickly polymerize and crosslink to form a unibody material. The polymerized unibody microcylindrical particle is taken out from the capillary. The unibody multilayered material has materials with high crosslinked density material (PEG2KUA) and lower crosslinked elastomer material (PEG100KUA) in an alternate sequence. To add further hardness rigidity and modulate other mechanical properties of crosslinked PEG2KUA material, a magnesium carbonate (an exemplary filler, 20 percent weight relative to total precursor solution) is added during precursor range and the unibody particle is made as described above. Non-crosslinked biodegradable polymer or non-crosslinked biostable polymer may also be added at the precursor stage in either precursors before crosslinking to modulate properties of the multilayred material as described above. Similar multilayered rod particles with alternate layers can also be made wherein PEG1OKUA and PEG35KUA in DMSO at 10 percent concentration along with 0.5 percent photoinitiator is used. In another modification, a polydimethylsiloxane based macromonomer with or without silicon dioxide as filler material is as an elastomeric block and hexamethylene diacrylate and methyl methacrylate solution is used as a hard block.

### Example 13-2

Fabrication of sheet

In a 100 ml conical flask, 5 g of PEG1OKUA macromonomer is dissolved in 50 ml of DMSO and 150 μl initiator stock solution is added. In another 100 ml flask, 5 g of PEG35KUA macromonomer is dissolved in 50 ml of DMSO until complete dissolution. 2 g of magnesium carbonate as a visualization agent and filler and 150 μl of initiator stock solution is added. The solutions are filled in separate silicone rubber mold with 2 mm by 2 mm by 2 mm size cavity. The molds are kept in the freezer at −20 degree C. for the solution to solidify. The frozen cubicle parts are removed from the mold and are arranged in a checkered alternate fashion to form a 5 by 5 matrix in another silicone mold of size 10.2 mm by 10.2 mm by 10.2 mm. The cube arrangement is tight and at least one of the surfaces of all the cubes generally are in contact with each other. The mold is warmed to room temperature and immediately exposed to long UV light for less than 60 seconds to effectively polymerize and crosslink the composition before substantial melting of the cube occurs. The fast polymerization prevents substantial mixing of precursors. Upon polymerization, zones of PEG1OKUA and PEG35KUA in a checkered fashion are seen in a single unibody structure. In one modification of the above embodiment, the mold is exposed to light when it is in a frozen or partially melted state.

Polyacrylamide gels made from acrylamide monomer and bisacrylamide crosslinker are used for gel electrophoresis in protein and biochemical analysis. The total percentage of acrylamide and bisacrylamide in the monomer mixture prior to crosslinking is usually 5-15% and 2.6-5% respectively. The gels are typically cast in Tris-HCI buffer (0.125 to 0.375M, pH 6.8 to 8.8). In a gradient gel, the total amount of acrylamide and bisacrylamide and/or crosslinker concentration is varied along the length of the gel. The amount of acrylamide is varied from 4 to 15 percent at a fixed bisacrylamide concentration. 11 solutions of acrylamide and bisacrylamide in 0.375M Tris-HCI (pH 8.8) along with photoinitiator are made and each solution has 5 to 15 percent acrylamide monomer concentration respectively. The solutions were frozen and cast into cubes as described above. 50 cubes for each solution concentration are arranged in a row and all 11 rows are joined together to create an acrylamide concentration gradient 5 to 15 percent along the column of the gel. The gels are irradiated with UV or visible light to crosslink in solid or solid-liquid state to form a unibody sheet wherein the acrylamide concentration is varied along the column height of the sheet. A wide variety of acrylamide gels with different amounts of crosslinking density in a single sheet of polyacrylamide can be made using this method. The size and thickness of the gel is changed by changing the size and number of frozen cubes. The arrangement and crosslinker concentration in the precursor stage is changed to make the desired density gradient along the length and breadth of the sheet material. The handling of toxic acrylamide in frozen state makes it easy to make acrylamide gels.

Yet in another modification of the above embodiment thermosensitive or pH sensitive macromonomer solutions in gel state are used to cast cubes or other shapes as described above and then the gels/cubes are arranged in a desired pattern in frozen and/or gel state. The patterned gels/cubes are crosslinked in solution or gel state or a combination of both states in any proportion. 35 percent (w/v) solution of F127LA macromonomer in cold water (0-10 degree C.) is mixed with photoinitiator Irgacure 2959 (final concentration 0.5 percent) and bupivacaine hydrochloride as a model drug (final concentration 10 percent relative to

F127LA weight). Similarly, another solution is made similar to as above except bupivacaine hydrochloride is substituted with bupivacaine microencapsulated in the PLGA polymer microspheres (1:1 PLGA molecular weight around 30000 Daltons, 20 percent drug loading, microspheres size 1-100 microns). Frozen cubes are arranged in a checkered fashion, the temperature is raised to 37 degree C. quickly to convert into a gel state (physically crosslinked), and the arranged cubes are then exposed to long UV light prior to substantial melting of the cube. The light exposure polymerizes and crosslink within and between the cube leading to a unibody crosslinked object. The unibody implant has two types of gels in a checkered fashion wherein one gel releases the drug at a faster rate (bupivacaine hydrochloride in un-encapsulated form) and the other gel releases the drug at a slower rate due to its encapsulation.

Yet in another modification gelatin methacrylate with and without magnesium carbonate are polymerized in a checkered fashion as described above. A 4 ml 10 percent solution of gelatin methacrylate in distilled water is made and Irgacure 2959 is added as a photoinitiator (0.1%). The prepared solution is divided into two parts. To one part magnesium carbonate (about 10 percent) as a model visualization agent that induces opacity to the gels. Magnesium carbonate filler may be substituted with drug encapsulated microparticles as a visualization agent as well as a drug delivery agent. The two parts are filled in separate 2 mm by 2 mm by 2 mm silicone rubber mold cavities and frozen. The frozen cubes of each part are arranged in a 4 by 4 alternate checkered pattern (total 16 cubes, an arrangement similar to shown in FIG. 14E) in cold condition in another silicone rubber mold. The arrangement is tight and at least one surface of the cube is in physical contact with the other cube in a frozen state. The cubes are then optionally warmed to room temperature until the melting of gelatin methacrylate on the surface of the cube just initiates and is then exposed to long UV light (360 nm) for 5 minutes. The exposed cubes undergo photopolymerization and crosslinking in solution state and/or frozen state or combination thereof forming a unibody structure with zones of gelatin methacrylate filled with (opaque gel) and without magnesium carbonate (clear gel). The presence of magnesium carbonate makes the gelatin methacrylate opaque. The crosslinked structure is incubated in water for 20 minutes at ambient temperature to test the formation of the crosslinked and unibody structure. Upon incubation in water, none of the cubes dissolved in water indicating that effective polymerization and crosslinking and all cubes were fused with each other indicating unibody formation (FIG. 14E1 and 14E2).

### EXAMPLE 14

Two layer contact lens comprising PVA and crosslinked PEG based material.

In 100 ml conical flask, 1 g polyvinyl alcohol (degree of saponification 99.9%, average molecular weight 146,000-186,000 g/mole) is added in 9 g DMSO:deionized water mixture (80:20). The solution is heated for 2 hours at 100 degree C. in oil bath while stirring until complete dissolution of PVA. 1 g of PEG20KUA macromonomer is added to the solution followed by 30 microliters ISS stock solution (solution 15A).

Another solution is made similar to solution 15A but was added with 1 g of jeffamine lactide (solution 15B). Solution 15A as described above is injected into contact lens mold cavity to occupy upto 30-90 percent of mold cavity (5-12 mm diameter) volume. The solution is then cooled to −20 degree C. to freeze the solution. The remaining space is then filled with solution 15B (about 9 mm diameter extension). The solution 15B occupies space on top of frozen solution 15A in the mold. Both the solutions are exposed to high intensity UV light to initiate polymerization and crosslinking of both the solutions to form a two layer unibody material. The crosslinking is done before substantial mixing of solutions 15A and 15B. The solution 15A and 15B may be solid, partially liquid or completely liquid during the crosslinking process. After crosslinking the solution is frozen again −20 degree C. and held there for 24 hours. This freezing and subsequent thawing step physically crosslinks the PVA in both layers. The DMSO in the lens is removed by incubation in water at 37 degree C. The jeffamine lactide in the outer layer can be loaded with an ophthalmic drug such as dexamethasone using solvent based methods as discussed in this invention. The contact lens thus produced is a composite of PEG based crosslinked macromonomer material and PVA that is crosslinked using freeze thaw process.

### EXAMPLE 15

Multilayered ophthalmic implant comprising biodegradable hydrogel.

Multilayered punctal implants comprising drug and/or visualization agent loaded microencapsulated microspheres.

100 mg of PLGA microspheres (average size around 10 microns) comprising 10 percent dexamethasone are made using methods described in this invention or in the literature. Similarly, 100 mg PLGA microspheres comprising 0.1 percent sodium fluorescein (illustrative visualization agent) are also made. In a 5 ml glass vial, 50.0 mg PEG35KL5A macromonomer is dissolved in 0.5 ml PBS (pH 7.4) and a 2 μl initiator stock solution (ISS) is added. To this solution 50 mg of dexamethasone PLGA microspheres are suspended. In a separate vial, PEG35KL5A macromonomer solution with photoinitiator and 50 mg sodium fluorescein PLGA microspheres are suspended. Both suspensions are loaded in a glass capillary (0.5 mm diameter, 0.5 mm length per solution). The solution is exposed to long UV light to initiate polymerization and crosslinking of PEG35KL5A macromonomer for 5 minutes. The crosslinked bilayered implant formed (length 1 mm with half of the implant comprising dexamethasone microspheres and rest with sodium fluorescein. Alternatively molds with 1 mm diameter and 0.5 mm length and 0.5 mm mm diameter with 0.5 mm length are filled with each suspension, frozen and frozen solids are taken out. The frozen solids are arranged on top of each other and then crosslinked using light in the frozen state and/or liquid state to make a unibody implant. The implant thus formed has half of the area dedicated for drug release and remaining half is dedicated for visualization agent. Due to the difference in diameter of frozen solids, the implant produced has a shape similar to shown in FIG. 16, B42 or B6. The ratio of drug to visualization agent in the implant may be varied from 5 percent to 95 percent depending on the ophthalmic application using the method described as above.

### EXAMPLE 16

Preparation of multi-compositional sheet like devices such as wound dressing.

Preparation of wound dressing comprising two or more hydrogel compositions in a grid format.

### Example 16A

Preparation of hydrogel using frozen solution technique.

A silicone rubber mold with 100 cubical cavities in a 10 by 10 array format (cavity size 2000 microns length, 2000 microns width and 2000 microns height) separated by 2 mm distance is prepared. In a 15 ml glass vial, 500.0 mg of PEG1OKUA macromonomer, 1 g of PLGA 10000-15000 Daltons and 100 mg bupivacaine base are dissolved in 5 ml DMSO to obtain a macromonomer solution. 15 microliters of 2-hydroxy-4′-(2-hydroxyethoxy-2-methylpropiophenone stock solution in n-vinyl pyrrolidone (ISS) is added to the solution and is mixed thoroughly (PEG10K-B). 500.0 mg of gelatin methacrylate macromonomer, 50 mg of sodium hyaluronate are dissolved in 5 ml PBS to obtain a macromonomer solution. 15 microliters (ISS) is added to the solution and is mixed thoroughly (GM-H). 500.0 mg of PEG1OKUA macromonomer, 25 mg hydroxypropyl methylcellulose (HPMC)in 5 ml PBS to obtain a macromonomer solution. 15 microliters of (ISS) is added to the solution and is mixed thoroughly (PEG1OK-HP). All three solutions are filled in the silicone mold and cooled to freeze the monomer solutions. The frozen cubes are taken out and stored in frozen conditions until use. Rectangular insulated plastic mold with 50.2 mm length, 50.2 mm breadth and 2 mm depth cavity is cooled to −80 degree C. The frozen cubes of PEG1OK-HP, PEG10K-B and GM-H are arranged in the mold in an alternative fashion to produce 25 by 25 matrix of cubes. The arrangement of cubes is tight and at least one surface of the cube is in physical contact with the other cube in the frozen state. The mold is brought to zero degree C. to room temperature and quickly exposed to high intensity UV light to polymerize and crosslink the monomers before substantial mixing of monomers. The polymerized unibody is taken out and exposed to PBS solution to wash out unpolymerized monomers and initiator fragments and to precipitate the PLGA polymer in the PEG matrix (PEG10K-B). The unibody hydrogel thus produced has surface and bulk comprising alternate 2 mm blocks of PEG1OK-HP, PEG1OK-B and GM-H hydrogels in a grid pattern. Bupivacaine in PEG10K-B block provides controlled release of bupivacaine base for local anesthetic effect. Entrapped hydroxypropyl methylcellulose and hyaluronic acid in crosslinked PEG based hydrogel provide wound healing properties and water absorption properties needed for wound healing.

In another modification of the above example, rectangular insulated plastic mold with 50.2 mm length, 50.2 mm breadth and 4 mm depth cavity is cooled to −80 degree C. GM-H cubes are arranged in the entire mold surface as the first layer. PEG1OK-HP layer was added on top of the GM-H layer. Both the layers are brought to room temperature and then exposed to light to initiate effective polymerization and crosslinking in both layers either in solid state or liquid state or combination thereof. Upon effective polymerization and crosslinking two layered unibody hydrogel is formed wherein one of the layers of sheet like material is made using PEG1OK-HP and other layer is made using GM-H. Yet is another modification, PEG1OK-HP is replaced with monomer composition comprising polydimethylsiloxane (polydimethylsiloxane terminated with methacrylate end groups, [methacryloxypropyl terminated polydimethylsiloxane], molecular weight 380-550 g/mole, Gelest Inc. catalog number DMS-R05). This produces an oxygen permeable layer comprising polydimethylsiloxane in the composite layer.

### Example 16B

Preparation of hydrogel wound dressing comprising polydimethylsiloxane.

Oxygen permeable hydrogel wound dressing comprising polydimethylsiloxane.

2 g of 3-(methacryloyloxy)propyltris(trimethylsiloxy) silane (TRIS), 2 g of N,N-dimethylacrylamide and 2 g of 1-vinyl-2-pyrrolidinone, 40 mg of ethylene glycol dimethacrylate (EGDMA) and 120 mg of Irgacure 2959 are mixed. The solution is degassed by bubbling nitrogen. The mixture is poured into a small glass petri dish to form a 1 mm thick solution layer. The solution is exposed to long UV light for 30 minutes under an inert atmosphere. The polymerized gel is incubated in water for 24 hours and washed with water 3 times. The hydrogel is incubated in PBS pH (7.4) for 24 h and sterilized.

In another modification of this example, TRIS is replaced with polydimethylsiloxane terminated with methacrylate end groups (methacryloxypropyl Terminated polydimethylsiloxane (molecular weight 380-550 g/mole, Gelest Inc. catalog number DMS-R05) with or without silica as a filler.

### Example 16C

Preparation of two or more layered unibody hydrogel wound dressing.

Preparation of two or more layered unibody hydrogel comprising polydimethylsiloxane.

500.0 mg of gelatin methacrylate macromonomer is dissolved in 5 ml PBS to obtain macromonomer solution. A 15 microliters initiator solution (ISS) is added to the solution and is mixed thoroughly (GM-H) and degassed to remove dissolved oxygen. The mixture is poured into a glass petri dish to form a 1 mm thick solution layer liquid. The solution is frozen to solid using liquid nitrogen or using a freezer. 2 g of 3-(methacryloyloxy)propyltris(trimethylsiloxy) silane (TRIS), 2 g of N,N-dimethylacrylamide and 2 g of 1-vinyl-2-pyrrolidinone, 40 mg of ethylene glycol dimethacrylate (EGDMA) and 120 mg of Irgacure 2959 are mixed. The solution is degassed using nitrogen bubbling and cooled to 0-10 degree C. The solution is added on top of the frozen solid gelatin methacrylate solution to produce 250 micron to 1 mm thick layer. The mixture is then exposed to UV light for 30 minutes under a nitrogen atmosphere to effectively polymerize and crosslink both layers. The polymerized unibody hydrogel with gelatin methacrylate at the bottom layer and top layer comprising silicone hydrogel is incubated in water to wash/remove uncrosslinked monomer and initiator fraction and to equilibrate the hydrogel with water. The presence of dimethylsiloxane incorporated via the TRIS monomer provides oxygen permeability to the gelatin hydrogel layer. Either silicone or gelatin layer can contact the wound surface, however gelatin hydrogel surface is preferred as a wound contacting surface.

### EXAMPLE 17

Preparation of two or more layered unibody material using frozen precursor microparticles/microspheres.

Preparation of two layered unibody hydrogel composite made using microparticles.

Frozen or dry PEG35KUA macromonomer comprising PLGA as NBP and lodixanol visualization agent (20 percent lodixanol relative to PLGA weight) and photoinitiator are prepared using the method as described in EXAMPLE 5 or 10 (PEG35KUA-R, size 100-500 microns). Using a similar procedure, frozen or dry PEG35KUA aqueous solution comprising PLGA and 10 percent bupivacaine base (relative to PLGA weight, PEG35KUA-D, size 100-500 microns) are made. PEG35KUA-R and PEG35KUA-D are uncrosslinked microspheres in frozen form. A 50 ml glass syringe with a ground glass plunger is modified. The syringe plunger is removed from the syringe barrel completely and about 1 cm portion of the syringe barrel is cut, removing the needle connector portion of the barrel. The plunger is pushed into a modified barrel portion until the end and is then kept in the freezer and cooled to −20 degree C. The cooled syringe is removed from the freezer. The syringe plunger is pulled down about 1 mm to create an empty space in the syringe barrel (about 1 mm height) and frozen PEG35KUA-R microspheres are added to fill the newly created space completely. The syringe plunger is pulled again to create additional space on top of PEG35KUA-R microspheres in the syringe barrel. Frozen PEG35KUA-D microspheres are added in the newly created space until all the volume is occupied. Both the precursor microspheres are then exposed to long UV light until effective polymerization and crosslinking of both the microspheres to form a unibody of crosslinked material wherein polymerization and crosslinking occurs between the microspheres and within the microspheres. The unibody material is pushed out of the barrel and stored until use. The crosslinked unibody material thus formed has one layer comprising a drug (PEG35KUA-D) and other layer comprising a visualization agent (PEG35KUA-R).

A modification of the above experiment is done in which 5 grams of PEG35KUA-D and PEG35KUA-R frozen microspheres are mixed in equal proportion and then the mixture is loaded in syringe barrel space as above and then irradiated to UV light to effectively crosslink and form a unibody object. The object is pushed out of the syringe barrel using the plunger. The crosslinked unibody object thus formed has one layer wherein PEG35KUA-D and PEG35KUA-R are randomly distributed in the layer.

### EXAMPLE 18

Elastomeric organogels.

200 mg PEG35KUA macromonomer, 100 mg PLGA 50:50 (45000 to 50000) are dissolved in 1.0 ml DMSO. 20 microliter of initiator stock solution in vinyl pyrrolidinone (100 mg Irgacure 2959 dissolved in 900 mg vinyl pyrrolidinone) is added to macromonomer solution. The solution is filled in a glass tube and exposed to long UV light for 5 minutes. The polymerized tubular rod like gel implant is removed from the glass tube. The implant can be stretched up to 100 percent of its original length. The solvent is removed in the stretched condition to precipitate the PLGA present in the implant. Thus, by manipulating the OSG implant shape, PLGA implant shape can be manipulated. The same implant can be loaded with drug/visualization agent/s during polymerization or after polymerization via solvent diffusion as discussed in this invention.

In another experiment as above, biodegradable macromonomer 35KL5A is used in place of PEG35KUA to prepare a biodegradable crosslinked network

### EXAMPLE 19

Superabsorbent porous hydrogels for personal hygiene products

Use of frozen solutions to obtain porous hydrogels comprising polyacrylic acid salt (sodium salt)

1 gram of acrylic acid, 5.2 gram of sodium acrylate, 2.5 g gelatin methacrylate 0.2 g methylene bisacrylamide, 300 mg Irgacure 2959 and 30 g distilled water are mixed. The mixture is filled into a 10 ml glass syringe with a 21 g needle. The solution is pushed out the needle and collected in liquid nitrogen. The collected frozen droplets are collected. The droplets are brought to −20 degree C. temperature in the refrigerator. A small Petri dish is frozen at −20 degree C. and frozen droplets are added into the dish until about 1-3 mm height is obtained while maintaining the frozen condition. The frozen particles are then exposed to long UV light for 30 minutes while maintaining the temperature between −20 degree to 0 degree C. The polymerized unibody particles are removed and extracted with water to remove unreacted monomers and water soluble components. The frozen monomer solution has water crystallized as ice crystals. Polymerization does not takes place inside ice crystals due to absence of monomer within crystal structure. Ice crystals act as a porogen which provides porosity to the crosslinked material, which also helps in improving water absorption capacity of the crosslinked material. The space between frozen particles (similar to FIG. 13F3) also provides another type of porosity that is useful for the distribution of biological fluids during usage as a personal hygiene product.

In another variation of the same embodiment, the frozen monomer solution as above is prepared in dimethyl sulfoxide or 1,4-dioxane without gelatin methacrylate and frozen to make frozen monomer solution droplets. Frozen droplets are added to the petri dish to make a monolayer of frozen microspheres in the petri dish. Cold PEG100UMA solution (1 to 10 degree C.) in distilled water (20 percent concentration with one percent Irgacure 2959 photoinitiator) is added on top of frozen microspheres until all the frozen microspheres are completely submerged. The solution is then irradiated with UV light to crosslink

PEG100UMA and frozen monomer composition in DMSO. The crosslinked unibody produced has elastomeric crosslinked hydrogel PEG100UMA embedded with highly water absorbent sodium acrylate/acrylic acid hydrogel microspheres. The product is dried or lyophilized and cut to a desired size and used to make personal hygiene products. Alternatively, photopolymerizable compositions comprising acrylic acid salt preferably sodium salt are polymerized using 3D polymerization printing process in a desirable shape and size. Preferably each part can be incorporated in dry form in personal hygiene products such as diaper is printed. Two, 3, 4, 5, 10, 50, 100 or more such parts can be made in a commercial 3D printer using projection based 3D printing machines available commercially as described before.

### EXAMPLE 20

Compositions for oral drug delivery

### Example 20A

Multilayered tablets for oral drug delivery

Preparation of 4 layered tablets oral drug delivery

Following solutions are prepared fresh and used immediately. Solution A: In a 15 ml glass vial, 1 g of PEG35KUA macromonomer is dissolved in 9 ml of PBS (pH 7.4) and 20 μl initiator stock solution (ISS) is added. Solution B: 2 ml of solution A and 200 mg of magnetic ferrite powder are mixed. Solution C: 2 ml of solution A and 20 mg of diclofenac sodium drug. Solution D: 2 ml of solution A, 20 mg of diclofenac sodium and 20 mg of hydroxypropylmethyl cellulose (Hypromellose, 220890SH-100SR) as a non-crosslinked biodegradable polymer (NBP for controlling the release of diclofenac sodium). Solution E: 2 ml of solution A and 20 mg of furosemide as a second drug. A polypropylene mold with 5 mm diameter cavity with 2 mm depth is used. A removable polypropylene or polylacticacid (PLA) spacer (0.5 mm thick) that divides the cavity in 4 equal chambers (FIG. 18C) is printed from a 3D printer. Using a pipette, solution B is then used to fill one of the cavities. Solutions C, D and E are then filled in the remaining cavities respectively. The spacer is removed from the cavity and the solutions are immediately exposed to 360 nm light to initiate crosslinking and polymerization for 5 minutes without substantial mixing. The polymerized gel with 5 mm diameter and 2 mm depth with 4 distinct layers (FIG. 18F) is removed from the mold and lyophilized/dried and stored in the refrigerator for further processing. The unibody object formed has one layer with magnetic particles to provide magnetic property to the object. Two layers have diclofenac sodium drug as an illustrative drug. One of the layer diclofenac sodium layers has no NBP and is used for quick/fast release of drug from the tablet upon oral ingestion. The other layer comprising diclofenac sodium has hydroxy propylmethyl cellulose as an illustrative NBP polymer to slow the release of diclofenac sodium upon ingestion.

In a similar experiment as above, the solutions are frozen at −20 degree C. before removing the spacer. The spacer is removed from the frozen solution, the frozen solids are pushed/compacted manually, the gap left by the removal of the spacer is bridged and surfaces of all the solutions are in contact with each other. The solution is warmed to room temperature until the surface edges just start to melt and then exposed to UV light for 5 minutes to complete polymerization and crosslinking in liquid/solid state. The polymerized unibody object with 4 distinct layers is removed from the mold. The object is dried/lyophilized and then coated with a sugar solution and dried for taste masking and then used for monitoring the release of drugs from the object.

### Example 20B

Preparation of oral capsules comprising crosslinked precursor multilayered gels. Following solutions are prepared fresh and used immediately. Solution F: In a 15 ml glass vial, 1 g of PEG1OKUA macromonomer is dissolved in 10 ml of polyethylene glycol dimethyl ether (molecular weight 550 Daltons, PEGDME) and 20 μl initiator stock solution (ISS) is added. To this solution 200 mg of furosemide drug is added. Solution G: In a 15 ml glass vial, 1 g of PEG1OKUA macromonomer is dissolved in 10 ml of polyethylene glycol dimethyl ether (molecular weight 550 Daltons, PEGDME) and 20 μl initiator stock solution (ISS) is added. To this solution 200 mg of diclofenac sodium is added. A 00 size commercial gelatin capsule is procured. Half of the capsule cavity is filled with solution F and then exposed to long UV light for 5 minutes to effectively crosslink the PEG1OKUA macromonomer in the capsule cavity. The other half of the capsule is filled with Solution G and exposed to UV light effectively crosslink the solution. The two halves with polymerized gels were joined together and heat sealed to form a sealed capsule. In another modification, polyethylene glycol molecular weight 1000 to 2000 or glycerol or their mixture is used as a solvent.

In another modification of the above examples, solutions C and D are used to fill the two halves. Briefly, the 0.3 ml solution C is added to one part of the empty capsule cavity and quickly frozen in liquid nitrogen before the water in the solution causes dissolution of the capsule wall. Similarly, the other half is filled with 0.3 ml solution D and quickly frozen. The frozen solutions are exposed to UV light around zero degree C. in frozen conditions. The polymerized halves are then dried/lyophilized and then joined together to make a complete sealed capsule for oral ingestion.

### Example 20C

Preparation of encapsulated hydrogel microparticles for oral drug delivery.

Preparation of hydrogel microspheres comprising Psyllium Husk.

Solution A: In a 15 ml glass vial, 1 g of gelatin methacrylate is dissolved in 9 ml of PBS (Ph 7.4) and 20 μl initiator stock solution (ISS) is added. One ml of the above solution is mixed with one gram of Psyllium Husk fine powder and the suspension is added to cold mineral oil or peanut oil and mixed vigorously. The suspension is added to liquid nitrogen and frozen. The frozen macromonomer droplets are exposed to UV light to effectively polymerize and crosslink in the frozen state. The crosslinked microspheres comprising Psyllium Husk are separated from oil, washed with hexane to remove traces of oil and lyophilized/dried. The dried powder with magnesium stearate as a binder (final concentration one percent relative to total mass) are added to a 10 mm tablet making mold and pressed using a hydraulic press at a pressure of 10 to 500 kg depending on the powder weight. The pressed tablets are taken out and used for further studies. In an alternative embodiment, in a 15 ml glass vial, 1 g of PEG35KUA macromonomer is dissolved in 9 ml tetrahydrofuran and 20 μl initiator stock solution (ISS) is added. One ml of the above solution is with 1 g of Psyllium Husk fine powder (size less than 200 mesh) and the suspension filled in the syringe. The suspension is added dropwise in liquid nitrogen and polymerized in frozen condition as described above. The crosslinked particles are dried to remove the THF and then compressed to fill into 00 size gelatin or vegetable capsules and sealed.

### Example 20D

Coating of Psyllium particles with crosslinked synthetic polymer hydrogels.

100 psyllium husk powder (average particle size less than 80 microns) is incubated for 2-5 minutes in 10 ml of 0.1 percent ethyl eosin (dissolved in ethanol) or eosin solution (prepared in distilled water). The eosin-stained psyllium husk powder is then separated from the solution and washed twice with 20 ml of PBS to remove loosely attached eosin. The particles are separated by filtration or centrifugation and resuspended in 10 ml of 10 percent macromonomer PEG1OKUA solution in PBS pH 7.4 comprising catalyst and cocatalyst for photopolymerization (triethanolamine 0.1M, vinyl pyrrolidone 0.1 percent). The suspended PLGA particles are then exposed to 514 nm laser light (100 mW per cm²) while continuously stirring. The macromonomer undergoes interfacial photopolymerization on the surface of husk particles. Depending on the exposure time, the thickness of the crosslinked polymer layer can be controlled. The coating thickness ranges from 1 micron to 2000 microns. The coated particles are then removed from the solution, washed with PBS and dried in a vacuum or lyophilized. In a 5 ml glass vial, 100 mg of dried coated particles are suspended in 1 ml distilled water. For comparison, 100 mg of uncoated husk particles in a 5 ml separate glass vial are also suspended in 1 ml distilled water. The uncoated particles formed a lump of hydrogel and coated/encapsulated particles were hydrated but did not form a lump after 30 minutes to 1 hour of incubation at room temperature. The coating or encapsulation matrix on the particle surface (microencapsulation) prevents the formation of lumps upon exposure to water or aqueous solution.

All references, patent applications and patents recited herein are incorporated herein by specific reference in their entirety.

### REFERENCES

1. M. Helminger et. al., Adv. Funct. Mater., volume 24, 3187 (2014).

2. Saha et. al., J. Mater. Chem. B, volume 3, page 3406, (2015).

3. A. Vintiloiu et. al., Journal of Controlled Release, Volume 125, Page 179 (2008).

4. C. Wischke et. al., International Journal of Pharmaceutics, Volume 364, Page 298, (2008).

5. B. D. Fairbanks et. al., Biomaterials, volume 30(35), Page 6702 (2009).

6. L. Klouda et. al., Eur J Pharm Biopharm., Volume 68(1), page 34 (2008).

7. A. Yadav et al. (Materials Today Chemistry 15 (2020).

8. Micromanipulator, https://en.wikipedia.org/wiki/Micromanipulator, Accessed on Oct. 5, 2021.

### Cited US Patents and Patent Applications

U.S. Pat. No. 5,410,016

U.S. Pat. No. 5,529,914

U.S. Pat. No. 5,874,500

U.S. Pat. No. 6,004,573

U.S. Pat. No. 6,534,591

U.S. Pat. No. 6,566,406

U.S. Pat. No. 6,887,974

U.S. Pat. No. 7,009,034

U.S. Pat. No. 7,740,877

U.S. Pat. No. 7,790,141

U.S. Pat. No. 8,067,031

U.S. Pat. No. 6,201,065

U.S. Pat. No. 9,023,379

U.S. Pat. No. 6,387,977

U.S. Pat. No. 9,789,073

U.S. Pat. No. 9,498,557

U.S. Pat. No. 6,201,065

U.S. Pat. No. 8,067,031

U.S. Pat. No. 8,506,856

U.S. Pat. No. 6,004,573

U.S. Pat. No. 7,740,877

U.S. Pat. No. 5,573934

U.S. Pat. No. 8,409,606

US Patent Application 20140256617

US Patent Application 20190046479

US Patent Application 20050069572

US Patent Application 20160166504

US Patent Application 20190046479

1. A method of making a microparticle, comprising:
providing a homogenous solution having at least one precursor configured for polymerization and crosslinking in a frozen state;
forming at least one particle having a geometric shape with the homogenous solution;
freezing the geometric shape of each particle to form frozen solid geometric shapes; and
crosslinking the at least one precursor in the frozen solid geometric shape to form the microparticle having the solid geometric shape.

2. The method of claim 1, wherein the freezing is at a temperature of less than or about 15° C.

3. The method of claim 1, wherein the solution includes a homogenous distribution of an active entity selected from a drug, nucleic acid, polypeptide, antibody, macromolecule, or cell, and the active entity is encapsulated in the microparticle.

4. The method of claim 3, wherein the drug is selected from the group consisting of: antiinfectives; antibiotics; antiviral agents; antifungal agents; antibacterial agents, antipruritics; anticancer agents; antipsychotics; cholesterol- or lipid-reducing agents; cell cycle inhibitors; antiparkinsonism drugs; HMG-CoA inhibitors; antirestenosis agents; antiinflammatory agents; antiasthmatic agents; anthelmintic; immunosuppressives; muscle relaxants; antidiuretic agents; vasodilators; nitric oxide; nitric oxide-releasing compounds; beta-blockers; hormones; antidepressants; decongestants; calcium channel blockers; growth factors; bone growth factors; bone morphogenic proteins; wound healing agents; analgesics; analgesic combinations; local anesthetic agents; antihistamines; sedatives; angiogenesis-promoting agents; angiogenesis-inhibiting agents; tranquilizers; and combinations thereof.

5. The method of claim 1, wherein the crosslinking is by free radical polymerization initiated by an electromagnetic radiation.

6. The method of claim 5, wherein the electromagnetic radiation is ultraviolet or visible light.

7. The method of claim 5, further comprising exposing the at least one precursor to the electromagnetic radiation for a time less than 5 minutes or from about 1 second to about 60 seconds.

8. The method of claim 1, further comprising:
placing the solution into a mold; and
freezing the solution in the mold to form the frozen solid geometric shape.

9. The method of claim 1, wherein the geometric shape is selected from the group consisting of cylinders, cubes, cuboids, cones, spheres, rectangular prisms, triangular prisms, hexagonal prisms, square pyramids, rectangular pyramid, triangular pyramid, hexagonal pyramid, torus, portions thereof, or the combinations thereof in any proportion.

10. The method of claim 1, comprising removing a solvent of the solution from the microparticle by lyophilization, evaporation, air drying, vacuum drying, sublimation or solvent exchange.

11. The method of claim 1, wherein the solution includes an aqueous solution buffered at a pH range of about 6 to about 9.

12. The method of claim 1, wherein the solution includes a biocompatible organic solvent.

13. The method of claim 12, wherein the organic solvent is selected from the group consisting of dimethyl carbonate, methyl ethyl ketone (MEK), tert-butyl acetate, acetone, acetonitrile, cyclohexanone, dimethyl sulfoxide (DMSO), n-methyl pyrrolidinone (NMP), dimethylacetamide (DMAC), dimethyl formamide (DMF), methanol, ethanol, isopropanol, PEG molecular weight 400-1000 g/mol, PEG endcapped with methyl ether, dichloromethane, trichloromethane, chloroform, dioxane, ethyl acetate, dimethyl ether (DME), tetraglycol, ethyl lactate, triethylene glycol dimethyl ether (triglyme), glycerol formal, ethylene glycol monoethyl ether acetate, benzyl alcohol, tributyrin, benzyl benzoate, acetic acid, diethylene glycol dimethyl ether (diglyme), ethyl benzoate, dimethyl isosorbide (DMI), polyethylene glycol dimethyl ether, glycofurol, glycerol, ethyl acetate, 1,3 propanediol, 1,4 butanediol, 1-6-hexanediol, tetrahydrofuran, and combinations thereof.

14. The method of claim 1, wherein the at least one precursor includes at least two polymerizable groups.

15. The method of claim 1, wherein the microparticle is biodegradable by the precursor being biodegradable.

16. The method of claim 1, wherein the microparticle is biostable.

17. The method of claim 1, wherein the homogenous solution includes a homogenous distribution of a visualization agent, and the visualization agent is encapsulated in the microparticle.

18. The method of claim 1, wherein the solution includes a homogenous distribution of a biodegradable polymer, and the biodegradable polymer is encapsulated in the microparticle.

19. The method of claim 18, wherein the biodegradable polymer is selected from the group consisting of polymers, dendrimers, copolymers or oligomers of: glycolide, dI-lactide, d-lactide, I-lactide, caprolactone, dioxanone and trimethylene carbonate, degradable polyurethanes, degradable polyurethanes made by block copolymers of degradable polylactone, polycaprolactone and polycarbonate, poly(hexamethylene carbonate), tyrosine-derived polycarbonates, tyrosine-derived polyacrylates, polyamides, polyesters, polypeptides, polyhydroxyacids, polylactic acid, polyglycolic acid, polyanhydrides, polylactones, PEG-polylactone copolymers, polyvinyl alcohol-co-polylactone copolymers, celluloses, modified celluloses, collagens, modified collagens, gelatins, albumin, fibrinogen, keratin, starches, hyaluronic acid, dextran, and combinations thereof.

20. The method of claim 1, further comprising freezing the solution having the geometric shape in a liquid medium.

21. The method of claim 20, wherein the liquid medium is a liquid at a freezing temperature and a gas at room temperature, further comprising removing the liquid medium from the microparticle by transition to the gas.

22. The method of claim 21, wherein the liquid medium is selected from nitrogen, argon or helium or an aqueous solution mixed with organic or inorganic salts.

23. The method of claim 20, further comprising spraying droplets of the solution into the liquid medium to freeze the droplets into the froze solid geometric shape.

24. The microparticle formed by the method of claim 1.

25. The method of claim 4, wherein the drug is present at a weight relative to the microparticle weight from about 0.1% to about 60%.

26. The microparticle of claim 1, wherein the volume of the frozen solid geometric shape is from about 1.5 ml to about 0.5 picolitres.

27. A method of making a drug delivery composition, comprising:
mixing a solution having a drug in a solvent with a macromonomer and a photo-initiator to form a mixture, wherein the macromonomer has at least two polymerizable groups;
forming at least one geometric shape of the mixture;
freezing the at least one geometric shape to form at least one frozen solid geometric shape; and
polymerizing the macromonomer in the frozen solid geometric shape with electromagnetic radiation.

28. The method of claim 27, wherein:
the freezing is at a temperature of less than or about 15° C.;
the electromagnetic radiation is ultraviolet or visible light; or
the polymerizing is by exposing the macromonomer to the electromagnetic radiation for a time less than 5 minutes or from about 1 second to about 60 seconds.

29. The method of claim 27, further comprising:
placing the solution into a mold; and
freezing the solution in the mold to form the frozen solid geometric shape.

30. The method of claim 27, further comprising freezing the solution having the geometric shape in a liquid medium, wherein the liquid medium is a liquid at a freezing temperature and a gas at room temperature, further comprising removing the liquid medium from the solid geometric shape by transition to the gas.

31. The method of claim 27, wherein the drug is selected from the group consisting of: antiinfectives; antibiotics; antiviral agents; antifungal agents; antibacterial agents, antipruritics; anticancer agents; antipsychotics; cholesterol- or lipid-reducing agents; cell cycle inhibitors; antiparkinsonism drugs; HMG-CoA inhibitors; antirestenosis agents; antiinflammatory agents; antiasthmatic agents; anthelmintic; immunosuppressives; muscle relaxants; antidiuretic agents; vasodilators; nitric oxide; nitric oxide-releasing compounds; beta-blockers; hormones; antidepressants; decongestants; calcium channel blockers; growth factors; bone growth factors; bone morphogenic proteins; wound healing agents; analgesics; analgesic combinations; local anesthetic agents; antihistamines; sedatives; angiogenesis-promoting agents; angiogenesis-inhibiting agents; tranquilizers; and combinations thereof.

32. The method of claim 27, wherein the solvent is an organic solvent selected from the group consisting of dimethyl carbonate, methyl ethyl ketone (MEK), tert-butyl acetate, acetone, acetonitrile, cyclohexanone, dimethyl sulfoxide (DMSO), n-methyl pyrrolidinone (NMP), dimethylacetamide (DMAC), dimethyl formamide (DMF), methanol, ethanol, isopropanol, PEG molecular weight 400-1000 g/mol, PEG endcapped with methyl ether, dichloromethane, trichloromethane, chloroform, dioxane, ethyl acetate, dimethyl ether (DME), tetraglycol, ethyl lactate, triethylene glycol dimethyl ether (triglyme), glycerol formal, ethylene glycol monoethyl ether acetate, benzyl alcohol, tributyrin, benzyl benzoate, acetic acid, diethylene glycol dimethyl ether (diglyme), ethyl benzoate, dimethyl isosorbide (DMI), polyethylene glycol dimethyl ether, glycofurol, glycerol, ethyl acetate, 1,3 propanediol, 1,4 butanediol, 1-6-hexanediol, tetrahydrofuran, and combinations thereof.

33. The method of claim 27, wherein is macromonomer is a derivative of polyethylene glycol, polyethylene oxide, polyethylene oxide-polypropylene block copolymers, polyvinyl alcohol, albumin, fibrinogen, collagen, alginate, gelatin, keratin, cellulose, hyaluronic acid, and combinations thereof.

34. The method of claim 27, wherein the geometric shape is selected from the group consisting of cylinders, cubes, cuboids, cones, spheres, rectangular prisms, triangular prisms, hexagonal prisms, square pyramids, rectangular pyramid, triangular pyramid, hexagonal pyramid, torus, portions thereof, or the combinations thereof in any proportion.

35. The method of claim 27, wherein the solution includes a homogenous distribution of a biodegradable polymer, and the biodegradable polymer is encapsulated in the microparticle, wherein the biodegradable polymer is selected from the group consisting of polymers, dendrimers, copolymers or oligomers of: glycolide, dI-lactide, d-lactide, I-lactide, caprolactone, dioxanone and trimethylene carbonate, degradable polyurethanes, degradable polyurethanes made by block copolymers of degradable polylactone, polycaprolactone and polycarbonate, poly(hexamethylene carbonate), tyrosine-derived polycarbonates, tyrosine-derived polyacrylates, polyamides, polyesters, polypeptides, polyhydroxyacids, polylactic acid, polyglycolic acid, polyanhydrides, polylactones, PEG-polylactone copolymers, polyvinyl alcohol-co-polylactone copolymers, celluloses, modified celluloses, collagens, modified collagens, gelatins, albumin, fibrinogen, keratin, starches, hyaluronic acid, dextran, and combinations thereof.
